SEQUENCE LISTING (112 sequences)

SEQ ID NO: 1 - protein, 436 aa, Artificial Sequence
  synthetic
MAKYLELEEGGVIMQAFYWDVPSGGIWWDTIRQKIPEWYDAGISAIWIPPASKGMSGGYSMGYDPYDYFDLGEYYQKGTVETRFGSKQELINMINTAHAYGIKVIADIVINHRAGGDLEWNPFVGDYTWTDFSKVASGKYTANYLDFHPNELHAGDSGTFGGYPDICHDKSWDQYWLWASQESYAAYLRSIGIDAWRFDYVKGYGAWVVKDWLNWWGGWAVGEYWDTNVDALLNWAYSSGAKVFDFPLYYKMDAAFDNKNIPALVEALKNGGTVVSRDPFKAVTFVANHDTDIIWNKYPAYAFILTYEGQPTIFYRDYEEWLNKDKLKNLIWIHDNLAGGSTSIVYYDSDEMIFVRNGYGSKPGLITYINLGSSKVGRWVYVPKFAGACIHEYTGNLGGWVDKYVYSSGWVYLEAPAYDPANGQYGYSVWSYCGVG

SEQ ID NO: 2 - DNA, 1308 bp, Artificial Sequence
  synthetic
atggccaagtacctggagctggaggagggcggcgtgatcatgcaggcgttctactgggacgtcccgagcggaggcatctggtgggacaccatccgccagaagatccccgagtggtacgacgccggcatctccgcgatctggataccgccagcttccaagggcatgtccgggggctactcgatgggctacgacccgtacgactacttcgacctcggcgagtactaccagaagggcacggtggagacgcgcttcgggtccaagcaggagctcatcaacatgatcaacacggcgcacgcctacggcatcaaggtcatcgcggacatcgtgatcaaccacagggccggcggcgacctggagtggaacccgttcgtcggcgactacacctggacggacttctccaaggtcgcctccggcaagtacaccgccaactacctcgacttccaccccaacgagctgcacgcgggcgactccggcacgttcggcggctacccggacatctgccacgacaagtcctgggaccagtactggctctgggcctcgcaggagtcctacgcggcctacctgcgctccatcggcatcgacgcgtggcgcttcgactacgtcaagggctacggggcctgggtggtcaaggactggctcaactggtggggcggctgggcggtgggcgagtactgggacaccaacgtcgacgcgctgctcaactgggcctactcctccggcgccaaggtgttcgacttccccctgtactacaagatggacgcggccttcgacaacaagaacatcccggcgctcgtcgaggccctgaagaacggcggcacggtggtctcccgcgacccgttcaaggccgtgaccttcgtcgccaaccacgacacggacatcatctggaacaagtacccggcgtacgccttcatcctcacctacgagggccagcccacgatcttctaccgcgactacgaggagtggctgaacaaggacaagctcaagaacctgatctggattcacgacaacctcgcgggcggctccactagtatcgtgtactacgactccgacgagatgatcttcgtccgcaacggctacggctccaagcccggcctgatcacgtacatcaacctgggctcctccaaggtgggccgctgggtgtacgtcccgaagttcgccggcgcgtgcatccacgagtacaccggcaacctcggcggctgggtggacaagtacgtgtactcctccggctgggtctacctggaggccccggcctacgaccccgccaacggccagtacggctactccgtgtggtcctactgcggcgtcggc

SEQ ID NO: 3 - protein, 800 aa, Artificial Sequence
  synthetic
MGHWYKHQRAYQFTGEDDFGKVAVVKLPMDLTKVGIIVRLNEWQAKDVAKDRFIEIKDGKAEVWILQGVEEIFYEKPDTSPRIFFAQARSNKVIEAFLTNPVDTKKKELFKVTVDGKEIPVSRVEKADPTDIDVTNYVRIVLSESLKEEDLRKDVELIIEGYKPARVIMMEILDDYYYDGELGAVYSPEKTIFRVWSPVSKWVKVLLFKNGEDTEPYQVVNMEYKGNGVWEAVVEGDLDGVFYLYQLENYGKIRTTVDPYSKAVYANNQESAVVNLARTNPEGWENDRGPKIEGYEDAIIYEIHIADITGLENSGVKNKGLYLGLTEENTKGPGGVTTGLSHLVELGVTHVHILPFFDFYTGDELDKDFEKYYNWGYDPYLFMVPEGRYSTDPKNPHTRIREVKEMVKALHKHGIGVIMDMVFPHTYGIGELSAFDQTVPYYFYRIDKTGAYLNESGCGNVIASERPMMRKFIVDTVTYWVKEYHIDGFRFDQMGLIDKKTMLEVERALHKIDPTIILYGEPWGGWGAPIRFGKSDVAGTHVAAFNDEFRDAIRGSVFNPSVKGFVMGGYGKETKIKRGVVGSINYDGKLIKSFALDPEETINYAACHDNHTLWDKNYLAAKADKKKEWTEEELKNAQKLAGAILLTSQGVPFLHGGQDFCRTTNFNDNSYNAPISINGFDYERKLQFIDVFNYHKGLIKLRKEHPAFRLKNAEEIKKHLEFLPGGRRIVAFMLKDHAGGDPWKDIVVIYNGNLEKTTYKLPEGKWNVVVNSQKAGTEVIETVEGTIELDPLSAYVLYRE

SEQ ID NO: 4 - DNA, 2400 bp, Artificial Sequence
  synthetic
atgggccactggtacaagcaccagcgcgcctaccagttcaccggcgaggacgacttcgggaaggtggccgtggtgaagctcccgatggacctcaccaaggtgggcatcatcgtgcgcctcaacgagtggcaggcgaaggacgtggccaaggaccgcttcatcgagatcaaggacggcaaggccgaggtgtggatactccagggcgtggaggagatcttctacgagaagccggacacctccccgcgcatcttcttcgcccaggcccgctccaacaaggtgatcgaggccttcctcaccaacccggtggacaccaagaagaaggagctgttcaaggtgaccgtcgacggcaaggagatcccggtgtcccgcgtggagaaggccgacccgaccgacatcgacgtgaccaactacgtgcgcatcgtgctctccgagtccctcaaggaggaggacctccgcaaggacgtggagctgatcatcgagggctacaagccggcccgcgtgatcatgatggagatcctcgacgactactactacgacggcgagctgggggcggtgtactccccggagaagaccatcttccgcgtgtggtccccggtgtccaagtgggtgaaggtgctcctcttcaagaacggcgaggacaccgagccgtaccaggtggtgaacatggagtacaagggcaacggcgtgtgggaggccgtggtggagggcgacctcgacggcgtgttctacctctaccagctggagaactacggcaagatccgcaccaccgtggacccgtactccaaggccgtgtacgccaacaaccaggagtctgcagtggtgaacctcgcccgcaccaacccggagggctgggagaacgaccgcggcccgaagatcgagggctacgaggacgccatcatctacgagatccacatcgccgacatcaccggcctggagaactccggcgtgaagaacaagggcctctacctcggcctcaccgaggagaacaccaaggccccgggcggcgtgaccaccggcctctcccacctcgtggagctgggcgtgacccacgtgcacatcctcccgttcttcgacttctacaccggcgacgagctggacaaggacttcgagaagtactacaactggggctacgacccgtacctcttcatggtgccggagggccgctactccaccgacccgaagaacccgcacacccgaattcgcgaggtgaaggagatggtgaaggccctccacaagcacggcatcggcgtgatcatggacatggtgttcccgcacacctacggcatcggcgagctgtccgccttcgaccagaccgtgccgtactacttctaccgcatcgacaagaccggcgcctacctcaacgagtccggctgcggcaacgtgatcgcctccgagcgcccgatgatgcgcaagttcatcgtggacaccgtgacctactgggtgaaggagtaccacatcgacggcttccgcttcgaccagatgggcctcatcgacaagaagaccatgctggaggtggagcgcgccctccacaagatcgacccgaccatcatcctctacggcgagccgtggggcggctggggggccccgatccgcttcggcaagtccgacgtggccggcacccacgtggccgccttcaacgacgagttccgcgacgccatccgcggctccgtgttcaacccgtccgtgaagggcttcgtgatgggcggctacggcaaggagaccaagatcaagcgcggcgtggtgggctccatcaactacgacggcaagctcatcaagtccttcgccctcgacccggaggagaccatcaactacgccgcctgccacgacaaccacaccctctgggacaagaactacctcgccgccaaggccgacaagaagaaggagtggaccgaggaggagctgaagaacgcccagaagctcgccggcgccatcctcctcactagtcagggcgtgccgttcctccacggcggccaggacttctgccgcaccaccaacttcaacgacaactcctacaacgccccgatctccatcaacggcttcgactacgagcgcaagctccagttcatcgacgtgttcaactaccacaagggcctcatcaagctccgcaaggagcacccggccttccgcctcaagaacgccgaggagatcaagaagcacctggagttcctcccgggcgggcgccgcatcgtggccttcatgctcaaggaccacgccggcggcgacccgtggaaggacatcgtggtgatctacaacggcaacctggagaagaccacctacaagctcccggagggcaagtggaacgtggtggtgaactcccagaaggccggcaccgaggtgatcgagaccgtggagggcaccatcgagctggacccgctctccgcctacgtgctctaccgcgag

SEQ ID NO: 5 - protein, 693 aa, Sulfolobus solfataricus
METIKIYENKGVYKVVIGEPFPPIEFPLEQKISSNKSLSELGLTIVQQGNKVIVEKSLDLKEHIIGLGEKAFELDRKRKRYVMYNVDAGAYKKYQDPLYVSIPLFISVKDGVATGYFFNSASKVIFDVGLEEYDKVIVTIPEDSVEFYVIEGPRIEDVLEKYTELTGKPFLPPMWAFGYMISRYSYYPQDKVVELVDIMQKEGFRVAGVFLDIHYMDSYKLFTWHPYRFPEPKKLIDELHKRNVKLITIVDHGIRVDQNYSPFLSGMGKFCEIESGELFVGKMWPGTTVYPDFFREDTREWWAGLISEWLSQGVDGIWLDMNEPTDFSRAIEIRDVLSSLPVQFRDDRLVTTFPDNVVHYLRGKRVKHEKVRNAYPLYEAMATFKGFRTSHRNEIFILSRAGYAGIQRYAFIWTGDNTPSWDDLKLQLQLVLGLSISGVPFVGCDIGGFQGRNFAEIDNSMDLLVKYYALALFFPFYRSHKATDGIDTEPVFLPDYYKEKVKEIVELRYKFLPYIYSLALEASEKGHPVIRPLFYEFQDDDDMYRIEDEYMVGKYLLYAPIVSKEESRLVTLPRGKWYNYWNGEIINGKSVVKSTHELPIYLREGSIIPLEGDELIVYGETSFKRYDNAEITSSSNEIKFSREIYVSKLTITSEKPVSKIIVDDSKEIQVEKTMQNTYVAKINQKIRGKINLE

SEQ ID NO: 6 - DNA, 2082 bp, Sulfolobus solfataricus
atggagaccatcaagatctacgagaacaagggcgtgtacaaggtggtgatcggcgagccgttcccgccgatcgagttcccgctcgagcagaagatctcctccaacaagtccctctccgagctgggcctcaccatcgtgcagcagggcaacaaggtgatcgtggagaagtccctcgacctcaaggagcacatcatcggcctcggcgagaaggccttcgagctggaccgcaagcgcaagcgctacgtgatgtacaacgtggacgccggcgcctacaagaagtaccaggacccgctctacgtgtccatcccgctcttcatctccgtgaaggacggcgtggccaccggctacttcttcaactccgcctccaaggtgatcttcgacgtgggcctcgaggagtacgacaaggtgatcgtgaccatcccggaggactccgtggagttctacgtgatcgagggcccgcgcatcgaggacgtgctcgagaagtacaccgagctgaccggcaagccgttcctcccgccgatgtgggccttcggctacatgatctcccgctactcctactacccgcaggacaaggtggtggagctggtggacatcatgcagaaggagggcttccgcgtggccggcgtgttcctcgacatccactacatggactcctacaagctcttcacctggcacccgtaccgcttcccggagccgaagaagctcatcgacgagctgcacaagcgcaacgtgaagctcatcaccatcgtggaccacggcatccgcgtggaccagaactactccccgttcctctccggcatgggcaagttctgcgagatcgagtccggcgagctgttcgtgggcaagatgtggccgggcaccaccgtgtacccggacttcttccgcgaggacacccgcgagtggtgggccggcctcatctccgagtggctctcccagggcgtggacggcatctggctcgacatgaacgagccgaccgacttctcccgcgccatcgagatccgcgacgtgctctcctccctcccggtgcagttccgcgacgaccgcctcgtgaccaccttcccggacaacgtggtgcactacctccgcggcaagcgcgtgaagcacgagaaggtgcgcaacgcctacccgctctacgaggcgatggccaccttcaagggcttccgcacctcccaccgcaacgagatcttcatcctctcccgcgccggctacgccggcatccagcgctacgccttcatctggaccggcgacaacaccccgtcctgggacgacctcaagctccagctccagctcgtgctcggcctctccatctccggcgtgccgttcgtgggctgcgacatcggcggcttccagggccgcaacttcgccgagatcgacaactcgatggacctcctcgtgaagtactacgccctcgccctcttcttcccgttctaccgctcccacaaggccaccgacggcatcgacaccgagccggtgttcctcccggactactacaaggagaaggtgaaggagatcgtggagctgcgctacaagttcctcccgtacatctactccctcgccctcgaggcctccgagaagggccacccggtgatccgcccgctcttctacgagttccaggacgacgacgacatgtaccgcatcgaggacgagtacatggtgggcaagtacctcctctacgccccgatcgtgtccaaggaggagtcccgcctcgtgaccctcccgcgcggcaagtggtacaactactggaacggcgagatcatcaacggcaagtccgtggtgaagtccacccacgagctgccgatctacctccgcgagggctccatcatcccgctcgagggcgacgagctgatcgtgtacggcgagacctccttcaagcgctacgacaacgccgagatcacctcctcctccaacgagatcaagttctcccgcgagatctacgtgtccaagctcaccatcacctccgagaagccggtgtccaagatcatcgtggacgactccaaggagatccaggtggagaagaccatgcagaacacctacgtggccaagatcaaccagaagatccgcggcaagatcaacctcgagtga

SEQ ID NO: 7 - DNA, 1818 bp, Artificial Sequence
  synthetic
atggcggctctggccacgtcgcagctcgtcgcaacgcgcgccggcctgggcgtcccggacgcgtccacgttccgccgcggcgccgcgcagggcctgaggggggcccgggcgtcggcggcggcggacacgctcagcatgcggaccagcgcgcgcgcggcgcccaggcaccagcaccagcaggcgcgccgcggggccaggttcccgtcgctcgtcgtgtgcgccagcgccggcatgaacgtcgtcttcgtcggcgccgagatggcgccgtggagcaagaccggaggcctcggcgacgtcctcggcggcctgccgccggccatggccgcgaacgggcaccgtgtcatggtcgtctctccccgctacgaccagtacaaggacgcctgggacaccagcgtcgtgtccgagatcaagatgggagacgggtacgagacggtcaggttcttccactgctacaagcgcggagtggaccgcgtgttcgttgaccacccactgttcctggagagggtttggggaaagaccgaggagaagatctacgggcctgtcgctggaacggactacagggacaaccagctgcggttcagcctgctatgccaggcagcacttgaagctccaaggatcctgagcctcaacaacaacccatacttctccggaccatacggggaggacgtcgtgttcgtctgcaacgactggcacaccggccctctctcgtgctacctcaagagcaactaccagtcccacggcatctacagggacgcaaagaccgctttctgcatccacaacatctcctaccagggccggttcgccttctccgactacccggagctgaacctccccgagagattcaagtcgtccttcgatttcatcgacggctacgagaagcccgtggaaggccggaagatcaactggatgaaggccgggatcctcgaggccgacagggtcctcaccgtcagcccctactacgccgaggagctcatctccggcatcgccaggggctgcgagctcgacaacatcatgcgcctcaccggcatcaccggcatcgtcaacggcatggacgtcagcgagtgggaccccagcagggacaagtacatcgccgtgaagtacgacgtgtcgacggccgtggaggccaaggcgctgaacaaggaggcgctgcaggcggaggtcgggctcccggtggaccggaacatcccgctggtggcgttcatcggcaggctggaagagcagaagggccccgacgtcatggcggccgccatcccgcagctcatggagatggtggaggacgtgcagatcgttctgctgggcacgggcaagaagaagttcgagcgcatgctcatgagcgccgaggagaagttcccaggcaaggtgcgcgccgtggtcaagttcaacgcggcgctggcgcaccacatcatggccggcgccgacgtgctcgccgtcaccagccgcttcgagccctgcggcctcatccagctgcaggggatgcgatacggaacgccctgcgcctgcgcgtccaccggtggactcgtcgacaccatcatcgaaggcaagaccgggttccacatgggccgcctcagcgtcgactgcaacgtcgtggagccggcggacgtcaagaaggtggccaccaccttgcagcgcgccatcaaggtggtcggcacgccggcgtacgaggagatggtgaggaactgcatgatccaggatctctcctggaagggccctgccaagaactgggagaacgtgctgctcagcctcggggtcgccggcggcgagccaggggttgaaggcgaggagatcgcgccgctcgccaaggagaacgtggccgcgccc

SEQ ID NO: 8 - protein, 606 aa, Artificial Sequence
  synthetic
MAALATSQLVATRAGLGVPDASTFRRGAAQGLRGARASAAADTLSMRTSARAAPRHQHQQARRGARFPSLVVCASAGMNVVFVGAEMAPWSKTGGLGDVLGGLPPAMAANGHRVMVVSPRYDQYKDAWDTSVVSEIKMGDGYETVRFFHCYKRGVDRVFVDHPLFLERVWGKTEEKIYGPVAGTDYRDNQLRFSLLCQAALEAPRILSLNNNPYFSGPYGEDVVFVCNDWHTGPLSCYLKSNYQSHGIYRDAKTAFCIHNISYQGRFAFSDYPELNLPERFKSSFDFIDGYEKPVEGRKINWMKAGILEADRVLTVSPYYAEELISGIARGCELDNIMRLTGITGIVNGMDVSEWDPSRDKYIAVKYDVSTAVEAKALNKEALQAEVGLPVDRNIPLVAFIGRLEEQKGPDVMAAAIPQLMEMVEDVQIVLLGTGKKKFERMLMSAEEKFPGKVRAVVKFNAALAHHIMAGADVLAVTSRFEPCGLIQLQGMRYGTPCACASTGGLVDTIIEGKTGFHMGRLSVDCNVVEPADVKKVATTLQRAIKVVGTPAYEEMVRNCMIQDLSWKGPAKNWENVLLSLGVAGGEPGVEGEEIAPLAKENVAAP

SEQ ID NO: 9 - DNA, 2223 bp, Artificial Sequence
  synthetic
atggccaagtacctggagctggaggagggcggcgtgatcatgcaggcgttctactgggacgtcccgagcggaggcatctggtgggacaccatccgccagaagatccccgagtggtacgacgccggcatctccgcgatctggataccgccagcttccaagggcatgtccgggggctactcgatgggctacgacccgtacgactacttcgacctcggcgagtactaccagaagggcacggtggagacgcgcttcgggtccaagcaggagctcatcaacatgatcaacacggcgcacgcctacggcatcaaggtcatcgcggacatcgtgatcaaccacagggccggcggcgacctggagtggaacccgttcgtcggcgactacacctggacggacttctccaaggtcgcctccggcaagtacaccgccaactacctcgacttccaccccaacgagctgcacgcgggcgactccggcacgttcggcggctacccggacatctgccacgacaagtcctgggaccagtactggctctgggcctcgcaggagtcctacgcggcctacctgcgctccatcggcatcgacgcgtggcgcttcgactacgtcaagggctacggggcctgggtggtcaaggactggctcaactggtggggcggctgggcggtgggcgagtactgggacaccaacgtcgacgcgctgctcaactgggcctactcctccggcgccaaggtgttcgacttccccctgtactacaagatggacgcggccttcgacaacaagaacatcccggcgctcgtcgaggccctgaagaacggcggcacggtggtctcccgcgacccgttcaaggccgtgaccttcgtcgccaaccacgacacggacatcatctggaacaagtacccggcgtacgccttcatcctcacctacgagggccagcccacgatcttctaccgcgactacgaggagtggctgaacaaggacaagctcaagaacctgatctggattcacgacaacctcgcgggcggctccactagtatcgtgtactacgactccgacgagatgatcttcgtccgcaacggctacggctccaagcccggcctgatcacgtacatcaacctgggctcctccaaggtgggccgctgggtgtacgtcccgaagttcgccggcgcgtgcatccacgagtacaccggcaacctcggcggctgggtggacaagtacgtgtactcctccggctgggtctacctggaggccccggcctacgaccccgccaacggccagtacggctactccgtgtggtcctactgcggcgtcggcacatcgattgctggcatcctcgaggccgacagggtcctcaccgtcagcccctactacgccgaggagctcatctccggcatcgccaggggctgcgagctcgacaacatcatgcgcctcaccggcatcaccggcatcgtcaacggcatggacgtcagcgagtgggaccccagcagggacaagtacatcgccgtgaagtacgacgtgtcgacggccgtggaggccaaggcgctgaacaaggaggcgctgcaggcggaggtcgggctcccggtggaccggaacatcccgctggtggcgttcatcggcaggctggaagagcagaagggccccgacgtcatggcggccgccatcccgcagctcatggagatggtggaggacgtgcagatcgttctgctgggcacgggcaagaagaagttcgagcgcatgctcatgagcgccgaggagaagttcccaggcaaggtgcgcgccgtggtcaagttcaacgcggcgctggcgcaccacatcatggccggcgccgacgtgctcgccgtcaccagccgcttcgagccctgcggcctcatccagctgcaggggatgcgatacggaacgccctgcgcctgcgcgtccaccggtggactcgtcgacaccatcatcgaaggcaagaccgggttccacatgggccgcctcagcgtcgactgcaacgtcgtggagccggcggacgtcaagaaggtggccaccaccttgcagcgcgccatcaaggtggtcggcacgccggcgtacgaggagatggtgaggaactgcatgatccaggatctctcctggaagggccctgccaagaactgggagaacgtgctgctcagcctcggggtcgccggcggcgagccaggggttgaaggcgaggagatcgcgccgctcgccaaggagaacgtggccgcgccc

SEQ ID NO: 10 - protein, 741 aa, Artificial Sequence
  synthetic
MAKYLELEEGGVIMQAFYWDVPSGGIWWDTIRQKIPEWYDAGISAIWIPPASKGMSGGYSMGYDPYDYFDLGEYYQKGTVETRFGSKQELINMINTAHAYGIKVIADIVINHRAGGDLEWNPFVGDYTWTDFSKVASGKYTANYLDFHPNELHAGDSGTFGGYPDICHDKSWDQYWLWASQESYAAYLRSIGIDAWRFDYVKGYGAWVVKDWLNWWGGWAVGEYWDTNVDALLNWAYSSGAKVFDFPLYYKMDAAFDNKNIPALVEALKNGGTVVSRDPFKAVTFVANHDTDIIWNKYPAYAFILTYEGQPTIFYRDYEEWLNKDKLKNLIWIHDNLAGGSTSIVYYDSDEMIFVRNGYGSKPGLITYINLGSSKVGRWVYVPKFAGACIHEYTGNLGGWVDKYVYSSGWVYLEAPAYDPANGQYGYSVWSYCGVGTSIAGILEADRVLTVSPYYAEELISGIARGCELDNIMRLTGITGIVNGMDVSEWDPSRDKYIAVKYDVSTAVEAKALNKEALQAEVGLPVDRNIPLVAFIGRLEEQKGPDVMAAAIPQLMEMVEDVQIVLLGTGKKKFERMLMSAEEKFPGKVRAVVKFNAALAHHIMAGADVLAVTSRFEPCGLIQLQGMRYGTPCACASTGGLVDTIIEGKTGFHMGRLSVDCNVVEPADVKKVATTLQRAIKVVGTPAYEEMVRNCMIQDLSWKGPAKNWENVLLSLGVAGGEPGVEGEEIAPLAKENVAAP

SEQ ID NO: 11 - DNA, 1515 bp, Zea mays
ggagagctatgagacgtatgtcctcaaagccactttgcattgtgtgaaaccaatatcgatctttgttacttcatcatgcatgaacatttgtggaaactactagcttacaagcattagtgacagctcagaaaaaagttatctatgaaaggtttcatgtgtaccgtgggaaatgagaaatgttgccaactcaaacaccttcaatatgttgtttgcaggcaaactcttctggaagaaaggtgtctaaaactatgaacgggttacagaaaggtataaaccacggctgtgcattttggaagtatcatctatagatgtctgttgaggggaaagccgtacgccaacgttatttactcagaaacagcttcaacacacagttgtctgctttatgatggcatctccacccaggcacccaccatcacctatctctcgtgcctgtttattttcttgccctttctgatcataaaaaaacattaagagtttgcaaacatgcataggcatatcaatatgctcatttattaatttgctagcagatcatcttcctactctttactttatttattgtttgaaaaatatgtcctgcacctagggagctcgtatacagtaccaatgcatcttcattaaatgtgaatttcagaaaggaagtaggaacctatgagagtatttttcaaaattaattagcggcttctattatgtttatagcaaaggccaagggcaaaattggaacactaatgatggttggttgcatgagtctgtcgattacttgcaagaaatgtgaacctttgtttctgtgcgtgggcataaaacaaacagcttctagcctcttttacggtacttgcacttgcaagaaatgtgaactccttttcatttctgtatgtggacataatgccaaagcatccaggctttttcatggttgttgatgtctttacacagttcatctccaccagtatgccctcctcatactctatataaacacatcaacagcatcgcaattagccacaagatcacttcgggaggcaagtgcgatttcgatctcgcagccacctttttttgttctgttgtaagtataccttcccttaccatctttatctgttagtttaatttgtaattgggaagtattagtggaaagaggatgagatgctatcatctatgtactctgcaaatgcatctgacgttatatgggctgcttcatataatttgaattgctccattcttgccgacaatatattgcaaggtatatgcctagttccatcaaaagttctgttttttcattctaaaagcattttagtggcacacaatttttgtccatgagggaaaggaaatctgttttggttactttgcttgaggtgcattcttcatatgtccagttttatggaagtaataaacttcagtttggtcataagatgtcatattaaagggcaaacatatattcaatgttcaattcatcgtaaatgttccctttttgtaaaagattgcatactcatttatttgagttgcaggtgtatctagtagttggaggag

SEQ ID NO: 12 - DNA, 673 bp, Zea mays
gatcatccaggtgcaaccgtataagtcctaaagtggtgaggaacacgaaacaaccatgcattggcatgtaaagctccaagaatttgttgtatccttaacaactcacagaacatcaaccaaaattgcacgtcaagggtattgggtaagaaacaatcaaacaaatcctctctgtgtgcaaagaaacacggtgagtcatgccgagatcatactcatctgatatacatgcttacagctcacaagacattacaaacaactcatattgcattacaaagatcgtttcatgaaaaataaaataggccggacaggacaaaaatccttgacgtgtaaagtaaatttacaacaaaaaaaaagccatatgtcaagctaaatctaattcgttttacgtagatcaacaacctgtagaaggcaacaaaactgagccacgcagaagtacagaatgattccagatgaaccatcgacgtgctacgtaaagagagtgacgagtcatatacatttggcaagaaaccatgaagctgcctacagccgtctcggtggcataagaacacaagaaattgtgttaattaatcaaagctataaataacgctcgcatgcctgtgcacttctccatcaccaccactgggtcttcagaccattagctttatctactccagagcgcagaagaacccgatcgaca

SEQ ID NO: 13 - protein, 454 aa, Artificial Sequence
  synthetic
MRVLLVALALLALAASATSAKYLELEEGGVIMQAFYWDVPSGGIWWDTIRQKIPEWYDAGISAIWIPPASKGMSGGYSMGYDPYDYFDLGEYYQKGTVETRFGSKQELINMINTAHAYGIKVIADIVINHRAGGDLEWNPFVGDYTWTDFSKVASGKYTANYLDFHPNELHAGDSGTFGGYPDICHDKSWDQYWLWASQESYAAYLRSIGIDAWRFDYVKGYGAWVVKDWLNWWGGWAVGEYWDTNVDALLNWAYSSGAKVFDFPLYYKMDAAFDNKNIPALVEALKNGGTVVSRDPFKAVTFVANHDTDIIWNKYPAYAFILTYEGQPTIFYRDYEEWLNKDKLKNLIWIHDNLAGGSTSIVYYDSDEMIFVRNGYGSKPGLITYINLGSSKVGRWVYVPKFAGACIHEYTGNLGGWVDKYVYSSGWVYLEAPAYDPANGQYGYSVWSYCGVG

SEQ ID NO: 14 - protein, 460 aa, Artificial Sequence
  synthetic
MRVLLVALALLALAASATSAKYLELEEGGVIMQAFYWDVPSGGIWWDTIRQKIPEWYDAGISAIWIPPASKGMSGGYSMGYDPYDYFDLGEYYQKGTVETRFGSKQELINMINTAHAYGIKVIADIVINHRAGGDLEWNPFVGDYTWTDFSKVASGKYTANYLDFHPNELHAGDSGTFGGYPDICHDKSWDQYWLWASQESYAAYLRSIGIDAWRFDYVKGYGAWVVKDWLNWWGGWAVGEYWDTNVDALLNWAYSSGAKVFDFPLYYKMDAAFDNKNIPALVEALKNGGTVVSRDPFKAVTFVANHDTDIIWNKYPAYAFILTYEGQPTIFYRDYEEWLNKDKLKNLIWIHDNLAGGSTSIVYYDSDEMIFVRNGYGSKPGLITYINLGSSKVGRWVYVPKFAGACIHEYTGNLGGWVDKYVYSSGWVYLEAPAYDPANGQYGYSVWSYCGVGSEKDEL

SEQ ID NO: 15 - protein, 518 aa, Artificial Sequence
  synthetic
MLAALATSQLVATRAGLGVPDASTFRRGAAQGLRGARASAAADTLSMRTSARAAPRHQHQQARRGARFPSLVVCASAGAMAKYLELEEGGVIMQAFYWDVPSGGIWWDTIRQKIPEWYDAGISAIWIPPASKGMSGGYSMGYDPYDYFDLGEYYQKGTVETRFGSKQELINMINTAHAYGIKVIADIVINHRAGGDLEWNPFVGDYTWTDFSKVASGKYTANYLDFHPNELHAGDSGTFGGYPDICHDKSWDQYWLWASQESYAAYLRSIGIDAWRFDYVKGYGAWVVKDWLNWWGGWAVGEYWDTNVDALLNWAYSSGAKVFDFPLYYKMDAAFDNKNIPALVEALKNGGTVVSRDPFKAVTFVANHDTDIIWNKYPAYAFILTYEGQPTIFYRDYEEWLNKDKLKNLIWIHDNLAGGSTSIVYYDSDEMIFVRNGYGSKPGLITYINLGSSKVGRWVYVPKFAGACIHEYTGNLGGWVDKYVYSSGWVYLEAPAYDPANGQYGYSVWSYCGVGTSI

SEQ ID NO: 16 - protein, 820 aa, Artificial Sequence
  synthetic
MLAALATSQLVATRAGLGVPDASTFRRGAAQGLRGARASAAADTLSMRTSARAAPRHQHQQARRGARFPSLVVCASAGAMAKYLELEEGGVIMQAFYWDVPSGGIWWDTIRQKIPEWYDAGISAIWIPPASKGMSGGYSMGYDPYDYFDLGEYYQKGTVETRFGSKQELINMINTAHAYGIKVIADIVINHRAGGDLEWNPFVGDYTWTDFSKVASGKYTANYLDFHPNELHAGDSGTFGGYPDICHDKSWDQYWLWASQESYAAYLRSIGIDAWRFDYVKGYGAWVVKDWLNWWGGWAVGEYWDTNVDALLNWAYSSGAKVFDFPLYYKMDAAFDNKNIPALVEALKNGGTVVSRDPFKAVTFVANHDTDIIWNKYPAYAFILTYEGQPTIFYRDYEEWLNKDKLKNLIWIHDNLAGGSTSIVYYDSDEMIFVRNGYGSKPGLITYINLGSSKVGRWVYVPKFAGACIHEYTGNLGGWVDKYVYSSGWVYLEAPAYDPANGQYGYSVWSYCGVGTSIAGILEADRVLTVSPYYAEELISGIARGCELDNIMRLTGITGIVNGMDVSEWDPSRDKYIAVKYDVSTAVEAKALNKEALQAEVGLPVDRNIPLVAFIGRLEEQKGPDVMAAAIPQLMEMVEDVQIVLLGTGKKKFERMLMSAEEKFPGKVRAVVKFNAALAHHIMAGADVLAVTSRFEPCGLIQLQGMRYGTPCACASTGGLVDTIIEGKTGFHMGRLSVDCNVVEPADVKKVATTLQRAIKVVGTPAYEEMVRNCMIQDLSWKGPAKNWENVLLSLGVAGGEPGVEGEEIAPLAKENVAAP

SEQ ID NO: 17 - protein, 19 aa, Artificial Sequence
  synthetic
MRVLLVALALLALAASATS

SEQ ID NO: 18 - protein, 444 aa, Thermotoga maritima
MAEFFPEIPKIQFEGKESTNPLAFRFYDPNEVIDGKPLKDHLKFSVAFWHTFVNEGRDPFGDPTAERPWNRFSDPMDKAFARVDALFEFCEKLNIEYFCFHDRDIAPEGKTLRETNKILDKVVERIKERMKDSNVKLLWGTANLFSHPRYMHGAATTCSADVFAYAAAQVKKALEITKELGGEGYVFWGGREGYETLLNTDLGLELENLARFLRMAVEYAKKIGFTGQFLIEPKPKEPTKHQYDFDVATAYAFLKNHGLDEYFKFNIEANHATLAGHTFQHELRMARILGKLGSIDANQGDLLLGWDTDQFPTNIYDTTLAMYEVIKAGGFTKGGLNFDAKVRRASYKVEDLFIGHIAGMDTFALGFKIAYKLAKDGVFDKFIEEKYRSFKEGIGKEIVEGKTDFEKLEEYIIDKEDIELPSGKQEYLESLLNSYIVKTIAELR

SEQ ID NO: 19 - DNA, 1335 bp, Thermotoga maritima
atggccgagttcttcccggagatcccgaagatccagttcgagggcaaggagtccaccaacccgctcgccttccgcttctacgacccgaacgaggtgatcgacggcaagccgctcaaggaccacctcaagttctccgtggccttctggcacaccttcgtgaacgagggccgcgacccgttcggcgacccgaccgccgagcgcccgtggaaccgcttctccgacccgatggacaaggccttcgcccgcgtggacgccctcttcgagttctgcgagaagctcaacatcgagtacttctgcttccacgaccgcgacatcgccccggagggcaagaccctccgcgagaccaacaagatcctcgacaaggtggtggagcgcatcaaggagcgcatgaaggactccaacgtgaagctcctctggggcaccgccaacctcttctcccacccgcgctacatgcacggcgccgccaccacctgctccgccgacgtgttcgcctacgccgccgcccaggtgaagaaggccctggagatcaccaaggagctgggcggcgagggctacgtgttctggggcggccgcgagggctacgagaccctcctcaacaccgacctcggcctggagctggagaacctcgcccgcttcctccgcatggccgtggagtacgccaagaagatcggcttcaccggccagttcctcatcgagccgaagccgaaggagccgaccaagcaccagtacgacttcgacgtggccaccgcctacgccttcctcaagaaccacggcctcgacgagtacttcaagttcaacatcgaggccaaccacgccaccctcgccggccacaccttccagcacgagctgcgcatggcccgcatcctcggcaagctcggctccatcgacgccaaccagggcgacctcctcctcggctgggacaccgaccagttcccgaccaacatctacgacaccaccctcgccatgtacgaggtgatcaaggccggcggcttcaccaagggcggcctcaacttcgacgccaaggtgcgccgcgcctcctacaaggtggaggacctcttcatcggccacatcgccggcatggacaccttcgccctcggcttcaagatcgcctacaagctcgccaaggacggcgtgttcgacaagttcatcgaggagaagtaccgctccttcaaggagggcatcggcaaggagatcgtggagggcaagaccgacttcgagaagctggaggagtacatcatcgacaaggaggacatcgagctgccgtccggcaagcaggagtacctggagtccctcctcaactcctacatcgtgaagaccatcgccgagctgcgctga

SEQ ID NO: 20 - protein, 444 aa, Thermotoga neapolitana
MAEFFPEIPKVQFEGKESTNPLAFKFYDPEEIIDGKPLKDHLKFSVAFWHTFVNEGRDPFGDPTADRPWNRYTDPMDKAFARVDALFEFCEKLNIEYFCFHDRDIAPEGKTLRETNKILDKVVERIKERMKDSNVKLLWGTANLFSHPRYMHGAATTCSADVFAYAAAQVKKALEITKELGGEGYVFWGGREGYETLLNTDLGFELENLARFLRMAVDYAKRIGFTGQFLIEPKPKEPTKHQYDFDVATAYAFLKSHGLDEYFKFNIEANHATLAGHTFQHELRMARILGKLGSIDANQGDLLLGWDTDQFPTNVYDTTLAMYEVIKAGGFTKGGLNFDAKVRRASYKVEDLFIGHIAGMDTFALGFKVAYKLVKDGVLDKFIEEKYRSFREGIGRDIVEGKVDFEKLEEYIIDKETIELPSGKQEYLESLINSYIVKTILELR

SEQ ID NO: 21 - DNA, 1335 bp, Thermotoga neapolitana
atggccgagttcttcccggagatcccgaaggtgcagttcgagggcaaggagtccaccaacccgctcgccttcaagttctacgacccggaggagatcatcgacggcaagccgctcaaggaccacctcaagttctccgtggccttctggcacaccttcgtgaacgagggccgcgacccgttcggcgacccgaccgccgaccgcccgtggaaccgctacaccgacccgatggacaaggccttcgcccgcgtggacgccctcttcgagttctgcgagaagctcaacatcgagtacttctgcttccacgaccgcgacatcgccccggagggcaagaccctccgcgagaccaacaagatcctcgacaaggtggtggagcgcatcaaggagcgcatgaaggactccaacgtgaagctcctctggggcaccgccaacctcttctcccacccgcgctacatgcacggcgccgccaccacctgctccgccgacgtgttcgcctacgccgccgcccaggtgaagaaggccctggagatcaccaaggagctgggcggcgagggctacgtgttctggggcggccgcgagggctacgagaccctcctcaacaccgacctcggcttcgagctggagaacctcgcccgcttcctccgcatggccgtggactacgccaagcgcatcggcttcaccggccagttcctcatcgagccgaagccgaaggagccgaccaagcaccagtacgacttcgacgtggccaccgcctacgccttcctcaagtcccacggcctcgacgagtacttcaagttcaacatcgaggccaaccacgccaccctcgccggccacaccttccagcacgagctgcgcatggcccgcatcctcggcaagctcggctccatcgacgccaaccagggcgacctcctcctcggctgggacaccgaccagttcccgaccaacgtgtacgacaccaccctcgccatgtacgaggtgatcaaggccggcggcttcaccaagggcggcctcaacttcgacgccaaggtgcgccgcgcctcctacaaggtggaggacctcttcatcggccacatcgccggcatggacaccttcgccctcggcttcaaggtggcctacaagctcgtgaaggacggcgtgctcgacaagttcatcgaggagaagtaccgctccttccgcgagggcatcggccgcgacatcgtggagggcaaggtggacttcgagaagctggaggagtacatcatcgacaaggagaccatcgagctgccgtccggcaagcaggagtacctggagtccctcatcaactcctacatcgtgaagaccatcctggagctgcgctga

SEQ ID NO: 22 - DNA, 28 bp, Artificial Sequence
  synthetic
agcgaattcatggcggctctggccacgt

SEQ ID NO: 23 - DNA, 29 bp, Artificial Sequence
  synthetic
agctaagcttcagggcgcggccacgttct

SEQ ID NO: 24 - protein, 825 aa, Artificial Sequence
  synthetic
MRVLLVALALLALAASATSAGHWYKHQRAYQFTGEDDFGKVAVVKLPMDLTKVGIIVRLNEWQAKDVAKDRFIEIKDGKAEVWILQGVEEIFYEKPDTSPRIFFAQARSNKVIEAFLTNPVDTKKKELFKVTVDGKEIPVSRVEKADPTDIDVTNYVRIVLSESLKEEDLRKDVELIIEGYKPARVIMMEILDDYYYDGELGAVYSPEKTIFRVWSPVSKWVKVLLFKNGEDTEPYQVVNMEYKGNGVWEAVVEGDLDGVFYLYQLENYGKIRTTVDPYSKAVYANNQESAVVNLARTNPEGWENDRGPKIEGYEDAIIYEIHIADITGLENSGVKNKGLYLGLTEENTKAPGGVTTGLSHLVELGVTHVHILPFFDFYTGDELDKDFEKYYNWGYDPYLFMVPEGRYSTDPKNPHTRIREVKEMVKALHKHGIGVIMDMVFPHTYGIGELSAFDQTVPYYFYRIDKTGAYLNESGCGNVIASERPMMRKFIVDTVTYWVKEYHIDGFRFDQMGLIDKKTMLEVERALHKIDPTIILYGEPWGGWGAPIRFGKSDVAGTHVAAFNDEFRDAIRGSVFNPSVKGFVMGGYGKETKIKRGVVGSINYDGKLIKSFALDPEETINYAACHDNHTLWDKNYLAAKADKKKEWTEEELKNAQKLAGAILLTSQGVPFLHGGQDFCRTTNFNDNSYNAPISINGFDYERKLQFIDVFNYHKGLIKLRKEHPAFRLKNAEEIKKHLEFLPGGRRIVAFMLKDHAGGDPWKDIVVIYNGNLEKTTYKLPEGKWNVVVNSQKAGTEVIETVEGTIELDPLSAYVLYRESEKDEL

SEQ ID NO: 25 - DNA, 2478 bp, Artificial Sequence
  synthetic
atgagggtgttgctcgttgccctcgctctcctggctctcgctgcgagcgccaccagcgctggccactggtacaagcaccagcgcgcctaccagttcaccggcgaggacgacttcgggaaggtggccgtggtgaagctcccgatggacctcaccaaggtgggcatcatcgtgcgcctcaacgagtggcaggcgaaggacgtggccaaggaccgcttcatcgagatcaaggacggcaaggccgaggtgtggatactccagggcgtggaggagatcttctacgagaagccggacacctccccgcgcatcttcttcgcccaggcccgctccaacaaggtgatcgaggccttcctcaccaacccggtggacaccaagaagaaggagctgttcaaggtgaccgtcgacggcaaggagatcccggtgtcccgcgtggagaaggccgacccgaccgacatcgacgtgaccaactacgtgcgcatcgtgctctccgagtccctcaaggaggaggacctccgcaaggacgtggagctgatcatcgagggctacaagccggcccgcgtgatcatgatggagatcctcgacgactactactacgacggcgagctgggggcggtgtactccccggagaagaccatcttccgcgtgtggtccccggtgtccaagtgggtgaaggtgctcctcttcaagaacggcgaggacaccgagccgtaccaggtggtgaacatggagtacaagggcaacggcgtgtgggaggccgtggtggagggcgacctcgacggcgtgttctacctctaccagctggagaactacggcaagatccgcaccaccgtggacccgtactccaaggccgtgtacgccaacaaccaggagtctgcagtggtgaacctcgcccgcaccaacccggagggctgggagaacgaccgcggcccgaagatcgagggctacgaggacgccatcatctacgagatccacatcgccgacatcaccggcctggagaactccggcgtgaagaacaagggcctctacctcggcctcaccgaggagaacaccaaggccccgggcggcgtgaccaccggcctctcccacctcgtggagctgggcgtgacccacgtgcacatcctcccgttcttcgacttctacaccggcgacgagctggacaaggacttcgagaagtactacaactggggctacgacccgtacctcttcatggtgccggagggccgctactccaccgacccgaagaacccgcacacccgaattcgcgaggtgaaggagatggtgaaggccctccacaagcacggcatcggcgtgatcatggacatggtgttcccgcacacctacggcatcggcgagctgtccgccttcgaccagaccgtgccgtactacttctaccgcatcgacaagaccggcgcctacctcaacgagtccggctgcggcaacgtgatcgcctccgagcgcccgatgatgcgcaagttcatcgtggacaccgtgacctactgggtgaaggagtaccacatcgacggcttccgcttcgaccagatgggcctcatcgacaagaagaccatgctggaggtggagcgcgccctccacaagatcgacccgaccatcatcctctacggcgagccgtggggcggctggggggccccgatccgcttcggcaagtccgacgtggccggcacccacgtggccgccttcaacgacgagttccgcgacgccatccgcggctccgtgttcaacccgtccgtgaagggcttcgtgatgggcggctacggcaaggagaccaagatcaagcgcggcgtggtgggctccatcaactacgacggcaagctcatcaagtccttcgccctcgacccggaggagaccatcaactacgccgcctgccacgacaaccacaccctctgggacaagaactacctcgccgccaaggccgacaagaagaaggagtggaccgaggaggagctgaagaacgcccagaagctcgccggcgccatcctcctcactagtcagggcgtgccgttcctccacggcggccaggacttctgccgcaccaccaacttcaacgacaactcctacaacgccccgatctccatcaacggcttcgactacgagcgcaagctccagttcatcgacgtgttcaactaccacaagggcctcatcaagctccgcaaggagcacccggccttccgcctcaagaacgccgaggagatcaagaagcacctggagttcctcccgggcgggcgccgcatcgtggccttcatgctcaaggaccacgccggcggcgacccgtggaaggacatcgtggtgatctacaacggcaacctggagaagaccacctacaagctcccggagggcaagtggaacgtggtggtgaactcccagaaggccggcaccgaggtgatcgagaccgtggagggcaccatcgagctggacccgctctccgcctacgtgctctaccgcgagtccgagaaggacgagctgtga

SEQ ID NO: 26 - protein, 718 aa, Artificial Sequence
  synthetic
MRVLLVALALLALAASATSMETIKIYENKGVYKVVIGEPFPPIEFPLEQKISSNKSLSELGLTIVQQGNKVIVEKSLDLKEHIIGLGEKAFELDRKRKRYVMYNVDAGAYKKYQDPLYVSIPLFISVKDGVATGYFFNSASKVIFDVGLEEYDKVIVTIPEDSVEFYVIEGPRIEDVLEKYTELTGKPFLPPMWAFGYMISRYSYYPQDKVVELVDIMQKEGFRVAGVFLDIHYMDSYKLFTWHPYRFPEPKKLIDELHKRNVKLITIVDHGIRVDQNYSPFLSGMGKFCEIESGELFVGKMWPGTTVYPDFFREDTREWWAGLISEWLSQGVDGIWLDMNEPTDFSRAIEIRDVLSSLPVQFRDDRLVTTFPDNVVHYLRGKRVKHEKVRNAYPLYEAMATFKGFRTSHRNEIFILSRAGYAGIQRYAFIWTGDNTPSWDDLKLQLQLVLGLSISGVPFVGCDIGGFQGRNFAEIDNSMDLLVKYYALALFFPFYRSHKATDGIDTEPVFLPDYYKEKVKEIVELRYKFLPYIYSLALEASEKGHPVIRPLFYEFQDDDDMYRIEDEYMVGKYLLYAPIVSKEESRLVTLPRGKWYNYWNGEIINGKSVVKSTHELPIYLREGSIIPLEGDELIVYGETSFKRYDNAEITSSSNEIKFSREIYVSKLTITSEKPVSKIIVDDSKEIQVEKTMQNTYVAKINQKIRGKINLESEKDEL

SEQ ID NO: 27 - protein, 712 aa, Artificial Sequence
  synthetic
MRVLLVALALLALAASATSMETIKIYENKGVYKVVIGEPFPPIEFPLEQKISSNKSLSELGLTIVQQGNKVIVEKSLDLKEHIIGLGEKAFELDRKRKRYVMYNVDAGAYKKYQDPLYVSIPLFISVKDGVATGYFFNSASKVIFDVGLEEYDKVIVTIPEDSVEFYVIEGPRIEDVLEKYTELTGKPFLPPMWAFGYMISRYSYYPQDKVVELVDIMQKEGFRVAGVFLDIHYMDSYKLFTWHPYRFPEPKKLIDELHKRNVKLITIVDHGIRVDQNYSPFLSGMGKFCEIESGELFVGKMWPGTTVYPDFFREDTREWWAGLISEWLSQGVDGIWLDMNEPTDFSRAIEIRDVLSSLPVQFRDDRLVTTFPDNVVHYLRGKRVKHEKVRNAYPLYEAMATFKGFRTSHRNEIFILSRAGYAGIQRYAFIWTGDNTPSWDDLKLQLQLVLGLSISGVPFVGCDIGGFQGRNFAEIDNSMDLLVKYYALALFFPFYRSHKATDGIDTEPVFLPDYYKEKVKEIVELRYKFLPYIYSLALEASEKGHPVIRPLFYEFQDDDDMYRIEDEYMVGKYLLYAPIVSKEESRLVTLPRGKWYNYWNGEIINGKSVVKSTHELPIYLREGSIIPLEGDELIVYGETSFKRYDNAEITSSSNEIKFSREIYVSKLTITSEKPVSKIIVDDSKEIQVEKTMQNTYVAKINQKIRGKINLE

SEQ ID NO: 28 - protein, 469 aa, Artificial Sequence
  synthetic
MRVLLVALALLALAASATSMAEFFPEIPKIQFEGKESTNPLAFRFYDPNEVIDGKPLKDHLKFSVAFWHTFVNEGRDPFGDPTAERPWNRFSDPMDKAFARVDALFEFCEKLNIEYFCFHDRDIAPEGKTLRETNKILDKVVERIKERMKDSNVKLLWGTANLFSHPRYMHGAATTCSADVFAYAAAQVKKALEITKELGGEGYVFWGGREGYETLLNTDLGLELENLARFLRMAVEYAKKIGFTGQFLIEPKPKEPTKHQYDFDVATAYAFLKNHGLDEYFKFNIEANHATLAGHTFQHELRMARILGKLGSIDANQGDLLLGWDTDQFPTNIYDTTLAMYEVIKAGGFTKGGLNFDAKVRRASYKVEDLFIGHIAGMDTFALGFKIAYKLAKDGVFDKFIEEKYRSFKEGIGKEIVEGKTDFEKLEEYIIDKEDIELPSGKQEYLESLLNSYIVKTIAELRSEKDEL

SEQ ID NO: 29 - protein, 469 aa, Artificial Sequence
  synthetic
MRVLLVALALLALAASATSMAEFFPEIPKVQFEGKESTNPLAFKFYDPEEIIDGKPLKDHLKFSVAFWHTFVNEGRDPFGDPTADRPWNRYTDPMDKAFARVDALFEFCEKLNIEYFCFHDRDIAPEGKTLRETNKILDKVVERIKERMKDSNVKLLWGTANLFSHPRYMHGAATTCSADVFAYAAAQVKKALEITKELGGEGYVFWGGREGYETLLNTDLGFELENLARFLRMAVDYAKRIGFTGQFLIEPKPKEPTKHQYDFDVATAYAFLKSHGLDEYFKFNIEANHATLAGHTFQHELRMARILGKLGSIDANQGDLLLGWDTDQFPTNVYDTTLAMYEVIKAGGFTKGGLNFDAKVRRASYKVEDLFIGHIAGMDTFALGFKVAYKLVKDGVLDKFIEEKYRSFREGIGRDIVEGKVDFEKLEEYIIDKETIELPSGKQEYLESLINSYIVKTILELRSEKDEL

SEQ ID NO: 30 - protein, 463 aa, Artificial Sequence
  synthetic
MRVLLVALALLALAASATSMAEFFPEIPKVQFEGKESTNPLAFKFYDPEEIIDGKPLKDHLKFSVAFWHTFVNEGRDPFGDPTADRPWNRYTDPMDKAFARVDALFEFCEKLNIEYFCFHDRDIAPEGKTLRETNKILDKVVERIKERMKDSNVKLLWGTANLFSHPRYMHGAATTCSADVFAYAAAQVKKALEITKELGGEGYVFWGGREGYETLLNTDLGFELENLARFLRMAVDYAKRIGFTGQFLIEPKPKEPTKHQYDFDVATAYAFLKSHGLDEYFKFNIEANHATLAGHTFQHELRMARILGKLGSIDANQGDLLLGWDTDQFPTNVYDTTLAMYEVIKAGGFTKGGLNFDAKVRRASYKVEDLFIGHIAGMDTFALGFKVAYKLVKDGVLDKFIEEKYRSFREGIGRDIVEGKVDFEKLEEYIIDKETIELPSGKQEYLESLINSYIVKTILELR

SEQ ID NO: 31 - protein, 25 aa, Artificial Sequence
  synthetic
MGKNGNLCCFSLLLLLLAGLASGHQ

SEQ ID NO: 32 - protein, 30 aa, Artificial Sequence
  synthetic
MGFVLFSQLPSFLLVSTLLLFLVISHSCRA

SEQ ID NO: 33 - protein, 460 aa, Artificial Sequence; identical to SEQ ID NO: 14
  synthetic

SEQ ID NO: 34 - protein, 825 aa, Artificial Sequence; identical to SEQ ID NO: 24
  synthetic

SEQ ID NO: 35 - protein, 460 aa, Artificial Sequence; identical to SEQ ID NO: 14
  synthetic

SEQ ID NO: 36 - protein, 718 aa, Artificial Sequence; identical to SEQ ID NO: 26
  synthetic

SEQ ID NO: 37 - DNA, 1434 bp, Thermotoga maritima
atgaaagaaaccgctgctgctaaattcgaacgccagcacatggacagcccagatctgggtaccctggtgccacgcggttccatggccgagttcttcccggagatcccgaagatccagttcgagggcaaggagtccaccaacccgctcgccttccgcttctacgacccgaacgaggtgatcgacggcaagccgctcaaggaccacctcaagttctccgtggccttctggcacaccttcgtgaacgagggccgcgacccgttcggcgacccgaccgccgagcgcccgtggaaccgcttctccgacccgatggacaaggccttcgcccgcgtggacgccctcttcgagttctgcgagaagctcaacatcgagtacttctgcttccacgaccgcgacatcgccccggagggcaagaccctccgcgagaccaacaagatcctcgacaaggtggtggagcgcatcaaggagcgcatgaaggactccaacgtgaagctcctctggggcaccgccaacctcttctcccacccgcgctacatgcacggcgccgccaccacctgctccgccgacgtgttcgcctacgccgccgcccaggtgaagaaggccctggagatcaccaaggagctgggcggcgagggctacgtgttctggggcggccgcgagggctacgagaccctcctcaacaccgacctcggcctggagctggagaacctcgcccgcttcctccgcatggccgtggagtacgccaagaagatcggcttcaccggccagttcctcatcgagccgaagccgaaggagccgaccaagcaccagtacgacttcgacgtggccaccgcctacgccttcctcaagaaccacggcctcgacgagtacttcaagttcaacatcgaggccaaccacgccaccctcgccggccacaccttccagcacgagctgcgcatggcccgcatcctcggcaagctcggctccatcgacgccaaccagggcgacctcctcctcggctgggacaccgaccagttcccgaccaacatctacgacaccaccctcgccatgtacgaggtgatcaaggccggcggcttcaccaagggcggcctcaacttcgacgccaaggtgcgccgcgcctcctacaaggtggaggacctcttcatcggccacatcgccggcatggacaccttcgccctcggcttcaagatcgcctacaagctcgccaaggacggcgtgttcgacaagttcatcgaggagaagtaccgctccttcaaggagggcatcggcaaggagatcgtggagggcaagaccgacttcgagaagctggaggagtacatcatcgacaaggaggacatcgagctgccgtccggcaagcaggagtacctggagtccctcctcaactcctacatcgtgaagaccatcgccgagctgcgctccgagaaggacgagctgtga

SEQ ID NO: 38 - protein, 477 aa, Thermotoga maritima
MKETAAAKFERQHMDSPDLGTLVPRGSMAEFFPEIPKIQFEGKESTNPLAFRFYDPNEVIDGKPLKDHLKFSVAFWHTFVNEGRDPFGDPTAERPWNRFSDPMDKAFARVDALFEFCEKLNIEYFCFHDRDIAPEGKTLRETNKILDKVVERIKERMKDSNVKLLWGTANLFSHPRYMHGAATTCSADVFAYAAAQVKKALEITKELGGEGYVFWGGREGYETLLNTDLGLELENLARFLRMAVEYAKKIGFTGQFLIEPKPKEPTKHQYDFDVATAYAFLKNHGLDEYFKFNIEANHATLAGHTFQHELRMARILGKLGSIDANQGDLLLGWDTDQFPTNIYDTTLAMYEVIKAGGFTKGGLNFDAKVRRASYKVEDLFIGHIAGMDTFALGFKIAYKLAKDGVFDKFIEEKYRSFKEGIGKEIVEGKTDFEKLEEYIIDKEDIELPSGKQEYLESLLNSYIVKTIAELRSEKDEL

SEQ ID NO: 39 - DNA, 1434 bp, Thermotoga neapolitana
atgaaagaaaccgctgctgctaaattcgaacgccagcacatggacagcccagatctgggtaccctggtgccacgcggttccatggccgagttcttcccggagatcccgaaggtgcagttcgagggcaaggagtccaccaacccgctcgccttcaagttctacgacccggaggagatcatcgacggcaagccgctcaaggaccacctcaagttctccgtggccttctggcacaccttcgtgaacgagggccgcgacccgttcggcgacccgaccgccgaccgcccgtggaaccgctacaccgacccgatggacaaggccttcgcccgcgtggacgccctcttcgagttctgcgagaagctcaacatcgagtacttctgcttccacgaccgcgacatcgccccggagggcaagaccctccgcgagaccaacaagatcctcgacaaggtggtggagcgcatcaaggagcgcatgaaggactccaacgtgaagctcctctggggcaccgccaacctcttctcccacccgcgctacatgcacggcgccgccaccacctgctccgccgacgtgttcgcctacgccgccgcccaggtgaagaaggccctggagatcaccaaggagctgggcggcgagggctacgtgttctggggcggccgcgagggctacgagaccctcctcaacaccgacctcggcttcgagctggagaacctcgcccgcttcctccgcatggccgtggactacgccaagcgcatcggcttcaccggccagttcctcatcgagccgaagccgaaggagccgaccaagcaccagtacgacttcgacgtggccaccgcctacgccttcctcaagtcccacggcctcgacgagtacttcaagttcaacatcgaggccaaccacgccaccctcgccggccacaccttccagcacgagctgcgcatggcccgcatcctcggcaagctcggctccatcgacgccaaccagggcgacctcctcctcggctgggacaccgaccagttcccgaccaacgtgtacgacaccaccctcgccatgtacgaggtgatcaaggccggcggcttcaccaagggcggcctcaacttcgacgccaaggtgcgccgcgcctcctacaaggtggaggacctcttcatcggccacatcgccggcatggacaccttcgccctcggcttcaaggtggcctacaagctcgtgaaggacggcgtgctcgacaagttcatcgaggagaagtaccgctccttccgcgagggcatcggccgcgacatcgtggagggcaaggtggacttcgagaagctggaggagtacatcatcgacaaggagaccatcgagctgccgtccggcaagcaggagtacctggagtccctcatcaactcctacatcgtgaagaccatcctggagctgcgctccgagaaggacgagctgtga

SEQ ID NO: 40 - protein, 477 aa, Thermotoga neapolitana
MKETAAAKFERQHMDSPDLGTLVPRGSMAEFFPEIPKVQFEGKESTNPLAFKFYDPEEIIDGKPLKDHLKFSVAFWHTFVNEGRDPFGDPTADRPWNRYTDPMDKAFARVDALFEFCEKLNIEYFCFHDRDIAPEGKTLRETNKILDKVVERIKERMKDSNVKLLWGTANLFSHPRYMHGAATTCSADVFAYAAAQVKKALEITKELGGEGYVFWGGREGYETLLNTDLGFELENLARFLRMAVDYAKRIGFTGQFLIEPKPKEPTKHQYDFDVATAYAFLKSHGLDEYFKFNIEANHATLAGHTFQHELRMARILGKLGSIDANQGDLLLGWDTDQFPTNVYDTTLAMYEVIKAGGFTKGGLNFDAKVRRASYKVEDLFIGHIAGMDTFALGFKVAYKLVKDGVLDKFIEEKYRSFREGIGRDIVEGKVDFEKLEEYIIDKETIELPSGKQEYLESLINSYIVKTILELRSEKDEL

SEQ ID NO: 41 - DNA, 1435 bp, Thermotoga maritima
atgggcagcagccatcatcatcatcatcacagcagcggcctggtgccgcgcggcagccatatggctagcatgactggtggacagcaaatgggtcggatccccatggccgagttcttcccggagatcccgaagatccagttcgagggcaaggagtccaccaacccgctcgccttccgcttctacgacccgaacgaggtgatcgacggcaagccgctcaaggaccacctcaagttctccgtggccttctggcacaccttcgtgaacgagggccgcgacccgttcggcgacccgaccgccgagcgcccgtggaaccgcttctccgacccgatggacaaggccttcgcccgcgtggacgccctcttcgagttctgcgagaagctcaacatcgagtacttctgcttccacgaccgcgacatcccccggagggcaagaccctccgcgagaccaacaagatcctcgacaaggtggtggagcgcatcaaggagcgcatgaaggactccaacgtgaagctcctctggggcaccgccaacctcttctcccacccgcgctacatgcacggcgccgccaccacctgctccgccgacgtgttcgcctacgccgccgcccaggtgaagaaggccctggagatcaccaaggagctgggcggcgagggctacgtgttctggggcggccgcgagggctacgagaccctcctcaacaccgacctcggcctggagctggagaacctcgcccgcttcctccgcatggccgtggagtacgccaagaagatcggcttcaccggccagttcctcatcgagccgaagccgaaggagccgaccaagcaccagtacgcttcgacgtggccaccgcctacgccttcctcaagaaccacggcctcgacgagtacttcaagttcaacatcgaggccaaccacgccaccctcgccggccacaccttccagcacgagctgcgcatggcccgcatcctcggcaagctcggctccatcgacgccaaccagggcgacctcctcctcggctgggacaccgaccagttcccgaccaacatctacgacaccaccctcgccatgtacgaggtgatcaaggccggcggcttcaccaagggcggcctcaacttcgacgccaaggtgcgccgcgcctcctacaaggtggaggacctcttcatcggccacatcgccggcatggacaccttcgccctcggcttcaagatcgcctacaagctcgccaaggacggcgtgttcgacaagttcatcgaggagaagtaccgctccttcaaggagggcatcggcaaggagatcgtggagggcaagaccgacttcgagaagctggaggagtacatcatcgacaaggaggacatcgagctgccgtccggcaagcaggagtacctggagtccctcctcaactcctacatcgtgaagaccatcgccgagctgcgctga

SEQ ID NO: 42 - protein, 478 aa, Thermotoga maritima
MGSSHHHHHHSSGLVPRGSHMASMTGGQQMGRIPMAEFFPEIPKIQFEGKESTNPLAFRFYDPNEVIDGKPLKDHLKFSVAFWHTFVNEGRDPFGDPTAERPWNRFSDPMDKAFARVDALFEFCEKLNIEYFCFHDRDIAPEGKTLRETNKILDKVVERIKERMKDSNVKLLWGTANLFSHPRYMHGAATTCSADVFAYAAAQVKKALEITKELGGEGYVFWGGREGYETLLNTDLGLELENLARFLRMAVEYAKKIGFTGQFLIEPKPKEPTKHQYDFDVATAYAFLKNHGLDEYFKFNIEANHATLAGHTFQHELRMARILGKLGSIDANQGDLLLGWDTDQFPTNIYDTTLAMYEVIKAGGFTKGGLNFDAKVRRASYKVEDLFIGHIAGMDTFALGFKIAYKLAKDGVFDKFIEEKYRSFKEGIGKEIVEGKTDFEKLEEYIIDKEDIELPSGKQEYLESLLNSYIVKTIAELR

SEQ ID NO: 43 - DNA, 1436 bp, Thermotoga neapolitana
atgggcagcagccatcatcatcatcatcacagcagcggcctggtgccgcgcggcagccatatggctagcatgactggtggacagcaaatgggtcggatccccatggccgagttcttcccggagatcccgaaggtgcagttcgagggcaaggagtccaccaacccgctcgccttcaagttctacgacccggaggagatcatcgacggcaagccgctcaaggaccacctcaagttctccgtggccttctggcacaccttcgtgaacgagggccgcgacccgttcggcgacccgaccgccgaccgcccgtggaaccgctacaccgacccgatggacaaggccttcgcccgcgtggacgccctcttcgagttctgcgagaagctcaacatcgagtacttctgcttccacgaccgcgacatcccccggagggcaagaccctccgcgagaccaacaagatcctcgacaaggtggtggagcgcatcaaggagcgcatgaaggactccaacgtgaagctcctctggggcaccgccaacctcttctcccacccgcgctacatgcacggcgccgccaccacctgctccgccgacgtgttcgcctacgccgccgcccaggtgaagaaggccctggagatcaccaaggagctgggcggcgagggctacgtgttctggggcggccgcgagggctacgagaccctcctcaacaccgacctcggcttcgagctggagaacctcgcccgcttcctccgcatggccgtggactacgccaagcgcatcggcttcaccggccagttcctcatcgagccgaagccgaaggagccgaccaagcaccagtacgacttcgacgtggccaccgcctacgccttcctcaagtcccacggcctcgacgagtacttcaagttcaacatcgaggccaaccacgccaccctcgccggccacaccttccagcacgagctgcgcatggcccgcatcctcggcaagctcggctccatcgacgccaaccagggcgacctcctcctcggctgggacaccgaccagttcccgaccaacgtgtacgacaccaccctcgccatgtacgaggtgatcaaggccggcggcttcaccaagggcggcctcaacttcgacgccaaggtgcgccgcgcctcctacaaggtggaggacctcttcatcggccacatcgccggcatggacaccttcgccctcggcttcaaggtggcctacaagctcgtgaaggacggcgtgctcgacaagttcatcgaggagaagtaccgctccttccgcgagggcatcggccgcgacatcgtggagggcaaggtggacttcgagaagctggaggagtacatcatcgacaaggagaccatcgagctgccgtccggcaagcaggagtacctggagtccctcatcaactcctacatcgtgaagaccatcctggagctgcgctga

SEQ ID NO: 44 - protein, 478 aa, Thermotoga neapolitana
MGSSHHHHHHSSGLVPRGSHMASMTGGQQMGRIPMAEFFPEIPKVQFEGKESTNPLAFKFYDPEEIIDGKPLKDHLKFSVAFWHTFVNEGRDPFGDPTADRPWNRYTDPMDKAFARVDALFEFCEKLNIEYFCFHDRDIAPEGKTLRETNKILDKVVERIKERMKDSNVKLLWGTANLFSHPRYMHGAATTCSADVFAYAAAQVKKALEITKELGGEGYVFWGGREGYETLLNTDLGFELENLARFLRMAVDYAKRIGFTGQFLIEPKPKEPTKHQYDFDVATAYAFLKSHGLDEYFKFNIEANHATLAGHTFQHELRMARILGKLGSIDANQGDLLLGWDTDQFPTNVYDTTLAMYEVIKAGGFTKGGLNFDAKVRRASYKVEDLFIGHIAGMDTFALGFKVAYKLVKDGVLDKFIEEKYRSFREGIGRDIVEGKVDFEKLEEYIIDKETIELPSGKQEYLESLINSYIVKTILELR

SEQ ID NO: 45 - protein, 1095 aa, Aspergillus shirousami
ATPADWRSQSIYFLLTDRFARTDGSTTATCNTADQKYCGGTWQGIIDKLDYIQGMGFTAIWITPVTAQLPQTTAYGDAYHGYWQQDIYSLNENYGTADDLKALSSALHERGMYLMVDVVANHMGYDGAGSSVDYSVFKPFSSQDYFHPFCFIQNYEDQTQVEDCWLGDNTVSLPDLDTTKDVVKNEWYDWVGSLVSNYSIDGLRIDTVKHVQKDFWPGYNKAAGVYCIGEVLDVDPAYTCPYQNVMDGVLNYPIYYPLLNAFKSTSGSMDDLYNMINTVKSDCPDSTLLGTFVENHDNPRFASYTNDIALAKNVAAFIILNDGIPIIYAGQEQHYAGGNDPANREATWLSGYPTDSELYKLIASANAIRNYAISKDTGFVTYKNWPIYKDDTTIAMRKGTDGSQIVTILSNKGASGDSYTLSLSGAGYTAGQQLTEVIGCTTVTVGSDGNVPVPMAGGLPRVLYPTEKLAGSKICSSSKPATLDSWLSNEATVARTAILNNIGADGAWVSGADSGIVVASPSTDNPDYFYTWTRDSGIVLKTLVDLFRNGDTDLLSTIEHYISSQAIIQGVSNPSGDLSSGGLGEPKFNVDETAYAGSWGRPQRDGPALRATAMIGFGQWLLDNGYTSAATEIVWPLVRNDLSYVAQYWNQTGYDLWEEVNGSSFFTIAVQHRALVEGSAFATAVGSSCSWCDSQAPQILCYLQSFWTGSYILANFDSSRSGKDTNTLLGSIHTFDPEAGCDDSTFQPCSPRALANHKEVVDSFRSIYTLNDGLSDSEAVAVGRYPEDSYYNGNPWFLCTLAAAEQLYDALYQWDKQGSLEITDVSLDFFKALYSGAATGTYSSSSSTYSSIVSAVKTFADGFVSIVETHAASNGSLSEQFDKSDGDELSARDLTWSYAALLTANNRRNSVVPPSWGETSASSVPGTCAATSASGTYSSVTVTSWPSIVATGGTTTTATTTGSGGVTSTSKTTTTASKTSTTTSSTSCTTPTAVAVTFDLTATTTYGENIYLVGSISQLGDWETSDGIALSADKYTSSNPPWYVTVTLPAGESFEYKFIRVESDDSVEWESDPNREYTVPQACGESTATVTDTWR

SEQ ID NO: 46 - DNA, 3285 bp, Aspergillus shirousami
gccaccccggccgactggcgctcccagtccatctacttcctcctcaccgaccgcttcgcccgcaccgacggctccaccaccgccacctgcaacaccgccgaccagaagtactgcggcggcacctggcagggcatcatcgacaagctcgactacatccagggcatgggcttcaccgccatctggatcaccccggtgaccgcccagctcccgcagaccaccgcctacggcgacgcctaccacggctactggcagcaggacatctactccctcaacgagaactacggcaccgccgacgacctcaaggccctctcctccgccctccacgagcgcggcatgtacctcatggtggacgtggtggccaaccacatgggctacgacggcgccggctcctccgtggactactccgtgttcaagccgttctcctcccaggactacttccacccgttctgcttcatccagaactacgaggaccagacccaggtggaggactgctggctcggcgacaacaccgtgtccctcccggacctcgacaccaccaaggacgtggtgaagaacgagtggtacgactgggtgggctccctcgtgtccaactactccatcgacggcctccgcatcgacaccgtgaagcacgtgcagaaggacttctggccgggctacaacaaggccgccggcgtgtactgcatcggcgaggtgctcgacgtggacccggcctacacctgcccgtaccagaacgtgatggacggcgtgctcaactacccgatctactacccgctcctcaacgccttcaagtccacctccggctcgatggacgacctctacaacatgatcaacaccgtgaagtccgactgcccggactccaccctcctcggcaccttcgtggagaaccacgacaacccgcgcttcgcctcctacaccaacgacatcgccctcgccaagaacgtggccgccttcatcatcctcaacgacggcatcccgatcatctacgccggccaggagcagcactacgccggcggcaacgacccggccaaccgcgaggccacctggctctccggctacccgaccgactccgagctgtacaagctcatcgcctccgccaacgccatccgcaactacgccatctccaaggacaccggcttcgtgacctacaagaactggccgatctacaaggacgacaccaccatcgccatgcgcaagggcaccgacggctcccagatcgtgaccatcctctccaacaagggcgcctccggcgactcctacaccctctccctctccggcgccggctacaccgccggccagcagctcaccgaggtgatcggctgcaccaccgtgaccgtgggctccgacggcaacgtgccggtgccgatggccggcggcctcccgcgcgtgctctacccgaccgagaagctcgccggctccaagatatgctcctcctccaagccggccaccctcgactcctggctctccaacgaggccaccgtggcccgcaccgccatcctcaacaacatcggcgccgacggcgcctgggtgtccggcgccgactccggcatcgtggtggcctccccgtccaccgacaacccggactacttctacacctggacccgcgactccggcatcgtgctcaagaccctcgtggacctcttccgcaacggcgacaccgacctcctctccaccatcgagcactacatctcctcccaggccatcatccagggcgtgtccaacccgtccggcgacctctcctccggcggcctcggcgagccgaagttcaacgtggacgagaccgcctacgccggctcctggggccgcccgcagcgcgacggcccggccctccgcgccaccgccatgatcggcttcggccagtggctcctcgacaacggctacacctccgccgccaccgagatcgtgtggccgctcgtgcgcaacgacctctcctacgtggcccagtactggaaccagaccggctacgacctctgggaggaggtgaacggctcctccttcttcaccatcgccgtgcagcaccgcgccctcgtggagggctccgccttcgccaccgccgtgggctcctcctgctcctggtgcgactcccaggccccgcagatcctctgctacctccagtccttctggaccggctcctacatcctcgccaacttcgactcctcccgctccggcaaggacaccaacaccctcctcggctccatccacaccttcgacccggaggccggctgcgacgactccaccttccagccgtgctccccgcgcgccctcgccaaccacaaggaggtggtggactccttccgctccatctacaccctcaacgacggcctctccgactccgaggccgtggccgtgggccgctacccggaggactcctactacaacggcaacccgtggttcctctgcaccctcgccgccgccgagcagctctacgacgccctctaccagtgggacaagcagggctccctggagatcaccgacgtgtccctcgacttcttcaaggccctctactccggcgccgccaccggcacctactcctcctcctcctccacctactcctccatcgtgtccgccgtgaagaccttcgccgacggcttcgtgtccatcgtggagacccacgccgcctccaacggctccctctccgagcagttcgacaagtccgacggcgacgagctgtccgcccgcgacctcacctggtcctacgccgccctcctcaccgccaacaaccgccgcaactccgtggtgccgccgtcctggggcgagacctccgcctcctccgtgccgggcacctgcgccgccacctccgcctccggcacctactcctccgtgaccgtgacctcctggccgtccatcgtggccaccggcggcaccaccaccaccgccaccaccaccggctccggcggcgtgacctccacctccaagaccaccaccaccgcctccaagacctccaccaccacctcctccacctcctgcaccaccccgaccgccgtggccgtgaccttcgacctcaccgccaccaccacctacggcgagaacatctacctcgtgggctccatctcccagctcggcgactgggagacctccgacggcatcgccctctccgccgacaagtacacctcctccaacccgccgtggtacgtgaccgtgaccctcccggccggcgagtccttcgagtacaagttcatccgcgtggagtccgacgactccgtggagtgggagtccgacccgaaccgcgagtacaccgtgccgcaggcctgcggcgagtccaccgccaccgtgaccgacacctggcgc

SEQ ID NO: 47 - protein, 679 aa, Thermoanaerobacterium thermosaccharolyticum
VLSGCSNNVSSIKIDRFNNISAVNGPGEEDTWASAQKQGVGTANNYVSRVWFTLANGAISEVYYPTIDTADVKEIKFIVTDGKSFVSDETKDAISKVEKFTDKSLGYKLVNTDKKGRYRITKEIFTDVKRNSLIMKAKFEALEGSIHDYKLYLAYDPHIKNQGSYNEGYVIKANNNEMLMAKRDNVYTALSSNIGWKGYSIGYYKVNDIMTDLDENKQMTKHYDSARGNIIEGAEIDLTKNSEFEIVLSFGGSDSEAAKTALETLGEDYNNLKNNYIDEWTKYCNTLNNFNGKANSLYYNSMMILKASEDKTNKGAYIASLSIPWGDGQRDDNTGGYHLVWSRDLYHVANAFIAAGDVDSANRSLDYLAKVVKDNGMIPQNTWISGKPYWTSIQLDEQADPIILSYRLKRYDLYDSLVKPLADFIIKIGPKTGQERWEEIGGYSPATMAAEVAGLTCAAYIAEQNKDYESAQKYQEKADNWQKLIDNLTYTENGPLGNGQYYIRIAGLSDPNADFMINIANGGGVYDQKEIVDPSFLELVRLGVKSADDPKILNTLKVVDSTIKVDTPKGPSWYRYNHDGYGEPSKTELYHGAGKGRLWPLLTGERGMYEIAAGKDATPYVKAMEKFANEGGIISEQVWEDTGLPTDSASPLNWAHAEYVILFASNIEHKVLDMPDIVY

SEQ ID NO: 48 - DNA, 2037 bp, Thermoanaerobacterium thermosaccharolyticum
  synthetic
gtgctctccggctgctccaacaacgtgtcctccatcaagatcgaccgcttcaacaacatctccgccgtgaacggcccgggcgaggaggacacctgggcctccgcccagaagcagggcgtgggcaccgccaacaactacgtgtcccgcgtgtggttcaccctcgccaacggcgccatctccgaggtgtactacccgaccatcgacaccgccgacgtgaaggagatcaagttcatcgtgaccgacggcaagtccttcgtgtccgacgagaccaaggacgccatctccaaggtggagaagttcaccgacaagtccctcggctacaagctcgtgaacaccgacaagaagggccgctaccgcatcaccaaggaaatcttcaccgacgtgaagcgcaactccctcatcatgaaggccaagttcgaggccctcgagggctccatccacgactacaagctctacctcgcctacgacccgcacatcaagaaccagggctcctacaacgagggctacgtgatcaaggccaacaacaacgagatgctcatggccaagcgcgacaacgtgtacaccgccctctcctccaacatcggctggaagggctactccatcggctactacaaggtgaacgacatcatgaccgacctcgacgagaacaagcagatgaccaagcactacgactccgcccgcggcaacatcatcgagggcgccgagatcgacctcaccaagaactccgagttcgagatcgtgctctccttcggcggctccgactccgaggccgccaagaccgccctcgagaccctcggcgaggactacaacaacctcaagaacaactacatcgacgagtggaccaagtactgcaacaccctcaacaacttcaacggcaaggccaactccctctactacaactccatgatgatcctcaaggcctccgaggacaagaccaacaagggcgcctacatcgcctccctctccatcccgtggggcgacggccagcgcgacgacaacaccggcggctaccacctcgtgtggtcccgcgacctctaccacgtggccaacgccttcatcgccgccggcgacgtggactccgccaaccgctccctcgactacctcgccaaggtggtgaaggacaacggcatgatcccgcagaacacctggatctccggcaagccgtactggacctccatccagctcgacgagcaggccgacccgatcatcctctcctaccgcctcaagcgctacgacctctacgactccctcgtgaagccgctcgccgacttcatcatcaagatcggcccgaagaccggccaggagcgctgggaggagatcggcggctactccccggccacgatggccgccgaggtggccggcctcacctgcgccgcctacatcgccgagcagaacaaggactacgagtccgcccagaagtaccaggagaaggccgacaactggcagaagctcatcgacaacctcacctacaccgagaacggcccgctcggcaacggccagtactacatccgcatcgccggcctctccgacccgaacgccgacttcatgatcaacatcgccaacggcggcggcgtgtacgaccagaaggagatcgtggacccgtccttcctcgagctggtgcgcctcggcgtgaagtccgccgacgacccgaagatcctcaacaccctcaaggtggtggactccaccatcaaggtggacaccccgaagggcccgtcctggtatcgctacaaccacgacggctacggcgagccgtccaagaccgagctgtaccacggcgccggcaagggccgcctctggccgctcctcaccggcgagcgcggcatgtacgagatcgccgccggcaaggacgccaccccgtacgtgaaggcgatggagaagttcgccaacgagggcggcatcatctccgagcaggtgtgggaggacaccggcctcccgaccgactccgcctccccgctcaactgggcccacgccgagtacgtgatcctcttcgcctccaacatcgagcacaaggtgctcgacatgccggacatcgtgtac

SEQ ID NO: 49 - protein, 579 aa, Rhizopus oryzae
ASIPSSASVQLDSYNYDGSTFSGKIYVKNIAYSKKVTVIYADGSDNWNNNGNTIAASYSAPISGSNYEYWTFSASINGIKEFYIKYEVSGKTYYDNNNSANYQVSTSKPTTTTATATTTTAPSTSTTTPPSRSEPATFPTGNSTISSWIKKQEGISRFAMLRNINPPGSATGFIAASLSTAGPDYYYAWTRDAALTSNVIVYEYNTTLSGNKTILNVLKDYVTFSVKTQSTSTVCNCLGEPKFNPDASGYTGAWGRPQNDGPAERATTFILFADSYLTQTKDASYVTGTLKPAIFKDLDYVVNVWSNGCFDLWEEVNGVHFYTLMVMRKGLLLGADFAKRNGDSTRASTYSSTASTIANKISSFWVSSNNWIQVSQSVTGGVSKKGLDVSTLLAANLGSVDDGFFTPGSEKILATAVAVEDSFASLYPINKNLPSYLGNSIGRYPEDTYNGNGNSQGNSWFLAVTGYAELYYRAIKEWIGNGGVTVSSISLPFFKKFDSSATSGKKYTVGTSDFNNLAQNIALAADRFLSTVQLHAHNNGSLAEEFDRTTGLSTGARDLTWSHASLITASYAKAGAPAA

SEQ ID NO: 50 - DNA, 1737 bp, Rhizopus oryzae
gcctccatcccgtcctccgcctccgtgcagctcgactcctacaactacgacggctccaccttctccggcaaaatctacgtgaagaacatcgcctactccaagaaggtgaccgtgatctacgccgacggctccgacaactggaacaacaacggcaacaccatcgccgcctcctactccgccccgatctccggctccaactacgagtactggaccttctccgcctccatcaacggcatcaaggagttctacatcaagtacgaggtgtccggcaagacctactacgacaacaacaactccgccaactaccaggtgtccacctccaagccgaccaccaccaccgccaccgccaccaccaccaccgccccgtccacctccaccaccaccccgccgtcccgctccgagccggccaccttcccgaccggcaactccaccatctcctcctggatcaagaagcaggagggcatctcccgcttcgccatgctccgcaacatcaacccgccgggctccgccaccggcttcatcgccgcctccctctccaccgccggcccggactactactacgcctggacccgcgacgccgccctcacctccaacgtgatcgtgtacgagtacaacaccaccctctccggcaacaagaccatcctcaacgtgctcaaggactacgtgaccttctccgtgaagacccagtccacctccaccgtgtgcaactgcctcggcgagccgaagttcaacccggacgcctccggctacaccggcgcctggggccgcccgcagaacgacggcccggccgagcgcgccaccaccttcatcctcttcgccgactcctacctcacccagaccaaggacgcctcctacgtgaccggcaccctcaagccggccatcttcaaggacctcgactacgtggtgaacgtgtggtccaacggctgcttcgacctctgggaggaggtgaacggcgtgcacttctacaccctcatggtgatgcgcaagggcctcctcctcggcgccgacttcgccaagcgcaacggcgactccacccgcgcctccacctactcctccaccgcctccaccatcgccaacaaaatctcctccttctgggtgtcctccaacaactggatacaggtgtcccagtccgtgaccggcggcgtgtccaagaagggcctcgacgtgtccaccctcctcgccgccaacctcggctccgtggacgacggcttcttcaccccgggctccgagaagatcctcgccaccgccgtggccgtggaggactccttcgcctccctctacccgatcaacaagaacctcccgtcctacctcggcaactccatcggccgctacccggaggacacctacaacggcaacggcaactcccagggcaactcctggttcctcgccgtgaccggctacgccgagctgtactaccgcgccatcaaggagtggatcggcaacggcggcgtgaccgtgtcctccatctccctcccgttcttcaagaagttcgactcctccgccacctccggcaagaagtacaccgtgggcacctccgacttcaacaacctcgcccagaacatcgccctcgccgccgaccgcttcctctccaccgtgcagctccacgcccacaacaacggctccctcgccgaggagttcgaccgcaccaccggcctctccaccggcgcccgcgacctcacctggtcccacgcctccctcatcaccgcctcctacgccaaggccggcgccccggccgcc

SEQ ID NO: 51 - protein, 439 aa, Artificial Sequence
  synthetic
MAKHLAAMCWCSLLVLVLLCLGSQLAQSQVLFQGFNWESWKKQGGWYNYLLGRVDDIAATGATHVWLPQPSHSVAPQGYMPGRLYDLDASKYGTHAELKSLTAAFHAKGVQCVADVVINHRCADYKDGRGIYCVFEGGTPDSRLDWGPDMICSDDTQYSNGRGHRDTGADFAAAPDIDHLNPRVQQELSDWLNWLKSDLGFDGWRLDFAKGYSAAVAKVYVDSTAPTFVVAEIWSSLHYDGNGEPSSNQDADRQELVNWAQAVGGPAAAFDFTTKGVLQAAVQGELWRMKDGNGKAPGMIGWLPEKAVTFVDNHDTGSTQNSWPFPSDKVMQGYAYILTHPGTPCIFYDHVFDWNLKQEISALSAVRSRNGIHPGSELNILAADGDLYVAKIDDKVIVKIGSRYDVGNLIPSDFHAVAHGNNYCVWEKHGLRVPAGRHH

SEQ ID NO: 52 - DNA, 1320 bp, Artificial Sequence
  synthetic
atggcgaagcacttggctgccatgtgctggtgcagcctcctagtgcttgtactgctctgcttgggctcccagctggcccaatcccaggtcctcttccaggggttcaactgggagtcgtggaagaagcaaggtgggtggtacaactacctcctggggcgggtggacgacatcgccgcgacgggggccacgcacgtctggctcccgcagccgtcgcactcggtggcgccgcaggggtacatgcccggccggctctacgacctggacgcgtccaagtacggcacccacgcggagctcaagtcgctcaccgcggcgttccacgccaagggcgtccagtgcgtcgccgacgtcgtgatcaaccaccgctgcgccgactacaaggacggccgcggcatctactgcgtcttcgagggcggcacgcccgacagccgcctcgactggggccccgacatgatctgcagcgacgacacgcagtactccaacgggcgcgggcaccgcgacacgggggccgacttcgccgccgcgcccgacatcgaccacctcaacccgcgcgtgcagcaggagctctcggactggctcaactggctcaagtccgacctcggcttcgacggctggcgcctcgacttcgccaagggctactccgccgccgtcgccaaggtgtacgtcgacagcaccgcccccaccttcgtcgtcgccgagatatggagctccctccactacgacggcaacggcgagccgtccagcaaccaggacgccgacaggcaggagctggtcaactgggcgcaggcggtgggcggccccgccgcggcgttcgacttcaccaccaagggcgtgctgcaggcggccgtccagggcgagctgtggcgcatgaaggacggcaacggcaaggcgcccgggatgatcggctggctgccggagaaggccgtcacgttcgtcgacaaccacgacaccggctccacgcagaactcgtggccattcccctccgacaaggtcatgcagggctacgcctatatcctcacgcacccaggaactccatgcatcttctacgaccacgttttcgactggaacctgaagcaggagatcagcgcgctgtctgcggtgaggtcaagaaacgggatccacccggggagcgagctgaacatcctcgccgccgacggggatctctacgtcgccaagattgacgacaaggtcatcgtgaagatcgggtcacggtacgacgtcgggaacctgatcccctcagacttccacgccgttgcccctggcaacaactactgcgtttgggagaagcacggtctgagagttccagcggggcggcaccactag

SEQ ID NO: 53 - protein, 45 aa, Artificial Sequence
  synthetic
ATGGTTTTATTTGSGGVTSTSKTTTTASKTSTTTSSTSCTTPTAV

SEQ ID NO: 54 - DNA, 137 bp, Artificial Sequence
  synthetic
gccaccggcggcaccaccaccaccgccaccaccaccggctccggcggcgtgacctccacctccaagaccaccaccaccgcctccaagacctccaccaccacctcctccacctcctgcaccaccccgaccgccgtgtc

SEQ ID NO: 55 - protein, 300 aa, Pyrococcus furiosus
IYFVEKYHTSEDKSTSNTSSTPPQTTLSTTKVLKIRYPDDGEWPGAPIDKDGDGNPEFYIEINLWNILNATGFAEMTYNLTSGVLHYVQQLDNIVLRDRSNWVHGYPEIFYGNKPWNANYATDGPIPLPSKVSNLTDFYLTISYKLEPKNGLPINFAIESWLTREAWRTTGINSDEQEVMIWIYYDGLQPAGSKVKEIVVPIIVNGTPVNATFEVWKANIGWEYVAFRIKTPIKEGTVTIPYGAFISVAANISSLPNYTELYLEDVEIGTEFGTPSTTSAHLEWWITNITLTPLDRPLIS

SEQ ID NO: 56 - DNA, 903 bp, Pyrococcus furiosus
atctacttcgtggagaagtaccacacctccgaggacaagtccacctccaacacctcctccaccccgccgcagaccaccctctccaccaccaaggtgctcaagatccgctacccggacgacggcgagtggcccggcgccccgatcgacaaggacggcgacggcaacccggagttctacatcgagatcaacctctggaacatcctcaacgccaccggcttcgccgagatgacctacaacctcactagtggcgtgctccactacgtgcagcagctcgacaacatcgtgctccgcgaccgctccaactgggtgcacggctacccggaaatcttctacggcaacaagccgtggaacgccaactacgccaccgacggcccgatcccgctcccgtccaaggtgtccaacctcaccgacttctacctcaccatctcctacaagctcgagccgaagaacggtctcccgatcaacttcgccatcgagtcctggctcacccgcgaggcctggcgcaccaccggcatcaactccgacgagcaggaggtgatgatctggatctactacgacggcctccagcccgcgggctccaaggtgaaggagatcgtggtgccgatcatcgtgaacggcaccccggtgaacgccaccttcgaggtgtggaaggccaacatcggctgggagtacgtggccttccgcatcaagaccccgatcaaggagggcaccgtgaccatcccgtacggcgccttcatctccgtggccgccaacatctcctccctcccgaactacaccgagaagtacctcgaggacgtggagatcggcaccgagttcggcaccccgtccaccacctccgcccacctcgagtggtggatcaccaacatcaccctcaccccgctcgaccgcccgctcatctcctag

SEQ ID NO: 57 - protein, 387 aa, Thermus flavus
MYEPKPEHRFTFGLWTVDNVDRDPFGDTVRERLDPVYVVHKLAELGAYGVNLHDEDLIPRGTPPQERDQIVRRFKKALDETVLKVPMVTANLFSEPAFRDGASTTRDPWVWAYALRKSLETMDLGAELGAEIYMFWMVRERSEVESTDKTRKVWDWVRETLNFMTAYTEDQGYGYRFSVEPKPNEPRGDIYFTTVGSMLALIHTLDRPERFGLNPEFAHETMAGLNFDHAVAQAVDAGKLFHIDLNDQRMSRFDQDLRFGSENLKAGFFLVDLLESSGYQGPRHFEAHALRTEDEEGVWTFVRVCMRTYLIIKVRAETFREDPEVKELLAAYYQEDPATLALLDPYSREKAEALKRAELPLETKRRRGYALERLDQLAVEYLLGVRG

SEQ ID NO: 58 - DNA, 978 bp, Artificial Sequence
  synthetic
atggggaagaacggcaacctgtgctgcttctctctgctgctgcttcttctcgccgggttggcgtccggccatcaaatctacttcgtggagaagtaccacacctccgaggacaagtccacctccaacacctcctccaccccgccgcagaccaccctctccaccaccaaggtgctcaagatccgctacccggacgacggtgagtggcccggcgccccgatcgacaaggacggcgacggcaacccggagttctacatcgagatcaacctctggaacatcctcaacgccaccggcttcgccgagatgacctacaacctcactagtggcgtgctccactacgtgcagcagctcgacaacatcgtgctccgcgaccgctccaactgggtgcacggctacccggaaatcttctacggcaacaagccgtggaacgccaactacgccaccgacggcccgatcccgctcccgtccaaggtgtccaacctcaccgacttctacctcaccatctcctacaagctcgagccgaagaacggtctcccgatcaacttcgccatcgagtcctggctcacccgcgaggcctggcgcaccaccggcatcaactccgacgagcaggaggtgatgatctggatctactacgacggcctccagcccgcgggctccaaggtgaaggagatcgtggtgccgatcatcgtgaacggcaccccggtgaacgccaccttcgaggtgtggaaggccaacatcggctgggagtacgtggccttccgcatcaagaccccgatcaaggagggcaccgtgaccatcccgtacggcgccttcatctccgtggccgccaacatctcctccctcccgaactacaccgagaagtacctcgaggacgtggagatcggcaccgagttcggcaccccgtccaccacctccgcccacctcgagtggtggatcaccaacatcaccctcaccccgctcgaccgcccgctcatctcctag

SEQ ID NO: 59 - DNA, 1920 bp, Aspergillus niger
atgtccttccgctccctcctcgccctctccggcctcgtgtgcaccggcctcgccaacgtgatctccaagcgcgccaccctcgactcctggctctccaacgaggccaccgtggcccgcaccgccatcctcaacaacatcggcgccgacggcgcctgggtgtccggcgccgactccggcatcgtggtggcctccccgtccaccgacaacccggactacttctacacctggacccgcgactccggcctcgtgctcaagaccctcgtggacctcttccgcaacggcgacacctccctcctctccaccatcgagaactacatctccgcccaggccatcgtgcagggcatctccaacccgtccggcgacctctcctccggcgccggcctcggcgagccgaagttcaacgtggacgagaccgcctacaccggctcctggggccgcccgcagcgcgacggcccggccctccgcgccaccgccatgatcggcttcggccagtggctcctcgacaacggctacacctccaccgccaccgacatcgtgtggccgctcgtgcgcaacgacctctcctacgtggcccagtactggaaccagaccggctacgacctctgggaggaggtgaacggctcctccttcttcaccatcgccgtgcagcaccgcgccctcgtggagggctccgccttcgccaccgccgtgggctcctcctgctcctggtgcgactcccaggccccggagatcctctgctacctccagtccttctggaccggctccttcatcctcgccaacttcgactcctcccgctccggcaaggacgccaacaccctcctcggctccatccacaccttcgacccggaggccgcctgcgacgactccaccttccagccgtgctccccgcgcgccctcgccaaccacaaggaggtggtggactccttccgctccatctacaccctcaacgacggcctctccgactccgaggccgtggccgtgggccgctacccggaggacacctactacaacggcaacccgtggttcctctgcaccctcgccgccgccgagcagctctacgacgccctctaccagtgggacaagcagggctccctcgaggtgaccgacgtgtccctcgacttcttcaaggccctctactccgacgccgccaccggcacctactcctcctcctcctccacctactcctccatcgtggacgccgtgaagaccttcgccgacggcttcgtgtccatcgtggagacccacgccgcctccaacggctccatgtccgagcagtacgacaagtccgacggcgagcagctctccgcccgcgacctcacctggtcctacgccgccctcctcaccgccaacaaccgccgcaactccgtggtgccggcctcctggggcgagacctccgcctcctccgtgccgggcacctgcgccgccacctccgccatcggcacctactcctccgtgaccgtgacctcctggccgtccatcgtggccaccggcggcaccaccaccaccgccaccccgaccggctccggctccgtgacctccacctccaagaccaccgccaccgcctccaagacctccacctccacctcctccacctcctgcaccaccccgaccgccgtggccgtgaccttcgacctcaccgccaccaccacctacggcgagaacatctacctcgtgggctccatctcccagctcggcgactgggagacctccgacggcatcgccctctccgccgacaagtacacctcctccgacccgctctggtacgtgaccgtgaccctcccggccggcgagtccttcgagtacaagttcatccgcatcgagtccgacgactccgtggagtgggagtccgacccgaaccgcgagtacaccgtgccgcaggcctgcggcacctccaccgccaccgtgaccgacacctggcgc

SEQ ID NO: 60 - protein, 6 aa, Artificial Sequence
  synthetic
SEKDEL

SEQ ID NO: 61 - DNA, 561 bp, Artificial Sequence
  Xylanase BD7436
atggctagcaccttctactggcatttgtggaccgacggcatcggcaccgtgaacgctaccaacggcagcgacggcaactacagcgtgagctggagcaactgcggcaacttcgtggtgggcaagggctggaccaccggcagcgctaccagggtgatcaactacaacgctcatgctttcagcgtggtgggcaacgcttacttggctttgtacggctggaccaggaacagcttgatcgagtactacgtggtggacagctggggcacctacaggccaaccggcacctacaagggcaccgtgaccagcgacggcggcacctacgacatctacaccaccaccaggaccaacgctccaagcatcgacggcaacaacaccaccttcacccaattctggagcgtgaggcaaagcaagaggccaatcggcaccaacaacaccatcaccttcagcaaccatgtgaacgcttggaagagcaagggcatgaacttgggcagcagctggagctaccaagtgttggctaccgagggctaccaaagcagcggctacagcaacgtgaccgtgtggtag

SEQ ID NO: 62 - protein, 186 aa, Artificial Sequence
  Synthetic Construct
MASTFYWHLWTDGIGTVNATNGSDGNYSVSWSNCGNFVVGKGWTTGSATRVINYNAHAFSVVGNAYLALYGWTRNSLIEYYVVDSWGTYRPTGTYKGTVTSDGGTYDIYTTTRTNAPSIDGNNTTFTQFWSVRQSKRPIGTNNTITFSNHVNAWKSKGMNLGSSWSYQVLATEGYQSSGYSNVTVW

SEQ ID NO: 63 - DNA, 561 bp, Artificial Sequence
  Xylanase BD6002A
atggctagcaccgactactggcaaaactggaccgacggcggcggcaccgtgaacgctaccaacggcagcgacggcaactacagcgtgagctggagcaactgcggcaacttcgtggtgggcaagggctggaccaccggcagcgctaccagggtgatcaactacaacgctggcgctttcagcccaagcggcaacggctacttggctttgtacggctggaccaggaacagcttgatcgagtactacgtggtggacagctggggcacctacaggccaaccggcacctacaagggcaccgtgaccagcgacggcggcacctacgacatctacaccaccaccaggaccaacgctccaagcatcgacggcaacaacaccaccttcacccaattctggagcgtgaggcaaagcaagaggccaatcggcaccaacaacaccatcaccttcagcaaccatgtgaacgcttggaagagcaagggcatgaacttgggcagcagctggagctaccaagtgttggctaccgagggctaccaaagcagcggctacagcaacgtgaccgtgtggtag

SEQ ID NO: 64 - protein, 186 aa, Artificial Sequence
  Synthetic Construct
MASTDYWQNWTDGGGTVNATNGSDGNYSVSWSNCGNFVVGKGWTTGSATRVINYNAGAFSPSGNGYLALYGWTRNSLIEYYVVDSWGTYRPTGTYKGTVTSDGGTYDIYTTTRTNAPSIDGNNTTFTQFWSVRQSKRPIGTNNTITFSNHVNAWKSKGMNLGSSWSYQVLATEGYQSSGYSNVTVW

SEQ ID NO: 65 - DNA, 561 bp, Artificial Sequence
  Xylanase BD6002B
atggcctccaccgactactggcagaactggaccgacggcggcggcaccgtgaacgccaccaacggctccgacggcaactactccgtgtcctggtccaactgcggcaacttcgtggtgggcaagggctggaccaccggctccgccacccgcgtgatcaactacaacgccggcgccttctccccgtccggcaacggctacctcgccctctacggctggacccgcaactccctcatcgagtactacgtggtggactcctggggcacctaccgcccgaccggcacctacaagggcaccgtgacctccgacggcggcacctacgacatctacaccaccacccgcaccaacgccccgtccatcgacggcaacaacaccaccttcacccagttctggtccgtgcgccagtccaagcgcccgatcggcaccaacaacaccatcaccttctccaaccacgtgaacgcctggaagtccaagggcatgaacctcggctcctcctggtcctaccaggtgctcgccaccgagggctaccagtcctccggctactccaacgtgaccgtgtggtga

SEQ ID NO: 66 - protein, 186 aa, Artificial Sequence; identical to SEQ ID NO: 64
  Synthetic Construct

SEQ ID NO: 67 - DNA, 2071 bp, Oryza sativa
  misc_feature (1)..(2071): Promoter
tccatgctgtcctactacttgcttcatccccttctacattttgttctggtttttggcctgcatttcggatcatgatgtatgtgatttccaatctgctgcaatatgaatggagactctgtgctaaccatcaacaacatgaaatgcttatgaggcctttgctgagcagccaatcttgcctgtgtttatgtcttcacaggccgaattcctctgttttgtttttcaccctcaatatttggaaacatttatctaggttgtttgtgtccaggcctataaatcatacatgatgttgtcgtattggatgtgaatgtggtggcgtgttcagtgccttggatttgagtttgatgagagttgcttctgggtcaccactcaccattatcgatgctcctcttcagcataaggtaaaagtcttccctgtttacgttattttacccactatggttgcttgggttggttttttcctgattgcttatgccatggaaagtcatttgatatgttgaacttgaattaactgtagaattgtatacatgttccatttgtgttgtacttccttcttttctattagtagcctcagatgagtgtgaaaaaaacagattatataacttgccctataaatcatttgaaaaaaatattgtacagtgagaaattgatatatagtgaatttttaagagcatgttttcctaaagaagtatatattttctatgtacaaaggccattgaagtaattgtagatacaggataatgtagactttttggacttacactgctacctttaagtaacaatcatgagcaatagtgttgcaatgatatttaggctgcattcgtttactctcttgatttccatgagcacgcttcccaaactgttaaactctgtgttttttgccaaaaaaaaatgcataggaaagttgcttttaaaaaatcatatcaatccattttttaagttatagctaatacttaattaatcatgcgctaataagtcactctgtttttcgtactagagagattgttttgaaccagcactcaagaacacagccttaacccagccaaataatgctacaacctaccagtccacacctcttgtaaagcatttgttgcatggaaaagctaagatgacagcaacctgttcaggaaaacaactgacaaggtcatagggagagggagcttttggaaaggtgccgtgcagttcaaacaattagttagcagtagggtgttggtttttgctcacagcaataagaagttaatcatggtgtaggcaacccaaataaaacaccaaaatatgcacaaggcagtttgttgtattctgtagtacagacaaaactaaaagtaatgaaagaagatgtggtgttagaaaaggaaacaatatcatgagtaatgtgtgggcattatgggaccacgaaataaaaagaacattttgatgagtcgtgtatcctcgatgagcctcaaaagttctctcaccccggataagaaacccttaagcaatgtgcaaagtttgcattctccactgacataatgcaaaataagatatcatcgatgacatagcaactcatgcatcatatcatgcctctctcaacctattcattcctactcatctacataagtatcttcagctaaatgttagaacataaacccataagtcacgtttgatgagtattaggcgtgacacatgacaaatcacagactcaagcaagataaagcaaaatgatgtgtacataaaactccagagctatatgtcatattgcaaaaagaggagagcttataagacaaggcatgactcacaaaaattcatttgcctttcgtgtcaaaaagaggagggctttacattatccatgtcatattgcaaaagaaagagagaaagaacaacacaatgctgcgtcaattatacatatctgtatgtccatcattattcatccacctttcgtgtaccacacttcatatatcatgagtcacttcatgtctggacattaacaaactctatcttaacatttagatgcaagagcctttatctcactataaatgcacgatgatttctcattgtttctcacaaaaagcattcagttcattagtcctacaacaac

SEQ ID NO: 68 - protein, 79 aa, Zea mays
  SIGNAL (1)..(79): Maize waxy signal sequence.
MLAALATSQLVATRAGLGVPDASTFRRGAAQGLRGARASAAADTLSMRTSARAAPRHQHQQARRGARFPSLVVCASAGA

SEQ ID NO: 69 - DNA, 1005 bp, Artificial Sequence
  Synthetic Bromelain Sequence
atggcctggaaggtgcaggtggtgttcctcttcctcttcctctgcgtgatgtgggcctccccgtccgccgcctccgcggacgagccgtccgacccgatgatgaagcgcttcgaggagtggatggtggagtacggccgcgtgtacaaggacaacgacgagaagatgcgccgcttccagatcttcaagaacaacgtgaaccacatcgagaccttcaactcccgcaacgagaactcctacaccctcggcatcaaccagttcaccgacatgaccaacaacgagttcatcgcccagtacaccggcggcatctcccgcccgctcaacatcgagcgcgagccggtggtgtccttcgacgacgtggacatctccgccgtgccgcagtccatcgactggcgcgactacggcgccgtgacctccgtgaagaaccagaacccgtgcggcgcctgctgggccttcgccgccatcgccaccgtggagtccatctacaagatcaagaagggcatcctcgagccgctctccgagcagcaggtgctcgactgcgccaagggctacggctgcaagggcggctgggagttccgcgccttcgagttcatcatctccaacaagggcgtggcctccggcgccatctacccgtacaaggccgccaagggcacctgcaagaccgacggcgtgccgaactccgcctacatcaccggctacgcccgcgtgccgcgcaacaacgagtcctccatgatgtacgccgtgtccaagcagccgatcaccgtggccgtggacgccaacgccaacttccagtactacaagtccggcgtgttcaacggcccgtgcggcacctccctcaaccacgccgtgaccgccatcggctacggccaggactccatcatctacccgaagaagtggggcgccaagtggggcgaggccggctacatccgcatggcccgcgacgtgtcctcctcctccggcatctgcggcatcgccatcgacccgctctacccgaccctcgaggagtag

SEQ ID NO: 70 - protein, 334 aa, Artificial Sequence
  Synthetic Construct
MAWKVQVVFLFLFLCVMWASPSAASADEPSDPMMKRFEEWMVEYGRVYKDNDEKMRRFQIFKNNVNHIETFNSRNENSYTLGINQFTDMTNNEFIAQYTGGISRPLNIEREPVVSFDDVDISAVPQSIDWRDYGAVTSVKNQNPCGACWAFAAIATVESIYKIKKGILEPLSEQQVLDCAKGYGCKGGWEFRAFEFIISNKGVASGAIYPYKAAKGTCKTDGVPNSAYITGYARVPRNNESSMMYAVSKQPITVAVDANANFQYYKSGVFNGPCGTSLNHAVTAIGYGQDSIIYPKKWGAKWGEAGYIRMARDVSSSSGICGIAIDPLYPTLEE

SEQ ID NO: 71 - DNA, 78 bp, Artificial Sequence
  Bromealin signal sequence
atggcctggaaggtgcaggtggtgttcctcttcctcttcctctgcgtgatgtgggcctccccgtccgccgcctccgcc

SEQ ID NO: 72 - protein, 26 aa, Artificial Sequence
  Bromealin signal peptide
MAWKVQVVFLFLFLCVMWASPSAASA

SEQ ID NO: 73 - DNA, 1050 bp, Artificial Sequence
  pSYN11000
atggcctggaaggtgcaggtggtgttcctcttcctcttcctctgcgtgatgtgggcctccccgtccgccgcctccgcggacgagccgtccgacccgatgatgaagcgcttcgaggagtggatggtggagtacggccgcgtgtacaaggacaacgacgagaagatgcgccgcttccagatcttcaagaacaacgtgaaccacatcgagaccttcaactcccgcaacgagaactcctacaccctcggcatcaaccagttcaccgacatgaccaacaacgagttcatcgcccagtacaccggcggcatctcccgcccgctcaacatcgagcgcgagccggtggtgtccttcgacgacgtggacatctccgccgtgccgcagtccatcgactggcgcgactacggcgccgtgacctccgtgaagaaccagaacccgtgcggcgcctgctgggccttcgccgccatcgccaccgtggagtccatctacaagatcaagaagggcatcctcgagccgctctccgagcagcaggtgctcgactgcgccaagggctacggctgcaagggcggctgggagttccgcgccttcgagttcatcatctccaacaagggcgtggcctccggcgccatctacccgtacaaggccgccaagggcacctgcaagaccgacggcgtgccgaactccgcctacatcaccggctacgcccgcgtgccgcgcaacaacgagtcctccatgatgtacgccgtgtccaagcagccgatcaccgtggccgtggacgccaacgccaacttccagtactacaagtccggcgtgttcaacggcccgtgcggcacctccctcaaccacgccgtgaccgccatcggctacggccaggactccatcatctacccgaagaagtggggcgccaagtggggcgaggccggctacatccgcatggcccgcgacgtgtcctcctcctccggcatctgcggcatcgccatcgacccgctctacccgaccctcgaggaggtgttcgccgaggccatcgccgccaactccaccctcgtggccgagtag

SEQ ID NO: 74 - DNA, 1067 bp, Artificial Sequence
  pSYN11589
tggcctggaaggtgcaggtggtgttcctcttcctcttcctctgcgtgatgtgggcctccccgtccgccgcctccgcctcctcctcctccttcgccgactccaacccgatccgcccggtgaccgaccgcgccgcctccaccgacgagccgtccgacccgatgatgaagcgcttcgaggagtggatggtggagtacggccgcgtgtacaaggacaacgacgagaagatgcgccgcttccagatcttcaagaacaacgtgaaccacatcgagaccttcaactcccgcaacgagaactcctacaccctcggcatcaaccagttcaccgacatgaccaacaacgagttcatcgcccagtacaccggcggcatctcccgcccgctcaacatcgagcgcgagccggtggtgtccttcgacgacgtggacatctccgccgtgccgcagtccatcgactggcgcgactacggcgccgtgacctccgtgaagaaccagaacccgtgcggcgcctgctgggccttcgccgccatcgccaccgtggagtccatctacaagatcaagaagggcatcctcgagccgctctccgagcagcaggtgctcgactgcgccaagggctacggctgcaagggcggctgggagttccgcgccttcgagttcatcatctccaacaagggcgtggcctccggcgccatctacccgtacaaggccgccaagggcacctgcaagaccgacggcgtgccgaactccgcctacatcaccggctacgcccgcgtgccgcgcaacaacgagtcctccatgatgtacgccgtgtccaagcagccgatcaccgtggccgtggacgccaacgccaacttccagtactacaagtccggcgtgttcaacggcccgtgcggcacctccctcaaccacgccgtgaccgccatcggctacggccaggactccatcatctacccgaagaagtggggcgccaagtggggcgaggccggctacatccgcatggcccgcgacgtgtcctcctcctccggcatctgcggcatcgccatcgacccgctctacccgaccctcgaggagtag

SEQ ID NO: 75 - DNA, 1023 bp, Artificial Sequence
  pSYN11587 Sequence
atggcctggaaggtgcaggtggtgttcctcttcctcttcctctgcgtgatgtgggcctccccgtccgccgcctccgcggacgagccgtccgacccgatgatgaagcgcttcgaggagtggatggtggagtacggccgcgtgtacaaggacaacgacgagaagatgcgccgcttccagatcttcaagaacaacgtgaaccacatcgagaccttcaactcccgcaacgagaactcctacaccctcggcatcaaccagttcaccgacatgaccaacaacgagttcatcgcccagtacaccggcggcatctcccgcccgctcaacatcgagcgcgagccggtggtgtccttcgacgacgtggacatctccgccgtgccgcagtccatcgactggcgcgactacggcgccgtgacctccgtgaagaaccagaacccgtgcggcgcctgctgggccttcgccgccatcgccaccgtggagtccatctacaagatcaagaagggcatcctcgagccgctctccgagcagcaggtgctcgactgcgccaagggctacggctgcaagggcggctgggagttccgcgccttcgagttcatcatctccaacaagggcgtggcctccggcgccatctacccgtacaaggccgccaagggcacctgcaagaccgacggcgtgccgaactccgcctacatcaccggctacgcccgcgtgccgcgcaacaacgagtcctccatgatgtacgccgtgtccaagcagccgatcaccgtggccgtggacgccaacgccaacttccagtactacaagtccggcgtgttcaacggcccgtgcggcacctccctcaaccacgccgtgaccgccatcggctacggccaggactccatcatctacccgaagaagtggggcgccaagtggggcgaggccggctacatccgcatggcccgcgacgtgtcctcctcctccggcatctgcggcatcgccatcgacccgctctacccgaccctcgaggagtccgagaaggacgagctgtag

SEQ ID NO: 76 - DNA, 990 bp, Artificial Sequence
  pSYN12169 Sequence
atgagggtgttgctcgttgccctcgctctcctggctctcgctgcgagcgccacctccatggcggacgagccgtccgacccgatgatgaagcgcttcgaggagtggatggtggagtacggccgcgtgtacaaggacaacgacgagaagatgcgccgcttccagatcttcaagaacaacgtgaaccacatcgagaccttcaactcccgcaacgagaactcctacaccctcggcatcaaccagttcaccgacatgaccaacaacgagttcatcgcccagtacaccggcggcatctcccgcccgctcaacatcgagcgcgagccggtggtgtccttcgacgacgtggacatctccgccgtgccgcagtccatcgactggcgcgactacggcgccgtgacctccgtgaagaaccagaacccgtgcggcgcctgctgggccttcgccgccatcgccaccgtggagtccatctacaagatcaagaagggcatcctcgagccgctctccgagcagcaggtgctcgactgcgccaagggctacggctgcaagggcggctgggagttccgcgccttcgagttcatcatctccaacaagggcgtggcctccggcgccatctacccgtacaaggccgccaagggcacctgcaagaccgacggcgtgccgaactccgcctacatcaccggctacgcccgcgtgccgcgcaacaacgagtcctccatgatgtacgccgtgtccaagcagccgatcaccgtggccgtggacgccaacgccaacttccagtactacaagtccggcgtgttcaacggcccgtgcggcacctccctcaaccacgccgtgaccgccatcggctacggccaggactccatcatctacccgaagaagtggggcgccaagtggggcgaggccggctacatccgcatggcccgcgacgtgtcctcctcctccggcatctgcggcatcgccatcgacccgctctacccgaccctcgaggagtag

SEQ ID NO: 77 - DNA, 1170 bp, Artificial Sequence
  pSYN12575 Sequence
atgctggcggctctggccacgtcgcagctcgtcgcaacgcgcgccggcctgggcgtcccggacgcgtccacgttccgccgcggcgccgcgcagggcctgaggggggcccgggcgtcggcggcggcggacacgctcagcatgcggaccagcgcgcgcgcggcgcccaggcaccagcaccagcaggcgcgccgcggggccaggttcccgtcgctcgtcgtgtgcgccagcgccggcgccatggcggacgagccgtccgacccgatgatgaagcgcttcgaggagtggatggtggagtacggccgcgtgtacaaggacaacgacgagaagatgcgccgcttccagatcttcaagaacaacgtgaaccacatcgagaccttcaactcccgcaacgagaactcctacaccctcggcatcaaccagttcaccgacatgaccaacaacgagttcatcgcccagtacaccggcggcatctcccgcccgctcaacatcgagcgcgagccggtggtgtccttcgacgacgtggacatctccgccgtgccgcagtccatcgactggcgcgactacggcgccgtgacctccgtgaagaaccagaacccgtgcggcgcctgctgggccttcgccgccatcgccaccgtggagtccatctacaagatcaagaagggcatcctcgagccgctctccgagcagcaggtgctcgactgcgccaagggctacggctgcaagggcggctgggagttccgcgccttcgagttcatcatctccaacaagggcgtggcctccggcgccatctacccgtacaaggccgccaagggcacctgcaagaccgacggcgtgccgaactccgcctacatcaccggctacgcccgcgtgccgcgcaacaacgagtcctccatgatgtacgccgtgtccaagcagccgatcaccgtggccgtggacgccaacgccaacttccagtactacaagtccggcgtgttcaacggcccgtgcggcacctccctcaaccacgccgtgaccgccatcggctacggccaggactccatcatctacccgaagaagtggggcgccaagtggggcgaggccggctacatccgcatggcccgcgacgtgtcctcctcctccggcatctgcggcatcgccatcgacccgctctacccgaccctcgaggagtag

SEQ ID NO: 78 - DNA, 1068 bp, Artificial Sequence
  pSM270 Sequence
atggcctggaaggtgcaggtggtgttcctcttcctcttcctctgcgtgatgtgggcctccccgtccgccgcctccgcctcctcctcctccttcgccgactccaacccgatccgcccggtgaccgaccgcgccgcctccaccgacgagccgtccgacccgatgatgaagcgcttcgaggagtggatggtggagtacggccgcgtgtacaaggacaacgacgagaagatgcgccgcttccagatcttcaagaacaacgtgaaccacatcgagaccttcaactcccgcaacgagaactcctacaccctcggcatcaaccagttcaccgacatgaccaacaacgagttcatcgcccagtacaccggcggcatctcccgcccgctcaacatcgagcgcgagccggtggtgtccttcgacgacgtggacatctccgccgtgccgcagtccatcgactggcgcgactacggcgccgtgacctccgtgaagaaccagaacccgtgcggcgcctgctgggccttcgccgccatcgccaccgtggagtccatctacaagatcaagaagggcatcctcgagccgctctccgagcagcaggtgctcgactgcgccaagggctacggctgcaagggcggctgggagttccgcgccttcgagttcatcatctccaacaagggcgtggcctccggcgccatctacccgtacaaggccgccaagggcacctgcaagaccgacggcgtgccgaactccgcctacatcaccggctacgcccgcgtgccgcgcaacaacgagtcctccatgatgtacgccgtgtccaagcagccgatcaccgtggccgtggacgccaacgccaacttccagtactacaagtccggcgtgttcaacggcccgtgcggcacctccctcaaccacgccgtgaccgccatcggctacggccaggactccatcatctacccgaagaagtggggcgccaagtggggcgaggccggctacatccgcatggcccgcgacgtgtcctcctcctccggcatctgcggcatcgccatcgacccgctctacccgaccctcgaggagtag

SEQ ID NO: 79 - DNA, 1497 bp, Trichoderma reesei
  CDS (1)..(1497): Trichoderma reesei cellobiohyrodlase I
atgcagtcggcgtgtactctccaatcggagactcacccgcctctgacatggcagaaatgctcgtctggtggcacgtgcactcaacagacaggctccgtggtcatcgacgccaactggcgctggactcacgctacgaacagcagcacgaactgctacgatggcaacacttggagctcgaccctatgtcctgacaacgagacctgcgcgaagaactgctgtctggacggtgccgcctacgcgtccacgtacggagttaccacgagcggtaacagcctctccattggctttgtcacccagtctgcgcagaagaacgttggcgctcgcctttaccttatggcgagcgacacgacctaccaggaattcaccctgcttggcaacgagttctctttcgatgttgatgtttcgcagctgccgtgcggcttgaacggagctctctacttcgtgtccatggacgcggatggtggcgtgagcaagtatcccaccaacaccgctggcgccaagtacggcacggggtactgtgacagccagtgtccccgcgatctgaagttcatcaatggccaggccaacgttgagggctgggagccgtcatccaacaacgcgaacacgggcattggaggacacggaagctgctgctctgagatggatatctgggaggccaactccatctccgaggctcttaccccccacccttgcacgactgtcggccaggagatctgcgagggtgatgggtgcggcggaacttactccgataacagatatggcggcacttgcgatcccgatggctgcgactggaacccataccgcctgggcaacaccagcttctacggccctggctctagctttaccctcgataccaccaagaaattgaccgttgtcacccagttcgagacgtcgggtgccatcaaccgatactatgtccagaatggcgtcactttccagcagcccaacgccgagcttggtagttactctggcaacgagctcaacgatgattactgcacagctgaggaggcagaattcggcggatcctctttctcagacaagggcggcctgactcagttcaagaaggctacctctggcggcatggttctggtcatgagtctgtgggatgattactacgccaacatgctgtggctggactccacctacccgacaaacgagacctcctccacacccggtgccgtgcgcggaagctgctccaccagctccggtgtccctgctcaggtcgaatctcagtctcccaacgccaaggtcaccttctccaacatcaagttcggacccattggcagcaccggcaaccctagcggcggcaaccctcccggcggaaacccgcctggcaccaccaccacccgccgcccagccactaccactggaagctctcccggacctacccagtctcactacggccagtgcggcggtattggctacagcggccccacggtctgcgccagcggcacaacttgccaggtcctgaacccttactactctcagtgcctgtaa

SEQ ID NO: 80 - protein, 498 aa, Trichoderma reesei
MQSACTLQSETHPPLTWQKCSSGGTCTQQTGSVVIDANWRWTHATNSSTNCYDGNTWSSTLCPDNETCAKNCCLDGAAYASTYGVTTSGNSLSIGFVTQSAQKNVGARLYLMASDTTYQEFTLLGNEFSFDVDVSQLPCGLNGALYFVSMDADGGVSKYPTNTAGAKYGTGYCDSQCPRDLKFINGQANVEGWEPSSNNANTGIGGHGSCCSEMDIWEANSISEALTPHPCTTVGQEICEGDGCGGTYSDNRYGGTCDPDGCDWNPYRLGNTSFYGPGSSFTLDTTKKLTVVTQFETSGAINRYYVQNGVTFQQPNAELGSYSGNELNDDYCTAEEAEFGGSSFSDKGGLTQFKKATSGGMVLVMSLWDDYYANMLWLDSTYPTNETSSTPGAVRGSCSTSSGVPAQVESQSPNAKVTFSNIKFGPIGSTGNPSGGNPPGGNPPGTTTTRRPATTTGSSPGPTQSHYGQCGGIGYSGPTVCASGTTCQVLNPYYSQCL

SEQ ID NO: 81 - DNA, 1365 bp, Trichoderma reesei
  CDS (1)..(1365): trichoderma reesei cellobiohydrolase II
atggtgcctctagaggagcggcaagcttgctcaagcgtctggggccaatgtggtggccagaattggtcgggtccgacttgctgtgcttccggaagcacatgcgtctactccaacgactattactcccagtgtcttcccggcgctgcaagctcaagctcgtccacgcgcgccgcgtcgacgacttcacgagtatcccccacaacatcccggtcgagctccgcgacgcctccacctggttctaccactaccagagtacctccagtcggatcgggaaccgctacgtattcaggcaacccttttgttggggtcactccttgggccaatgcatattacgcctctgaagttagcagcctcgctattcctagcttgactggagccatggccactgctgcagcagctgtcgcaaaggttccctcttttatgtggctagatactcttgacaagacccctctcatggagcaaaccttggccgacatccgcaccgccaacaagaatggcggtaactatgccggacagtttgtggtgtatgacttgccggatcgcgattgcgctgcccttgcctcgaatggcgaatactctattgccgatggtggcgtcgccaaatataagaactatatcgacaccattcgtcaaattgtcgtggaatattccgatatccggaccctcctggttattgagcctgactctcttgccaacctggtgaccaacctcggtactccaaagtgtgccaatgctcagtcagcctaccttgagtgcatcaactacgccgtcacacagctgaaccttccaaatgttgcgatgtatttggacgctggccatgcaggatggcttggctggccggcaaaccaagacccggccgctcagctatttgcaaatgtttacaagaatgcatcgtctccgagagctcttcgcggattggcaaccaatgtcgccaactacaacgggtggaacattaccagccccccatcgtacacgcaaggcaacgctgtctacaacgagaagctgtacatccacgctattggacctcttcttgccaatcacggctggtccaacgccttcttcatcactgatcaaggtcgatcgggaaagcagcctaccggacagcaacagtggggagactggtgcaatgtgatcggcaccggatttggtattcgcccatccgcaaacactggggactcgttgctggattcgtttgtctgggtcaagccaggcggcgagtgtgacggcaccagcgacagcagtgcgccacgatttgactcccactgtgcgctcccagatgccttgcaaccggcgcctcaagctggtgcttggttccaagcctactttgtgcagcttctcacaaacgcaaacccatcgttcctgtag

SEQ ID NO: 82 - protein, 454 aa, Trichoderma reesei
MVPLEERQACSSVWGQCGGQNWSGPTCCASGSTCVYSNDYYSQCLPGAASSSSSTRAASTTSRVSPTTSRSSSATPPPGSTTTRVPPVGSGTATYSGNPFVGVTPWANAYYASEVSSLAIPSLTGAMATAAAAVAKVPSFMWLDTLDKTPLMEQTLADIRTANKNGGNYAGQFVVYDLPDRDCAALASNGEYSIADGGVAKYKNYIDTIRQIVVEYSDIRTLLVIEPDSLANLVTNLGTPKCANAQSAYLECINYAVTQLNLPNVAMYLDAGHAGWLGWPANQDPAAQLFANVYKNASSPRALRGLATNVANYNGWNITSPPSYTQGNAVYNEKLYIHAIGPLLANHGWSNAFFITDQGRSGKQPTGQQQWGDWCNVIGTGFGIRPSANTGDSLLDSFVWVKPGGECDGTSDSSAPRFDSHCALPDALQPAPQAGAWFQAYFVQLLTNANPSFL

SEQ ID NO: 83 - DNA, 1317 bp, Trichoderma reesei
  CDS (1)..(1317): Trichoderma reesei endoglucanase I
atgcagcaaccgggaaccagcacccccgaggtccatcccaagttgacaacctacaagtgcacaaagtccggggggtgcgtggcccaggacacctcggtggtccttgactggaactaccgctggatgcacgacgcaaactacaactcgtgcaccgtcaacggcggcgtcaacaccacgctctgccctgacgaggcgacctgtggcaagaactgcttcatcgagggcgtcgactacgccgcctcgggcgtcacgacctcgggcagcagcctcaccatgaaccagtacatgcccagcagctctggcggctacagcagcgtctctcctcggctgtatctcctggactctgacggtgagtacgtgatgctgaagctcaacggccaggagctgagcttcgacgtcgacctctctgctctgccgtgtggagagaacggctcgctctacctgtctcagatggacgagaacgggggcgccaaccagtataacacggccggtgccaactacgggagcggctactgcgatgctcagtgccccgtccagacatggaggaacggcaccctcaacactagccaccagggcttctgctgcaacgagatggatatcctggagggcaactcgagggcgaatgccttgacccctcactcttgcacggccacggcctgcgactctgccggttgcggcttcaacccctatggcagcggctacaaaagctactacggccccggagataccgttgacacctccaagaccttcaccatcatcacccagttcaacacggacaacggctcgccctcgggcaaccttgtgagcatcacccgcaagtaccagcaaaacggcgtcgacatccccagcgcccagcccggcggcgacaccatctcgtcctgcccgtccgcctcagcctacggcggcctcgccaccatgggcaaggccctgagcagcggcatggtgctcgtgttcagcatttggaacgacaacagccagtacatgaactggctcgacagcggcaacgccggcccctgcagcagcaccgagggcaacccatccaacaccctggccaacaaccccaacacgcacgtcgtcttctccaacatccgctggggagacattgggtctactacgaactcgactgcgcccccgcccccgcctgcgtccagcacgacgttttcgactacacggaggagctcgacgacttcgagcagcccgagctgcacgcagactcactgggggcagtgcggtggcattgggtacagcgggtgcaagacgtgcacgtcgggcactacgtgccagtatagcaacgactactactcgcaatgcctttag

SEQ ID NO: 84 - protein, 438 aa, Trichoderma reesei
MQQPGTSTPEVHPKLTTYKCTKSGGCVAQDTSVVLDWNYRWMHDANYNSCTVNGGVNTTLCPDEATCGKNCFIEGVDYAASGVTTSGSSLTMNQYMPSSSGGYSSVSPRLYLLDSDGEYVMLKLNGQELSFDVDLSALPCGENGSLYLSQMDENGGANQYNTAGANYGSGYCDAQCPVQTWRNGTLNTSHQGFCCNEMDILEGNSRANALTPHSCTATACDSAGCGFNPYGSGYKSYYGPGDTVDTSKTFTIITQFNTDNGSPSGNLVSITRKYQQNGVDIPSAQPGGDTISSCPSASAYGGLATMGKALSSGMVLVFSIWNDNSQYMNWLDSGNAGPCSSTEGNPSNTLANNPNTHVVFSNIRWGDIGSTTNSTAPPPPPASSTTFSTTRRSSTTSSSPSCTQTHWGQCGGIGYSGCKTCTSGTTCQYSNDYYSQCL

SEQ ID NO: 85 - DNA, 954 bp, Artificial Sequence
  6GP1
atgggcgtggacccgttcgagcgcaacaagatcctcggccgcggcatcaacatcggcaacgccctggaggccccgaacgagggcgactggggcgtggtgatcaaggacgagttcttcgacatcatcaaggaggccggcttctcccacgtgcgcatcccgatccgctggtccacccacgcctacgccttcccgccgtacaagatcatggaccgcttcttcaagcgcgtggacgaggtgatcaacggcgccctcaagcgcggcctcgccgtggccatcaacatccaccactacgaggagctcatgaacgacccggaggagcacaaggagcgcttcctcgccctctggaagcagatcgccgaccgctacaaggactacccggagaccctcttcttcgagatcctcaacgagccgcacggcaacctcaccccggagaagtggaacgagctgctcgaggaggccctcaaggtgatccgctccatcgacaagaagcacaccatcatcattggcaccgcagagtggggaggcatctccgccctcgagaagctctccgtgccgaagtgggagaagaattccatcgtgaccatccactactacaacccgttcgagttcacgcaccagggcgccgagtgggtggagggctccgagaagtggcttggccgcaagtggggctccccggacgaccagaagcacctcatcgaggagttcaacttcatcgaggagtggtccaagaagaacaagcgcccgatctacatcggcgagtttggcgcctaccgcaaggccgacctcgagtcccgcatcaagtggacctccttcgtggtgcgtgagatggagaagcgccgctggtcctgggcctactgggagttctgctccggcttcggcgtgtacgacaccctccgcaagacctggaacaaggacctcctcgaggccctcatcggcggcgactccatcgagtag

SEQ ID NO: 86 - protein, 317 aa, Artificial Sequence
  Synthetic Construct
MGVDPFERNKILGRGINIGNALEAPNEGDWGVVIKDEFFDIIKEAGFSHVRIPIRWSTHAYAFPPYKIMDRFFKRVDEVINGALKRGLAVAINIHHYEELMNDPEEHKERFLALWKQIADRYKDYPETLFFEILNEPHGNLTPEKWNELLEEALKVIRSIDKKHTIIIGTAEWGGISALEKLSVPKWEKNSIVTIHYYNPFEFTHQGAEWVEGSEKWLGRKWGSPDDQKHLIEEFNFIEEWSKKNKRPIYIGEFGAYRKADLESRIKWTSFVVREMEKRRWSWAYWEFCSGFGVYDTLRKTWNKDLLEALIGGDSIE

SEQ ID NO: 87 - DNA, 1248 bp, Hordeum vulgare
  CDS (1)..(1248): Barley AmyI amylase
atggcacaccaagtcctctttcaggggttcaactgggagtcgtggaagcagagcggcgggtggtacaacatgatgatgggcaaggtcgacgacatcgccgctgccggagtcacccacgtctggctgccaccgccgtcgcactccgtctccaacgaaggttacatgcctggtcggctgtacgacatcgacgcgtccaagtacggcaacgcggcggagctcaagtcgctcatcggcgcgctccacggcaagggcgtgcaggccatcgccgacatcgtcatcaaccaccgctgcgccgactacaaggatagccgcggcatctactgcatcttcgagggcggcacctccgacggccgcctcgactggggcccccacatgatctgtcgcgacgacaccaaatactccgatggcaccgcaaacctcgacaccggagccgacttcgccgccgcgcccgacatcgaccacctcaacgaccgggtccagcgcgagctcaaggagtggctcctctggctcaagagcgacctcggcttcgacgcgtggcgccttgacttcgccaggggctactcgccggagatggccaaggtgtacatcgacggcacatccccgagcctcgccgtggccgaggtgtgggacaatatggccaccggcggcgacggcaagcccaactacgaccaggacgcgcaccggcagaatctggtgaactgggtggacaaggtgggcggcgcggcctcggcaggcatggtgttcgacttcacgaccaaagggatactgaacgctgccgtggagggcgagctgtggaggctgatcgacccgcaggggaaggcccccggcgtgatgggatggtggccggccaaggccgtcaccttcgtcgacaaccacgatacaggctccacgcaggccatgtggccattcccctccgacaaggtcatgcagggctacgcgtacatcctcacccaccccggcatcccatgcatcttctacgaccatttcttcaactgggggtttaaggaccagatcgcggcgctggtggcgatcaggaagcgcaacggcatcacggcgacgagcgctctgaagatcctcatgcacgaaggagatgcctacgtcgccgagatagacggcaaggtggtggtgaagatcgggtccaggtacgacgtcggggcggtgatcccggccgggttcgtgacctcggcacacggcaacgactacgccgtctgggagaagaacggtgccgcggcaacactacaacggagc

SEQ ID NO: 88 - protein, 416 aa, Hordeum vulgare
MAHQVLFQGFNWESWKQSGGWYNMMMGKVDDIAAAGVTHVWLPPPSHSVSNEGYMPGRLYDIDASKYGNAAELKSLIGALHGKGVQAIADIVINHRCADYKDSRGIYCIFEGGTSDGRLDWGPHMICRDDTKYSDGTANLDTGADFAAAPDIDHLNDRVQRELKEWLLWLKSDLGFDAWRLDFARGYSPEMAKVYIDGTSPSLAVAEVWDNMATGGDGKPNYDQDAHRQNLVNWVDKVGGAASAGMVFDFTTKGILNAAVEGELWRLIDPQGKAPGVMGWWPAKAVTFVDNHDTGSTQAMWPFPSDKVMQGYAYILTHPGIPCIFYDHFFNWGFKDQIAALVAIRKRNGITATSALKILMHEGDAYVAEIDGKVVVKIGSRYDVGAVIPAGFVTSAHGNDYAVWEKNGAAATLQRS

SEQ ID NO: 89 - DNA, 1401 bp, Artificial Sequence
  Trichoderma reesei B-Glucosidase 2
atgttgcccaaggactttcagtgggggttcgccacggctgcctaccagatcgagggcgccgtcgaccaggacggccgcggccccagcatctgggacacgttctgcgcgcagcccggcaagatcgccgacggctcgtcgggcgtgacggcgtgcgactcgtacaaccgcacggccgaggacattgcgctgctgaagtcgctcggggccaagagctaccgcttctccatctcgtggtcgcgcatcatccccgagggcggccgcggcgatgccgtcaaccaggcgggcatcgaccactacgtcaagttcgtcgacgacctgctcgacgccggcatcacgcccttcatcaccctcttccactgggacctgcccgagggcctgcatcagcggtacggggggctgctgaaccgcaccgagttcccgctcgactttgaaaactacgcccgcgtcatgttcagggcgctgcccaaggtgcgcaactggatcaccttcaacgagccgctgtgctcggccatcccgggctacggctccggcaccttcgcccccggccggcagagcacctcggagccgtggaccgtcggccacaacatcctcgtcgcccacggccgcgccgtcaaggcgtaccgcgacgacttcaagcccgccagcggcgacggccagatcggcatcgtcctcaacggcgacttcacctacccctgggacgccgccgacccggccgacaaggaggcggccgagcggcgcctcgagttcttcacggcctggttcgcggaccccatctacttgggcgactacccggcgtcgatgcgcaagcagctgggcgaccggctgccgacctttacgcccgaggagcgcgccctcgtccacggctccaacgacttttacggcatgaaccactacacgtccaactacatccgccaccgcagctcgcccgcctccgccgacgacaccgtcggcaacgtcgacgtgctcttcaccaacaagcagggcaactgcatcggccccgagacgcagtccccctggctgcgcccctgtgccgccggcttccgcgacttcctggtgtggatcagcaagaggtacggctacccgcccatctacgtgacggagaacggcacgagcatcaagggcgagagcgacttgcccaaggagaagattctcgaagatgacttcagggtcaagtactataacgagtacatccgtgccatggttaccgccgtggagctggacggggtcaacgtcaaggggtactttgcctggtcgctcatggacaactttgagtgggcggacggctacgtgacgaggtttggggttacgtatgtggattatgagaatgggcagaagcggttccccaagaagagcgcaaagagcttgaagccgctgtttgacgagctgattgcggcggcgtga

SEQ ID NO: 90 - protein, 466 aa, Artificial Sequence
  Synthetic Construct
MLPKDFQWGFATAAYQIEGAVDQDGRGPSIWDTFCAQPGKIADGSSGVTACDSYNRTAEDIALLKSLGAKSYRFSISWSRIIPEGGRGDAVNQAGIDHYVKFVDDLLDAGITPFITLFHWDLPEGLHQRYGGLLNRTEFPLDFENYARVMFRALPKVRNWITFNEPLCSAIPGYGSGTFAPGRQSTSEPWTVGHNILVAHGRAVKAYRDDFKPASGDGQIGIVLNGDFTYPWDAADPADKEAAERRLEFFTAWFADPIYLGDYPASMRKQLGDRLPTFTPEERALVHGSNDFYGMNHYTSNYIRHRSSPASADDTVGNVDVLFTNKQGNCIGPETQSPWLRPCAAGFRDFLVWISKRYGYPPIYVTENGTSIKGESDLPKEKILEDDFRVKYYNEYIRAMVTAVELDGVNVKGYFAWSLMDNFEWADGYVTRFGVTYVDYENGQKRFPKKSAKSLKPLFDELIAAA

SEQ ID NO: 91 - DNA, 2103 bp, Artificial Sequence
  Trichoderma reesei B-Glucosidase D
atgattctcggctgtgaaagcacaggtgtcatctctgccgtcaaacactttgtcgccaacgaccaggagcacgagcggcgagcggtcgactgtctcatcacccagcgggctctccgggaggtctatctgcgacccttccagatcgtagcccgagatgcaaggcccggcgcattgatgacatcctacaacaaggtcaatggcaagcacgtcgctgacagcgccgagttccttcagggcattctccggactgagtggaattgggaccctctcattgtcagcgactggtacggcacctacaccactattgatgccatcaaagccggccttgatctcgagatgccgggcgtttcacgatatcgcggcaaatacatcgagtctgctctgcaggcccgtttgctgaagcagtccactatcgatgagcgcgctcgccgcgtgctcaggttcgcccagaaggccagccatctcaaggtctccgaggtagagcaaggccgtgacttcccagaggatcgcgtcctcaaccgtcagatctgcggcagcagcattgtcctactgaagaatgagaactccatcttacctctccccaagtccgtcaagaaggtcgcccttgttggttcccacgtgcgtctaccggctatctcgggaggaggcagcgcctctcttgtcccttactatgccatatctctatacgatgccgtctctgaggtactagccggtgccacgatcacgcacgaggtcggtgcctatgcccaccaaatgctgcccgtcatcgacgcaatgatcagcaacgccgtaatccacttctacaacgaccccatcgatgtcaaagacagaaagctccttggcagtgagaacgtatcgtcgacatcgttccagctcatggattacaacaacatcccaacgctcaacaaggccatgttctggggtactctcgtgggcgagtttatccctaccgccacgggaatttgggaatttggcctcagtgtctttggcactgccgacctttatattgataatgagctcgtgattgaaaatacaacacatcagacgcgtggaaccgcctttttcggaaagggaacgacggaaaaagtcgctaccaggaggatggtggccggcagcacctacaagctgcgtctcgagtttgggtctgccaacacgaccaagatggagacgaccggtgttgtcaactttggcggcggtgccgtacacctgggtgcctgtctcaaggtcgacccacaggagatgattgcgcgggccgtcaaggccgcagccgatgccgactacaccatcatctgcacgggactcagcggcgagtgggagtctgagggttttgaccggcctcacatggacctgccccctggtgtggacaccatgatctcgcaagttcttgacgccgctcccaatgctgtagtcgtcaaccagtcaggcaccccagtgacaatgagctgggctcataaagcaaaggccattgtgcaggcttggtatggtggtaacgagacaggccacggaatctccgatgtgctctttggcaacgtcaacccgtcggggaaactctccctatcgtggccagtcgatgtgaagcacaacccagcatatctcaactacgccagcgttggtggacgggtcttgtatggcgaggatgtttacgttggctacaagttctacgacaaaacggagagggaggttctgtttccttttgggcatggcctgtcttacgctaccttcaagctcccagattctaccgtgaggacggtccccgaaaccttccacccggaccagcccacagtagccattgtcaagatcaagaacacgagcagtgtcccgggcgcccaggtcctgcagttatacatttcggccccaaactcgcctacacatcgcccggtcaaggagctgcacggattcgaaaaggtgtatcttgaagctggcgaggagaaggaggtacaaatacccattgaccagtacgctactagcttctgggacgagattgagagcatgtggaagagcgagaggggcatttatgatgtgcttgtaggattctcgagtcaggaaatctcgggcaaggggaagctgattgtgcctgaaacgcgattctggatggggctgtag

SEQ ID NO: 92 - protein, 700 aa, Artificial Sequence
  Synthetic Construct
MILGCESTGVISAVKHFVANDQEHERRAVDCLITQRALREVYLRPFQIVARDARPGALMTSYNKVNGKHVADSAEFLQGILRTEWNWDPLIVSDWYGTYTTIDAIKAGLDLEMPGVSRYRGKYIESALQARLLKQSTIDERARRVLRFAQKASHLKVSEVEQGRDFPEDRVLNRQICGSSIVLLKNENSILPLPKSVKKVALVGSHVRLPAISGGGSASLVPYYAISLYDAVSEVLAGATITHEVGAYAHQMLPVIDAMISNAVIHFYNDPIDVKDRKLLGSENVSSTSFQLMDYNNIPTLNKAMFWGTLVGEFIPTATGIWEFGLSVFGTADLYIDNELVIENTTHQTRGTAFFGKGTTEKVATRRMVAGSTYKLRLEFGSANTTKMETTGVVNFGGGAVHLGACLKVDPQEMIARAVKAAADADYTIICTGLSGEWESEGFDRPHMDLPPGVDTMISQVLDAAPNAVVVNQSGTPVTMSWAHKAKAIVQAWYGGNETGHGISDVLFGNVNPSGKLSLSWPVDVKHNPAYLNYASVGGRVLYGEDVYVGYKFYDKTEREVLFPFGHGLSYATFKLPDSTVRTVPETFHPDQPTVAIVKIKNTSSVPGAQVLQLYISAPNSPTHRPVKELHGFEKVYLEAGEEKEVQIPIDQYATSFWDEIESMWKSERGIYDVLVGFSSQEISGKGKLIVPETRFWMGL

SEQ ID NO: 93 - DNA, 1496 bp, Artificial Sequence
  Maize optimized CBHI
tgcagtccgcctgcaccctccagtccgagacccacccgccgctcacctggcagaagtgctcctccggcggcacctgcacccagcagaccggctccgtggtgatcgacgccaactggcgctggacccacgccaccaactcctccaccaactgctacgacggcaacacctggtcctccaccctctgcccggacaacgagacctgcgccaagaactgctgcctcgacggcgccgcctacgcctccacctacggcgtgaccacctccggcaactccctctccatcggcttcgtgacccagtccgcccagaagaacgtgggcgcccgcctctacctcatggcctccgacaccacctaccaggagttcaccctcctcggcaacgagttctccttcgacgtggacgtgtcccagctcccgtgcggcctcaacggcgccctctacttcgtgtccatggacgccgacggcggcgtgtccaagtacccgaccaacaccgccggcgccaagtacggcaccggctactgcgactcccagtgcccgcgcgacctcaagttcatcaacggccaggccaacgtggagggctgggagccgtcctccaacaacgccaacaccggcatcggcggccacggctcctgctgctccgagatggacatctgggaggccaactccatctccgaggccctcaccccgcacccgtgcaccaccgtgggccaggagatctgcgagggcgacggctgcggcggcacctactccgacaaccgctacggcggcacctgcgacccggacggctgcgactggaacccgtaccgcctcggcaacacctccttctacggcccgggctcctccttcaccctcgacaccaccaagaagctcaccgtggtgacccagttcgagacctccggcgccatcaaccgctactacgtgcagaacggcgtgaccttccagcagccgaacgccgagctcggctcctactccggcaacgagctcaacgacgactactgcaccgccgaggaggccgagttcggcggctcctccttctccgacaagggcggcctcacccagttcaagaaggccacctccggcggcatggtgctcgtgatgtccctctgggacgactactacgccaacatgctctggctcgactccacctacccgaccaacgagacctcctccaccccgggcgccgtgcgcggctcctgctccacctcctccggcgtgccggcccaggtggagtcccagtccccgaacgccaaggtgaccttctccaacatcaagttcggcccgatcggctccaccggcaacccgtccggcggcaacccgccgggcggcaacccgccgggcaccaccaccacccgccgcccggccaccaccaccggctcctccccgggcccgacccagtcccactacggccagtgcggcggcatcggctactccggcccgaccgtgtgcgcctccggcaccacctgccaggtgctcaacccgtactactcccagtgcctctag

SEQ ID NO: 94 - DNA, 1365 bp, Artificial Sequence
  Maize optimized CBHII
atggtgccgctcgaggagcgccaggcctgctcctccgtgtggggccagtgcggcggccagaactggtccggcccgacctgctgcgcctccggctccacctgcgtgtactccaacgactactactcccagtgcctcccgggcgccgcctcctcctcctcctccacccgcgccgcctccaccacctcccgcgtgtccccgaccacctcccgctcctcctccgccaccccgccgccgggctccaccaccacccgcgtgccgccggtgggctccggcaccgccacctactccggcaacccgttcgtgggcgtgaccccgtgggccaacgcctactacgcctccgaggtgtcctccctcgccatcccgtccctcaccggcgccatggccaccgccgccgccgccgtggccaaggtgccgtccttcatgtggctcgacaccctcgacaagaccccgctcatggagcagaccctcgccgacatccgcaccgccaacaagaacggcggcaactacgccggccagttcgtggtgtacgacctcccggaccgcgactgcgccgccctcgcctccaacggcgagtactccatcgccgacggcggcgtggccaagtacaagaactacatcgacaccatccgccagatcgtggtggagtactccgacatccgcaccctcctcgtgatcgagccggactccctcgccaacctcgtgaccaacctcggcaccccgaagtgcgccaacgcccagtccgcctacctcgagtgcatcaactacgccgtgacccagctcaacctcccgaacgtggccatgtacctcgacgccggccacgccggctggctcggctggccggccaaccaggacccggccgcccagctcttcgccaacgtgtacaagaacgcctcctccccgcgcgccctccgcggcctcgccaccaacgtggccaactacaacggctggaacatcacctccccgccgtcctacacccagggcaacgccgtgtacaacgagaagctctacatccacgccatcggcccgctcctcgccaaccacggctggtccaacgccttcttcatcaccgaccagggccgctccggcaagcagccgaccggccagcagcagtggggcgactggtgcaacgtgatcggcaccggcttcggcatccgcccgtccgccaacaccggcgactccctcctcgactccttcgtgtgggtgaagccgggcggcgagtgcgacggcacctccgactcctccgccccgcgcttcgactcccactgcgccctcccggacgccctccagccggccccgcaggccggcgcctggttccaggcctacttcgtgcagctcctcaccaacgccaacccgtccttcctctag

SEQ ID NO: 95 - DNA, 1317 bp, Artificial Sequence
  Maize optimized EGLI
atgcagcagccgggcacctccaccccggaggtgcacccgaagctcaccacctacaagtgcaccaagtccggcggctgcgtggcccaggacacctccgtggtgctcgactggaactaccgctggatgcacgacgccaactacaactcctgcaccgtgaacggcggcgtgaacaccaccctctgcccggacgaggccacctgcggcaagaactgcttcatcgagggcgtggactacgccgcctccggcgtgaccacctccggctcctccctcaccatgaaccagtacatgccgtcctcctccggcggctactcctccgtgtccccgcgcctctacctcctcgactccgacggcgagtacgtgatgctcaagctcaacggccaggagctctccttcgacgtggacctctccgccctcccgtgcggcgagaacggctccctctacctctcccagatggacgagaacggcggcgccaaccagtacaacaccgccggcgccaactacggctccggctactgcgacgcccagtgcccggtgcagacctggcgcaacggcaccctcaacacctcccaccagggcttctgctgcaacgagatggacatcctcgagggcaactcccgcgccaacgccctcaccccgcactcctgcaccgccaccgcctgcgactccgccggctgcggcttcaacccgtacggctccggctacaagtcctactacggcccgggcgacaccgtggacacctccaagaccttcaccatcatcacccagttcaacaccgacaacggctccccgtccggcaacctcgtgtccatcacccgcaagtaccagcagaacggcgtggacatcccgtccgcccagccgggcggcgacaccatctcctcctgcccgtccgcctccgcctacggcggcctcgccaccatgggcaaggccctctcctccggcatggtgctcgtgttctccatctggaacgacaactcccagtacatgaactggctcgactccggcaacgccggcccgtgctcctccaccgagggcaacccgtccaacaccctcgccaacaacccgaacacccacgtggtgttctccaacatccgctggggcgacatcggctccaccaccaactccaccgccccgccgccgccgccggcctcctccaccaccttctccaccacccgccgctcctccaccacctcctcctccccgtcctgcacccagacccactggggccagtgcggcggcatcggctactccggctgcaagacctgcacctccggcaccacctgccagtactccaacgactactactcccagtgcctctag

SEQ ID NO: 96 - DNA, 1401 bp, Artificial Sequence
  Maize optimized BGLII
atgctcccgaaggacttccagtggggcttcgccaccgccgcctaccagatcgagggcgccgtggaccaggacggccgcggcccgtccatctgggacaccttctgcgcccagccgggcaagatcgccgacggctcctccggcgtgaccgcctgcgactcctacaaccgcaccgccgaggacatcgccctcctcaagtccctcggcgccaagtcctaccgcttctccatctcctggtcccgcatcatcccggagggcggccgcggcgacgccgtgaaccaggccggcatcgaccactacgtgaagttcgtggacgacctcctcgacgccggcatcaccccgttcatcaccctcttccactgggacctcccggagggcctccaccagcgctacggcggcctcctcaaccgcaccgagttcccgctcgacttcgagaactacgcccgcgtgatgttccgcgccctcccgaaggtgcgcaactggatcaccttcaacgagccgctctgctccgccatcccgggctacggctccggcaccttcgccccgggccgccagtccacctccgagccgtggaccgtgggccacaacatcctcgtggcccacggccgcgccgtgaaggcctaccgcgacgacttcaagccggcctccggcgacggccagatcggcatcgtgctcaacggcgacttcacctacccgtgggacgccgccgacccggccgacaaggaggccgccgagcgccgcctcgagttcttcaccgcctggttcgccgacccgatctacctcggcgactacccggcctccatgcgcaagcagctcggcgaccgcctcccgaccttcaccccggaggagcgcgccctcgtgcacggctccaacgacttctacggcatgaaccactacacctccaactacatccgccaccgctcctccccggcctccgccgacgacaccgtgggcaacgtggacgtgctcttcaccaacaagcagggcaactgcatcggcccggagacccagtccccgtggctccgcccgtgcgccgccggcttccgcgacttcctcgtgtggatctccaagcgctacggctacccgccgatctacgtgaccgagaacggcacctccatcaagggcgagtccgacctcccgaaggagaagatcctcgaggacgacttccgcgtgaagtactacaacgagtacatccgcgccatggtgaccgccgtggagctcgacggcgtgaacgtgaagggctacttcgcctggtccctcatggacaacttcgagtgggccgacggctacgtgacccgcttcggcgtgacctacgtggactacgagaacggccagaagcgcttcccgaagaagtccgccaagtccctcaagccgctcttcgacgagctcatcgccgccgcctag

SEQ ID NO: 97 - DNA, 2103 bp, Artificial Sequence
  Maize optimized CEL3D
atgatcctcggctgcgagtccaccggcgtgatctccgccgtgaagcacttcgtggccaacgaccaggagcacgagcgccgcgccgtggactgcctcatcacccagcgcgccctccgcgaggtgtacctccgcccgttccagatcgtggcccgcgacgcccgcccgggcgccctcatgacctcctacaacaaggtgaacggcaagcacgtggccgactccgccgagttcctccagggcatcctccgcaccgagtggaactgggacccgctcatcgtgtccgactggtacggcacctacaccaccatcgacgccatcaaggccggcctcgacctcgagatgccgggcgtgtcccgctaccgcggcaagtacatcgagtccgccctccaggcccgcctcctcaagcagtccaccatcgacgagcgcgcccgccgcgtgctccgcttcgcccagaaggcctcccacctcaaggtgtccgaggtggagcagggccgcgacttcccggaggaccgcgtgctcaaccgccagatctgcggctcctccatcgtgctcctcaagaacgagaactccatcctcccgctcccgaagtccgtgaagaaggtggccctcgtgggctcccacgtgcgcctcccggccatctccggcggcggctccgcctccctcgtgccgtactacgccatctccctctacgacgccgtgtccgaggtgctcgccggcgccaccatcacccacgaggtgggcgcctacgcccaccagatgctcccggtgatcgacgccatgatctccaacgccgtgatccacttctacaacgacccgatcgacgtgaaggaccgcaagctcctcggctccgagaacgtgtcctccacctccttccagctcatggactacaacaacatcccgaccctcaacaaggccatgttctggggcaccctcgtgggcgagttcatcccgaccgccaccggcatctgggagttcggcctctccgtgttcggcaccgccgacctctacatcgacaacgagctcgtgatcgagaacaccacccaccagacccgcggcaccgccttcttcggcaagggcaccaccgagaaggtggccacccgccgcatggtggccggctccacctacaagctccgcctcgagttcggctccgccaacaccaccaagatggagaccaccggcgtggtgaacttcggcggcggcgccgtgcacctcggcgcctgcctcaaggtggacccgcaggagatgatcgcccgcgccgtgaaggccgccgccgacgccgactacaccatcatctgcaccggcctctccggcgagtgggagtccgagggcttcgaccgcccgcacatggacctcccgccgggcgtggacaccatgatctcccaggtgctcgacgccgccccgaacgccgtggtggtgaaccagtccggcaccccggtgaccatgtcctgggcccacaaggccaaggccatcgtgcaggcctggtacggcggcaacgagaccggccacggcatctccgacgtgctcttcggcaacgtgaacccgtccggcaagctctccctctcctggccggtggacgtgaagcacaacccggcctacctcaactacgcctccgtgggcggccgcgtgctctacggcgaggacgtgtacgtgggctacaagttctacgacaagaccgagcgcgaggtgctcttcccgttcggccacggcctctcctacgccaccttcaagctcccggactccaccgtgcgcaccgtgccggagaccttccacccggaccagccgaccgtggccatcgtgaagatcaagaacacctcctccgtgccgggcgcccaggtgctccagctctacatctccgccccgaactccccgacccaccgcccggtgaaggagctccacggcttcgagaaggtgtacctcgaggccggcgaggagaaggaggtgcagatcccgatcgaccagtacgccacctccttctgggacgagatcgagtccatgtggaagtccgagcgcggcatctacgacgtgctcgtgggcttctcctcccaggagatctccggcaagggcaagctcatcgtgccggagacccgcttctggatgggcctctag

SEQ ID NO: 98 - DNA, 420 bp, Zea mays
  Q protein promoter
gggctggtaaattacttgggagcaatggtatgcaaatcctttgcatgtacgcaaaactagctagttgtcacaagttgtatatcgattcgtcgcgtttcaacaactcatgcaacattacaaacaagtaacacaatattacaaagttagtttcatacaaagcaagaaaaggacaataatacttgacatgtaaagtgaagcttattatacttcctaatccaacacaaaacaaaaaaaagttgcacaaaggtccaaaaatccacatcaaccattaacctatacgtaaagtgagtgatgagtcacattatccaacaaatgtttatcaatgtggtatcatacaagcattgacatcccataaatgcaagaaattgtgccaacaaagctataagtaaccctcatatgtatttgcactcatgcatcaca

SEQ ID NO: 99 - DNA, 1188 bp, artificial sequence
  synthetic ferulic acid esterase
atggccgcctccctcccgaccatgccgccgtccggctacgaccaggtgcgcaacggcgtgccgcgcggccaggtggtgaacatctcctacttctccaccgccaccaactccacccgcccggcccgcgtgtacctcccgccgggctactccaaggacaagaagtactccgtgctctacctcctccacggcatcggcggctccgagaacgactggttcgagggcggcggccgcgccaacgtgatcgccgacaacctcatcgccgagggcaagatcaagccgctcatcatcgtgaccccgaacaccaacgccgccggcccgggcatcgccgacggctacgagaacttcaccaaggacctcctcaactccctcatcccgtacatcgagtccaactactccgtgtacaccgaccgcgagcaccgcgccatcgccggcctctctatgggcggcggccagtccttcaacatcggcctcaccaacctcgacaagttcgcctacatcggcccgatctccgccgccccgaacacctacccgaacgagcgcctcttcccggacggcggcaaggccgcccgcgagaagctcaagctcctcttcatcgcctgcggcaccaacgactccctcatcggcttcggccagcgcgtgcacgagtactgcgtggccaacaacatcaaccacgtgtactggctcatccagggcggcggccacgacttcaacgtgtggaagccgggcctctggaacttcctccagatggccgacgaggccggcctcacccgcgacggcaacaccccggtgccgaccccgtccccgaagccggccaacacccgcatcgaggccgaggactacgacggcatcaactcctcctccatcgagatcatcggcgtgccgccggagggcggccgcggcatcggctacatcacctccggcgactacctcgtgtacaagtccatcgacttcggcaacggcgccacctccttcaaggccaaggtggccaacgccaacacctccaacatcgagcttcgcctcaacggcccgaacggcaccctcatcggcaccctctccgtgaagtccaccggcgactggaacacctacgaggagcagacctgctccatctccaaggtgaccggcatcaacgacctctacctcgtgttcaagggcccggtgaacatcgactggttcaccttcggcgtgtag

SEQ ID NO: 100 - protein, 395 aa, artificial sequence
  synthetic ferulic acid esterase
MAASLPTMPPSGYDQVRNGVPRGQVVNISYFSTATNSTRPARVYLPPGYSKDKKYSVLYLLHGIGGSENDWFEGGGRANVIADNLIAEGKIKPLIIVTPNTNAAGPGIADGYENFTKDLLNSLIPYIESNYSVYTDREHRAIAGLSMGGGQSFNIGLTNLDKFAYIGPISAAPNTYPNERLFPDGGKAAREKLKLLFIACGTNDSLIGFGQRVHEYCVANNINHVYWLIQGGGHDFNVWKPGLWNFLQMADEAGLTRDGNTPVPTPSPKPANTRIEAEDYDGINSSSIEIIGVPPEGGRGIGYITSGDYLVYKSIDFGNGATSFKAKVANANTSNIELRLNGPNGTLIGTLSVKSTGDWNTYEEQTCSISKVTGINDLYLVFKGPVNIDWFTFGV

SEQ ID NO: 101 - DNA, 1188 bp, artificial sequence; identical to SEQ ID NO: 99
  plasmid 13036

SEQ ID NO: 102 - protein, 395 aa, artificial sequence; identical to SEQ ID NO: 100
  plasmid 13036

SEQ ID NO: 103 - DNA, 1245 bp, artificial sequence
  plasmid 13038
atgagggtgttgctcgttgccctcgctctcctggctctcgctgcgagcgccacctccatggccgcctccctcccgaccatgccgccgtccggctacgaccaggtgcgcaacggcgtgccgcgcggccaggtggtgaacatctcctacttctccaccgccaccaactccacccgcccggcccgcgtgtacctcccgccgggctactccaaggacaagaagtactccgtgctctacctcctccacggcatcggcggctccgagaacgactggttcgagggcggcggccgcgccaacgtgatcgccgacaacctcatcgccgagggcaagatcaagccgctcatcatcgtgaccccgaacaccaacgccgccggcccgggcatcgccgacggctacgagaacttcaccaaggacctcctcaactccctcatcccgtacatcgagtccaactactccgtgtacaccgaccgcgagcaccgcgccatcgccggcctctctatgggcggcggccagtccttcaacatcggcctcaccaacctcgacaagttcgcctacatcggcccgatctccgccgccccgaacacctacccgaacgagcgcctcttcccggacggcggcaaggccgcccgcgagaagctcaagctcctcttcatcgcctgcggcaccaacgactccctcatcggcttcggccagcgcgtgcacgagtactgcgtggccaacaacatcaaccacgtgtactggctcatccagggcggcggccacgacttcaacgtgtggaagccgggcctctggaacttcctccagatggccgacgaggccggcctcacccgcgacggcaacaccccggtgccgaccccgtccccgaagccggccaacacccgcatcgaggccgaggactacgacggcatcaactcctcctccatcgagatcatcggcgtgccgccggagggcggccgcggcatcggctacatcacctccggcgactacctcgtgtacaagtccatcgacttcggcaacggcgccacctccttcaaggccaaggtggccaacgccaacacctccaacatcgagcttcgcctcaacggcccgaacggcaccctcatcggcaccctctccgtgaagtccaccggcgactggaacacctacgaggagcagacctgctccatctccaaggtgaccggcatcaacgacctctacctcgtgttcaagggcccggtgaacatcgactggttcaccttcggcgtgtag

SEQ ID NO: 104 - protein, 414 aa, artificial sequence
  plasmid 13038 aa
MRVLLVALALLALAASATSMAASLPTMPPSGYDQVRNGVPRGQVVNISYFSTATNSTRPARVYLPPGYSKDKKYSVLYLLHGIGGSENDWFEGGGRANVIADNLIAEGKIKPLIIVTPNTNAAGPGIADGYENFTKDLLNSLIPYIESNYSVYTDREHRAIAGLSMGGGQSFNIGLTNLDKFAYIGPISAAPNTYPNERLFPDGGKAAREKLKLLFIACGTNDSLIGFGQRVHEYCVANNINHVYWLIQGGGHDFNVWKPGLWNFLQMADEAGLTRDGNTPVPTPSPKPANTRIEAEDYDGINSSSIEIIGVPPEGGRGIGYITSGDYLVYKSIDFGNGATSFKAKVANANTSNIELRLNGPNGTLIGTLSVKSTGDWNTYEEQTCSISKVTGINDLYLVFKGPVNIDWFTFGV

SEQ ID NO: 105 - DNA, 1425 bp, artificial sequence
  plasmid 13039
atgctggcggctctggccacgtcgcagctcgtcgcaacgcgcgccggcctgggcgtcccggacgcgtccacgttccgccgcggcgccgcgcagggcctgaggggggcccgggcgtcggcggcggcggacacgctcagcatgcggaccagcgcgcgcgcggcgcccaggcaccagcaccagcaggcgcgccgcggggccaggttcccgtcgctcgtcgtgtgcgccagcgccggcgccatggccgcctccctcccgaccatgccgccgtccggctacgaccaggtgcgcaacggcgtgccgcgcggccaggtggtgaacatctcctacttctccaccgccaccaactccacccgcccggcccgcgtgtacctcccgccgggctactccaaggacaagaagtactccgtgctctacctcctccacggcatcggcggctccgagaacgactggttcgagggcggcggccgcgccaacgtgatcgccgacaacctcatcgccgagggcaagatcaagccgctcatcatcgtgaccccgaacaccaacgccgccggcccgggcatcgccgacggctacgagaacttcaccaaggacctcctcaactccctcatcccgtacatcgagtccaactactccgtgtacaccgaccgcgagcaccgcgccatcgccggcctctctatgggcggcggccagtccttcaacatcggcctcaccaacctcgacaagttcgcctacatcggcccgatctccgccgccccgaacacctacccgaacgagcgcctcttcccggacggcggcaaggccgcccgcgagaagctcaagctcctcttcatcgcctgcggcaccaacgactccctcatcggcttcggccagcgcgtgcacgagtactgcgtggccaacaacatcaaccacgtgtactggctcatccagggcggcggccacgacttcaacgtgtggaagccgggcctctggaacttcctccagatggccgacgaggccggcctcacccgcgacggcaacaccccggtgccgaccccgtccccgaagccggccaacacccgcatcgaggccgaggactacgacggcatcaactcctcctccatcgagatcatcggcgtgccgccggagggcggccgcggcatcggctacatcacctccggcgactacctcgtgtacaagtccatcgacttcggcaacggcgccacctccttcaaggccaaggtggccaacgccaacacctccaacatcgagcttcgcctcaacggcccgaacggcaccctcatcggcaccctctccgtgaagtccaccggcgactggaacacctacgaggagcagacctgctccatctccaaggtgaccggcatcaacgacctctacctcgtgttcaagggcccggtgaacatcgactggttcaccttcggcgtgtag

SEQ ID NO: 106 - protein, 474 aa, artificial sequence
  plasmid 13039 aa
MLAALATSQLVATRAGLGVPDASTFRRGAAQGLRGARASAAADTLSMRTSARAAPRHQHQQARRGARFPSLVVCASAGAMAASLPTMPPSGYDQVRNGVPRGQVVNISYFSTATNSTRPARVYLPPGYSKDKKYSVLYLLHGIGGSENDWFEGGGRANVIADNLIAEGKIKPLIIVTPNTNAAGPGIADGYENFTKDLLNSLIPYIESNYSVYTDREHRAIAGLSMGGGQSFNIGLTNLDKFAYIGPISAAPNTYPNERLFPDGGKAAREKLKLLFIACGTNDSLIGFGQRVHEYCVANNINHVYWLIQGGGHDFNVWKPGLWNFLQMADEAGLTRDGNTPVPTPSPKPANTRIEAEDYDGINSSSIEIIGVPPEGGRGIGYITSGDYLVYKSIDFGNGATSFKAKVANANTSNIELRLNGPNGTLIGTLSVKSTGDWNTYEEQTCSISKVTGINDLYLVFKGPVNIDWFTFGV

SEQ ID NO: 107 - DNA, 1263 bp, artificial sequence
  plasmid 13347
atgagggtgttgctcgttgccctcgctctcctggctctcgctgcgagcgccacctccatggccgcctccctcccgaccatgccgccgtccggctacgaccaggtgcgcaacggcgtgccgcgcggccaggtggtgaacatctcctacttctccaccgccaccaactccacccgcccggcccgcgtgtacctcccgccgggctactccaaggacaagaagtactccgtgctctacctcctccacggcatcggcggctccgagaacgactggttcgagggcggcggccgcgccaacgtgatcgccgacaacctcatcgccgagggcaagatcaagccgctcatcatcgtgaccccgaacaccaacgccgccggcccgggcatcgccgacggctacgagaacttcaccaaggacctcctcaactccctcatcccgtacatcgagtccaactactccgtgtacaccgaccgcgagcaccgcgccatcgccggcctctctatgggcggcggccagtccttcaacatcggcctcaccaacctcgacaagttcgcctacatcggcccgatctccgccgccccgaacacctacccgaacgagcgcctcttcccggacggcggcaaggccgcccgcgagaagctcaagctcctcttcatcgcctgcggcaccaacgactccctcatcggcttcggccagcgcgtgcacgagtactgcgtggccaacaacatcaaccacgtgtactggctcatccagggcggcggccacgacttcaacgtgtggaagccgggcctctggaacttcctccagatggccgacgaggccggcctcacccgcgacggcaacaccccggtgccgaccccgtccccgaagccggccaacacccgcatcgaggccgaggactacgacggcatcaactcctcctccatcgagatcatcggcgtgccgccggagggcggccgcggcatcggctacatcacctccggcgactacctcgtgtacaagtccatcgacttcggcaacggcgccacctccttcaaggccaaggtggccaacgccaacacctccaacatcgagcttcgcctcaacggcccgaacggcaccctcatcggcaccctctccgtgaagtccaccggcgactggaacacctacgaggagcagacctgctccatctccaaggtgaccggcatcaacgacctctacctcgtgttcaagggcccggtgaacatcgactggttcaccttcggcgtgtccgagaaggacgaactctag

SEQ ID NO: 108 - protein, 420 aa, artificial sequence
  plasmid 13347
MRVLLVALALLALAASATSMAASLPTMPPSGYDQVRNGVPRGQVVNISYFSTATNSTRPARVYLPPGYSKDKKYSVLYLLHGIGGSENDWFEGGGRANVIADNLIAEGKIKPLIIVTPNTNAAGPGIADGYENFTKDLLNSLIPYIESNYSVYTDREHRAIAGLSMGGGQSFNIGLTNLDKFAYIGPISAAPNTYPNERLFPDGGKAAREKLKLLFIACGTNDSLIGFGQRVHEYCVANNINHVYWLIQGGGHDFNVWKPGLWNFLQMADEAGLTRDGNTPVPTPSPKPANTRIEAEDYDGINSSSIEIIGVPPEGGRGIGYITSGDYLVYKSIDFGNGATSFKAKVANANTSNIELRLNGPNGTLIGTLSVKSTGDWNTYEEQTCSISKVTGINDLYLVFKGPVNIDWFTFGVSEKDEL

SEQ ID NO: 109 - DNA, 1296 bp, artificial sequence
  plasmid 11267
atgagggtgttgctcgttgccctcgctctcctggctctcgctgcgagcgccaccagcgctgcgcagtccgagccggagctgaagctggagtccgtggtgatcgtgtcccgccacggcgtgcgcgccccgaccaaggccacccagctcatgcaggacgtgaccccggacgcctggccgacctggccggtgaagctcggcgagctgaccccgcgcggcggcgagctgatcgcctacctcggccactactggcgccagcgcctcgtggccgacggcctcctcccgaagtgcggctgcccgcagtccggccaggtggccatcatcgccgacgtggacgagcgcacccgcaagaccggcgaggccttcgccgccggcctcgccccggactgcgccatcaccgtgcacacccaggccgacacctcctccccggacccgctcttcaacccgctcaagaccggcgtgtgccagctcgacaacgccaacgtgaccgacgccatcctggagcgcgccggcggctccatcgccgacttcaccggccactaccagaccgccttccgcgagctggagcgcgtgctcaacttcccgcagtccaacctctgcctcaagcgcgagaagcaggacgagtcctgctccctcacccaggccctcccgtccgagctgaaggtgtccgccgactgcgtgtccctcaccggcgccgtgtccctcgcctccatgctcaccgaaatcttcctcctccagcaggcccagggcatgccggagccgggctggggccgcatcaccgactcccaccagtggaacaccctcctctccctccacaacgcccagttcgacctcctccagcgcaccccggaggtggcccgctcccgcgccaccccgctcctcgacctcatcaagaccgccctcaccccgcacccgccgcagaagcaggcctacggcgtgaccctcccgacctccgtgctcttcatcgccggccacgacaccaacctcgccaacctcggcggcgccctggagctgaactggaccctcccgggccagccggacaacaccccgccgggcggcgagctggtgttcgagcgctggcgccgcctctccgacaactcccagtggattcaggtgtccctcgtgttccagaccctccagcagatgcgcgacaagaccccgctctccctcaacaccccgccgggcgaggtgaagctcaccctcgccggctgcgaggagcgcaacgcccagggcatgtgctccctcgccggcttcacccagatcgtgaacgaggcccgcatcccggcctgctccctctaa

SEQ ID NO: 110 - protein, 431 aa, artificial sequence
  plasmid 11267 aa sequence
MRVLLVALALLALAASATSAAQSEPELKLESVVIVSRHGVRAPTKATQLMQDVTPDAWPTWPVKLGELTPRGGELIAYLGHYWRQRLVADGLLPKCGCPQSGQVAIIADVDERTRKTGEAFAAGLAPDCAITVHTQADTSSPDPLFNPLKTGVCQLDNANVTDAILERAGGSIADFTGHYQTAFRELERVLNFPQSNLCLKREKQDESCSLTQALPSELKVSADCVSLTGAVSLASMLTEIFLLQQAQGMPEPGWGRITDSHQWNTLLSLHNAQFDLLQRTPEVARSRATPLLDLIKTALTPHPPQKQAYGVTLPTSVLFIAGHDTNLANLGGALELNWTLPGQPDNTPPGGELVFERWRRLSDNSQWIQVSLVFQTLQQMRDKTPLSLNTPPGEVKLTLAGCEERNAQGMCSLAGFTQIVNEARIPACSL

SEQ ID NO: 111 - DNA, 1314 bp, artificial sequence
  plasmid 11268
atgagggtgttgctcgttgccctcgctctcctggctctcgctgcgagcgccaccagcgctgcgcagtccgagccggagctgaagctggagtccgtggtgatcgtgtcccgccacggcgtgcgcgccccgaccaaggccacccagctcatgcaggacgtgaccccggacgcctggccgacctggccggtgaagctcggcgagctgaccccgcgcggcggcgagctgatcgcctacctcggccactactggcgccagcgcctcgtggccgacggcctcctcccgaagtgcggctgcccgcagtccggccaggtggccatcatcgccgacgtggacgagcgcacccgcaagaccggcgaggccttcgccgccggcctcgccccggactgcgccatcaccgtgcacacccaggccgacacctcctccccggacccgctcttcaacccgctcaagaccggcgtgtgccagctcgacaacgccaacgtgaccgacgccatcctggagcgcgccggcggctccatcgccgacttcaccggccactaccagaccgccttccgcgagctggagcgcgtgctcaacttcccgcagtccaacctctgcctcaagcgcgagaagcaggacgagtcctgctccctcacccaggccctcccgtccgagctgaaggtgtccgccgactgcgtgtccctcaccggcgccgtgtccctcgcctccatgctcaccgaaatcttcctcctccagcaggcccagggcatgccggagccgggctggggccgcatcaccgactcccaccagtggaacaccctcctctccctccacaacgcccagttcgacctcctccagcgcaccccggaggtggcccgctcccgcgccaccccgctcctcgacctcatcaagaccgccctcaccccgcacccgccgcagaagcaggcctacggcgtgaccctcccgacctccgtgctcttcatcgccggccacgacaccaacctcgccaacctcggcggcgccctggagctgaactggaccctcccgggccagccggacaacaccccgccgggcggcgagctggtgttcgagcgctggcgccgcctctccgacaactcccagtggattcaggtgtccctcgtgttccagaccctccagcagatgcgcgacaagaccccgctctccctcaacaccccgccgggcgaggtgaagctcaccctcgccggctgcgaggagcgcaacgcccagggcatgtgctccctcgccggcttcacccagatcgtgaacgaggcccgcatcccggcctgctccctctccgagaaggacgagctgtaa

SEQ ID NO: 112 - protein, 437 aa, artificial sequence
  plasmid 11268 amino acid sequence
MRVLLVALALLALAASATSAAQSEPELKLESVVIVSRHGVRAPTKATQLMQDVTPDAWPTWPVKLGELTPRGGELIAYLGHYWRQRLVADGLLPKCGCPQSGQVAIIADVDERTRKTGEAFAAGLAPDCAITVHTQADTSSPDPLFNPLKTGVCQLDNANVTDAILERAGGSIADFTGHYQTAFRELERVLNFPQSNLCLKREKQDESCSLTQALPSELKVSADCVSLTGAVSLASMLTEIFLLQQAQGMPEPGWGRITDSHQWNTLLSLHNAQFDLLQRTPEVARSRATPLLDLIKTALTPHPPQKQAYGVTLPTSVLFIAGHDTNLANLGGALELNWTLPGQPDNTPPGGELVFERWRRLSDNSQWIQVSLVFQTLQQMRDKTPLSLNTPPGEVKLTLAGCEERNAQGMCSLAGFTQIVNEARIPACSLSEKDEL

(19) United States
(12) Patent Application Publication
(10) Pub. No.: US 2008/0289066 A1
(43) Pub. Date: Nov. 20, 2008

(54) Self-Processing Plants and Plant Parts

(76) Inventors: Michael B. Lanahan, Research Triangle Park, NC (US); Shib S. Basu, Apex, NC (US); Christopher J. Batie, Durham, NC (US); Wen Chen, Cary, NC (US); Joyce Craig, Pittsboro, NC (US); Mark Kinkema, Durham, NC (US)

Correspondence Address: SYNGENTA BIOTECHNOLOGY, INC.;PATENT DEPARTMENT, 3054 CORNWALLIS ROAD, P.O. BOX 12257, RESEARCH TRIANGLE PARK, NC, 27709-2257, US

(21) Appl. No.: 10/591,870

(22) PCT Filed: Mar. 8, 2004
(86) PCT No.: PCT/US04/07182

§ 371 Date: Sep. 7, 2006

(51) Int. Cl. C12N 15/56; C12N 1/21; A01H 5/00; C12P 19/00

(52) U.S. Cl. 800/298; 536/23.2; 435/252.2; 800/321; 800/320.1; 800/320; 800/320.2; 800/320.3; 435/72

(57) ABSTRACT

The invention provides polynucleotides, preferably synthetic polynucleotides, which encode processing enzymes that are optimized for expression in plants. The polynucleotides encode mesophilic, thermophilic, or hyperthermophilic processing enzymes, which are activated under suitable activating conditions to act upon the desired substrate. Also provided are “self-processing” transgenic plants, and plant parts, e.g., grain, which express one or more of these enzymes and have an altered composition that facilitates plant and grain processing. Methods for making and using these plants, e.g., to produce food products having improved taste and to produce fermentable substrates for the production of ethanol and fermented beverages are also provided.

### RELATED APPLICATIONS

This application is a continuation-in-part of U.S. patent application Ser. No. 10/228,063, filed Aug. 27, 2002, which claims priority to Application Ser. No. 60/315,281, filed Aug. 27, 2001, each of which is herein incorporated by reference in their entirety.

### FIELD OF THE INVENTION

The present invention generally relates to the field of plant molecular biology, and more specifically, to the creation of plants that express a processing enzyme which provides a desired characteristic to the plant or products thereof.

### BACKGROUND OF THE INVENTION

Enzymes are used to process a variety of agricultural products such as wood, fruits and vegetables, starches, juices, and the like. Typically, processing enzymes are produced and recovered on an industrial scale from various sources, such as microbial fermentation (Bacillus α-amylase), or isolation from plants (coffee β-galactosidase or papain from plant parts). Enzyme preparations are used in different processing applications by mixing the enzyme and the substrate under the appropriate conditions of moisture, temperature, time, and mechanical mixing such that the enzymatic reaction is achieved in a commercially viable manner. The methods involve separate steps of enzyme production, manufacture of an enzyme preparation, mixing the enzyme and substrate, and subjecting the mixture to the appropriate conditions to facilitate the enzymatic reaction. A method that reduces or eliminates the time, energy, mixing, capital expenses, and/or enzyme production costs, or results in improved or novel products, would be useful and beneficial. One example of where such improvements are needed is in the area of corn milling.

Today corn is milled to obtain cornstarch and other corn-milling co-products such as corn gluten feed, corn gluten meal, and corn oil. The starch obtained from the process is often further processed into other products such as derivatized starches and sugars, or fermented to make a variety of products including alcohols or lactic acid. Processing of cornstarch often involves the use of enzymes, in particular, enzymes that hydrolyze and convert starch into fermentable sugars or fructose (α- and gluco-amylase, α-glucosidase, glucose isomerase, and the like). The process used commercially today is capital intensive as construction of very large mills is required to process corn on scales required for reasonable cost-effectiveness. In addition the process requires the separate manufacture of starch-hydrolyzing or modifying enzymes and then the machinery to mix the enzyme and substrate to produce the hydrolyzed starch products.

The process of starch recovery from corn grain is well known and involves a wet-milling process. Corn wet-milling includes the steps of steeping the corn kernel, grinding the corn kernel and separating the components of the kernel. The kernels are steeped in a steep tank with a countercurrent flow of water at about 120° F. and the kernels remain in the steep tank for 24 to 48 hours. This steepwater typically contains sulfur dioxide at a concentration of about 0.2% by weight. Sulfur dioxide is employed in the process to help reduce microbial growth and also to reduce disulfide bonds in endosperm proteins to facilitate more efficient starch-protein separation. Normally, about 0.59 gallons of steepwater is used per bushel of corn. The steepwater is considered waste and often contains undesirable levels of residual sulfur dioxide.

The steeped kernels are then dewatered and subjected to sets of attrition type mills. The first set of attrition type mills rupture the kernels releasing the germ from the rest of the kernel. A commercial attrition type mill suitable for the wet milling business is sold under the brand name Bauer. Centrifugation is used to separate the germ from the rest of the kernel. A typical commercial centrifugation separator is the Merco centrifugal separator. Attrition mills and centrifugal separators are large expensive items that use energy to operate.

In the next step of the process, the remaining kernel components including the starch, hull, fiber, and gluten are subjected to another set of attrition mills and passed through a set of wash screens to separate the fiber components from the starch and gluten (endosperm protein). The starch and gluten pass through the screens while the fiber does not. Centrifugation or a third grind followed by centrifugation is used to separate the starch from the endosperm protein. Centrifugation produces a starch slurry which is dewatered, then washed with fresh water and dried to about 12% moisture. The substantially pure starch is typically further processed by the use of enzymes.

The separation of starch from the other components of the grain is performed because removing the seed coat, embryo and endosperm proteins allows one to efficiently contact the starch with processing enzymes, and the resulting hydrolysis products are relatively free from contaminants from the other kernel components. Separation also ensures that other components of the grain are effectively recovered and can be subsequently sold as co-products to increase the revenues from the mill.

After the starch is recovered from the wet-milling process it typically undergoes the processing steps of gelatinization, liquefaction and dextrinization for maltodextrin production, and subsequent steps of saccharification, isomerization and refining for the production of glucose, maltose and fructose.

Gelatinization is employed in the hydrolysis of starch because currently available enzymes cannot rapidly hydrolyze crystalline starch. To make the starch available to the hydrolytic enzymes, the starch is typically made into a slurry with water (20-40% dry solids) and heated at the appropriate gelling temperature. For cornstarch this temperature is between 105-110° C. The gelatinized starch is typically very viscous and is therefore thinned in the next step called liquefaction. Liquefaction breaks some of the bonds between the glucose molecules of the starch and is accomplished enzymatically or through the use of acid. Heat-stable endo α-amylase enzymes are used in this step, and in the subsequent step of dextrinization. The extent of hydrolysis is controlled in the dextrinization step to yield hydrolysis products of the desired percentage of dextrose.

Further hydrolysis of the dextrin products from the liquefaction step is carried out by a number of different exo-amylases and debranching enzymes, depending on the products that are desired. And finally if fructose is desired then immobilized glucose isomerase enzyme is typically employed to convert glucose into fructose.

Dry-mill processes of making fermentable sugars (and then ethanol, for example) from cornstarch facilitate efficient contacting of exogenous enzymes with starch. These processes are less capital intensive than wet-milling but significant cost advantages are still desirable, as often the co-products derived from these processes are not as valuable as those derived from wet-milling. For example, in dry milling corn, the kernel is ground into a powder to facilitate efficient contact of starch by degrading enzymes. After enzyme hydrolysis of the corn flour the residual solids have some feed value as they contain proteins and some other components. Eckhoff recently described the potential for improvements and the relevant issues related to dry milling in a paper entitled “Fermentation and costs of fuel ethanol from corn with quick-germ process” (Appl. Biochem. Biotechnol., 94: 41 (2001)). The “quick germ” method allows for the separation of the oil-rich germ from the starch using a reduced steeping time.

One example where the regulation and/or level of endogenous processing enzymes in a plant can result in a desirable product is sweet corn. Typical sweet corn varieties are distinguished from field corn varieties by the fact that sweet corn is not capable of normal levels of starch biosynthesis. Genetic mutations in the genes encoding enzymes involved in starch biosynthesis are typically employed in sweet corn varieties to limit starch biosynthesis. Such mutations are in the genes encoding starch synthases and ADP-glucose pyrophosphorylases (such as the sugary and super-sweet mutations). Fructose, glucose and sucrose, which are the simple sugars necessary for producing the palatable sweetness that consumers of edible fresh corn desire, accumulate in the developing endosperm of such mutants. However, if the level of starch accumulation is too high, such as when the corn is left to mature for too long (late harvest) or the corn is stored for an excessive period before it is consumed, the product loses sweetness and takes on a starchy taste and mouthfeel. The harvest window for sweet corn is therefore quite narrow, and shelf-life is limited.

Another significant drawback to the farmer who plants sweet corn varieties is that the usefulness of these varieties is limited exclusively to edible food. If a farmer wanted to forego harvesting his sweet corn for use as edible food during seed development, the crop would be essentially a loss. The grain yield and quality of sweet corn is poor for two fundamental reasons. The first reason is that mutations in the starch biosynthesis pathway cripple the starch biosynthetic machinery and the grains do not fill out completely, causing the yield and quality to be compromised. Secondly, due to the high levels of sugars present in the grain and the inability to sequester these sugars as starch, the overall sink strength of the seed is reduced, which exacerbates the reduction of nutrient storage in the grain. The endosperms of sweet corn variety seeds are shrunken and collapsed, do not undergo proper desiccation, and are susceptible to diseases. The poor quality of the sweet corn grain has further agronomic implications; as poor seed viability, poor germination, seedling disease susceptibility, and poor early seedling vigor result from the combination of factors caused by inadequate starch accumulation. Thus, the poor quality issues of sweet corn impact the consumer, farmer/grower, distributor, and seed producer.

Thus, for dry-milling, there is a need for a method which improves the efficiency of the process and/or increases the value of the co-products. For wet-milling, there is a need for a method of processing starch that does not require the equipment necessary for prolonged steeping, grinding, milling, and/or separating the components of the kernel. For example, there is a need to modify or eliminate the steeping step in wet milling as this would reduce the amount of waste water requiring disposal, thereby saving energy and time, and increasing mill capacity (kernels would spend less time in steep tanks). There is also a need to eliminate or improve the process of separating the starch-containing endosperm from the embryo.

### SUMMARY OF THE INVENTION

The present invention is directed to self-processing plants and plant parts and methods of using the same. The self-processing plant and plant parts of the present invention are capable of expressing and activating enzyme(s) (mesophilic, thermophilic, and/or hyperthermophilic). Upon activation of the enzyme(s) (mesophilic, thermophilic, or hyperthermophilic) the plant or plant part is capable of self-processing the substrate upon which it acts to obtain the desired result.

The present invention is directed to an isolated polynucleotide a) comprising SEQ ID NO: 2, 4, 6, 9, 19, 21, 25, 37, 39, 41, 43, 46, 48, 50, 52, or 59 or the complement thereof, or a polynucleotide which hybridizes to the complement of any one of SEQ ID NO: 2, 4, 6, 9, 19, 21, 25, 37, 39, 41, 43, 46, 48, 50, 52, or 59 under low stringency hybridization conditions and encodes a polypeptide having α-amylase, pullulanase, α-glucosidase, glucose isomerase, or glucoamylase activity or b) encoding a polypeptide comprising SEQ ID NO: 10, 13, 14, 15, 16, 18, 20 24, 26, 27, 28, 29, 30, 33, 34, 35, 36, 38, 40, 42, 44, 45, 47, 49, or 51 or an enzymatically active fragment thereof. Preferably, the isolated polynucleotide encodes a fusion polypeptide comprising a first polypeptide and a second peptide, wherein said first polypeptide has α-amylase, pullulanase, α-glucosidase, glucose isomerase, or glucoamylase activity. Most preferably, the second peptide comprises a signal sequence peptide, which may target the first polypeptide to a vacuole, endoplasmic reticulum, chloroplast, starch granule, seed or cell wall of a plant. For example, the signal sequence may be an N-terminal signal sequence from waxy, an N-terminal signal sequence from γ-zein, a starch binding domain, or a C-terminal starch binding domain. Polynucleotides that hybridize to the complement of any one of SEQ ID NO: 2, 9, or 52 under low stringency hybridization conditions and encodes a polypeptide having α-amylase activity; to the complement of SEQ ID NO: 4 or 25 under low stringency hybridization conditions and encodes a polypeptide having pullulanase activity; to the complement of SEQ ID NO:6 and encodes a polypeptide having α-glucosidase activity; to the complement of any one of SEQ ID NO: 19, 21, 37, 39, 41, or 43 under low stringency hybridization conditions and encodes a polypeptide having glucose isomerase activity; to the complement of any one of SEQ ID NO: 46, 48, 50, or 59 under low stringency hybridization conditions and encodes a polypeptide having glucoamylase activity are further encompassed.

The present invention is also directed to an isolated polynucleotide a) comprising SEQ ID NO: 61, 63, 65, 79, 81, 83, 85, 87, 89, 91, 93, 94, 95, 96, 97, 99, 108, and 110 or the complement thereof, or a polynucleotide which hybridizes to the complement of any one of SEQ ID NO: 61, 63, 65, 79, 81, 83, 85, 87, 89, 91, 93, 94, 95, 96, 97, 99, 108, or 110 under low stringency hybridization conditions and encodes a polypeptide having xylanase, cellulase, glucanase, beta glucosidase, esterase or phytase activity b) encoding a polypeptide comprising SEQ ID NO: 62, 64, 66, 70, 80, 82, 84, 86, 88, 90, 92, 109, or 111 or an enzymatically active fragment thereof. The isolated polynucleotide may encode a fusion polypeptide comprising a first polypeptide and a second peptide, wherein said first polypeptide has xylanase, cellulase, glucanase, beta glucosidase, protease, or phytase activity. The second peptide may comprises a signal sequence peptide, which may target the first polypeptide to a vacuole, endoplasmic reticulum, chloroplast, starch granule, seed or cell wall of a plant. For example, the signal sequence may be an N-terminal signal sequence from waxy, an N-terminal signal sequence from γ-zein, a starch binding domain, or a C-terminal starch binding domain.

Exemplary xylanases provided and useful in the invention include those encoded by SEQ ID NO: 61, 63, or 65. An exemplary protease, namely bromelain, encoded by SEQ ID NO: 69 is also provided. Exemplary cellulases include cellobiohydrolase I and II as provided herein and encoded by SEQ ID NO: 79, 81, 93, and 94. An exemplary glucanase is provides as 6GP1 described herein encoded by SEQ ID NO: 85. Exemplary beta glucosidases include beta glucosidase 2 and D, as described herein and encoded by SEQ ID NO: 96 and 97. An exemplary esterase is also provided, namely ferulic acid esterase as encoded by SEQ ID NO:99. And, an exemplary phytase, Nov9X as encoded by SEQ ID NO: 109-112 is also provided.

Also included are expression cassettes comprising a polynucleotide a) having SEQ ID NO: 2, 4, 6, 9, 19, 21, 25, 37, 39, 41, 43, 46, 48, 50, 52, or 59 or the complement thereof, or a polynucleotide which hybridizes to the complement of any one of SEQ ID NO: 2, 4, 6, 9, 19, 21, 25, 37, 39, 41, 43, 46, 48, 50, 52, or 59 or under low stringency hybridization conditions and encodes an polypeptide having α-amylase, pullulanase, α-glucosidase, glucose isomerase, or glucoamylase activity or b) encoding a polypeptide comprising SEQ ID NO: 10, 13, 14, 15, 16, 18, 20, 24, 26, 27, 28, 29, 30, 33, 34, 35, 36, 38, 40, 42, 44, 45, 47, 49, or 51, or an enzymatically active fragment thereof. The expression cassette further comprises a promoter operably linked to the polynucleotide, such as an inducible promoter, tissue-specific promoter, or preferably an endosperm-specific promoter. Preferably, the endosperm-specific promoter is a maize γ-zein promoter or a maize ADP-gpp promoter or a maize Q promoter promoter or a rice glutelin-1 promoter. In a preferred embodiment, the promoter comprises SEQ ID NO: 11 or SEQ ID NO: 12 or SEQ ID NO: 67 or SEQ ID NO: 98. Moreover, in another preferred embodiment the polynucleotide is oriented in sense orientation relative to the promoter. The expression cassette of the present invention may further encode a signal sequence which is operably linked to the polypeptide encoded by the polynucleotide. The signal sequence preferably targets the operably linked polypeptide to a vacuole, endoplasmic reticulum, chloroplast, starch granule, seed or cell wall of a plant. The signal sequences include an N-terminal signal sequence from waxy, an N-terminal signal sequence from γ-zein, or a starch binding domain.

Moreover, an expression cassette comprising a polynucleotide a) having SEQ ID NO: 61, 63, 65, 79, 81, 83, 85, 87, 89, 91, 93, 94, 95, 96, 97, 99, 108, and 110 or the complement thereof, or a polynucleotide which hybridizes to the complement of any one of SEQ ID NO: 61, 63, 65, 79, 81, 83, 85, 87, 89, 91, 93, 94, 95, 96, 97, 99, 108, and 110 or under low stringency hybridization conditions and encodes an polypeptide having xylanase, cellulase, glucanase, beta glucosidase, esterase or phytase activity or b) encoding a polypeptide comprising SEQ ID NO: 62, 64, 66, 70, 80, 82, 84, 86, 88, 90, 92, 109, or 111, or an enzymatically active fragment thereof. The expression cassette further comprises a promoter operably linked to the polynucleotide, such as an inducible promoter, tissue-specific promoter, or preferably an endosperm-specific promoter. The endosperm-specific promoter may be a maize γ-zein promoter or a maize ADP-gpp promoter or a maize Q promoter promoter or a rice glutelin-1 promoter. In an embodiment, the promoter comprises SEQ ID NO: 11 or SEQ ID NO: 12 or SEQ ID NO: 67 or SEQ ID NO: 98. Moreover, in another embodiment the polynucleotide is oriented in sense orientation relative to the promoter. The expression cassette of the present invention may further encode a signal sequence which is operably linked to the polypeptide encoded by the polynucleotide. The signal sequence preferably targets the operably linked polypeptide to a vacuole, endoplasmic reticulum, chloroplast, starch granule, seed or cell wall of a plant. The signal sequences include an N-terminal signal sequence from waxy, an N-terminal signal sequence from γ-zein, or a starch binding domain.

The present invention is further directed to a vector or cell comprising the expression cassettes of the present invention. The cell may be selected from the group consisting of an Agrobacterium, a monocot cell, a dicot cell, a Liliopsida cell, a Panicoideae cell, a maize cell, and a cereal cell, such as a rice cell.

Moreover, the present invention encompasses a plant stably transformed with the vectors of the present invention. A plant stably transformed with a vector comprising an α-amylase having an amino acid sequence of any of SEQ ID NO: 1, 10, 13, 14, 15, 16, 33, 35 or 88 or encoded by a polynucleotide comprising any of SEQ ID NO: 2, 9, or 87 is provided.

In another embodiment, a plant stably transformed with a vector comprising a pullulanase having an amino acid sequence of any of SEQ ID NO: 24 or 34, or encoded by a polynucleotide comprising any of SEQ ID NO: 4 or 25 is provided. A plant stably transformed with a vector comprising an α-glucosidase having an amino acid sequence of any of SEQ ID NO: 26 or 27, or encoded by a polynucleotide comprising SEQ ID NO:6 is further provided. A plant stably transformed with a vector comprising an glucose isomerase having an amino acid sequence of any of SEQ ID NO: 18, 20, 28, 29, 30, 38, 40, 42, or 44, or encoded by a polynucleotide comprising any of SEQ ID NO:19, 21, 37, 39, 41, or 43 is further described herein. In another embodiment, a plant stably transformed with a vector comprising a glucose amylase having an amino acid sequence of any of SEQ ID NO: 45, 47, or 49, or encoded by a polynucleotide comprising any of SEQ ID NO:46, 48, 50, or 59 is described.

An additional embodiment provides a plant stably transformed with a vector comprising a xylanase having an amino acid sequence of any of SEQ ID NO: 62, 64 or 66, or encoded by a polynucleotide comprising any of SEQ ID NO: 61, 63, or 65. A plant stably transformed with a vector comprising a protease is also provided. The protease may be bromelain having an amino acid sequence as set forth in SEQ ID NO: 70, or encoded by a polynucleotide having SEQ ID NO: 69. In another embodiment, a plant stably transformed with a vector comprising a cellulase is provided. The cellulase may be a cellobiohydrolase encoded by a polynucleotide comprising any of SEQ ID NO: 79, 80, 81, 82, 93 or 94.

An additional embodiment provides a plant stably transformed with a vector comprising a glucanase, such as an endoglucanase. The endoglucanase may be endoglucanase I which has an amino acid sequence as in SEQ ID NO: 84, or encoded by a polynucleotide comprising SEQ ID NO: 83. A plant stably transformed with a vector comprising a beta glucosidase is also provided. The beta glucosidase is may be beta glucosidase 2 or beta glucosidase D, which have an amino acid sequence set forth in SEQ ID NO: 90 or 92, or encoded by a polynucleotide having SEQ ID NO: 89 or 91. In another embodiment, a plant stably transformed with a vector comprising an esterase is provided. The esterase may be a ferulic acid esterase encoded by a polynucleotide comprising SEQ ID NO: 99.

Plant products, such as seed, fruit or grain from the stably transformed plants of the present invention are further provided.

In another embodiment, the invention is directed to a transformed plant, the genome of which is augmented with a recombinant polynucleotide encoding at least one processing enzyme operably linked to a promoter sequence, the sequence of which polynucleotide is optimized for expression in the plant. The plant may be a monocot, such as maize or rice, or a dicot. The plant may be a cereal plant or a commercially grown plant. The processing enzyme is selected from the group consisting of an α-amylase, glucoamylase, glucose isomerase, glucanase, βamylase, α-glucosidase, isoamylase, pullulanase, neo-pullulanase, iso-pullulanase, amylopullulanase, cellulase, exo-1,4-β-cellobiohydrolase, exo-1,3-β-D-glucanase, β-glucosidase, endoglucanase, L-arabinase, α-arabinosidase, galactanase, galactosidase, mannanase, mannosidase, xylanase, xylosidase, protease, glucanase, xylanase, esterase, phytase, and lipase. The processing enzyme is a starch-processing enzyme selected from the group consisting of α-amylase, glucoamylase, glucose isomerase, β-amylase, α-glucosidase, isoamylase, pullulanase, neo-pullulanase, iso-pullulanase, and amylopullulanase. The enzyme may be selected from α-amylase, glucoamylase, glucose isomerase, glucose isomerase, α-glucosidase, and pullulanase. The processing enzyme may be hyperthermophilic. In accordance with this aspect of the invention, the enzyme may be a non-starch degrading enzyme selected from the group consisting of protease, glucanase, xylanase, esterase, phytase, cellulase, beta glucosidase, and lipase. Such enzymes may be hyperthermophilic. In an embodiment, the enzyme accumulates in the vacuole, endoplasmic reticulum, chloroplast, starch granule, seed or cell wall of a plant. Moreover, in another embodiment, the genome of plant may be further augmented with a second recombinant polynucleotide comprising a non-hyperthermophilic enzyme.

In another aspect of the invention, provided is a transformed plant, the genome of which is augmented with a recombinant polynucleotide encoding at least one processing enzyme selected from the group consisting of α-amylase, glucoamylase, glucose isomerase, α-glucosidase, pullulanase, xylanase, cellulase, protease, glucanase, beta glucosidase, esterase, phytase or lipase operably linked to a promoter sequence, the sequence of which polynucleotide is optimized for expression in the plant.

Another embodiment is directed to a transformed maize plant, the genome of which is augmented with a recombinant polynucleotide encoding at least one processing enzyme selected from the group consisting of α-amylase, glucoamylase, glucose isomerase, α-glucosidase, pullulanase, xylanase, cellulase, protease, glucanase, phytase, beta glucosidase, esterase, or lipase operably linked to a promoter sequence, the sequence of which polynucleotide is optimized for expression in the maize plant.

A transformed plant, the genome of which is augmented with a recombinant polynucleotide having SEQ ID NO: 83 operably linked to a promoter and to a signal sequence is provided. Additionally, a transformed plant, the genome of which is augmented with a recombinant polynucleotide having the SEQ ID NO: 93 or 94 operably linked to a promoter and to a signal sequence is described. In another embodiment, a transformed plant, the genome of which is augmented with a recombinant polynucleotide having SEQ ID NO: 95, operably linked to a promoter and to a signal sequence. Moreover, a transformed plant, the genome of which is augmented with a recombinant polynucleotide having SEQ ID NO: 96 is described. Also described is a transformed plant, the genome of which is augmented with a recombinant polynucleotide having SEQ ID NO: 97. Also described is a transformed plant, the genome of which is augmented with a recombinant polypeptide having SEQ ID NO: 99.

Products of the transformed plants are further envisioned herein. The product for example, include seed, fruit, or grain. The product may alternatively be the processing enzyme, starch or sugar.

A plant obtained from a stably transformed plant of the present invention is further described. In this aspect, the plant may be a hybrid plant or an inbred plant.

A starch composition is a further embodiment of the invention comprising at least one processing enzyme which is a protease, glucanase, or esterase.

Grain is another embodiment of the invention comprising at least one processing enzyme, which is an α-amylase, pullulanase, α-glucosidase, glucoamylase, glucose isomerase, xylanase, cellulase, glucanase, beta glucosidase, esterase, protease, lipase or phytase.

In another embodiment, a method of preparing starch granules, comprising treating grain which comprises at least one non-starch processing enzyme under conditions which activate the at least one enzyme, yielding a mixture comprising starch granules and non-starch degradation products, wherein the grain is obtained from a transformed plant, the genome of which is augmented with an expression cassette encoding the at least one enzyme; and separating starch granules from the mixture is provided. Therein, the enzyme may be a protease, glucanase, xylanase, phytase, lipase, beta glucosidase, cellulase or esterase. Moreover, the enzyme is preferably hyperthermophilic. The grain may be cracked grain and/or may be treated under low or high moisture conditions. Alternatively, the grain may treated with sulfur dioxide. The present invention may further comprise separating non-starch products from the mixture. The starch products and non-starch products obtained by this method are further described.

In yet another embodiment, a method to produce hypersweet corn comprising treating transformed corn or a part thereof, the genome of which is augmented with and expresses in the endosperm an expression cassette encoding at least one starch-degrading or starch-isomerizing enzyme, under conditions which activate the at least one enzyme so as to convert polysaccharides in the corn into sugar, yielding hypersweet corn is provided. The expression cassette may further comprises a promoter operably linked to the polynucleotide encoding the enzyme. The promoter may be a constitutive promoter, seed-specific promoter, or endosperm-specific promoter, for example. The enzyme may be hyperthermophilic and may be an α-amylase. The expression cassette used herein may further comprise a polynucleotide which encodes a signal sequence operably linked to the at least one enzyme. The signal sequence may direct the enzyme to the apoplast or the endoplasmic reticulum, for example. The enzyme comprises any one of SEQ ID NO: 13, 14, 15, 16, 33, or 35. The enzyme may also comprise SEQ ID NO: 87.

In a most preferred embodiment, a method of producing hypersweet corn comprising treating transformed corn or a part thereof, the genome of which is augmented with and expresses in the endosperm an expression cassette encoding an α-amylase, under conditions which activate the at least one enzyme so as to convert polysaccharides in the corn into sugar, yielding hypersweet corn is described. The enzyme may be hyperthermophilic and the hyperthermophilic α-amylase may comprise the amino acid sequence of any of SEQ ID NO: 10, 13, 14, 15, 16, 33, or 35, or an enzymatically active fragment thereof having α-amylase activity. The enzyme comprise SEQ ID NO: 87.

A method to prepare a solution of hydrolyzed starch product comprising; treating a plant part comprising starch granules and at least one processing enzyme under conditions which activate the at least one enzyme thereby processing the starch granules to form an aqueous solution comprising hydrolyzed starch product, wherein the plant part is obtained from a transformed plant, the genome of which is augmented with an expression cassette encoding the at least one starch processing enzyme; and

collecting the aqueous solution comprising the hydrolyzed starch product is described herein. The hydrolyzed starch product may comprise a dextrin, maltooligosaccharide, glucose and/or mixtures thereof. The enzyme may be α-amylase, α-glucosidase, glucoamylase, pullulanase, amylopullulanase, glucose isomerase, or any combination thereof. Moreover, the enzyme may be hyperthermophilic. In another aspect, the genome of the plant part may be further augmented with an expression cassette encoding a non-hyperthermophilic starch processing enzyme. The non-hyperthermophilic starch processing enzyme may be selected from the group consisting of amylase, glucoamylase, α-glucosidase, pullulanase, glucose isomerase, or a combination thereof. In yet another aspect, the processing enzyme is preferably expressed in the endosperm. The plant part may be grain, and from corn, wheat, barley, rye, oat, sugar cane or rice. The at least one processing enzyme is operably linked to a promoter and to a signal sequence that targets the enzyme to the starch granule or the endoplasmic reticulum, or to the cell wall. The method may further comprise isolating the hydrolyzed starch product and/or fermenting the hydrolyzed starch product.

In another aspect of the invention, a method of preparing hydrolyzed starch product comprising treating a plant part comprising starch granules and at least one starch processing enzyme under conditions which activate the at least one enzyme thereby processing the starch granules to form an aqueous solution comprising a hydrolyzed starch product, wherein the plant part is obtained from a transformed plant, the genome of which is augmented with an expression cassette encoding at least one α-amylase; and

collecting the aqueous solution comprising hydrolyzed starch product is described. The α-amylase may be hyperthermophilic and the hyperthermophilic α-amylase comprises the amino acid sequence of any of SEQ ID NO: 1, 10, 13, 14, 15, 16, 33, or 35, or an active fragment thereof having α-amylase activity. The expression cassette may comprise a polynucleotide selected from any of SEQ ID NO: 2, 9, 46, or 52, a complement thereof, or a polynucleotide that hybridizes to any of SEQ ID NO: 2, 9, 46, or 52 under low stringency hybridization conditions and encodes a polypeptide having α-amylase activity. Moreover, the invention further provides for the genome of the transformed plant further comprising a polynucleotide encoding a non-thermophilic starch-processing enzyme. Alternatively, the plant part may be treated with a non-hyperthermophilic starch-processing enzyme.

The present invention is further directed to a transformed plant part comprising at least one starch-processing enzyme present in the cells of the plant, wherein the plant part is obtained from a transformed plant, the genome of which is augmented with an expression cassette encoding the at least one starch processing enzyme. Preferably, the enzyme is a starch-processing enzyme selected from the group consisting of α-amylase, glucoamylase, glucose isomerase, β-amylase, α-glucosidase, isoamylase, pullulanase, neo-pullulanase, iso-pullulanase, and amylopullulanase. Moreover, the enzyme may be hyperthermophilic. The plant may be any plant, such as corn or rice for example.

Another embodiment of the invention is a transformed plant part comprising at least one non-starch processing enzyme present in the cell wall or the cells of the plant, wherein the plant part is obtained from a transformed plant, the genome of which is augmented with an expression cassette encoding the at least one non-starch processing enzyme or at least one non-starch polysaccharide processing enzyme. The enzyme may be hyperthermophilic. Moreover, the non-starch processing enzyme may be a protease, glucanase, xylanase, esterase, phytase, beta glucosidase, cellulase or lipase. The plant part can be any plant part, but preferably is an ear, seed, fruit, grain, stover, chaff, or bagasse.

The present invention is also directed to transformed plant parts. For example, a transformed plant part comprising an α-amylase having an amino acid sequence of any of SEQ ID NO: 1, 10, 13, 14, 15, 16, 33, or 35, or encoded by a polynucleotide comprising any of SEQ ID NO: 2, 9, 46, or 52, a transformed plant part comprising an α-glucosidase having an amino acid sequence of any of SEQ ID NO: 5, 26 or 27, or encoded by a polynucleotide comprising SEQ ID NO:6, a transformed plant part comprising a glucose isomerase having the amino acid sequence of any one of SEQ ID NO: 28, 29, 30, 38, 40, 42, or 44, or encoded by a polynucleotide comprising any one of SEQ ID NO: 19, 21, 37, 39, 41, or 43, a transformed plant part comprising a glucoamylase having the amino acid sequence of SEQ ID NO:45 or SEQ ID NO:47, or SEQ ID NO:49, or encoded by a polynucleotide comprising any of SEQ ID NO: 46, 48, 50, or 59, and a transformed plant part comprising a pullulanase encoded by a polynucleotide comprising any of SEQ ID NO: 4 or 25 are described.

The present invention is also directed to transformed plant parts. For example, a transformed plant part comprising a xylanase having an amino acid sequence of any of SEQ ID NO: 62, 64 or 66, or encoded by a polynucleotide comprising any of SEQ ID NO: 61, 63, or 65. A transformed plant part comprising a protease is also provided. The protease may be bromelain having an amino acid sequence as set forth in SEQ ID NO: 70, or encoded by a polynucleotide having SEQ ID NO: 69. In another embodiment, a transformed plant part comprising a cellulase is provided. The cellulase may be a cellobiohydrolase encoded by a polynucleotide comprising any of SEQ ID NO: 79, 80, 81, 82, 93 or 94.

An additional embodiment provides a transformed plant part a glucanase, such as an endoglucanase. The endoglucanase may be endoglucanase I which has an amino acid sequence as in SEQ ID NO: 84, or encoded by a polynucleotide comprising SEQ ID NO: 83. A transformed plant part comprising a beta glucosidase is also provided. The beta glucosidase is may be beta glucosidase 2 or beta glucosidase D, which have an amino acid sequence set forth in SEQ ID NO: 90 or 92, or encoded by a polynucleotide having SEQ ID NO: 89 or 91. In another embodiment, a transformed plant part comprising an esterase is provided. The esterase may be a ferulic acid esterase encoded by a polynucleotide comprising SEQ ID NO: 99.

Another embodiment is a method of converting starch in the transformed plant part comprising activating the starch processing enzyme contained therein. The starch, dextrin, maltooligosaccharide or sugar produced according to this method is further described.

The present invention further describes a method of using a transformed plant part comprising at least one non-starch processing enzyme in the cell wall or the cell of the plant part, comprising treating a transformed plant part comprising at least one non-starch polysaccharide processing enzyme under conditions so as to activate the at least one enzyme thereby digesting non-starch polysaccharide to form an aqueous solution comprising oligosaccharide and/or sugars, wherein the plant part is obtained from a transformed plant, the genome of which is augmented with an expression cassette encoding the at least one non-starch polysaccharide processing enzyme; and collecting the aqueous solution comprising the oligosaccharides and/or sugars. The non-starch polysaccharide processing enzyme may be hyperthermophilic.

A method of using transformed seeds comprising at least one processing enzyme, comprising treating transformed seeds which comprise at least one protease or lipase under conditions so as the activate the at least one enzyme yielding an aqueous mixture comprising amino acids and fatty acids, wherein the seed is obtained from a transformed plant, the genome of which is augmented with an expression cassette encoding the at least one enzyme; and collecting the aqueous mixture. The amino acids, fatty acids or both are preferably isolated. The at least one protease or lipase may be hyperthermophilic.

A method to prepare ethanol comprising treating a plant part comprising at least one polysaccharide processing enzyme under conditions to activate the at least one enzyme thereby digesting polysaccharide to form oligosaccharide or fermentable sugar, wherein the plant part is obtained from a transformed plant, the genome of which is augmented with an expression cassette encoding the at least one polysaccharide processing enzyme; and incubating the fermentable sugar under conditions that promote the conversion of the fermentable sugar or oligosaccharide into ethanol. The plant part may be a grain, fruit, seed, stalks, wood, vegetable or root. The plant part may be obtained from a plant selected from the group consisting of oats, barley, wheat, berry, grapes, rye, corn, rice, potato, sugar beet, sugar cane, pineapple, grasses and trees.

In another preferred embodiment, the polysaccharide processing enzyme is α-amylase, glucoamylase, α-glucosidase, glucose isomerase, pullulanase, or a combination thereof.

A method to prepare ethanol comprising treating a plant part comprising at least one enzyme selected from the group consisting of α-amylase, glucoamylase, α-glucosidase, glucose isomerase, or pullulanase, or a combination thereof, with heat for an amount of time and under conditions to activate the at least one enzyme thereby digesting polysaccharide to form fermentable sugar, wherein the plant part is obtained from a transformed plant, the genome of which is augmented with an expression cassette encoding the at least one enzyme; and incubating the fermentable sugar under conditions that promote the conversion of the fermentable sugar into ethanol is provided. The at least one enzyme may be hyperthermophilic or mesophilic.

In another embodiment, a method to prepare ethanol comprising treating a plant part comprising at least one non-starch processing enzyme under conditions to activate the at least one enzyme thereby digesting non-starch polysaccharide to oligosaccharide and fermentable sugar, wherein the plant part is obtained from a transformed plant, the genome of which is augmented with an expression cassette encoding the at least one enzyme; and incubating the fermentable sugar under conditions that promote the conversion of the fermentable sugar into ethanol is provided. The non-starch processing enzyme may be a xylanase, cellulase, glucanase, beta glucosidase, protease, esterase, lipase or phytase.

A method to prepare ethanol comprising treating a plant part comprising at least one enzyme selected from the group consisting of α-amylase, glucoamylase, α-glucosidase, glucose isomerase, or pullulanase, or a combination thereof, under conditions to activate the at least one enzyme thereby digesting polysaccharide to form fermentable sugar, wherein the plant part is obtained from a transformed plant, the genome of which is augmented with an expression cassette encoding the at least one enzyme; and incubating the fermentable sugar under conditions that promote the conversion of the fermentable sugar into ethanol is further provided. The enzyme may be hyperthermophilic.

Moreover, a method to produce a sweetened farinaceous food product without adding additional sweetener comprising treating a plant part comprising at least one starch processing enzyme under conditions which activate the at least one enzyme, thereby processing starch granules in the plant part to sugars so as to form a sweetened product, wherein the plant part is obtained from a transformed plant, the genome of which is augmented with an expression cassette encoding the at least one enzyme; and processing the sweetened product into a farinaceous food product is described. The farinaceous food product may be formed from the sweetened product and water. Moreover, the farinaceous food product may contain malt, flavorings, vitamins, minerals, coloring agents or any combination thereof. The at least one enzyme may be hyperthermophilic. The enzyme may be selected from α-amylase, α-glucosidase, glucoamylase, pullulanase, glucose isomerase, or any combination thereof. The plant may further be selected from the group consisting of soybean, rye, oats, barley, wheat, corn, rice and sugar cane. The farinaceous food product may be a cereal food, a breakfast food, a ready to eat food, or a baked food. The processing may include baking, boiling, heating, steaming, electrical discharge or any combination thereof.

The present invention is further directed to a method to sweeten a starch-containing product without adding sweetener comprising treating starch comprising at least one starch processing enzyme under conditions to activate the at least one enzyme thereby digesting the starch to form a sugar to form sweetened starch, wherein the starch is obtained from a transformed plant, the genome of which is augmented with an expression cassette encoding the at least one enzyme; and adding the sweetened starch to a product to produce a sweetened starch containing product. The transformed plant may be selected from the group consisting of corn, soybean, rye, oats, barley, wheat, rice and sugar cane. The at least one enzyme may be hyperthermophilic. The at least one enzyme may be α-amylase, α-glucosidase, glucoamylase, pullulanase, glucose isomerase, or any combination thereof.

A farinaceous food product and sweetened starch-containing product is provided for herein.

The invention is also directed to a method to sweeten a polysaccharide-containing fruit or vegetable comprising treating a fruit or vegetable comprising at least one polysaccharide processing enzyme under conditions which activate the at least one enzyme, thereby processing the polysaccharide in the fruit or vegetable to form sugar, yielding a sweetened fruit or vegetable, wherein the fruit or vegetable is obtained from a transformed plant, the genome of which is augmented with an expression cassette encoding the at least one polysaccharide processing enzyme. The fruit or vegetable is selected from the group consisting of potato, tomato, banana, squash, peas, and beans. The at least one enzyme may be hyperthermophilic.

The present invention is further directed to a method of preparing an aqueous solution comprising sugar comprising treating starch granules obtained from the plant part under conditions which activate the at least one enzyme, thereby yielding an aqueous solution comprising sugar.

Another embodiment is directed to a method of preparing starch derived products from grain that does not involve wet or dry milling grain prior to recovery of starch-derived products comprising treating a plant part comprising starch granules and at least one starch processing enzyme under conditions which activate the at least one enzyme thereby processing the starch granules to form an aqueous solution comprising dextrins or sugars, wherein the plant part is obtained from a transformed plant, the genome of which is augmented with an expression cassette encoding the at least one starch processing enzyme; and collecting the aqueous solution comprising the starch derived product. The at least one starch processing enzyme may be hyperthermophilic.

A method of isolating an α-amylase, glucoamylase, glucose isomerase, α-glucosidase, and pullulanase comprising culturing a transformed plant containing the α-amylase, glucoamylase, glucose isomerase, α-glucosidase, or pullulanase and isolating the α-amylase, glucoamylase, glucose isomerase, α-glucosidase or pullulanase therefrom is further provided. Also provided is a method of isolating a xylanase, cellulase, glucanase, beta glucosidase, protease, esterase, phytase or lipase comprising culturing a transformed plant containing the xylanase, cellulase, glucanase, beta glucosidase, protease, esterase, phytase or lipase and isolating the xylanase, cellulase, glucanase, esterase, beta glucosidase, protease, esterase, phytase or lipase.

A method of preparing maltodextrin comprising mixing transgenic grain with water, heating said mixture, separating solid from the dextrin syrup generated, and collecting the maltodextrin. The transgenic grain comprises at least one starch processing enzyme. The starch processing enzyme may be α-amylase, glucoamylase, α-glucosidase, and glucose isomerase. Moreover, maltodextrin produced by the method is provided as well as composition produced by this method.

A method of preparing dextrins, or sugars from grain that does not involve mechanical disruption of the grain prior to recovery of starch-derived comprising:

treating a plant part comprising starch granules and at least one starch processing enzyme under conditions which activate the at least one enzyme thereby processing the starch granules to form an aqueous solution comprising dextrins or sugars, wherein the plant part is obtained from a transformed plant, the genome of which is augmented with an expression cassette encoding the at least one processing enzyme; and
collecting the aqueous solution comprising sugar and/or dextrins is provided.

The present invention is further directed to a method of producing fermentable sugar comprising treating a plant part comprising starch granules and at least one starch processing enzyme under conditions which activate the at least one enzyme thereby processing the starch granules to form an aqueous solution comprising dextrins or sugars, wherein the plant part is obtained from a transformed plant, the genome of which is augmented with an expression cassette encoding the at least one processing enzyme; and

collecting the aqueous solution comprising the fermentable sugar.

Moreover, a maize plant stably transformed with a vector comprising a hyperthermophlic α-amylase is provided herein. For example, preferably, a maize plant stably transformed with a vector comprising a polynucleotide sequence that encodes α-amylase that is greater than 60% identical to SEQ ID NO: 1 or SEQ ID NO: 51 is encompassed.

### BRIEF DESCRIPTION OF THE FIGURES

FIGS. 1A and 1B illustrate the activity of α-amylase expressed in corn kernels and in the endosperm from segregating T1 kernels from pNOV6201 plants and from six pNOV6200 lines.

FIG. 2 illustrates the activity of α-amylase in segregating T1 kernels from pNOV6201 lines.

FIG. 3 depicts the amount of ethanol produced upon fermentation of mashes of transgenic corn containing thermostable 797GL3 alpha amylase that were subjected to liquefaction times of up to 60 minutes at 85° C. and 95° C. This figure illustrates that the ethanol yield at 72 hours of fermentation was almost unchanged from 15 minutes to 60 minutes of liquefaction. Moreover, it shows that mash produced by liquefaction at 95° C. produced more ethanol at each time point than mash produced by liquefaction at 85° C.

FIG. 4 depicts the amount of residual starch (%) remaining after fermentation of mashes of transgenic corn containing thermostable alpha amylase that were subjected to a liquefaction time of up to 60 minutes at 85° C. and 95° C. This figure illustrates that the ethanol yield at 72 hours of fermentation was almost unchanged from 15 minutes to 60 minutes of liquefaction. Moreover, it shows that mash produced by liquefaction at 95° C. produced more ethanol at each time point than mash produced by liquefaction at 85° C.

FIG. 5 depicts the ethanol yields for mashes of a transgenic corn, control corn, and various mixtures thereof prepared at 85° C. and 95° C. This figure illustrates that the transgenic corn comprising α-amylase results in significant improvement in making starch available for fermentation since there was a reduction of starch left over after fermentation.

FIG. 6 depicts the amount of residual starch measured in dried stillage following fermentation for mashes of a transgenic grain, control corn, and various mixtures thereof at prepared at 85° C. and 95° C.

FIG. 7 depicts the ethanol yields as a function of fermentation time of a sample comprising 3% transgenic corn over a period of 20-80 hours at various pH ranges from 5.2-6.4. The figure illustrates that the fermentation conducted at a lower pH proceeds faster than at a pH of 6.0 or higher.

FIG. 8 depicts the ethanol yields during fermentation of a mash comprising various weight percentages of transgenic corn from 0-12 wt % at various pH ranges from 5.2-6.4. This figure illustrates that the ethanol yield was independent of the amount of transgenic grain included in the sample.

FIG. 9 shows the analysis of T2 seeds from different events transformed with pNOV 7005. High expression of pullulanase activity, compared to the non-transgenic control, can be detected in a number of events.

FIGS. 10A and 10B show the results of the HPLC analysis of the hydrolytic products generated by expressed pullulanase from starch in the transgenic corn flour. Incubation of the flour of pullulanase expressing corn in reaction buffer at 75° C. for 30 minutes results in production of medium chain oligosaccharides (degree of polymerization (DP)˜10-30) and short amylose chains (DP˜100-200) from cornstarch. FIGS. 10A and 10B also show the effect of added calcium ions on the activity of the pullulanase.

FIGS. 11A and 11B depict the data generated from HPLC analysis of the starch hydrolysis product from two reaction mixtures. The first reaction indicated as ‘Amylase’ contains a mixture [1:1 (w/w)] of corn flour samples of α-amylase expressing transgenic corn and non-transgenic corn A188; and the second reaction mixture ‘Amylase+Pullulanase’ contains a mixture [1:1 (w/w)] of corn flour samples of α-amylase expressing transgenic corn and pullulanase expressing transgenic corn.

FIG. 12 depicts the amount of sugar product in μg in 25 μl of reaction mixture for two reaction mixtures. The first reaction indicated as ‘Amylase’ contains a mixture [1:1 (w/w)] of corn flour samples of α-amylase expressing transgenic corn and non-transgenic corn A188; and the second reaction mixture ‘Amylase+Pullulanase’, contains a mixture [1:1 (w/w)] of corn flour samples of α-amylase expressing transgenic corn and pullulanase expressing transgenic corn.

FIGS. 13A and 13B shows the starch hydrolysis product from two sets of reaction mixtures at the end of 30 minutes incubation at 85° C. and 95° C. For each set there are two reaction mixtures; the first reaction indicated as ‘Amylase X Pullulanase’ contains flour from transgenic corn (generated by cross pollination) expressing both the α-amylase and the pullulanase, and the second reaction indicated as ‘Amylase’ mixture of corn flour samples of α-amylase expressing transgenic corn and non-transgenic corn A188 in a ratio so as to obtain same amount of α-amylase activity as is observed in the cross (Amylase ×Pullulanase).

FIG. 14 depicts the degradation of starch to glucose using non-transgenic corn seed (control), transgenic corn seed comprising the 797GL3 α-amylase, and a combination of 797GL3 transgenic corn seed with Mal A α-glucosidase.

FIG. 15 depicts the conversion of raw starch at room temperature or 30° C. In this figure, the reaction mixtures 1 and 2 are a combination of water and starch at room temperature and 30° C., respectively. Reaction mixtures 3 and 4 are a combination of barley α-amylase and starch at room temperature and at 30° C., respectively. Reaction mixtures 5 and 6 are combinations of Thermoanaerobacterium glucoamylase and starch at room temperature and 30° C., respectively. Reactions mixtures 7 and 8 are combinations of barley α-amylase (sigma) and Thermoanaerobacterium glucoamylase and starch at room temperature and 30° C., respectively. Reaction mixtures 9 and 10 are combinations of Barley alpha-amylase (sigma) control, and starch at room temperature and 30° C., respectively. The degree of polymerization (DP) of the products of the Thermoanaerobacterium glucoamylase is indicated.

FIG. 16 depicts the production of fructose from amylase transgenic corn flour using a combination of alpha amylase, alpha glucosidase, and glucose isomerase as described in Example 19. Amylase corn flour was mixed with enzyme solutions plus water or buffer. All reactions contained 60 mg amylase flour and a total of 600 μl of liquid and were incubated for 2 hours at 90° C.

FIG. 17 depicts the peak areas of the products of reaction with 100% amylase flour from a self-processing kernel as a function of incubation time from 0-1200 minutes at 90° C.

FIG. 18 depicts the peak areas of the products of reaction with 10% transgenic amylase flour from a self-processing kernel and 90% control corn flour as a function of incubation time from 0-1200 minutes at 90° C.

FIG. 19 provides the results of the HPLC analysis of transgenic amylase flour incubated at 70°, 80°, 90°, or 100° C. for up to 90 minutes to assess the effect of temperature on starch hydrolysis.

FIG. 20 depicts ELSD peak area for samples containing 60 mg transgenic amylase flour mixed with enzyme solutions plus water or buffer under various reaction conditions. One set of reactions was buffered with 50 mM MOPS, pH 7.0 at room temperature, plus 10 mM MgSO4 and 1 mM CoCl₂; in a second set of reactions the metal-containing buffer solution was replaced by water. All reactions were incubated for 2 hours at 90° C.

### DETAILED DESCRIPTION OF THE INVENTION

In accordance with the present invention, a “self-processing” plant or plant part has incorporated therein an isolated polynucleotide encoding a processing enzyme capable of processing, e.g., modifying, starches, polysaccharides, lipids, proteins, and the like in plants, wherein the processing enzyme can be mesophilic, thermophilic or hyperthermophilic, and may be activated by grinding, addition of water, heating, or otherwise providing favorable conditions for function of the enzyme. The isolated polynucleotide encoding the processing enzyme is integrated into a plant or plant part for expression therein. Upon expression and activation of the processing enzyme, the plant or plant part of the present invention processes the substrate upon which the processing enzyme acts. Therefore, the plant or plant parts of the present invention are capable of self-processing the substrate of the enzyme upon activation of the processing enzyme contained therein in the absence of or with reduced external sources normally required for processing these substrates. As such, the transformed plants, transformed plant cells, and transformed plant parts have “built-in” processing capabilities to process desired substrates via the enzymes incorporated therein according to this invention. Preferably, the processing enzyme-encoding polynucleotide are “genetically stable,” i.e., the polynucleotide is stably maintained in the transformed plant or plant parts of the present invention and stably inherited by progeny through successive generations.

In accordance with the present invention, methods which employ such plants and plant parts can eliminate the need to mill or otherwise physically disrupt the integrity of plant parts prior to recovery of starch-derived products. For example, the invention provides improved methods for processing corn and other grain to recover starch-derived products. The invention also provides a method which allows for the recovery of starch granules that contain levels of starch degrading enzymes, in or on the granules, that are adequate for the hydrolysis of specific bonds within the starch without the requirement for adding exogenously produced starch hydrolyzing enzymes. The invention also provides improved products from the self-processing plant or plant parts obtained by the methods of the invention.

In addition, the “self-processing” transformed plant part, e.g., grain, and transformed plant avoid major problems with existing technology, i.e., processing enzymes are typically produced by fermentation of microbes, which requires isolating the enzymes from the culture supernatants, which costs money; the isolated enzyme needs to be formulated for the particular application, and processes and machinery for adding, mixing and reacting the enzyme with its substrate must be developed. The transformed plant of the invention or a part thereof is also a source of the processing enzyme itself as well as substrates and products of that enzyme, such as sugars, amino acids, fatty acids and starch and non-starch polysaccharides. The plant of the invention may also be employed to prepare progeny plants such as hybrids and inbreds.

### Processing Enzymes and Polynucleotides Encoding Them

A polynucleotide encoding a processing enzyme (mesophilic, thermophilic, or hyperthermophilic) is introduced into a plant or plant part. The processing enzyme is selected based on the desired substrate upon which it acts as found in plants or transgenic plants and/or the desired end product. For example, the processing enzyme may be a starch-processing enzyme, such as a starch-degrading or starch-isomerizing enzyme, or a non-starch processing enzyme. Suitable processing enzymes include, but are not limited to, starch degrading or isomerizing enzymes including, for example, α-amylase, endo or exo-1,4, or 1,6-α-D, glucoamylase, glucose isomerase, β-amylases, α-glucosidases, and other exo-amylases; and starch debranching enzymes, such as isoamylase, pullulanase, neo-pullulanase, iso-pullulanase, amylopullulanase and the like, glycosyl transferases such as cyclodextrin glycosyltransferase and the like, cellulases such as exo-1,4-β-cellobiohydrolase, exo-1,3-β-D-glucanase, hemicellulase, β-glucosidase and the like; endoglucanases such as endo-1,3-β-glucanase and endo-1,4-β-glucanase and the like; L-arabinases, such as endo-1,5-α-L-arabinase, α-arabinosidases and the like; galactanases such as endo-1,4-β-D-galactanase, endo-1,3-β-D-galactanase, β-galactosidase, α-galactosidase and the like; mannanases, such as endo-1,4-β-D-mannanase, β-mannosidase, α-mannosidase and the like; xylanases, such as endo-1,4-β-xylanase, β-D-xylosidase, 1,3-β-D-xylanase, and the like; and pectinases; and non-starch processing enzymes, including protease, glucanase, xylanase, thioredoxin/thioredoxin reductase, esterase, phytase, and lipase.

In one embodiment, the processing enzyme is a starch-degrading enzyme selected from the group of α-amylase, pullulanase, α-glucosidase, glucoamylase, amylopullulanase, glucose isomerase, or combinations thereof. According to this embodiment, the starch-degrading enzyme is able to allow the self-processing plant or plant part to degrade starch upon activation of the enzyme contained in the plant or plant part, as will be further described herein. The starch-degrading enzyme(s) is selected based on the desired end-products. For example, a glucose-isomerase may be selected to convert the glucose (hexose) into fructose. Alternatively, the enzyme may be selected based on the desired starch-derived end product with various chain lengths based on, e.g., a function of the extent of processing or with various branching patterns desired. For example, an α-amylase, glucoamylase, or amylopullulanase can be used under short incubation times to produce dextrin products and under longer incubation times to produce shorter chain products or sugars. A pullulanase can be used to specifically hydrolyze branch points in the starch yielding a high-amylose starch, or a neopullulanase can be used to produce starch with stretches of α1,4 linkages with interspersed α1,6 linkages. Glucosidases could be used to produce limit dextrins, or a combination of different enzymes to make other starch derivatives.

In another embodiment, the processing enzyme is a non-starch processing enzyme selected from protease, glucanase, xylanase, phytase, lipase, cellulase, beta glucosidase and esterase. These non-starch degrading enzymes allow the self-processing plant or plant part of the present invention to incorporate in a targeted area of the plant and, upon activation, disrupt the plant while leaving the starch granule therein intact. For example, in a preferred embodiment, the non-starch degrading enzymes target the endosperm matrix of the plant cell and, upon activation, disrupt the endosperm matrix while leaving the starch granule therein intact and more readily recoverable from the resulting material.

Combinations of processing enzymes are further envisioned by the present invention. For example, starch-processing and non-starch processing enzymes may be used in combination. Combinations of processing enzymes may be obtained by employing the use of multiple gene constructs encoding each of the enzymes. Alternatively, the individual transgenic plants stably transformed with the enzymes may be crossed by known methods to obtain a plant containing both enzymes. Another method includes the use of exogenous enzyme(s) with the transgenic plant.

The processing enzymes may be isolated or derived from any source and the polynucleotides corresponding thereto may be ascertained by one having skill in the art. For example, the processing enzyme, such as α-amylase, is derived from the Pyrococcus (e.g., Pyrococcus furiosus), Thermus, Thermococcus (e.g., Thermococcus hydrothermalis), Sulfolobus (e.g., Sulfolobus solfataricus) Thermotoga (e.g., Thermotoga maritima and Thermotoga neapolitana), Thermoanaerobacterium (e.g. Thermoanaerobacter tengcongensis), Aspergillus (e.g., Aspergillus shirousami and Aspergillus niger), Rhizopus (eg., Rhizopus oryzae), Thermoproteales, Desulfurococcus (e.g. Desulfurococcus amylolyticus), Methanobacterium thermoautotrophicum, Methanococcus jannaschii, Methanopyrus kandleri, Thermosynechococcus elongatus, Thermoplasma acidophilum, Thermoplasma volcanium, Aeropyrum pernix and plants such as corn, barley, and rice.

The processing enzymes of the present invention are capable of being activated after being introduced and expressed in the genome of a plant. Conditions for activating the enzyme are determined for each individual enzyme and may include varying conditions such as temperature, pH, hydration, presence of metals, activating compounds, inactivating compounds, etc. For example, temperature-dependent enzymes may include mesophilic, thermophilic, and hyperthermophilic enzymes. Mesophilic enzymes typically have maximal activity at temperatures between 20°-65° C. and are inactivated at temperatures greater than 70° C. Mesophilic enzymes have significant activity at 30 to 37° C., the activity at 30° C. is preferably at least 10% of maximal activity, more preferably at least 20% of maximal activity.

Thermophilic enzymes have a maximal activity at temperatures of between 50 and 80° C. and are inactivated at temperatures greater than 80° C. A thermophilic enzyme will preferably have less than 20% of maximal activity at 30° C., more preferably less than 10% of maximal activity.

A “hyperthermophilic” enzyme has activity at even higher temperatures. Hyperthermophilic enzymes have a maximal activity at temperatures greater than 80° C. and retain activity at temperatures at least 80° C., more preferably retain activity at temperatures of at least 90° C. and most preferably retain activity at temperatures of at least 95° C. Hyperthermophilic enzymes also have reduced activity at low temperatures. A hyperthermophilic enzyme may have activity at 30° C. that is less than 10% of maximal activity, and preferably less than 5% of maximal activity.

The polynucleotide encoding the processing enzyme is preferably modified to include codons that are optimized for expression in a selected organism such as a plant (see, e.g., Wada et al., Nucl. Acids Res., 18:2367 (1990), Murray et al., Nucl. Acids Res., 17:477 (1989), U.S. Pat. Nos. 5,096,825, 5,625,136, 5,670,356 and 5,874,304). Codon optimized sequences are synthetic sequences, i.e., they do not occur in nature, and preferably encode the identical polypeptide (or an enzymatically active fragment of a full length polypeptide which has substantially the same activity as the full length polypeptide) encoded by the non-codon optimized parent polynucleotide which encodes a processing enzyme. It is preferred that the polypeptide is biochemically distinct or improved, e.g., via recursive mutagenesis of DNA encoding a particular processing enzyme, from the parent source polypeptide such that its performance in the process application is improved. Preferred polynucleotides are optimized for expression in a target host plant and encode a processing enzyme. Methods to prepare these enzymes include mutagenesis, e.g., recursive mutagenesis and selection. Methods for mutagenesis and nucleotide sequence alterations are well-known in the art. See, for example, Kunkel, Proc. Natl. Acad. Sci. USA, 82:488, (1985); Kunkel et al., Methods in Enzymol., 154:367 (1987); U.S. Pat. No. 4,873,192; Walker and Gaastra, eds. (1983) Techniques in Molecular Biology (MacMillan Publishing Company, New York) and the references cited therein and Arnold et al., Chem. Eng. Sci, 51:5091 (1996)). Methods to optimize the expression of a nucleic acid segment in a target plant or organism are well-known in the art. Briefly, a codon usage table indicating the optimal codons used by the target organism is obtained and optimal codons are selected to replace those in the target polynucleotide and the optimized sequence is then chemically synthesized. Preferred codons for maize are described in U.S. Pat. No. 5,625,136.

Complementary nucleic acids of the polynucleotides of the present invention are further envisioned. An example of low stringency conditions for hybridization of complementary nucleic acids which have more than 100 complementary residues on a filter in a Southern or Northern blot is 50% formamide, e.g., hybridization in 50% formamide, 1 M NaCl, 1% SDS at 37° C., and a wash in 0.1×SSC at 60° C. to 65° C. Exemplary low stringency conditions include hybridization with a buffer solution of 30 to 35% formamide, 1 M NaCl, 1% SDS (sodium dodecyl sulphate) at 37° C., and a wash in 1× to 2×SSC (20×SSC=3.0 M NaCl/0.3 M trisodium citrate) at 50 to 55° C. Exemplary moderate stringency conditions include hybridization in 40 to 45% formamide, 1.0 M NaCl, 1% SDS at 37° C., and a wash in 0.5× to 1×SSC at 55 to 60° C.

Moreover, polynucleotides encoding an “enzymatically active” fragment of the processing enzymes are further envisioned. As used herein, “enzymatically active” means a polypeptide fragment of the processing enzyme that has substantially the same biological activity as the processing enzyme to modify the substrate upon which the processing enzyme normally acts under appropriate conditions.

In a preferred embodiment, the polynucleotide of the present invention is a maize-optimized polynucleotide encoding α-amylase, such as provided in SEQ ID NOs:2, 9, 46, and 52. In another preferred embodiment, the polynucleotide is a maize-optimized polynucleotide encoding pullulanase, such as provided in SEQ ID NOs: 4 and 25. In yet another preferred embodiment, the polynucleotide is a maize-optimized polynucleotide encoding α-glucosidase as provided in SEQ ID NO:6. Another preferred polynucleotide is the maize-optimized polynucleotide encoding glucose isomerase having SEQ ID NO: 19, 21, 37, 39, 41, or 43. In another embodiment, the maize-optimized polynucleotide encoding glucoamylase as set forth in SEQ ID NO: 46, 48, or 50 is preferred. Moreover, a maize-optimized polynucleotide for glucanase/mannanase fusion polypeptide is provided in SEQ ID NO: 57. The invention further provides for complements of such polynucleotides, which hybridize under moderate, or preferably under low stringency, hybridization conditions and which encodes a polypeptide having α-amylase, pullulanase, α-glucosidase, glucose isomerase, glucoamylase, glucanase, or mannanase activity, as the case may be.

The polynucleotide may be used interchangeably with “nucleic acid” or “polynucleic acid” and refers to deoxyribonucleotides or ribonucleotides and polymers thereof in either single- or double-stranded form, composed of monomers (nucleotides) containing a sugar, phosphate and a base, which is either a purine or pyrimidine. Unless specifically limited, the term encompasses nucleic acids containing known analogs of natural nucleotides, which have similar binding properties as the reference nucleic acid and are metabolized in a manner similar to naturally occurring nucleotides. Unless otherwise indicated, a particular nucleic acid sequence also implicitly encompasses conservatively modified variants thereof (e.g., degenerate codon substitutions) and complementary sequences as well as the sequence explicitly indicated. Specifically, degenerate codon substitutions may be achieved by generating sequences in which the third position of one or more selected (or all) codons is substituted with mixed-base and/or deoxyinosine residues.

“Variants” or substantially similar sequences are further encompassed herein. For nucleotide sequences, variants include those sequences that, because of the degeneracy of the genetic code, encode the identical amino acid sequence of the native protein. Naturally occurring allelic variants such as these can be identified with the use of well-known molecular biology techniques, as, for example, with polymerase chain reaction (PCR), hybridization techniques, and ligation reassembly techniques. Variant nucleotide sequences also include synthetically derived nucleotide sequences, such as those generated, for example, by using site-directed mutagenesis, which encode the native protein, as well as those that encode a polypeptide having amino acid substitutions. Generally, nucleotide sequence variants of the invention will have at least 40%, 50%, 60%, preferably 70%, more preferably 80%, even more preferably 90%, most preferably 99%, and single unit percentage identity to the native nucleotide sequence based on these classes. For example, 71%, 72%, 73% and the like, up to at least the 90% class. Variants may also include a full-length gene corresponding to an identified gene fragment.

### Regulatory Sequences: Promoters/Signal Sequences/Selectable Markers

The polynucleotide sequences encoding the processing enzyme of the present invention may be operably linked to polynucleotide sequences encoding localization signals or signal sequence (at the N- or C-terminus of a polypeptide), e.g., to target the hyperthermophilic enzyme to a particular compartment within a plant. Examples of such targets include, but are not limited to, the vacuole, endoplasmic reticulum, chloroplast, amyloplast, starch granule, or cell wall, or to a particular tissue, e.g., seed. The expression of a polynucleotide encoding a processing enzyme having a signal sequence in a plant, in particular, in conjunction with the use of a tissue-specific or inducible promoter, can yield high levels of localized processing enzyme in the plant. Numerous signal sequences are known to influence the expression or targeting of a polynucleotide to a particular compartment or outside a particular compartment. Suitable signal sequences and targeting promoters are known in the art and include, but are not limited to, those provided herein.

For example, where expression in specific tissues or organs is desired, tissue-specific promoters may be used. In contrast, where gene expression in response to a stimulus is desired, inducible promoters are the regulatory elements of choice. Where continuous expression is desired throughout the cells of a plant, constitutive promoters are utilized. Additional regulatory sequences upstream and/or downstream from the core promoter sequence may be included in expression constructs of transformation vectors to bring about varying levels of expression of heterologous nucleotide sequences in a transgenic plant.

A number of plant promoters have been described with various expression characteristics. Examples of some constitutive promoters which have been described include the rice actin 1 (Wang et al., Mol. Cell. Biol., 12:3399 (1992); U.S. Pat. No. 5,641,876), CaMV 35S (Odell et al., Nature, 313:810 (1985)), CaMV 19S (Lawton et al., 1987), nos (Ebert et al., 1987), Adh (Walker et al., 1987), sucrose synthase (Yang & Russell, 1990), and the ubiquitin promoters.

Vectors for use in tissue-specific targeting of genes in transgenic plants will typically include tissue-specific promoters and may also include other tissue-specific control elements such as enhancer sequences. Promoters which direct specific or enhanced expression in certain plant tissues will be known to those of skill in the art in light of the present disclosure. These include, for example, the rbcS promoter, specific for green tissue; the ocs, nos and mas promoters which have higher activity in roots or wounded leaf tissue; a truncated (−90 to +8) 35S promoter which directs enhanced expression in roots, an α-tubulin gene that directs expression in roots and promoters derived from zein storage protein genes which direct expression in endosperm.

Tissue specific expression may be functionally accomplished by introducing a constitutively expressed gene (all tissues) in combination with an antisense gene that is expressed only in those tissues where the gene product is not desired. For example, a gene coding for a lipase may be introduced such that it is expressed in all tissues using the 35S promoter from Cauliflower Mosaic Virus. Expression of an antisense transcript of the lipase gene in a maize kernel, using for example a zein promoter, would prevent accumulation of the lipase protein in seed. Hence the protein encoded by the introduced gene would be present in all tissues except the kernel.

Moreover, several tissue-specific regulated genes and/or promoters have been reported in plants. Some reported tissue-specific genes include the genes encoding the seed storage proteins (such as napin, cruciferin, beta-conglycinin, and phaseolin) zein or oil body proteins (such as oleosin), or genes involved in fatty acid biosynthesis (including acyl carrier protein, stearoyl-ACP desaturase, and fatty acid desaturases (fad 2-1)), and other genes expressed during embryo development (such as Bce4, see, for example, EP 255378 and Kridl et al., Seed Science Research, 1:209 (1991)). Examples of tissue-specific promoters, which have been described include the lectin (Vodkin, Prog. Clin. Biol. Res., 138; 87 (1983); Lindstrom et al., Der. Genet., 11:160 (1990)), corn alcohol dehydrogenase 1 (Vogel et al., 1989; Dennis et al., Nucleic Acids Res., 12:3983 (1984)), corn light harvesting complex (Simpson, 1986; Bansal et al., Proc. Natl. Acad. Sci. USA, 89:3654 (1992)), corn heat shock protein (Odell et al., 1985; Rochester et al., 1986), pea small subunit RuBP carboxylase (Poulsen et al., 1986; Cashmore et al., 1983), Ti plasmid mannopine synthase (Langridge et al., 1989), Ti plasmid nopaline synthase (Langridge et al., 1989), petunia chalcone isomerase (vanTunen et al., EMBO J., 7; 1257 (1988)), bean glycine rich protein 1 (Keller et al., Genes Dev., 3:1639 (1989)), truncated CaMV 35S (Odell et al., Nature, 313:810 (1985)), potato patatin (Wenzler et al., Plant Mol. Biol., 13:347 (1989)), root cell (Yamamoto et al., Nucleic Acids Res., 18:7449 (1990)), maize zein (Reina et al., Nucleic Acids Res., 18:6425 (1990); Kriz et al., Mol. Gen. Genet., 207:90 (1987); Wandelt et al., Nucleic Acids Res., 17:2354 (1989); Langridge et al., Cell, 34:1015 (1983); Reina et al., Nucleic Acids Res., 18:7449 (1990)), globulin-1 (Belanger et al., Genetics, 129:863 (1991)), α-tubulin, cab (Sullivan et al., Mol. Gen. Genet., 215:431 (1989)), PEPCase (Hudspeth & Grula, 1989), R gene complex-associated promoters (Chandler et al., Plant Cell, 1:1175 (1989)), and chalcone synthase promoters (Franken et al., EMBO J., 10:2605 (1991)). Particularly useful for seed-specific expression is the pea vicilin promoter (Czako et al., Mol. Gen. Genet., 235:33 (1992). (See also U.S. Pat. No. 5,625,136, herein incorporated by reference.) Other useful promoters for expression in mature leaves are those that are switched on at the onset of senescence, such as the SAG promoter from Arabidopsis (Gan et al., Science, 270:1986 (1995).

A class of fruit-specific promoters expressed at or during anthesis through fruit development, at least until the beginning of ripening, is discussed in U.S. Pat. No. 4,943,674, the disclosure of which is hereby incorporated by reference. cDNA clones that are preferentially expressed in cotton fiber have been isolated (John et al., Proc. Natl. Acad. Sci. USA, 89:5769 (1992). cDNA clones from tomato displaying differential expression during fruit development have been isolated and characterized (Mansson et al., Gen. Genet., 200:356 (1985), Slater et al., Plant Mol. Biol., 5:137 (1985)). The promoter for polygalacturonase gene is active in fruit ripening. The polygalacturonase gene is described in U.S. Pat. No. 4,535,060, U.S. Pat. No. 4,769,061, U.S. Pat. No. 4,801,590, and U.S. Pat. No. 5,107,065, which disclosures are incorporated herein by reference.

Other examples of tissue-specific promoters include those that direct expression in leaf cells following damage to the leaf (for example, from chewing insects), in tubers (for example, patatin gene promoter), and in fiber cells (an example of a developmentally-regulated fiber cell protein is E6 (John et al., Proc. Natl. Acad. Sci. USA, 89:5769 (1992). The E6 gene is most active in fiber, although low levels of transcripts are found in leaf, ovule and flower.

The tissue-specificity of some “tissue-specific” promoters may not be absolute and may be tested by one skilled in the art using the diphtheria toxin sequence. One can also achieve tissue-specific expression with “leaky” expression by a combination of different tissue-specific promoters (Beals et al., Plant Cell, 9:1527 (1997)). Other tissue-specific promoters can be isolated by one skilled in the art (see U.S. Pat. No. 5,589,379).

In one embodiment, the direction of the product from a polysaccharide hydrolysis gene, such as α-amylase, may be targeted to a particular organelle such as the apoplast rather than to the cytoplasm. This is exemplified by the use of the maize γ-zein N-terminal signal sequence (SEQ ID NO:17), which confers apoplast-specific targeting of proteins. Directing the protein or enzyme to a specific compartment will allow the enzyme to be localized in a manner that it will not come into contact with the substrate. In this manner the enzymatic action of the enzyme will not occur until the enzyme contacts its substrate. The enzyme can be contacted with its substrate by the process of milling (physical disruption of the cell integrity), or heating the cells or plant tissues to disrupt the physical integrity of the plant cells or organs that contain the enzyme. For example a mesophilic starch-hydrolyzing enzyme can be targeted to the apoplast or to the endoplasmic reticulum and so as not to come into contact with starch granules in the amyloplast. Milling of the grain will disrupt the integrity of the grain and the starch hydrolyzing enzyme will then contact the starch granules. In this manner the potential negative effects of co-localization of an enzyme and its substrate can be circumvented.

In another embodiment, a tissue-specific promoter includes the endosperm-specific promoters such as the maize γ-zein promoter (exemplified by SEQ ID NO:12) or the maize ADP-gpp promoter (exemplified by SEQ ID NO:11, which includes a 5′ untranslated and an intron sequence) or a Q protein promoter (exemplified by SEQ ID NO: 98) or a rice glutelin 1 promoter (exemplified in SEQ ID NO:67). Thus, the present invention includes an isolated polynucleotide comprising a promoter comprising SEQ ID NO:11, 12, 67, or 98, a polynucleotide which hybridizes to the complement thereof under low stringency hybridization conditions, or a fragment thereof which has promoter activity, e.g., at least 10%, and preferably at least 50%, the activity of a promoter having SEQ ID NO: 11, 12, 67, or 98.

In another embodiment of the invention, the polynucleotide encodes a hyperthermophilic processing enzyme that is operably linked to a chloroplast (amyloplast) transit peptide (CTP) and a starch binding domain, e.g., from the waxy gene. An exemplary polynucleotide in this embodiment encodes SEQ ID NO:10 (α-amylase linked to the starch binding domain from waxy). Other exemplary polynucleotides encode a hyperthermophilic processing enzyme linked to a signal sequence that targets the enzyme to the endoplasmic reticulum and secretion to the apoplast (exemplified by a polynucleotide encoding SEQ ID NO:13, 27, or 30, which comprises the N-terminal sequence from maize γ-zein operably linked to α-amylase, α-glucosidase, glucose isomerase, respectively), a hyperthermophilic processing enzyme linked to a signal sequence which retains the enzyme in the endoplasmic reticulum (exemplified by a polynucleotide encoding SEQ ID NO:14, 26, 28, 29, 33, 34, 35, or 36, which comprises the N-terminal sequence from maize γ-zein operably linked to the hyperthermophilic enzyme, which is operably linked to SEKDEL, wherein the enzyme is α-amylase, malA α-glucosidase, T. maritima glucose isomerase, T. neapolitana glucose isomerase), a hyperthermophilic processing enzyme linked to an N-terminal sequence that targets the enzyme to the amyloplast (exemplified by a polynucleotide encoding SEQ ID NO:15, which comprises the N-terminal amyloplast targeting sequence from waxy operably linked to α-amylase), a hyperthermophilic fusion polypeptide which targets the enzyme to starch granules (exemplified by a polynucleotide encoding SEQ ID NO:16, which comprises the N-terminal amyloplast targeting sequence from waxy operably linked to an α-amylase/waxy fusion polypeptide comprising the waxy starch binding domain), a hyperthermophilic processing enzyme linked to an ER retention signal (exemplified by a polynucleotide encoding SEQ ID NO:38 and 39). Moreover, a hyperthermophilic processing enzyme may be linked to a raw-starch binding site having the amino acid sequence (SEQ ID NO:53), wherein the polynucleotide encoding the processing enzyme is linked to the maize-optimized nucleic acid sequence (SEQ ID NO:54) encoding this binding site.

Several inducible promoters have been reported. Many are described in a review by Gatz, in Current Opinion in Biotechnology, 7:168 (1996) and Gatz, C., Annu. Rev. Plant Physiol. Plant Mol. Biol., 48:89 (1997). Examples include tetracycline repressor system, Lac repressor system, copper-inducible systems, salicylate-inducible systems (such as the PR1a system), glucocorticoid-inducible (Aoyama T. et al., N-H Plant Journal, 11:605 (1997)) and ecdysone-inducible systems. Other inducible promoters include ABA- and turgor-inducible promoters, the promoter of the auxin-binding protein gene (Schwob et al., Plant J., 4:423 (1993)), the UDP glucose flavonoid glycosyl-transferase gene promoter (Ralston et al., Genetics, 119:185 (1988)), the MPI proteinase inhibitor promoter (Cordero et al., Plant J., 6:141 (1994)), and the glyceraldehyde-3-phosphate dehydrogenase gene promoter (Kohler et al., Plant Mol. Biol., 29; 1293 (1995); Quigley et al., J. Mol. Evol., 29:412 (1989); Martinez et al., J. Mol. Biol., 208:551 (1989)). Also included are the benzene sulphonamide-inducible (U.S. Pat. No. 5,364,780) and alcohol-inducible (WO 97/06269 and WO 97/06268) systems and glutathione S-transferase promoters.

Other studies have focused on genes inducibly regulated in response to environmental stress or stimuli such as increased salinity, drought, pathogen and wounding. (Graham et al., J. Biol. Chem., 260:6555 (1985); Graham et al., J. Biol. Chem., 260:6561 (1985), Smith et al., Planta, 168:94 (1986)). Accumulation of metallocarboxypeptidase-inhibitor protein has been reported in leaves of wounded potato plants (Graham et al., Biochem. Biophys. Res. Comm., 101:1164 (1981)). Other plant genes have been reported to be induced by methyl jasmonate, elicitors, heat-shock, anaerobic stress, or herbicide safeners.

Regulated expression of a chimeric transacting viral replication protein can be further regulated by other genetic strategies, such as, for example, Cre-mediated gene activation (Odell et al. Mol. Gen. Genet., 113:369 (1990)). Thus, a DNA fragment containing 3′ regulatory sequence bound by lox sites between the promoter and the replication protein coding sequence that blocks the expression of a chimeric replication gene from the promoter can be removed by Cre-mediated excision and result in the expression of the trans-acting replication gene. In this case, the chimeric Cre gene, the chimeric trans-acting replication gene, or both can be under the control of tissue- and developmental-specific or inducible promoters. An alternate genetic strategy is the use of tRNA suppressor gene. For example, the regulated expression of a tRNA suppressor gene can conditionally control expression of a trans-acting replication protein coding sequence containing an appropriate termination codon (Ulmasov et al. Plant Mol. Biol., 35:417 (1997)). Again, either the chimeric tRNA suppressor gene, the chimeric transacting replication gene, or both can be under the control of tissue- and developmental-specific or inducible promoters.

Preferably, in the case of a multicellular organism, the promoter can also be specific to a particular tissue, organ or stage of development. Examples of such promoters include, but are not limited to, the Zea mays ADP-gpp and the Zea mays γ-zein promoter and the Zea mays globulin promoter.

Expression of a gene in a transgenic plant may be desired only in a certain time period during the development of the plant. Developmental timing is frequently correlated with tissue specific gene expression. For example, expression of zein storage proteins is initiated in the endosperm about 15 days after pollination.

Additionally, vectors may be constructed and employed in the intracellular targeting of a specific gene product within the cells of a transgenic plant or in directing a protein to the extracellular environment. This will generally be achieved by joining a DNA sequence encoding a transit or signal peptide sequence to the coding sequence of a particular gene. The resultant transit, or signal, peptide will transport the protein to a particular intracellular, or extracellular destination, respectively, and will then be post-translationally removed. Transit or signal peptides act by facilitating the transport of proteins through intracellular membranes, e.g., vacuole, vesicle, plastid and mitochondrial membranes, whereas signal peptides direct proteins through the extracellular membrane.

A signal sequence such as the maize γ-zein N-terminal signal sequence for targeting to the endoplasmic reticulum and secretion into the apoplast may be operably linked to a polynucleotide encoding a hyperthermophilic processing enzyme in accordance with the present invention (Torrent et al., 1997). For example, SEQ ID NOs:13, 27, and 30 provides for a polynucleotide encoding a hyperthermophilic enzyme operably linked to the N-terminal sequence from maize γ-zein protein. Another signal sequence is the amino acid sequence SEKDEL for retaining polypeptides in the endoplasmic reticulum (Munro and Pelham, 1987). For example, a polynucleotide encoding SEQ ID NOS:14, 26, 28, 29, 33, 34, 35, or 36, which comprises the N-terminal sequence from maize γ-zein operably linked to a processing enzyme which is operably linked to SEKDEL. A polypeptide may also be targeted to the amyloplast by fusion to the waxy amyloplast targeting peptide (Klosgen et al., 1986) or to a starch granule. For example, the polynucleotide encoding a hyperthermophilic processing enzyme may be operably linked to a chloroplast (amyloplast) transit peptide (CTP) and a starch binding domain, e.g., from the waxy gene. SEQ ID NO:10 exemplifies α-amylase linked to the starch binding domain from waxy. SEQ ID NO:15 exemplifies the N-terminal sequence amyloplast targeting sequence from waxy operably linked to α-amylase. Moreover, the polynucleotide encoding the processing enzyme may be fused to target starch granules using the waxy starch binding domain. For example, SEQ ID NO:16 exemplifies a fusion polypeptide comprising the N-terminal amyloplast targeting sequence from waxy operably linked to an α-amylase/waxy fusion polypeptide comprising the waxy starch binding domain.

The polynucleotides of the present invention, in addition to processing signals, may further include other regulatory sequences, as is known in the art. “Regulatory sequences” and “suitable regulatory sequences” each refer to nucleotide sequences located upstream (5′ non-coding sequences), within, or downstream (3′ non-coding sequences) of a coding sequence, and which influence the transcription, RNA processing or stability, or translation of the associated coding sequence. Regulatory sequences include enhancers, promoters, translation leader sequences, introns, and polyadenylation signal sequences. They include natural and synthetic sequences as well as sequences, which may be a combination of synthetic and natural sequences.

Selectable markers may also be used in the present invention to allow for the selection of transformed plants and plant tissue, as is well-known in the art. One may desire to employ a selectable or screenable marker gene as, or in addition to, the expressible gene of interest. “Marker genes” are genes that impart a distinct phenotype to cells expressing the marker gene and thus allow such transformed cells to be distinguished from cells that do not have the marker. Such genes may encode either a selectable or screenable marker, depending on whether the marker confers a trait which one can select for by chemical means, i.e., through the use of a selective agent (e.g., a herbicide, antibiotic, or the like), or whether it is simply a trait that one can identify through observation or testing, i.e., by screening (e.g., the R-locus trait). Of course, many examples of suitable marker genes are known to the art and can be employed in the practice of the invention.

Included within the terms selectable or screenable marker genes are also genes which encode a “secretable marker” whose secretion can be detected as a means of identifying or selecting for transformed cells. Examples include markers which encode a secretable antigen that can be identified by antibody interaction, or even secretable enzymes which can be detected by their catalytic activity. Secretable proteins fall into a number of classes, including small, diffusible proteins detectable, e.g., by ELISA; small active enzymes detectable in extracellular solution (e.g., α-amylase, β-lactamase, phosphinothricin acetyltransferase); and proteins that are inserted or trapped in the cell wall (e.g., proteins that include a leader sequence such as that found in the expression unit of extensin or tobacco PR-S).

With regard to selectable secretable markers, the use of a gene that encodes a protein that becomes sequestered in the cell wall, and which protein includes a unique epitope is considered to be particularly advantageous. Such a secreted antigen marker would ideally employ an epitope sequence that would provide low background in plant tissue, a promoter-leader sequence that would impart efficient expression and targeting across the plasma membrane, and would produce protein that is bound in the cell wall and yet accessible to antibodies. A normally secreted wall protein modified to include a unique epitope would satisfy all such requirements.

One example of a protein suitable for modification in this manner is extensin, or hydroxyproline rich glycoprotein (HPRG). For example, the maize HPRG (Steifel et al., The Plant Cell, 2:785 (1990)) molecule is well characterized in terms of molecular biology, expression and protein structure. However, any one of a variety of extensins and/or glycine-rich wall proteins (Keller et al., EMBO Journal, 8:1309 (1989)) could be modified by the addition of an antigenic site to create a screenable marker.

a. Selectable Markers

Possible selectable markers for use in connection with the present invention include, but are not limited to, a neo or nptII gene (Potrykus et al., Mol. Gen. Genet., 199:183 (1985)) which codes for kanamycin resistance and can be selected for using kanamycin, G418, and the like; a bar gene which confers resistance to the herbicide phosphinothricin; a gene which encodes an altered EPSP synthase protein (Hinchee et al., Biotech., 6:915 (1988)) thus conferring glyphosate resistance; a nitrilase gene such as bxn from Klebsiella ozaenae which confers resistance to bromoxynil (Stalker et al., Science, 242:419 (1988)); a mutant acetolactate synthase gene (ALS) which confers resistance to imidazolinone, sulfonylurea or other ALS-inhibiting chemicals (European Patent Application 154, 204, 1985); a methotrexate-resistant DHFR gene (Thillet et al., J. Biol. Chem., 263:12500 (1988)); a dalapon dehalogenase gene that confers resistance to the herbicide dalapon; a phosphomannose isomerase (PMI) gene; a mutated anthranilate synthase gene that confers resistance to 5-methyl tryptophan; the hph gene which confers resistance to the antibiotic hygromycin; or the mannose-6-phosphate isomerase gene (also referred to herein as the phosphomannose isomerase gene), which provides the ability to metabolize mannose (U.S. Pat. Nos. 5,767,378 and 5,994,629). One skilled in the art is capable of selecting a suitable selectable marker gene for use in the present invention. Where a mutant EPSP synthase gene is employed, additional benefit may be realized through the incorporation of a suitable chloroplast transit peptide, CTP (European Patent Application 0,218,571, 1987).

An illustrative embodiment of a selectable marker gene capable of being used in systems to select transformants are the genes that encode the enzyme phosphinothricin acetyltransferase, such as the bar gene from Streptomyces hygroscopicus or the pat gene from Streptomyces viridochromogenes. The enzyme phosphinothricin acetyl transferase (PAT) inactivates the active ingredient in the herbicide bialaphos, phosphinothricin (PPT). PPT inhibits glutamine synthetase, (Murakami et al., Mol. Gen. Genet., 205:42 (1986); Twell et al., Plant Physiol., 91:1270 (1989)) causing rapid accumulation of ammonia and cell death. The success in using this selective system in conjunction with monocots was particularly surprising because of the major difficulties which have been reported in transformation of cereals (Potrykus, Trends Biotech., 7:269 (1989)).

Where one desires to employ a bialaphos resistance gene in the practice of the invention, a particularly useful gene for this purpose is the bar or pat genes obtainable from species of Streptomyces (e.g., ATCC No. 21,705). The cloning of the bar gene has been described (Murakami et al., Mol. Gen. Genet., 205:42 (1986); Thompson et al., EMBO Journal, 6:2519 (1987)) as has the use of the bar gene in the context of plants other than monocots (De Block et al., EMBO Journal, 6:2513 (1987); De Block et al., Plant Physiol., 91:694 (1989)).

b. Screenable Markers

Screenable markers that may be employed include, but are not limited to, a glucuronidase or uidA gene (GUS) which encodes an enzyme for which various chromogenic substrates are known; an R-locus gene, which encodes a product that regulates the production of anthocyanin pigments (red color) in plant tissues (Dellaporta et al., in Chromosome Structure and Function, pp. 263-282 (1988)); a β-lactamase gene (Sutcliffe, PNAS USA, 75:3737 (1978)), which encodes an enzyme for which various chromogenic substrates are known (e.g., PADAC, a chromogenic cephalosporin); a xylE gene (Zukowsky et al., PNAS USA, 80:1101 (1983)) which encodes a catechol dioxygenase that can convert chromogenic catechols; an α-amylase gene (Ikuta et al., Biotech., 8:241 (1990)); a tyrosinase gene (Katz et al., J. Gen. Microbiol., 129:2703 (1983)) which encodes an enzyme capable of oxidizing tyrosine to DOPA and dopaquinone which in turn condenses to form the easily detectable compound melanin; a β-galactosidase gene, which encodes an enzyme for which there are chromogenic substrates; a luciferase (lux) gene (Ow et al., Science, 234:856 (1986)), which allows for bioluminescence detection; or an aequorin gene (Prasher et al., Biochem. Biophys. Res. Comm., 126:1259 (1985)), which may be employed in calcium-sensitive bioluminescence detection, or a green fluorescent protein gene (Niedz et al., Plant Cell Reports, 14: 403 (1995)).

Genes from the maize R gene complex are contemplated to be particularly useful as screenable markers. The R gene complex in maize encodes a protein that acts to regulate the production of anthocyanin pigments in most seed and plant tissue. A gene from the R gene complex is suitable for maize transformation, because the expression of this gene in transformed cells does not harm the cells. Thus, an R gene introduced into such cells will cause the expression of a red pigment and, if stably incorporated, can be visually scored as a red sector. If a maize line carries dominant allelles for genes encoding the enzymatic intermediates in the anthocyanin biosynthetic pathway (C2, A1, A2, Bz1 and Bz2), but carries a recessive allele at the R locus, transformation of any cell from that line with R will result in red pigment formation. Exemplary lines include Wisconsin 22 which contains the rg-Stadler allele and TR112, a K55 derivative which is r-g, b, P1. Alternatively any genotype of maize can be utilized if the C1 and R alleles are introduced together. A further screenable marker contemplated for use in the present invention is firefly luciferase, encoded by the lux gene. The presence of the lux gene in transformed cells may be detected using, for example, X-ray film, scintillation counting, fluorescent spectrophotometry, low-light video cameras, photon counting cameras or multiwell luminometry. It is also envisioned that this system may be developed for populational screening for bioluminescence, such as on tissue culture plates, or even for whole plant screening.

The polynucleotides used to transform the plant may include, but is not limited to, DNA from plant genes and non-plant genes such as those from bacteria, yeasts, animals or viruses. The introduced DNA can include modified genes, portions of genes, or chimeric genes, including genes from the same or different maize genotype. The term “chimeric gene” or “chimeric DNA” is defined as a gene or DNA sequence or segment comprising at least two DNA sequences or segments from species which do not combine DNA under natural conditions, or which DNA sequences or segments are positioned or linked in a manner which does not normally occur in the native genome of the untransformed plant.

Expression cassettes comprising the polynucleotide encoding a hyperthermophilic processing enzyme, and preferably a codon-optimized polynucleotide is further provided. It is preferred that the polynucleotide in the expression cassette (the first polynucleotide) is operably linked to regulatory sequences, such as a promoter, an enhancer, an intron, a termination sequence, or any combination thereof, and, optionally, to a second polynucleotide encoding a signal sequence (N- or C-terminal) which directs the enzyme encoded by the first polynucleotide to a particular cellular or subcellular location. Thus, a promoter and one or more signal sequences can provide for high levels of expression of the enzyme in particular locations in a plant, plant tissue or plant cell. Promoters can be constitutive promoters, inducible (conditional) promoters or tissue-specific promoters, e.g., endosperm-specific promoters such as the maize γ-zein promoter (exemplified by SEQ ID NO:12) or the maize ADP-gpp promoter (exemplified by SEQ ID NO:11, which includes a 5′ untranslated and an intron sequence). The invention also provides an isolated polynucleotide comprising a promoter comprising SEQ ID NO:11 or 12, a polynucleotide which hybridizes to the complement thereof under low stringency hybridization conditions, or a fragment thereof which has promoter activity, e.g., at least 10%, and preferably at least 50%, the activity of a promoter having SEQ ID NO:11 or 12. Also provided are vectors which comprise the expression cassette or polynucleotide of the invention and transformed cells comprising the polynucleotide, expression cassette or vector of the invention. A vector of the invention can comprise a polynucleotide sequence which encodes more than one hyperthermophilic processing enzyme of the invention, which sequence can be in sense or antisense orientation, and a transformed cell may comprise one or more vectors of the invention. Preferred vectors are those useful to introduce nucleic acids into plant cells.

### Transformation

The expression cassette, or a vector construct containing the expression cassette may be inserted into a cell. The expression cassette or vector construct may be carried episomally or integrated into the genome of the cell. The transformed cell may then be grown into a transgenic plant. Accordingly, the invention provides the products of the transgenic plant. Such products may include, but are not limited to, the seeds, fruit, progeny, and products of the progeny of the transgenic plant.

A variety of techniques are available and known to those skilled in the art for introduction of constructs into a cellular host. Transformation of bacteria and many eukaryotic cells may be accomplished through use of polyethylene glycol, calcium chloride, viral infection, phage infection, electroporation and other methods known in the art. Techniques for transforming plant cells or tissue include transformation with DNA employing A. tumefaciens or A. rhizogenes as the transforming agent, electroporation, DNA injection, microprojectile bombardment, particle acceleration, etc. (See, for example, EP 295959 and EP 138341).

In one embodiment, binary type vectors of Ti and Ri plasmids of Agrobacterium spp. Ti-derived vectors are used to transform a wide variety of higher plants, including monocotyledonous and dicotyledonous plants, such as soybean, cotton, rape, tobacco, and rice (Pacciotti et al. Bio/Technology, 3:241 (1985): Byrne et al. Plant Cell Tissue and Organ Culture, 8:3 (1987); Sukhapinda et al. Plant Mol. Biol., 8:209 (1987); Lorz et al. Mol. Gen. Genet., 199:178 (1985); Potrykus Mol. Gen. Genet., 199:183 (1985); Park et al., J. Plant Biol., 38:365 (1985): Hiei et al., Plant J., 6:271 (1994)). The use of T-DNA to transform plant cells has received extensive study and is amply described (EP 120516; Hoekema, In: The Binary Plant Vector System. Offset-drukkerij Kanters B. V.; Alblasserdam (1985), Chapter V; Knauf, et al., Genetic Analysis of Host Range Expression by Agrobacterium In: Molecular Genetics of the Bacteria-Plant Interaction, Puhler, A. ed., Springer-Verlag, New York, 1983, p. 245; and An. et al., EMBO J., 4:277 (1985)).

Other transformation methods are available to those skilled in the art, such as direct uptake of foreign DNA constructs (see EP 295959), techniques of electroporation (Fromm et al. Nature (London), 319:791 (1986), or high velocity ballistic bombardment with metal particles coated with the nucleic acid constructs (Kline et al. Nature (London) 327:70 (1987), and U.S. Pat. No. 4,945,050). Once transformed, the cells can be regenerated by those skilled in the art. Of particular relevance are the recently described methods to transform foreign genes into commercially important crops, such as rapeseed (De Block et al., Plant Physiol. 91:694-701 (1989)), sunflower (Everett et al., Bio/Technology, 5:1201 (1987)), soybean (McCabe et al., Bio/Technology, 6:923 (1988); Hinchee et al., Bio/Technology, 6:915 (1988); Chee et al., Plant Physiol., 91:1212 (1989); Christou et al., Proc. Natl. Acad. Sci. USA, 86:7500 (1989) EP 301749), rice (Hiei et al., Plant J., 6:271 (1994)), and corn (Gordon Kamm et al., Plant Cell, 2:603 (1990); Fromm et al., Biotechnology, 8:833, (1990)).

Expression vectors containing genomic or synthetic fragments can be introduced into protoplasts or into intact tissues or isolated cells. Preferably expression vectors are introduced into intact tissue. General methods of culturing plant tissues are provided, for example, by Maki et al. “Procedures for Introducing Foreign DNA into Plants” in Methods in Plant Molecular Biology & Biotechnology, Glich et al. (Eds.), pp. 67-88 CRC Press (1993); and by Phillips et al. “Cell-Tissue Culture and In-Vitro Manipulation” in Corn & Corn Improvement, 3rd Edition 10, Sprague et al. (Eds.) pp. 345-387, American Society of Agronomy Inc. (1988).

In one embodiment, expression vectors may be introduced into maize or other plant tissues using a direct gene transfer method such as microprojectile-mediated delivery, DNA injection, electroporation and the like. Expression vectors are introduced into plant tissues using the microprojectile media delivery with the biolistic device. See, for example, Tomes et al. “Direct DNA transfer into intact plant cells via microprojectile bombardment” in Gamborg and Phillips (Eds.) Plant Cell, Tissue and Organ Culture: Fundamental Methods, Springer Verlag, Berlin (1995). Nevertheless, the present invention contemplates the transformation of plants with a hyperthermophilic processing enzyme in accord with known transforming methods. Also see, Weissinger et al., Annual Rev. Genet., 22:421 (1988); Sanford et al., Particulate Science and Technology, 5:27 (1987) (onion); Christou et al., Plant Physiol., 87:671 (1988) (soybean); McCabe et al., Bio/Technology, 6:923 (1988) (soybean); Datta et al., Bio/Technology, 8:736 (1990) (rice); Klein et al., Proc. Natl. Acad. Sci. USA, 85:4305 (1988) (maize); Klein et al., Bio/Technology, 6:559 (1988) (maize); Klein et al., Plant Physiol., 91:440 (1988) (maize); Fromm et al., Bio/Technology, 8:833 (1990) (maize); and Gordon-Kamm et al., Plant Cell, 2, 603 (1990) (maize); Svab et al., Proc. Natl. Acad. Sci. USA, 87:8526 (1990) (tobacco chloroplast); Koziel et al., Biotechnology, 11:194 (1993) (maize); Shimamoto et al., Nature, 338:274 (1989) (rice); Christou et al., Biotechnology, 2:957 (1991) (rice); European Patent Application EP 0 332 581 (orchardgrass and other Pooideae); Vasil et al., Biotechnology, 11:1553 (1993) (wheat); Weeks et al., Plant Physiol., 102:1077 (1993) (wheat). Methods in Molecular Biology, 82. Arabidopsis Protocols Ed. Martinez-Zapater and Salinas 1998 Humana Press (Arabidopsis).

Transformation of plants can be undertaken with a single DNA molecule or multiple DNA molecules (i.e., co-transformation), and both these techniques are suitable for use with the expression cassettes and constructs of the present invention. Numerous transformation vectors are available for plant transformation, and the expression cassettes of this invention can be used in conjunction with any such vectors. The selection of vector will depend upon the preferred transformation technique and the target species for transformation.

Ultimately, the most desirable DNA segments for introduction into a monocot genome may be homologous genes or gene families which encode a desired trait (e.g., hydrolysis of proteins, lipids or polysaccharides) and which are introduced under the control of novel promoters or enhancers, etc., or perhaps even homologous or tissue specific (e.g., root-, collar/sheath-, whorl-, stalk-, earshank-, kernel- or leaf-specific) promoters or control elements. Indeed, it is envisioned that a particular use of the present invention will be the targeting of a gene in a constitutive manner or in an inducible manner.

Examples of Suitable Transformation Vectors

Numerous transformation vectors available for plant transformation are known to those of ordinary skill in the plant transformation arts, and the genes pertinent to this invention can be used in conjunction with any such vectors known in the art. The selection of vector will depend upon the preferred transformation technique and the target species for transformation.

a. Vectors Suitable for Agrobacterium Transformation

Many vectors are available for transformation using Agrobacterium tumefaciens. These typically carry at least one T-DNA border sequence and include vectors such as pBIN19 (Bevan, Nucl. Acids Res. (1984)). Below, the construction of two typical vectors suitable for Agrobacterium transformation is described.

pCIB200 and pCIB2001

The binary vectors pcIB200 and pCIB2001 are used for the construction of recombinant vectors for use with Agrobacterium and are constructed in the following manner. pTJS75kan is created by NarI digestion of pTJS75 (Schmidhauser & Helinski, J. Bacteriol., 164: 446 (1985)) allowing excision of the tetracycline-resistance gene, followed by insertion of an AccI fragment from pUC4K carrying an NPTII (Messing & Vierra, Gene, 19: 259 (1982): Bevan et al., Nature, 304: 184 (1983): McBride et al., Plant Molecular Biology, 14: 266 (1990)). XhoI linkers are ligated to the EcoRV fragment of PCIB7 which contains the left and right T-DNA borders, a plant selectable nos/nptII chimeric gene and the pUC polylinker (Rothstein et al., Gene, 53: 153 (1987)), and the Xho1-digested fragment are cloned into SalI-digested pTJS75kan to create pCIB200 (see also EP 0 332 104, example 19). pCIB200 contains the following unique polylinker restriction sites: EcoRI, SstI, KpnI, BglII, XbaI, and SalI. pCIB2001 is a derivative of pCIB200 created by the insertion into the polylinker of additional restriction sites. Unique restriction sites in the polylinker of pCIB2001 are EcoRI, SstI, KpnI, BglII, XbaI, SalI, MluI, BclI, AvrII, ApaI, HpaI, and StuI. pCIB2001, in addition to containing these unique restriction sites also has plant and bacterial kanamycin selection, left and right T-DNA borders for Agrobacterium-mediated transformation, the RK2-derived trfA function for mobilization between E. coli and other hosts, and the OriT and OriV functions also from RK2. The pCIB2001 polylinker is suitable for the cloning of plant expression cassettes containing their own regulatory signals.

pCIB10 and Hygromycin Selection Derivatives thereof:

The binary vector pCIB10 contains a gene encoding kanamycin resistance for selection in plants and T-DNA right and left border sequences and incorporates sequences from the wide host-range plasmid pRK252 allowing it to replicate in both E. coli and Agrobacterium. Its construction is described by Rothstein et al. (Gene, 53: 153 (1987)). Various derivatives of pCIB10 are constructed which incorporate the gene for hygromycin B phosphotransferase described by Gritz et al. (Gene, 25: 179 (1983)). These derivatives enable selection of transgenic plant cells on hygromycin only (pCIB743), or hygromycin and kanamycin (pCIB715, pCIB717).

b. Vectors Suitable for Non-Agrobacterium Transformation

Transformation without the use of Agrobacterium tumefaciens circumvents the requirement for T-DNA sequences in the chosen transformation vector and consequently vectors lacking these sequences can be utilized in addition to vectors such as the ones described above which contain T-DNA sequences. Transformation techniques that do not rely on Agrobacterium include transformation via particle bombardment, protoplast uptake (e.g., PEG and electroporation) and microinjection. The choice of vector depends largely on the preferred selection for the species being transformed. Non-limiting examples of the construction of typical vectors suitable for non-Agrobacterium transformation is further described.

pCIB3064

pCIB3064 is a pUC-derived vector suitable for direct gene transfer techniques in combination with selection by the herbicide basta (or phosphinothricin). The plasmid pCIB246 comprises the CaMV 35S promoter in operational fusion to the E. coli GUS gene and the CaMV 35S transcriptional terminator and is described in the PCT published application WO 93/07278. The 35S promoter of this vector contains two ATG sequences 5′ of the start site. These sites are mutated using standard PCR techniques in such a way as to remove the ATGs and generate the restriction sites SspI and PvuII. The new restriction sites are 96 and 37 bp away from the unique SalI site and 101 and 42 bp away from the actual start site. The resultant derivative of pCIB246 is designated pCIB3025. The GUS gene is then excised from pCIB3025 by digestion with SalI and SacI, the termini rendered blunt and religated to generate plasmid pCIB3060. The plasmid pJIT82 may be obtained from the John Innes Centre, Norwich and the a 400 bp SmaI fragment containing the bar gene from Streptomyces viridochromogenes is excised and inserted into the HpaI site of pCIB3060 (Thompson et al., EMBO J. 6: 2519 (1987)). This generated pCIB3064, which comprises the bar gene under the control of the CaMV 35S promoter and terminator for herbicide selection, a gene for ampicillin resistance (for selection in E. coli) and a polylinker with the unique sites SphI, PstI, HindIII, and BamHI. This vector is suitable for the cloning of plant expression cassettes containing their own regulatory signals.

pSOG19 and pSOG35:

The plasmid pSOG35 is a transformation vector that utilizes the E. coli gene dihydrofolate reductase (DHFR) as a selectable marker conferring resistance to methotrexate. PCR is used to amplify the 35S promoter (−800 bp), intron 6 from the maize Adh1 gene (−550 bp) and 18 bp of the GUS untranslated leader sequence from pSOG10. A 250-bp fragment encoding the E. coli dihydrofolate reductase type II gene is also amplified by PCR and these two PCR fragments are assembled with a SacI-PstI fragment from pB1221 (Clontech) which comprises the pUC19 vector backbone and the nopaline synthase terminator. Assembly of these fragments generates pSOG19 which contains the 35S promoter in fusion with the intron 6 sequence, the GUS leader, the DHFR gene and the nopaline synthase terminator. Replacement of the GUS leader in pSOG19 with the leader sequence from Maize Chlorotic Mottle Virus (MCMV) generates the vector pSOG35. pSOG19 and pSOG35 carry the pUC gene for ampicillin resistance and have HindIII, SphI, PstI and EcoRI sites available for the cloning of foreign substances.

c. Vector Suitable for Chloroplast Transformation

For expression of a nucleotide sequence of the present invention in plant plastids, plastid transformation vector pPH143 (WO 97/32011, example 36) is used. The nucleotide sequence is inserted into pPH143 thereby replacing the PROTOX coding sequence. This vector is then used for plastid transformation and selection of transformants for spectinomycin resistance. Alternatively, the nucleotide sequence is inserted in pPH143 so that it replaces the aadH gene. In this case, transformants are selected for resistance to PROTOX inhibitors.

### Plant Hosts Subject to Transformation Methods

Any plant tissue capable of subsequent clonal propagation, whether by organogenesis or embryogenesis, may be transformed with a construct of the present invention. The term organogenesis means a process by which shoots and roots are developed sequentially from meristematic centers while the term embryogenesis means a process by which shoots and roots develop together in a concerted fashion (not sequentially), whether from somatic cells or gametes. The particular tissue chosen will vary depending on the clonal propagation systems available for, and best suited to, the particular species being transformed. Exemplary tissue targets include differentiated and undifferentiated tissues or plants, including but not limited to leaf disks, roots, stems, shoots, leaves, pollen, seeds, embryos, cotyledons, hypocotyls, megagametophytes, callus tissue, existing meristematic tissue (e.g., apical meristems, axillary buds, and root meristems), and induced meristem tissue (e.g., cotyledon meristem and hypocotyl meristem), tumor tissue, and various forms of cells and culture such as single cells, protoplast, embryos, and callus tissue. The plant tissue may be in plants or in organ, tissue or cell culture.

Plants of the present invention may take a variety of forms. The plants may be chimeras of transformed cells and non-transformed cells; the plants may be clonal transformants (e.g., all cells transformed to contain the expression cassette); the plants may comprise grafts of transformed and untransformed tissues (e.g., a transformed root stock grafted to an untransformed scion in citrus species). The transformed plants may be propagated by a variety of means, such as by clonal propagation or classical breeding techniques. For example, first generation (or T1) transformed plants may be selfed to give homozygous second generation (or T2) transformed plants, and the T2 plants further propagated through classical breeding techniques. A dominant selectable marker (such as npt U) can be associated with the expression cassette to assist in breeding.

The present invention may be used for transformation of any plant species, including monocots or dicots, including, but not limited to, corn (Zea mays), Brassica sp. (e.g., B. napus, B. rapa, B. juncea), particularly those Brassica species useful as sources of seed oil, alfalfa (Medicago sativa), rice (Oryza sativa), rye (Secale cereale), sorghum (Sorghum bicolor, Sorghum vulgare), millet (e.g., pearl millet (Pennisetum glaucum), proso millet (Panicum miliaceum), foxtail millet (Setaria italica), finger millet (Eleusine coracana)), sunflower (Helianthus annuus), safflower (Carthamus tinctorius), wheat (Triticum aestivum), soybean (Glycine max), tobacco (Nicotiana tabacum), potato (Solanum tuberosum), peanuts (Arachis hypogaea), cotton (Gossypium barbadense, Gossypium hirsutum), sweet potato (Ipomoea batatus), cassaya (Manihot esculenta), coffee (Cofea spp.), coconut (Cocos nucifera), pineapple (Ananas comosus), citrus trees (Citrus spp.), cocoa (Theobroma cacao), tea (Camellia sinensis), banana (Musa spp.), avocado (Persea americana), fig (Ficus casica), guava (Psidium guajava), mango (Mangifera indica), olive (Olea europaea), papaya (Carica papaya), cashew (Anacardium occidentale), macadamia (Macadamia integrifolia), almond (Prunus amygdalus), sugar beets (Beta vulgaris), sugarcane (Saccharum spp.), oats, barley, vegetables, ornamentals, woody plants such as conifers and deciduous trees, squash, pumpkin, hemp, zucchini, apple, pear, quince, melon, plum, cherry, peach, nectarine, apricot, strawberry, grape, raspberry, blackberry, soybean, sorghum, sugarcane, rapeseed, clover, carrot, and Arabidopsis thaliana.

Vegetables include tomatoes (Lycopersicon esculentum), lettuce (e.g., Lactuca sativa), green beans (Phaseolus vulgaris), lima beans (Phaseolus limensis), peas (Lathyrus spp.), cauliflower, broccoli, turnip, radish, spinach, asparagus, onion, garlic, pepper, celery, and members of the genus Cucumis such as cucumber (C. sativus), cantaloupe (C. cantalupensis), and musk melon (C. melo). Ornamentals include azalea (Rhododendron spp.), hydrangea (Macrophylla hydrangea), hibiscus (Hibiscus rosasanensis), roses (Rosa spp.), tulips (Tulipa spp.), daffodils (Narcissus spp.), petunias (Petunia hybrida), carnation (Dianthus caryophyllus), poinsettia (Euphorbia pulcherrima), and chrysanthemum. Conifers that may be employed in practicing the present invention include, for example, pines such as loblolly pine (Pinus taeda), slash pine (Pinus elliotii), ponderosa pine (Pinus ponderosa), lodgepole pine (Pinus contorta), and Monterey pine (Pinus radiata), Douglas-fir (Pseudotsuga menziesii); Western hemlock (Tsuga canadensis); Sitka spruce (Picea glauca); redwood (Sequoia sempervirens); true firs such as silver fir (Abies amabilis) and balsam fir (Abies balsamea); and cedars such as Western red cedar (Thuja plicata) and Alaska yellow-cedar (Chamaecyparis nootkatensis). Leguminous plants include beans and peas. Beans include guar, locust bean, fenugreek, soybean, garden beans, cowpea, mungbean, lima bean, fava bean, lentils, chickpea, etc. Legumes include, but are not limited to, Arachis, e.g., peanuts, Vicia, e.g., crown vetch, hairy vetch, adzuki bean, mung bean, and chickpea, Lupinus, e.g., lupine, trifolium, Phaseolus, e.g., common bean and lima bean, Pisum, e.g., field bean, Melilotus, e.g., clover, Medicago, e.g., alfalfa, Lotus, e.g., trefoil, lens, e.g., lentil, and false indigo. Preferred forage and turf grass for use in the methods of the invention include alfalfa, orchard grass, tall fescue, perennial ryegrass, creeping bent grass, and redtop.

Preferably, plants of the present invention include crop plants, for example, corn, alfalfa, sunflower, Brassica, soybean, cotton, safflower, peanut, sorghum, wheat, millet, tobacco, barley, rice, tomato, potato, squash, melons, legume crops, etc. Other preferred plants include Liliopsida and Panicoideae.

Once a desired DNA sequence has been transformed into a particular plant species, it may be propagated in that species or moved into other varieties of the same species, particularly including commercial varieties, using traditional breeding techniques.

Below are descriptions of representative techniques for transforming both dicotyledonous and monocotyledonous plants, as well as a representative plastid transformation technique.

a. Transformation of Dicotyledons

Transformation techniques for dicotyledons are well known in the art and include Agrobacterium-based techniques and techniques that do not require Agrobacterium. Non-Agrobacterium techniques involve the uptake of exogenous genetic material directly by protoplasts or cells. This can be accomplished by PEG or electroporation mediated uptake, particle bombardment-mediated delivery, or microinjection. Examples of these techniques are described by Paszkowski et al., EMBO J. 3: 2717 (1984), Potrykus et al., Mol. Gen. Genet., 199: 169 (1985), Reich et al., Biotechnology, 4: 1001 (1986), and Klein et al., Nature, 327: 70 (1987). In each case the transformed cells are regenerated to whole plants using standard techniques known in the art.

Agrobacterium-mediated transformation is a preferred technique for transformation of dicotyledons because of its high efficiency of transformation and its broad utility with many different species. Agrobacterium transformation typically involves the transfer of the binary vector carrying the foreign DNA of interest (e.g. pCIB200 or pCIB2001) to an appropriate Agrobacterium strain which may depend on the complement of vir genes carried by the host Agrobacterium strain either on a co-resident Ti plasmid or chromosomally (e.g., strain CIB542 for pCIB200 and pCIB2001 (Uknes et al., Plant Cell, 5: 159 (1993)). The transfer of the recombinant binary vector to Agrobacterium is accomplished by a triparental mating procedure using E. coli carrying the recombinant binary vector, a helper E. coli strain which carries a plasmid such as pRK2013 and which is able to mobilize the recombinant binary vector to the target Agrobacterium strain. Alternatively, the recombinant binary vector can be transferred to Agrobacterium by DNA transformation (Höfgen & Willmitzer, Nucl. Acids Res., 16: 9877 (1988)).

Transformation of the target plant species by recombinant Agrobacterium usually involves co-cultivation of the Agrobacterium with explants from the plant and follows protocols well known in the art. Transformed tissue is regenerated on selectable medium carrying the antibiotic or herbicide resistance marker present between the binary plasmid T-DNA borders.

The vectors may be introduced to plant cells in known ways. Preferred cells for transformation include Agrobacterium, monocot cells and dicots cells, including Liliopsida cells and Panicoideae cells. Preferred monocot cells are cereal cells, e.g., maize (corn), barley, and wheat, and starch accumulating dicot cells, e.g., potato.

Another approach to transforming a plant cell with a gene involves propelling inert or biologically active particles at plant tissues and cells. This technique is disclosed in U.S. Pat. Nos. 4,945,050, 5,036,006, and 5,100,792. Generally, this procedure involves propelling inert or biologically active particles at the cells under conditions effective to penetrate the outer surface of the cell and afford incorporation within the interior thereof. When inert particles are utilized, the vector can be introduced into the cell by coating the particles with the vector containing the desired gene. Alternatively, the target cell can be surrounded by the vector so that the vector is carried into the cell by the wake of the particle. Biologically active particles (e.g., dried yeast cells, dried bacterium or a bacteriophage, each containing DNA sought to be introduced) can also be propelled into plant cell tissue.

b. Transformation of Monocotyledons

Transformation of most monocotyledon species has now also become routine. Preferred techniques include direct gene transfer into protoplasts using polyethylene glycol (PEG) or electroporation techniques, and particle bombardment into callus tissue. Transformations can be undertaken with a single DNA species or multiple DNA species (i.e., co-transformation) and both these techniques are suitable for use with this invention. Co-transformation may have the advantage of avoiding complete vector construction and of generating transgenic plants with unlinked loci for the gene of interest and the selectable marker, enabling the removal of the selectable marker in subsequent generations, should this be regarded desirable. However, a disadvantage of the use of co-transformation is the less than 100% frequency with which separate DNA species are integrated into the genome (Schocher et al., Biotechnology, 4: 1093 1986)).

Patent Applications EP 0 292 435, EP 0 392 225, and WO 93/07278 describe techniques for the preparation of callus and protoplasts from an elite inbred line of maize, transformation of protoplasts using PEG or electroporation, and the regeneration of maize plants from transformed protoplasts. Gordon-Kamm et al. (Plant Cell, 2: 603 (1990)) and Fromm et al. (Biotechnology, 8: 833 (1990)) have published techniques for transformation of A188-derived maize line using particle bombardment. Furthermore, WO 93/07278 and Koziel et al. (Biotechnology, 11: 194 (1993)) describe techniques for the transformation of elite inbred lines of maize by particle bombardment. This technique utilizes immature maize embryos of 1.5-2.5 mm length excised from a maize ear 14-15 days after pollination and a PDS-1000He Biolistics device for bombardment.

Transformation of rice can also be undertaken by direct gene transfer techniques utilizing protoplasts or particle bombardment. Protoplast-mediated transformation has been described for Japonica-types and Indica-types (Zhang et al., Plant Cell Rep, 7: 379 (1988); Shimamoto et al., Nature, 338: 274 (1989); Datta et al., Biotechnology, 8: 736 (1990)). Both types are also routinely transformable using particle bombardment (Christou et al., Biotechnology, 9: 957 (1991)). Furthermore, WO 93/21335 describes techniques for the transformation of rice via electroporation. Patent Application EP 0 332 581 describes techniques for the generation, transformation and regeneration of Pooideae protoplasts. These techniques allow the transformation of Dactylis and wheat. Furthermore, wheat transformation has been described by Vasil et al. (Biotechnology, 10: 667 (1992)) using particle bombardment into cells of type C long-term regenerable callus, and also by Vasil et al. (Biotechnology, 1: 1553 (1993)) and Weeks et al. (Plant Physiol., 102: 1077 (1993)) using particle bombardment of immature embryos and immature embryo-derived callus. A preferred technique for wheat transformation, however, involves the transformation of wheat by particle bombardment of immature embryos and includes either a high sucrose or a high maltose step prior to gene delivery. Prior to bombardment, any number of embryos (0.75-1 mm in length) are plated onto MS medium with 3% sucrose (Murashiga & Skoog, Physiologia Plantarum, 15: 473 (1962)) and 3 mg/l 2,4-D for induction of somatic embryos, which is allowed to proceed in the dark. On the chosen day of bombardment, embryos are removed from the induction medium and placed onto the osmoticum (i.e., induction medium with sucrose or maltose added at the desired concentration, typically 15%). The embryos are allowed to plasmolyze for 2-3 hours and are then bombarded. Twenty embryos per target plate is typical, although not critical. An appropriate gene-carrying plasmid (such as pCIB3064 or pSG35) is precipitated onto micrometer size gold particles using standard procedures. Each plate of embryos is shot with the DuPont Biolistics® helium device using a burst pressure of about 1000 psi using a standard 80 mesh screen. After bombardment, the embryos are placed back into the dark to recover for about 24 hours (still on osmoticum). After 24 hours, the embryos are removed from the osmoticum and placed back onto induction medium where they stay for about a month before regeneration. Approximately one month later the embryo explants with developing embryogenic callus are transferred to regeneration medium (MS+1 mg/liter NAA, 5 mg/liter GA), further containing the appropriate selection agent (10 mg/l basta in the case of pCIB3064 and 2 mg/l methotrexate in the case of pSOG35). After approximately one month, developed shoots are transferred to larger sterile containers known as “GA7s” which contain half-strength MS, 2% sucrose, and the same concentration of selection agent.

Transformation of monocotyledons using Agrobacterium has also been described. See, WO 94/00977 and U.S. Pat. No. 5,591,616, both of which are incorporated herein by reference.

c. Transformation of Plastids

Seeds of Nicotiana tabacum c.v. ‘Xanthi nc’ are germinated seven per plate in a 1″ circular array on T agar medium and bombarded 12-14 days after sowing with 1 μm tungsten particles (M10, Biorad, Hercules, Calif.) coated with DNA from plasmids pPH143 and pPH145 essentially as described (Svab and Maliga, PNAS, 90:913 (1993)). Bombarded seedlings are incubated on T medium for two days after which leaves are excised and placed abaxial side up in bright light (350-500 μmol photons/m²/s) on plates of RMOP medium (Svab, Hajdukiewicz and Maliga, PNAS, 87:8526 (1990)) containing 500 μg/ml spectinomycin dihydrochloride (Sigma, St. Louis, Mo.). Resistant shoots appearing underneath the bleached leaves three to eight weeks after bombardment are subcloned onto the same selective medium, allowed to form callus, and secondary shoots isolated and subcloned. Complete segregation of transformed plastid genome copies (homoplasmicity) in independent subclones is assessed by standard techniques of Southern blotting (Sambrook et al., Molecular Cloning: A Laboratory Manual, Cold Spring Harbor Laboratory, Cold Spring Harbor (1989)). BamHI/EcoRI-digested total cellular DNA (Mettler, I. J. Plant Mol Biol Reporter, 5:346 (1987)) is separated on 1% Tris-borate (TBE) agarose gels, transferred to nylon membranes (Amersham) and probed with ³²P-labeled random primed DNA sequences corresponding to a 0.7 kb BamHI/HindIII DNA fragment from pC8 containing a portion of the rps7/12 plastid targeting sequence. Homoplasmic shoots are rooted aseptically on spectinomycin-containing MS/IBA medium (McBride et al., PNAS, 91:7301 (1994)) and transferred to the greenhouse.

### Production and Characterization of Stably Transformed Plants

Transformed plant cells are then placed in an appropriate selective medium for selection of transgenic cells, which are then grown to callus. Shoots are grown from callus and plantlets generated from the shoot by growing in rooting medium. The various constructs normally will be joined to a marker for selection in plant cells. Conveniently, the marker may be resistance to a biocide (particularly an antibiotic, such as kanamycin, G418, bleomycin, hygromycin, chloramphenicol, herbicide, or the like). The particular marker used will allow for selection of transformed cells as compared to cells lacking the DNA which has been introduced. Components of DNA constructs, including transcription/expression cassettes of this invention, may be prepared from sequences, which are native (endogenous) or foreign (exogenous) to the host. By “foreign” it is meant that the sequence is not found in the wild-type host into which the construct is introduced. Heterologous constructs will contain at least one region, which is not native to the gene from which the transcription-initiation-region is derived.

To confirm the presence of the transgenes in transgenic cells and plants, a Southern blot analysis can be performed using methods known to those skilled in the art. Integration of a polynucleic acid segment into the genome can be detected and quantitated by Southern blot, since they can be readily distinguished from constructs containing the segments through use of appropriate restriction enzymes. Expression products of the transgenes can be detected in any of a variety of ways, depending upon the nature of the product, and include Western blot and enzyme assay. One particularly useful way to quantitate protein expression and to detect replication in different plant tissues is to use a reporter gene, such as GUS. Once transgenic plants have been obtained, they may be grown to produce plant tissues or parts having the desired phenotype. The plant tissue or plant parts may be harvested, and/or the seed collected. The seed may serve as a source for growing additional plants with tissues or parts having the desired characteristics.

The invention thus provides a transformed plant or plant part, such as an ear, seed, fruit, grain, stover, chaff, or bagasse comprising at least one polynucleotide, expression cassette or vector of the invention, methods of making such a plant and methods of using such a plant or a part thereof. The transformed plant or plant part expresses a processing enzyme, optionally localized in a particular cellular or subcellular compartment of a certain tissue or in developing grain. For instance, the invention provides a transformed plant part comprising at least one starch processing enzyme present in the cells of the plant, wherein the plant part is obtained from a transformed plant, the genome of which is augmented with an expression cassette encoding the at least one starch processing enzyme. The processing enzyme does not act on the target substrate unless activated by methods such as heating, grinding, or other methods, which allow the enzyme to contact the substrate under conditions where the enzyme is active

### Exemplary Methods of the Present Invention

The self-processing plants and plant parts of the present invention may be used in various methods employing the processing enzymes (mesophilic, thermophilic, or hyperthermophilic) expressed and activated therein. In accordance with the present invention, a transgenic plant part obtained from a transgenic plant the genome of which is augmented with at least one processing enzyme, is placed under conditions in which the processing enzyme is expressed and activated. Upon activation, the processing enzyme is activated and functions to act on the substrate in which it normally acts to obtained the desired result. For example, the starch-processing enzymes act upon starch to degrade, hydrolyze, isomerize, or otherwise modify to obtain the desired result upon activation. Non-starch processing enzymes may be used to disrupt the plant cell membrane in order to facilitate the extraction of starch, lipids, amino acids, or other products from the plants. Moreover, non-hyperthermophilic and hyperthermophilic enzymes may be used in combination in the self-processing plant or plant parts of the present invention. For example, a mesophilic non-starch degrading enzyme may be activated to disrupt the plant cell membrane for starch extraction, and subsequently, a hyperthermophilic starch-degrading enzyme may then be activated in the self-processing plant to degrade the starch.

Enzymes expressed in grain can be activated by placing the plant or plant part containing them in conditions in which their activity is promoted. For example, one or more of the following techniques may be used: The plant part may be contacted with water, which provides a substrate for a hydrolytic enzyme and thus will activate the enzyme. The plant part may be contacted with water which will allow enzyme to migrate from the compartment into which it was deposited during development of the plant part and thus to associate with its substrate. Movement of the enzyme is possible because compartmentalization is breached during maturation, drying of grain and re-hydration. The intact or cracked grain may be contacted with water which will allow enzyme to migrate from the compartment into which it was deposited during development of the plant part and thus to associate with its substrate. Enzymes can also be activated by addition of an activating compound. For example, a calcium-dependent enzyme can be activated by addition of calcium. Other activating compounds may determined by those skilled in the art. Enzymes can be activated by removal of an inactivator. For example, there are known peptide inhibitors of amylase enzymes, the amylase could be co-expressed with an amylase inhibitor and then activated by addition of a protease. Enzymes can be activated by alteration of pH to one at which the enzyme is most active. Enzymes can also be activated by increasing temperature. An enzyme generally increases in activity up to the maximal temperature for that enzyme. A mesophilic enzyme will increase in activity from the level of activity ambient temperature up to the temperature at which it loses activity which is typically less than or equal to 70° C. Similarly thermophilic and hyperthermophilic enzymes can also be activated by increasing temperature. Thermophilic enzymes can be activated by heating to temperatures up to the maximal temperature of activity or of stability. For a thermophilic enzyme the maximal temperatures of stability and activity will generally be between 70 and 85° C. Hyperthermophilic enzymes will have the even greater relative activation than mesophilic or thermophilic enzymes because of the greater potential change in temperature from 25° C. up to 85° C. to 95° C. or even 100° C. The increased temperature may be achieved by any method, for example by heating such as by baking, boiling, heating, steaming, electrical discharge or any combination thereof. Moreover, in plants expressing mesophilic or thermophilic enzyme(s), activation of the enzyme may be accomplished by grinding, thereby allowing the enzyme to contact the substrate.

The optimal conditions, e.g., temperature, hydration, pH, etc, may be determined by one having skill in the art and may depend upon the individual enzyme being employed and the desired application of the enzyme.

The present invention further provides for the use of exogenous enzymes that may assist in a particular process. For example, the use of a self-processing plant or plant part of the present invention may be used in combination with an exogenously provided enzyme to facilitate the reaction. As an example, transgenic α-amylase corn may be used in combination with other starch-processing enzymes, such as pullulanase, α-glucosidase, glucose isomerase, mannanases, hemicellulases, etc., to hydrolyze starch or produce ethanol. In fact, it has been found that combinations of the transgenic α-amylase corn with such enzymes has unexpectedly provided superior degrees of starch conversion relative to the use of transgenic α-amylase corn alone.

Example of suitable methods contemplated herein are provided.

a. Starch Extraction from Plants

The invention provides for a method of facilitating the extraction of starch from plants. In particular, at least one polynucleotide encoding a processing enzyme that disrupts the physically restraining matrix of the endosperm (cell walls, non-starch polysaccharide, and protein matrix) is introduced to a plant so that the enzyme is preferably in close physical proximity to starch granules in the plant. In this embodiment of the invention, transformed plants express one or more protease, glucanase, xylanase, thioredoxin/thioredoxin reductase, cellulase, phytase, lipase, beta glucosidase, esterase and the like, but not enzymes that have any starch degrading activity, so as to maintain the integrity of the starch granules. The expression of these enzymes in a plant part such as grain thus improves the process characteristics of grain. The processing enzyme may be mesophilic, thermophilic, or hyperthermophilic. In one example, grain from a transformed plant of the invention is heat dried, likely inactivating non-hyperthermophilic processing enzymes and improving seed integrity. Grain (or cracked grain) is steeped at low temperatures or high temperatures (where time is of the essence) with high or low moisture content or conditions (see Primary Cereal Processing, Gordon and Willm, eds., pp. 319-337 (1994), the disclosure of which is incorporated herein), with or without sulphur dioxide. Upon reaching elevated temperatures, optionally at certain moisture conditions, the integrity of the endosperm matrix is disrupted by activating the enzymes, e.g., proteases, xylanases, phytase or glucanases which degrade the proteins and non-starch polysaccharides present in the endosperm leaving the starch granule therein intact and more readily recoverable from the resulting material. Further, the proteins and non-starch polysaccharides in the effluent are at least partially degraded and highly concentrated, and so may be used for improved animal feed, food, or as media components for the fermentation of microorganisms. The effluent is considered a corn-steep liquor with improved composition.

Thus, the invention provides a method to prepare starch granules. The method comprises treating grain, for example cracked grain, which comprises at least one non-starch processing enzyme under conditions which activate the at least one enzyme, yielding a mixture comprising starch granules and non-starch degradation products, e.g., digested endosperm matrix products. The non-starch processing enzyme may be mesophilic, thermophilic, or hyperthermophilic. After activation of the enzyme, the starch granules are separated from the mixture. The grain is obtained from a transformed plant, the genome of which comprises (is augmented with) an expression cassette encoding the at least one processing enzyme. For example, the processing enzyme may be a protease, glucanase, xylanase, phytase, thiroredoxin/thioredoxin reductase, esterase cellulase, lipase, or a beta glucosidase. The processing enzyme may be hyperthermophilic. The grain can be treated under low or high moisture conditions, in the presence or absence of sulfur dioxide. Depending on the activity and expression level of the processing enzyme in the grain from the transgenic plant, the transgenic grain may be mixed with commodity grain prior to or during processing. Also provided are products obtained by the method such as starch, non-starch products and improved steepwater comprising at least one additional component.

b. Starch-Processing Methods

Transformed plants or plant parts of the present invention may comprise starch-degrading enzymes as disclosed herein that degrade starch granules to dextrins, other modified starches, or hexoses (e.g., α-amylase, pullulanase, α-glucosidase, glucoamylase, amylopullulanase) or convert glucose into fructose (e.g., glucose isomerase). Preferably, the starch-degrading enzyme is selected from α-amylase, α-glucosidase, glucoamylase, pullulanase, neopullulanase, amylopullulanase, glucose isomerase, and combinations thereof is used to transform the grain. Moreover, preferably, the enzyme is operably linked to a promoter and to a signal sequence that targets the enzyme to the starch granule, an amyloplast, the apoplast, or the endoplasmic reticulum. Most preferably, the enzyme is expressed in the endosperm, and particularly, corn endosperm, and localized to one or more cellular compartments, or within the starch granule itself. The preferred plant part is grain. Preferred plant parts are those from corn, wheat, barley, rye, oat, sugar cane, or rice.

In accordance with one starch-degrading method of the present invention, the transformed grain accumulates the starch-degrading enzyme in starch granules, is steeped at conventional temperatures of 50° C.-60° C., and wet-milled as is known in the art. Preferably, the starch-degrading enzyme is hyperthermophilic. Because of sub-cellular targeting of the enzyme to the starch granule, or by virtue of the association of the enzyme with the starch granule, by contacting the enzyme and starch granule during the wet-milling process at the conventional temperatures, the processing enzyme is co-purified with the starch granules to obtain the starch granules/enzyme mixture. Subsequent to the recovery of the starch granules/enzyme mixture, the enzyme is then activated by providing favorable conditions for the activity of the enzyme. For example, the processing may be performed in various conditions of moisture and/or temperature to facilitate the partial (in order to make derivatized starches or dextrins) or complete hydrolysis of the starch into hexoses. Syrups containing high dextrose or fructose equivalents are obtained in this manner. This method effectively reduces the time, energy, and enzyme costs and the efficiency with which starch is converted to the corresponding hexose, and the efficiency of the production of products, like high sugar steepwater and higher dextrose equivalent syrups, are increased.

In another embodiment, a plant, or a product of the plant such as a fruit or grain, or flour made from the grain that expresses the enzyme is treated to activate the enzyme and convert polysaccharides expressed and contained within the plant into sugars. Preferably, the enzyme is fused to a signal sequence that targets the enzyme to a starch granule, an amyloplast, the apoplast or to the endoplasmic reticulum as disclosed herein. The sugar produced may then be isolated or recovered from the plant or the product of the plant. In another embodiment, a processing enzyme able to convert polysaccharides into sugars is placed under the control of an inducible promoter according to methods known in the art and disclosed herein. The processing enzyme may be mesophilic, thermophilic or hyperthermophilic. The plant is grown to a desired stage and the promoter is induced causing expression of the enzyme and conversion of the polysaccharides, within the plant or product of the plant, to sugars. Preferably the enzyme is operably linked to a signal sequence that targets the enzyme to a starch granule, an amyloplast, an apoplast or to the endoplasmic reticulum. In another embodiment, a transformed plant is produced that expresses a processing enzyme able to convert starch into sugar. The enzyme is fused to a signal sequence that targets the enzyme to a starch granule within the plant. Starch is then isolated from the transformed plant that contains the enzyme expressed by the transformed plant. The enzyme contained in the isolated starch may then be activated to convert the starch into sugar. The enzyme may be mesophilic, thermophilic, or hyperthermophilic. Examples of hyperthermophilic enzymes able to convert starch to sugar are provided herein. The methods may be used with any plant which produces a polysaccharide and that can express an enzyme able to convert a polysaccharide into sugars or hydrolyzed starch product such as dextrin, maltooligosaccharide, glucose and/or mixtures thereof.

The invention provides a method to produce dextrins and altered starches from a plant, or a product from a plant, that has been transformed with a processing enzyme which hydrolyses certain covalent bonds of a polysaccharide to form a polysaccharide derivative. In one embodiment, a plant, or a product of the plant such as a fruit or grain, or flour made from the grain that expresses the enzyme is placed under conditions sufficient to activate the enzyme and convert polysaccharides contained within the plant into polysaccharides of reduced molecular weight. Preferably, the enzyme is fused to a signal sequence that targets the enzyme to a starch granule, an amyloplast, the apoplast or to the endoplasmic reticulum as disclosed herein. The dextrin or derivative starch produced may then be isolated or recovered from the plant or the product of the plant. In another embodiment, a processing enzyme able to convert polysaccharides into dextrins or altered starches is placed under the control of an inducible promoter according to methods known in the art and disclosed herein. The plant is grown to a desired stage and the promoter is induced causing expression of the enzyme and conversion of the polysaccharides, within the plant or product of the plant, to dextrins or altered starches. Preferably the enzyme is α-amylase, pullulanase, iso or neo-pullulanase and is operably linked to a signal sequence that targets the enzyme to a starch granule, an amyloplast, the apoplast or to the endoplasmic reticulum. In one embodiment, the enzyme is targeted to the apoplast or to the endoreticulum. In yet another embodiment, a transformed plant is produced that expresses an enzyme able to convert starch into dextrins or altered starches. The enzyme is fused to a signal sequence that targets the enzyme to a starch granule within the plant. Starch is then isolated from the transformed plant that contains the enzyme expressed by the transformed plant. The enzyme contained in the isolated starch may then be activated under conditions sufficient for activation to convert the starch into dextrins or altered starches. Examples of hyperthermophilic enzymes, for example, able to convert starch to hydrolyzed starch products are provided herein. The methods may be used with any plant which produces a polysaccharide and that can express an enzyme able to convert a polysaccharide into sugar.

In another embodiment, grain from transformed plants of the invention that accumulate starch-degrading enzymes that degrade linkages in starch granules to dextrins, modified starches or hexose (e.g., α-amylase, pullulanase, α-glucosidase, glucoamylase, amylopullulanase) is steeped under conditions favoring the activity of the starch degrading enzyme for various periods of time. The resulting mixture may contain high levels of the starch-derived product. The use of such grain: 1) eliminates the need to mill the grain, or otherwise process the grain to first obtain starch granules, 2) makes the starch more accessible to enzymes by virtue of placing the enzymes directly within the endosperm tissue of the grain, and 3) eliminates the need for microbially produced starch-hydrolyzing enzymes. Thus, the entire process of wet-milling prior to hexose recovery is eliminated by simply heating grain, preferably corn grain, in the presence of water to allow the enzymes to act on the starch.

This process can also be employed for the production of ethanol, high fructose syrups, hexose (glucose) containing fermentation media, or any other use of starch that does not require the refinement of grain components.

The invention further provides a method of preparing dextrin, maltooligosaccharides, and/or sugar involving treating a plant part comprising starch granules and at least one starch processing enzyme under conditions so as to activate the at least one enzyme thereby digesting starch granules to form an aqueous solution comprising sugars. The plant part is obtained from a transformed plant, the genome of which is augmented with an expression cassette encoding the at least one processing enzyme. The aqueous solution comprising dextrins, maltooligosaccharides, and/or sugar is then collected. In one embodiment, the processing enzyme is α-amylase, α-glucosidase, pullulanase, glucoamylase, amylopullulanase, glucose isomerase, or any combination thereof. Preferably, the enzyme is hyperthermophilic. In another embodiment, the method further comprises isolating the dextrins, maltooligosaccharides, and/or sugar.

c. Improved Corn Varieties

The invention also provides for the production of improved corn varieties (and varieties of other crops) that have normal levels of starch accumulation, and accumulate sufficient levels of amylolytic enzyme(s) in their endosperm, or starch accumulating organ, such that upon activation of the enzyme contained therein, such as by boiling or heating the plant or a part thereof in the case of a hyperthermophilic enzyme, the enzyme(s) is activated and facilitates the rapid conversion of the starch into simple sugars. These simple sugars (primarily glucose) will provide sweetness to the treated corn. The resulting corn plant is an improved variety for dual use as a grain producing hybrid and as sweet corn. Thus, the invention provides a method to produce hyper-sweet corn, comprising treating transformed corn or a part thereof, the genome of which is augmented with and expresses in endosperm an expression cassette comprising a promoter operably linked to a first polynucleotide encoding at least one amylolytic enzyme, conditions which activate the at least one enzyme so as to convert polysaccharides in the corn into sugar, yielding hypersweet corn. The promoter may be a constitutive promoter, a seed-specific promoter, or an endosperm-specific promoter which is linked to a polynucleotide sequence which encodes a processing enzyme such as α-amylase, e.g., one comprising SEQ ID NO: 13, 14, or 16. Preferably, the enzyme is hyperthermophilic. In one embodiment, the expression cassette further comprises a second polynucleotide which encodes a signal sequence operably linked to the enzyme encoded by the first polynucleotide. Exemplary signal sequences in this embodiment of the invention direct the enzyme to apoplast, the endoplasmic reticulum, a starch granule, or to an amyloplast. The corn plant is grown such that the ears with kernels are formed and then the promoter is induced to cause the enzyme to be expressed and convert polysaccharide contained within the plant into sugar.

d. Self-Fermenting Plants

In another embodiment of the invention, plants, such as corn, rice, wheat, or sugar cane are engineered to accumulate large quantities of processing enzymes in their cell walls, e.g., xylanases, cellulases, hemicellulases, glucanases, pectinases, lipases, esterases, beta glucosidases, phytases, proteases and the like (non-starch polysaccharide degrading enzymes). Following the harvesting of the grain component (or sugar in the case of sugar cane), the stover, chaff, or bagasse is used as a source of the enzyme, which was targeted for expression and accumulation in the cell walls, and as a source of biomass. The stover (or other left-over tissue) is used as a feedstock in a process to recover fermentable sugars. The process of obtaining the fermentable sugars consists of activating the non-starch polysaccharide degrading enzyme. For example, activation may comprise heating the plant tissue in the presence of water for periods of time adequate for the hydrolysis of the non-starch polysaccharide into the resulting sugars. Thus, this self-processing stover produces the enzymes required for conversion of polysaccharides into monosaccharides, essentially at no incremental cost as they are a component of the feedstock. Further, the temperature-dependent enzymes have no detrimental effects on plant growth and development, and cell wall targeting, even targeting into polysaccharide microfibrils by virtue of cellulose/xylose binding domains fused to the protein, improves the accessibility of the substrate to the enzyme.

Thus, the invention also provides a method of using a transformed plant part comprising at least one non-starch polysaccharide processing enzyme in the cell wall of the cells of the plant part. The method comprises treating a transformed plant part comprising at least one non-starch polysaccharide processing enzyme under conditions which activate the at least one enzyme thereby digesting starch granules to form an aqueous solution comprising sugars, wherein the plant part is obtained from a transformed plant, the genome of which is augmented with an expression cassette encoding the at least one non-starch polysaccharide processing enzyme; and collecting the aqueous solution comprising the sugars. The invention also includes a transformed plant or plant part comprising at least one non-starch polysaccharide processing enzyme present in the cell or cell wall of the cells of the plant or plant part. The plant part is obtained from a transformed plant, the genome of which is augmented with an expression cassette encoding the at least one non-starch processing enzyme, e.g., a xylanase, cellulase, glucanase, pectinase, lipase, esterase, beta glucosidase, phytase, protease or any combination thereof.

e. Aqueous Phase High in Protein and Sugar Content

In yet another embodiment, proteases and lipases are engineered to accumulate in seeds, e.g., soybean seeds. After activation of the protease or lipase, such as, for example, by heating, these enzymes in the seeds hydrolyze the lipid and storage proteins present in soybeans during processing. Soluble products comprising amino acids, which can be used as feed, food or fermentation media, and fatty acids, can thus be obtained. Polysaccharides are typically found in the insoluble fraction of processed grain. However, by combining polysaccharide degrading enzyme expression and accumulation in seeds, proteins and polysaccharides can be hydrolyzed and are found in the aqueous phase. For example, zeins from corn and storage protein and non-starch polysaccharides from soybean can be solubilized in this manner. Components of the aqueous and hydrophobic phases can be easily separated by extraction with organic solvent or supercritical carbon dioxide. Thus, what is provided is a method for producing an aqueous extract of grain that contains higher levels of protein, amino acids, sugars or saccharides.

f. Self-Processing Fermentation

The invention provides a method to produce ethanol, a fermented beverage, or other fermentation-derived product(s). The method involves obtaining a plant, or the product or part of a plant, or plant derivative such as grain flour, wherein a processing enzyme that converts polysaccharides into sugar is expressed. The plant, or product thereof, is treated such that sugar is produced by conversion of the polysaccharide as described above. The sugars and other components of the plant are then fermented to form ethanol or a fermented beverage, or other fermentation-derived products, according to methods known in the art. See, for example, U.S. Pat. No. 4,929,452. Briefly the sugar produced by conversion of polysaccharides is incubated with yeast under conditions that promote conversion of the sugar into ethanol. A suitable yeast includes high alcohol-tolerant and high-sugar tolerant strains of yeast, such as, for example, the yeast, S. cerevisiae ATCC No. 20867. This strain was deposited with the American Type Culture Collection, Rockville, Md., on Sep. 17, 1987 and assigned ATCC No. 20867. The fermented product or fermented beverage may then be distilled to isolate ethanol or a distilled beverage, or the fermentation product otherwise recovered. The plant used in this method may be any plant that contains a polysaccharide and is able to express an enzyme of the invention. Many such plants are disclosed herein. Preferably the plant is one that is grown commercially. More preferably the plant is one that is normally used to produce ethanol or fermented beverages, or fermented products, such as, for example, wheat, barley, corn, rye, potato, grapes or rice.

The method comprises treating a plant part comprising at least one polysaccharide processing enzyme under conditions to activate the at least one enzyme thereby digesting polysaccharide in the plant part to form fermentable sugar. The polysaccharide processing enzyme may be mesophilic, thermophilic, or hyperthermophilic. The plant part is obtained from a transformed plant, the genome of which is augmented with an expression cassette encoding the at least one polysaccharide processing enzyme. Plant parts for this embodiment of the invention include, but are not limited to, grain, fruit, seed, stalk, wood, vegetable or root. Plants include but are not limited to oat, barley, wheat, berry, grape, rye, corn, rice, potato, sugar beet, sugar cane, pineapple, grass and tree. The plant part may be combined with commodity grain or other commercially available substrates; the source of the substrate for processing may be a source other than the self-processing plant. The fermentable sugar is then incubated under conditions that promote the conversion of the fermentable sugar into ethanol, e.g., with yeast and/or other microbes. In an embodiment, the plant part is derived from corn transformed with α-amylase, which has been found to reduce the amount of time and cost of fermentation.

It has been found that the amount of residual starch is reduced when transgenic corn made in accordance with the present invention expressing a thermostable α-amylase, for example, is used in fermentation. This indicates that more starch is solubilized during fermentation. The reduced amount of residual starch results in the distillers' grains having higher protein content by weight and higher value. Moreover, the fermentation of the transgenic corn of the present invention allows the liquefaction process to be performed at a lower pH, resulting in savings in the cost of chemicals used to adjust the pH, at a higher temperature, e.g., greater than 85° C., preferably, greater than 90° C., more preferably, 95° C. or higher, resulting in shorter liquefaction times and more complete solubilization of starch, and reduction of liquefaction times, all resulting in efficient fermentation reactions with higher yields of ethanol.

Moreover, it has been found that contacting conventional plant parts with even a small portion of the transgenic plant made in accordance with the present invention may reduce the fermentation time and costs associated therewith. As such, the present invention relates to the reduction in the fermentation time for plants comprising subjecting a transgenic plant part from a plant comprising a polysaccharide processing enzyme that converts polysaccharides into sugar relative to the use of a plant part not comprising the polysaccharide processing enzyme.

g. Raw Starch Processing Enzymes and Polynucleotides Encoding them

A polynucleotide encoding a mesophilic processing enzyme(s) is introduced into a plant or plant part. In an embodiment, the polynucleotide of the present invention is a maize-optimized polynucleotide such as provided in SEQ ID NOs: 48, 50, and 59, encoding a glucoamylase, such as provided in SEQ ID NOs: 47, and 49. In another embodiment, the polynucleotide of the present invention is a maize-optimized polynucleotide such as provided in SEQ ID NO: 52, encoding an alpha-amylase, such as provided in SEQ ID NO: 51. Moreover, fusion products of processing enzymes are further contemplated. In one embodiment, the polynucleotide of the present invention is a maize-optimized polynucleotide such as provided in SEQ ID NO: 46, encoding an alpha-amylase and glucoamylase fusion, such as provided in SEQ ID NO: 45. Combinations of processing enzymes are further envisioned by the present invention. For example, a combination of starch-processing enzymes and non-starch processing enzymes is contemplated herein. Such combinations of processing enzymes may be obtained by employing the use of multiple gene constructs encoding each of the enzymes. Alternatively, the individual transgenic plants stably transformed with the enzymes may be crossed by known methods to obtain a plant containing both enzymes. Another method includes the use of exogenous enzyme(s) with the transgenic plant.

The source of the starch-processing and non-starch processing enzymes may be isolated or derived from any source and the polynucleotides corresponding thereto may be ascertained by one having skill in the art. The α-amylase may be derived from Aspergillus (e.g., Aspergillus shirousami and Aspergillus niger), Rhizopus (eg., Rhizopus oryzae), and plants such as corn, barley, and rice. The glucoamylase may be derived from Aspergillus (e.g., Aspergillus shirousami and Aspergillus niger), Rhizopus (eg., Rhizopus oryzae), and Thermoanaerobacter (eg., Thermoanaerobacter thermosaccharolyticum).

In another embodiment of the invention, the polynucleotide encodes a mesophilic starch-processing enzyme that is operably linked to a maize-optimized polynucleotide such as provided in SEQ ID NO: 54, encoding a raw starch binding domain, such as provided in SEQ ID NO: 53.

In another embodiment, a tissue-specific promoter includes the endosperm-specific promoters such as the maize γ-zein promoter (exemplified by SEQ ID NO:12) or the maize ADP-gpp promoter (exemplified by SEQ ID NO:11, which includes a 5′ untranslated and an intron sequence) or a Q protein promoter (exemplified by SEQ ID NO: 98) or a rice glutelin promoter (exemplified by SEQ ID NO: 67). Thus, the present invention includes an isolated polynucleotide comprising a promoter comprising SEQ ID NO: 11, 12, 67, or 98, a polynucleotide which hybridizes to the complement thereof under low stringency hybridization conditions, or a fragment thereof which has promoter activity, e.g., at least 10%, and preferably at least 50%, the activity of a promoter having SEQ ID NO:11, 12, 67 or 98.

In one embodiment, the product from a starch-hydrolysis gene, such as α-amylase, glucoamylase, or α-amylase/glucoamylase fusion may be targeted to a particular organelle or location such as the endoplasmic reticulum or apoplast, rather than to the cytoplasm. This is exemplified by the use of the maize γ-zein N-terminal signal sequence (SEQ ID NO:17), which confers apoplast-specific targeting of proteins, and the use of the γ-zein N-terminal signal sequence (SEQ ID NO:17) which is operably linked to the processing enzyme that is operably linked to the sequence SEKDEL for retention in the endoplasmic reticulum. Directing the protein or enzyme to a specific compartment will allow the enzyme to be localized in a manner that it will not come into contact with the substrate. In this manner the enzymatic action of the enzyme will not occur until the enzyme contacts its substrate. The enzyme can be contacted with its substrate by the process of milling (physical disruption of the cell integrity) and hydrating. For example, a mesophilic starch-hydrolyzing enzyme can be targeted to the apoplast or to the endoplasmic reticulum and will therefore not come into contact with starch granules in the amyloplast. Milling of the grain will disrupt the integrity of the grain and the starch hydrolyzing enzyme will then contact the starch granules. In this manner the potential negative effects of co-localization of an enzyme and its substrate can be circumvented.

h. Food Products without Added Sweetener

Also provided is a method to produce a sweetened farinaceous food product without adding additional sweetener. Examples of farinaceous products include, but are not limited to, breakfast food, ready to eat food, baked food, pasta and cereal products such as breakfast cereal. The method comprises treating a plant part comprising at least one starch processing enzyme under conditions which activate the starch processing enzyme, thereby processing starch granules in the plant part to sugars so as to form a sweetened product, e.g., relative to the product produced by processing starch granules from a plant part which does not comprise the hyperthermophilic enzyme. Preferably, the starch processing enzyme is hyperthermophilic and is activated by heating, such as by baking, boiling, heating, steaming, electrical discharge, or any combination thereof. The plant part is obtained from a transformed plant, for instance from transformed soybean, rye, oat, barley, wheat, corn, rice or sugar cane, the genome of which is augmented with an expression cassette encoding the at least one hyperthermophilic starch processing enzyme, e.g., α-amylase, α-glucosidase, glucoamylase, pullulanase, glucose isomerase, or any combination thereof. The sweetened product is then processed into a farinaceous food product. The invention also provides a farinaceous food product, e.g., a cereal food, a breakfast food, a ready to eat food, or a baked food, produced by the method. The farinaceous food product may be formed from the sweetened product and water, and may contain malt, flavorings, vitamins, minerals, coloring agents or any combination thereof.

The enzyme may be activated to convert polysaccharides contained within the plant material into sugar prior to inclusion of the plant material into the cereal product or during the processing of the cereal product. Accordingly, polysaccharides contained within the plant material may be converted into sugar by activating the material, such as by heating in the case of a hyperthermophilic enzyme, prior to inclusion in the farinaceous product. The plant material containing sugar produced by conversion of the polysaccharides is then added to the product to produce a sweetened product. Alternatively, the polysaccharides may be converted into sugars by the enzyme during the processing of the farinaceous product. Examples of processes used to make cereal products are well known in the art and include heating, baking, boiling and the like as described in U.S. Pat. Nos. 6,183,788; 6,159,530; 6,149,965; 4,988,521 and 5,368,870.

Briefly, dough may be prepared by blending various dry ingredients together with water and cooking to gelatinize the starchy components and to develop a cooked flavor. The cooked material can then be mechanically worked to form a cooked dough, such as cereal dough. The dry ingredients may include various additives such as sugars, starch, salt, vitamins, minerals, colorings, flavorings, salt and the like. In addition to water, various liquid ingredients such as corn (maize) or malt syrup can be added. The farinaceous material may include cereal grains, cut grains, grits or flours from wheat, rice, corn, oats, barley, rye, or other cereal grains and mixtures thereof from that a transformed plant of the invention. The dough may then be processed into a desired shape through a process such as extrusion or stamping and further cooked using means such as a James cooker, an oven or an electrical discharge device.

Further provided is a method to sweeten a starch containing product without adding sweetener. The method comprises treating starch comprising at least one starch processing enzyme conditions to activate the at least one enzyme thereby digesting the starch to form a sugar thereby forming a treated (sweetened) starch, e.g., relative to the product produced by treating starch which does not comprise the hyperthermophilic enzyme. The starch of the invention is obtained from a transformed plant, the genome of which is augmented with an expression cassette encoding the at least one processing enzyme. Enzymes include α-amylase, α-glucosidase, glucoamylase, pullulanase, glucose isomerase, or any combination thereof. The enzyme may be hyperthermophilic and activated with heat. Preferred transformed plants include corn, soybean, rye, oat, barley, wheat, rice and sugar cane. The treated starch is then added to a product to produce a sweetened starch containing product, e.g., a farinaceous food product. Also provided is a sweetened starch containing product produced by the method.

The invention further provides a method to sweeten a polysaccharide containing fruit or vegetable comprising: treating a fruit or vegetable comprising at least one polysaccharide processing enzyme under conditions which activate the at least one enzyme, thereby processing the polysaccharide in the fruit or vegetable to form sugar, yielding a sweetened fruit or vegetable, e.g., relative to a fruit or vegetable from a plant which does not comprise the polysaccharide processing enzyme. The fruit or vegetable of the invention is obtained from a transformed plant, the genome of which is augmented with an expression cassette encoding the at least one polysaccharide processing enzyme. Fruits and vegetables include potato, tomato, banana, squash, pea, and bean. Enzymes include α-amylase, α-glucosidase, glucoamylase, pullulanase, glucose isomerase, or any combination thereof. The enzyme may be hyperthermophilic.

i. Sweetening a Polysaccharide Containing Plant or Plant Product

The method involves obtaining a plant that expresses a polysaccharide processing enzyme which converts a polysaccharide into a sugar as described above. Accordingly the enzyme is expressed in the plant and in the products of the plant, such as in a fruit or vegetable. In one embodiment, the enzyme is placed under the control of an inducible promoter such that expression of the enzyme may be induced by an external stimulus. Such inducible promoters and constructs are well known in the art and are described herein. Expression of the enzyme within the plant or product thereof causes polysaccharide contained within the plant or product thereof to be converted into sugar and to sweeten the plant or product thereof. In another embodiment, the polysaccharide processing enzyme is constitutively expressed. Thus, the plant or product thereof may be activated under conditions sufficient to activate the enzyme to convert the polysaccharides into sugar through the action of the enzyme to sweeten the plant or product thereof. As a result, this self-processing of the polysaccharide in the fruit or vegetable to form sugar yields a sweetened fruit or vegetable, e.g., relative to a fruit or vegetable from a plant which does not comprise the polysaccharide processing enzyme. The fruit or vegetable of the invention is obtained from a transformed plant, the genome of which is augmented with an expression cassette encoding the at least one polysaccharide processing enzyme. Fruits and vegetables include potato, tomato, banana, squash, pea, and bean. Enzymes include α-amylase, α-glucosidase, glucoamylase, pullulanase, glucose isomerase, or any combination thereof. The polysaccharide processing enzyme may be hyperthermophilic.

j. Isolation of Starch from Transformed Grain that Contains a Enzyme which Disrupts the Endosperm Matrix

The invention provides a method to isolate starch from a transformed grain wherein an enzyme is expressed that disrupts the endosperm matrix. The method involves obtaining a plant that expresses an enzyme which disrupts the endosperm matrix by modification of, for example, cell walls, non-starch polysaccharides and/or proteins. Examples of such enzymes include, but are not limited to, proteases, glucanases, thioredoxin, thioredoxin reductase, phytases, lipases, cellulases, beta glucosidases, xylanases and esterases. Such enzymes do not include any enzyme that exhibits starch-degrading activity so as to maintain the integrity of the starch granules. The enzyme may be fused to a signal sequence that targets the enzyme to the starch granule. In one embodiment the grain is heat dried to activate the enzyme and inactivate the endogenous enzymes contained within the grain. The heat treatment causes activation of the enzyme, which acts to disrupt the endosperm matrix which is then easily separated from the starch granules. In another embodiment, the grain is steeped at low or high temperature, with high or low moisture content, with or without sulfur dioxide. The grain is then heat treated to disrupt the endosperm matrix and allow for easy separation of the starch granules. In another embodiment, proper temperature and moisture conditions are created to allow proteases to enter into the starch granules and degrade proteins contained within the granules. Such treatment would produce starch granules with high yield and little contaminating protein.

k. Syrup Having a High Sugar Equivalent and Use of the Syrup to Produce Ethanol or a Fermented Beverage

The method involves obtaining a plant that expresses a polysaccharide processing enzyme which converts a polysaccharide into a sugar as described above. The plant, or product thereof, is steeped in an aqueous stream under conditions where the expressed enzyme converts polysaccharide contained within the plant, or product thereof, into dextrin, maltooligosaccharide, and/or sugar. The aqueous stream containing the dextrin, maltooligosaccharide, and/or sugar produced through conversion of the polysaccharide is then separated to produce a syrup having a high sugar equivalent. The method may or may not include an additional step of wet-milling the plant or product thereof to obtain starch granules. Examples of enzymes that may be used within the method include, but are not limited to, α-amylase, glucoamylase, pullulanase and α-glucosidase. The enzyme may be hyperthermophilic. Sugars produced according to the method include, but are not limited to, hexose, glucose and fructose. Examples of plants that may be used with the method include, but are not limited to, corn, wheat or barley. Examples of products of a plant that may be used include, but are not limited to, fruit, grain and vegetables. In one embodiment, the polysaccharide processing enzyme is placed under the control of an inducible promoter. Accordingly, prior to or during the steeping process, the promoter is induced to cause expression of the enzyme, which then provides for the conversion of polysaccharide into sugar. Examples of inducible promoters and constructs containing them are well known in the art and are provided herein. Thus, where the polysaccharide processing is hyperthermophilic, the steeping is performed at a high temperature to activate the hyperthermophilic enzyme and inactivate endogenous enzymes found within the plant or product thereof. In another embodiment, a hyperthermophilic enzyme able to convert polysaccharide into sugar is constitutively expressed. This enzyme may or may not be targeted to a compartment within the plant through use of a signal sequence. The plant, or product thereof, is steeped under high temperature conditions to cause the conversion of polysaccharides contained within the plant into sugar.

Also provided is a method to produce ethanol or a fermented beverage from syrup having a high sugar equivalent. The method involves incubating the syrup with yeast under conditions that allow conversion of sugar contained within the syrup into ethanol or a fermented beverage. Examples of such fermented beverages include, but are not limited to, beer and wine. Fermentation conditions are well known in the art and are described in U.S. Pat. No. 4,929,452 and herein. Preferably the yeast is a high alcohol-tolerant and high-sugar tolerant strain of yeast such as S. cerevisiae ATCC No. 20867. The fermented product or fermented beverage may be distilled to isolate ethanol or a distilled beverage.

l. Accumulation of Hyperthermophilic Enzyme in the Cell Wall of a Plant

The invention provides a method to accumulate a hyperthermophilic enzyme in the cell wall of a plant. The method involves expressing within a plant a hyperthermophilic enzyme that is fused to a cell wall targeting signal such that the targeted enzyme accumulates in the cell wall. Preferably the enzyme is able to convert polysaccharides into monosaccharides. Examples of targeting sequences include, but are not limited to, a cellulose or xylose binding domain. Examples of hyperthermophilic enzymes include those listed in SEQ ID NO: 1, 3, 5, 10, 13, 14, or 16. Plant material containing cell walls may be added as a source of desired enzymes in a process to recover sugars from the feedstock or as a source of enzymes for the conversion of polysaccharides originating from other sources to monosaccharides. Additionally, the cell walls may serve as a source from which enzymes may be purified. Methods to purify enzymes are well known in the art and include, but are not limited to, gel filtration, ion-exchange chromatography, chromatofocusing, isoelectric focusing, affinity chromatography, FPLC, HPLC, salt precipitation, dialysis, and the like. Accordingly, the invention also provides purified enzymes isolated from the cell walls of plants.

m. Method of Preparing and Isolating Processing Enzymes

In accordance with the present invention, recombinantly-produced processing enzymes of the present invention may be prepared by transforming plant tissue or plant cell comprising the processing enzyme of the present invention capable of being activated in the plant, selected for the transformed plant tissue or cell, growing the transformed plant tissue or cell into a transformed plant, and isolating the processing enzyme from the transformed plant or part thereof. The recombinantly-produced enzyme may be an α-amylase, glucoamylase, glucose isomerase, α-glucosidase, pullulinase, xylanase, protease, glucanase, beta glucosidase, esterase, lipase, or phytase. The enzyme may be encoded by the polynucleotide selected from any of SEQ ID NO: 2, 4, 6, 9, 19, 21, 25, 37, 39, 41, 43, 46, 48, 50, 52, 59, 61, 63, 65, 79, 81, 83, 85, 87, 89, 91, 93, 94, 95, 96, 97, or 99.

The invention will be further described by the following examples, which are not intended to limit the scope of the invention in any manner.

### EXAMPLES

### Example 1

### Construction of Maize-Optimized Genes for Hyperthermophilic Starch-Processing/Isomerization Enzymes

The enzymes, α-amylase, pullulanase, α-glucosidase, and glucose isomerase, involved in starch degradation or glucose isomerization were selected for their desired activity profiles. These include, for example, minimal activity at ambient temperature, high temperature activity/stability, and activity at low pH. The corresponding genes were then designed by using maize preferred codons as described in U.S. Pat. No. 5,625,136 and synthesized by Integrated DNA Technologies, Inc. (Coralville, Iowa).

The 797GL3 α-amylase, having the amino acid sequence SEQ ID NO:1, was selected for its hyperthermophilic activity. This enzyme's nucleic acid sequence was deduced and maize-optimized as represented in SEQ ID NO:2. Similarly, the 6gp3 pullulanase was selected having the amino acid sequence set forth in SEQ ID NO:3. The nucleic acid sequence for the 6gp3 pullulanase was deduced and maize-optimized as represented in SEQ ID NO:4.

The amino acid sequence for malA α-glucosidase from Sulfolobus solfataricus was obtained from the literature, J. Bact. 177:482-485 (1995); J. Bact. 180:1287-1295 (1998). Based on the published amino acid sequence of the protein (SEQ ID NO:5), the maize-optimized synthetic gene (SEQ ID NO:6) encoding the malA α-glucosidase was designed.

Several glucose isomerase enzymes were selected. The amino acid sequence (SEQ ID NO:18) for glucose isomerase derived from Thermotoga maritima was predicted based on the published DNA sequence having Accession No. NC_{—}000853 and a maize-optimized synthetic gene was designed (SEQ ID NO: 19). Similarly the amino acid sequence (SEQ ID NO:20) for glucose isomerase derived from Thermotoga neapolitana was predicted based on the published DNA sequence from Appl. Envir. Microbiol. 61(5):1867-1875 (1995), Accession No. L38994. A maize-optimized synthetic gene encoding the Thermotoga neapolitana glucose isomerase was designed (SEQ ID NO:21).

### Example 2

### Expression of Fusion of 797GL3 α-amylase and Starch Encapsulating Region in E. Coli

A construct encoding hyperthermophilic 797GL3 α-amylase fused to the starch encapsulating region (SER) from maize granule-bound starch synthase (waxy) was introduced and expressed in E. coli. The maize granule-bound starch synthase cDNA (SEQ ID NO:7) encoding the amino acid sequence (SEQ ID NO:8) (Klosgen R B, et al. 1986) was cloned as a source of a starch binding domain, or starch encapsulating region (SER). The full-length cDNA was amplified by RT-PCR from RNA prepared from maize seed using primers SV57 (5′AGCGAATTCATGGCGGCTCTGGCCACGT 3′) (SEQ ID NO: 22) and SV58 (5′AGCTAAGCTTCAGGGCGCGGCCACGTTCT 3′) (SEQ ID NO: 23) designed from GenBank Accession No. X03935. The complete cDNA was cloned into pBluescript as an EcoRI/HindIII fragment and the plasmid designated pNOV4022.

The C-terminal portion (encoded by bp 919-1818) of the waxy cDNA, including the starch-binding domain, was amplified from pNOV4022 and fused in-frame to the 3′ end of the full-length maize-optimized 797GL3 gene (SEQ ID NO:2). The fused gene product, 797GL3/Waxy, having the nucleic acid SEQ ID NO:9 and encoding the amino acid sequence, SEQ ID NO:10, was cloned as an NcoI/XbaI fragment into pET28b (NOVAGEN, Madison, Wis.) that was cut with NcoI/NheI. The 797GL3 gene alone was also cloned into the pET28b vector as an NcoI/XbaI fragment.

The pET28/797GL3 and the pET28/797GL3/Waxy vectors were transformed into BL21/DE3 E. coli cells (NOVAGEN) and grown and induced according to the manufacturer's instruction. Analysis by PAGE/Coomassie staining revealed an induced protein in both extracts corresponding to the predicted sizes of the fused and unfused amylase, respectively.

Total cell extracts were analyzed for hyperthermophilic amylase activity as follows: 5 mg of starch was suspended in 20 μl of water then diluted with 25 μl of ethanol. The standard amylase positive control or the sample to be tested for amylase activity was added to the mixture and water was added to a final reaction volume of 500 μl. The reaction was carried out at 80° C. for 15-45 minutes. The reaction was then cooled down to room temperature, and 500 μl of o-dianisidine and glucose oxidase/peroxidase mixture (Sigma) was added. The mixture was incubated at 37° C. for 30 minutes. 500 μl of 12 N sulfuric acid was added to stop the reaction. Absorbance at 540 nm was measured to quantitate the amount of glucose released by the amylase/sample. Assay of both the fused and unfused amylase extracts gave similar levels of hyperthermophilic amylase activity, whereas control extracts were negative. This indicated that the 797GL3 amylase was still active (at high temperatures) when fused to the C-terminal portion of the waxy protein.

### Example 3

### Isolation of Promoter Fragments for Endosperm-Specific Expression in Maize

The promoter and 5′ noncoding region I (including the first intron) from the large subunit of Zea mays ADP-gpp (ADP-glucose pyrophosphorylase) was amplified as a 1515 base pair fragment (SEQ ID NO:11) from maize genomic DNA using primers designed from Genbank accession M81603. The ADP-gpp promoter has been shown to be endosperm-specific (Shaw and Hannah, 1992).

The promoter from the Zea mays γ-zein gene was amplified as a 673 bp fragment (SEQ ID NO:12) from plasmid pGZ27.3 (obtained from Dr. Brian Larkins). The γ-zein promoter has been shown to be endosperm-specific (Torrent et al. 1997).

### Example 4

### Construction of Transformation Vectors for the 797GL3 Hyperthermophilic α-amylase

Expression cassettes were constructed to express the 797GL3 hyperthermophilic amylase in maize endosperm with various targeting signals as follows:

pNOV6200 (SEQ ID NO:13) comprises the maize γ-zein N-terminal signal sequence (MRVLLVALALLALAASATS) (SEQ ID NO:17) fused to the synthetic 797GL3 amylase as described above in Example 1 for targeting to the endoplasmic reticulum and secretion into the apoplast (Torrent et al. 1997). The fusion was cloned behind the maize ADP-gpp promoter for expression specifically in the endosperm.

pNOV6201 (SEQ ID NO:14) comprises the γ-zein N-terminal signal sequence fused to the synthetic 797GL3 amylase with a C-terminal addition of the sequence SEKDEL for targeting to and retention in the endoplasmic reticulum (ER) (Munro and Pelham, 1987). The fusion was cloned behind the maize ADP-gpp promoter for expression specifically in the endosperm.

pNOV7013 comprises the γ-zein N-terminal signal sequence fused to the synthetic 797GL3 amylase with a C-terminal addition of the sequence SEKDEL for targeting to and retention in the endoplasmic reticulum (ER). PNOV7013 is the same as pNOV6201, except that the maize γ-zein promoter (SEQ ID NO:12) was used instead of the maize ADP-spp promoter in order to express the fusion in the endosperm.

pNOV4029 (SEQ ID NO:15) comprises the waxy amyloplast targeting peptide (Klosgen et al., 1986) fused to the synthetic 797GL3 amylase for targeting to the amyloplast. The fusion was cloned behind the maize ADP-gpp promoter for expression specifically in the endosperm.

pNOV4031 (SEQ ID NO:16) comprises the waxy amyloplast targeting peptide fused to the synthetic 797GL3/waxy fusion protein for targeting to starch granules. The fusion was cloned behind the maize ADP-gpp promoter for expression specifically in the endosperm.

Additional constructs were made with these fusions cloned behind the maize γ-zein promoter to obtain higher levels of enzyme expression. All expression cassettes were moved into a binary vector for transformation into maize via Agrobacterium infection. The binary vector contained the phosphomannose isomerase (PMI) gene which allows for selection of transgenic cells with mannose. Transformed maize plants were either self-pollinated or outcrossed and seed was collected for analysis.

Additional constructs were made with the targeting signals described above fused to either 6gp3 pullulanase or to 340g12 α-glucosidase in precisely the same manner as described for the α-amylase. These fusions were cloned behind the maize ADP-gpp promoter and/or the γ-zein promoter and transformed into maize as described above. Transformed maize plants were either self-pollinated or outcrossed and seed was collected for analysis.

Combinations of the enzymes can be produced either by crossing plants expressing the individual enzymes or by cloning several expression cassettes into the same binary vector to enable cotransformation.

### Example 5

### Construction of Plant Transformation Vectors for the 6GP3 Thermophillic Pullulanase

An expression cassette was constructed to express the 6GP3 thermophillic pullanase in the endoplasmic reticulum of maize endosperm as follows:

pNOV7005 (SEQ ID NOs:24 and 25) comprises the maize γ-zein N-terminal signal sequence fused to the synthetic 6GP3 pullulanase with a C-terminal addition of the sequence SEKDEL for targeting to and retention in the ER. The amino acid peptide SEKDEL was fused to the C-terminal end of the enzymes using PCR with primers designed to amplify the synthetic gene and simultaneously add the 6 amino acids at the C-terminal end of the protein. The fusion was cloned behind the maize γ-zein promoter for expresson specifically in the endosperm.

### Example 6

### Construction of Plant Transformation Vectors for the malA Hyperthermophilic α-glucosidase

Expression cassettes were constructed to express the Sulfolobus solfataricus malA hyperthermophilic α-glucosidase in maize endosperm with various targeting signals as follows:

pNOV4831 (SEQ ID NO:26) comprises the maize γ-zein N-terminal signal sequence (MRVLLVALALLALAASATS) (SEQ ID NO:17) fused to the synthetic malA α-glucosidase with a C-terminal addition of the sequence SEKDEL for targeting to and retention in the endoplasmic reticulum (ER) (Munro and Pelham, 1987). The fusion was cloned behind the maize γ-zein promoter for expresson specifically in the endosperm.

pNOV4839 (SEQ ID NO:27) comprises the maize γ-zein N-terminal signal sequence (MRVLLVALALLALAASATS) (SEQ ID NO:17) fused to the synthetic malA α-glucosidase for targeting to the endoplasmic reticulum and secretion into the apoplast (Torrent et al. 1997). The fusion was cloned behind the maize γ-zein promoter for expression specifically in the endosperm.

pNOV4837 comprises the maize γ-zein N-terminal signal sequence (MRVLLVALALLALAASATS) (SEQ ID NO:17) fused to the synthetic malA α-glucosidase with a C-terminal addition of the sequence SEKDEL for targeting to and retention in the ER. The fusion was cloned behind the maize ADPgpp promoter for expression specifically in the endosperm. The amino acid sequence for this clone is identical to that of pNOV4831 (SEQ ID NO:26).

### Example 7

### Construction of Plant Transformation Vectors for the Hyperthermophillic Thermotoga maritima and Thermotoga neapolitana Glucose Isomerases

Expression cassettes were constructed to express the Thermotoga maritima and Thermotoga neapolitana hyperthermophilic glucose isomerases in maize endosperm with various targeting signals as follows:

pNOV4832 (SEQ ID NO:28) comprises the maize γ-zein N-terminal signal sequence (MRVLLVALALLALAASATS) (SEQ ID NO:17) fused to the synthetic Thermotoga maritima glucose isomerase with a C-terminal addition of the sequence SEKDEL for targeting to and retention in the ER. The fusion was cloned behind the maize γ-zein promoter for expression specifically in the endosperm.

pNOV4833 (SEQ ID NO:29) comprises the maize γ-zein N-terminal signal sequence (MRVLLVALALLALAASATS) (SEQ ID NO:17) fused to the synthetic Thermotoga neapolitana glucose isomerase with a C-terminal addition of the sequence SEKDEL for targeting to and retention in the ER. The fusion was cloned behind the maize γ-zein promoter for expression specifically in the endosperm.

pNOV4840 (SEQ ID NO:30) comprises the maize γ-zein N-terminal signal sequence (MRVLLVALALLALAASATS) (SEQ ID NO:17) fused to the synthetic Thermotoga neapolitana glucose isomerase for targeting to the endoplasmic reticulum and secretion into the apoplast (Torrent et al. 1997). The fusion was cloned behind the maize γ-zein promoter for expression specifically in the endosperm.

pNOV4838 comprises the maize γ-zein N-terminal signal sequence (MRVLLVALALLALAASATS) (SEQ ID NO:17) fused to the synthetic Thermotoga neapolitana glucose isomerase with a C-terminal addition of the sequence SEKDEL for targeting to and retention in the ER. The fusion was cloned behind the maize ADPgpp promoter for expression specifically in the endosperm. The amino acid sequence for this clone is identical to that of pNOV4833 (SEQ ID NO:29).

### Example 8

### Construction of Plant Transformation Vectors for the Expression of the Hyperthermophillic Glucanase EglA

pNOV4800 (SEQ ID NO:58) comprises the barley alpha amylase AMY32b signal sequence (MGKNGNLCCFSLLLLLLAGLASGHQ) (SEQ ID NO:31) fused with the EglA mature protein sequence for localization to the apoplast. The fusion was cloned behind the maize γ-zein promoter for expression specifically in the endosperm.

### Example 9

### Construction of Plant Transformation Vectors for the Expression of Multiple Hyperthermophillic Enzymes

pNOV4841 comprises a double gene construct of a 797GL3 α-amylase fusion and a 6GP3 pullulanase fusion. Both 797GL3 fusion (SEQ ID NO:33) and 6GP3 fusion (SEQ ID NO:34) possessed the maize γ-zein N-terminal signal sequence and SEKDEL sequence for targeting to and retention in the ER. Each fusion was cloned behind a separate maize γ-zein promoter for expression specifically in the endosperm.

pNOV4842 comprises a double gene construct of a 797GL3 α-amylase fusion and a malA α-glucosidase fusion. Both the 797GL3 fusion polypeptide (SEQ ID NO:35) and malA α-glucosidase fusion polypeptide (SEQ ID NO:36) possess the maize γ-zein N-terminal signal sequence and SEKDEL sequence for targeting to and retention in the ER. Each fusion was cloned behind a separate maize γ-zein promoter for expression specifically in the endosperm.

pNOV4843 comprises a double gene construct of a 797GL3 α-amylase fusion and a malA α-glucosidase fusion. Both the 797GL3 fusion and malA α-glucosidase fusion possess the maize γ-zein N-terminal signal sequence and SEKDEL sequence for targeting to and retention in the ER. The 797GL3 fusion was cloned behind the maize γ-zein promoter and the malA fusion was cloned behind the maize ADPgpp promoter for expression specifically in the endosperm. The amino acid sequences of the 797GL3 fusion and the malA fusion are identical to those of pNOV4842 (SEQ ID Nos: 35 and 36, respectively).

pNOV4844 comprises a triple gene construct of a 797GL3 α-amylase fusion, a 6GP3 pullulanase fusion, and a malA α-glucosidase fusion. 797GL3, malA, and 6GP3 all possess the maize γ-zein N-terminal signal sequence and SEKDEL sequence for targeting to and retention in the ER. The 797GL3 and malA fusions were cloned behind 2 separate maize γ-zein promoters, and the 6GP3 fusion was cloned behind the maize ADPgpp promoter for expression specifically in the endosperm. The amino acid sequences for the 797GL3 and malA fusions are identical to those of pNOV4842 (SEQ ID Nos: 35 and 36, respectively). The amino acid sequence for the 6GP3 fusion is identical to that of the 6GP3 fusion in pNOV4841 (SEQ ID NO:34).

All expression cassettes set forth in this Example as well as in the Examples that follow were moved into the binary vector pNOV2117 for transformation into maize via Agrobacterium infection. pNOV2117 contains the phosphomannose isomerase (PMI) gene allowing for selection of transgenic cells with mannose. pNOV2117 is a binary vector with both the pVS1 and ColE1 origins of replication. This vector contains the constitutive VirG gene from pAD1289 (Hansen, G., et al., PNAS USA 91:7603-7607 (1994), incorporated by reference herein) and a spectinomycin resistance gene from Tn7. Cloned into the polylinker between the right and left borders are the maize ubiquitin promoter, PMI coding region and nopaline synthase terminator of pNOV117 (Negrotto, D., et al., Plant Cell Reports 19:798-803 (2000), incorporated by reference herein). Transformed maize plants will either be self-pollinated or outcrossed and seed collected for analysis. Combinations of the different enzymes can be produced either by crossing plants expressing the individual enzymes or by transforming a plant with one of the multi-gene cassettes.

### Example 10

### Construction of Bacterial and Pichia Expression Vectors

Expression cassettes were constructed to express the hyperthermophilic α-glucosidase and glucose isomerases in either Pichia or bacteria as follows:

pNOV4829 (SEQ ID NOS: 37 and 38) comprises a synthetic Thermotoga maritima glucose isomerase fusion with ER retention signal in the bacterial expression vector pET29a. The glucose isomerase fusion gene was cloned into the NcoI and SacI sites of pET29a, which results in the addition of an N-terminal S-tag for protein purification.

pNOV4830 (SEQ ID NOS: 39 and 40) comprises a synthetic Thermotoga neapolitana glucose isomerase fusion with ER retention signal in the bacterial expression vector pET29a. The glucose isomerase fusion gene was cloned into the NcoI and SacI sites of pET29a, which results in the addition of an N-terminal S-tag for protein purification.

pNOV4835 (SEQ ID NO: 41 and 42) comprises the synthetic Thermotoga maritima glucose isomerase gene cloned into the BamHI and EcoRI sites of the bacterial expression vector pET28C. This resulted in the fusion of a His-tag (for protein purification) to the N-terminal end of the glucose isomerase.

pNOV4836 (SEQ ID NO: 43 AND 44) comprises the synthetic Thermotoga neapolitana glucose isomerase gene cloned into the BamHI and EcoRI sites of the bacterial expression vector pET28C. This resulted in the fusion of a His-tag (for protein purification) to the N-terminal end of the glucose isomerase.

### Example 11

Transformation of immature maize embryos was performed essentially as described in Negrotto et al., Plant Cell Reports 19: 798-803. For this example, all media constituents are as described in Negrotto et al., supra. However, various media constituents described in the literature may be substituted.

### A. Transformation Plasmids and Selectable Marker

The genes used for transformation were cloned into a vector suitable for maize transformation. Vectors used in this example contained the phosphomannose isomerase (PMI) gene for selection of transgenic lines (Negrotto et al. (2000) Plant Cell Reports 19: 798-803).

### B. Preparation of Agrobacterium tumefaciens

Agrobacterium strain LBA4404 (pSB1) containing the plant transformation plasmid was grown on YEP (yeast extract (5 g/L), peptone (10 g/L), NaCl (5 g/L), 15 g/l agar, pH 6.8) solid medium for 2-4 days at 28° C. Approximately 0.8×10⁹ Agrobacterium were suspended in LS-inf media supplemented with 100 μM As (Negrotto et al., (2000) Plant Cell Rep 19: 798-803). Bacteria were pre-induced in this medium for 30-60 minutes.

### C. Inoculation

Immature embryos from A188 or other suitable genotype were excised from 8-12 day old ears into liquid LS-inf+100 μM As. Embryos were rinsed once with fresh infection medium. Agrobacterium solution was then added and embryos were vortexed for 30 seconds and allowed to settle with the bacteria for 5 minutes. The embryos were then transferred scutellum side up to LSAs medium and cultured in the dark for two to three days. Subsequently, between 20 and 25 embryos per petri plate were transferred to LSDc medium supplemented with cefotaxime (250 mg/l) and silver nitrate (1.6 mg/l) and cultured in the dark for 28° C. for 10 days.

### D. Selection of Transformed Cells and Regeneration of Transformed Plants

Immature embryos producing embryogenic callus were transferred to LSD1M0.5S medium. The cultures were selected on this medium for 6 weeks with a subculture step at 3 weeks. Surviving calli were transferred to Reg1 medium supplemented with mannose. Following culturing in the light (16 hour light/8 hour dark regiment), green tissues were then transferred to Reg2 medium without growth regulators and incubated for 1-2 weeks. Plantlets are transferred to Magenta GA-7 boxes (Magenta Corp, Chicago Ill.) containing Reg3 medium and grown in the light. After 2-3 weeks, plants were tested for the presence of the PMI genes and other genes of interest by PCR. Positive plants from the PCR assay were transferred to the greenhouse.

### Example 12

### Analysis of T1 Seed from Maize Plants Expressing the α-amylase Targeted to Apoplast or to the ER

T1 seed from self-pollinated maize plants transformed with either pNOV6200 or pNOV6201 as described in Example 4 were obtained. Starch accumulation in these kernels appeared to be normal, based on visual inspection and on normal staining for starch with an iodine solution prior to any exposure to high temperature. Immature kernels were dissected and purified endosperms were placed individually in microfuge tubes and immersed in 200 μl of 50 mM NaPO₄ buffer. The tubes were placed in an 85° C. water bath for 20 minutes, then cooled on ice. Twenty microliters of a 1% iodine solution was added to each tube and mixed. Approximately 25% of the segregating kernels stained normally for starch. The remaining 75% failed to stain, indicating that the starch had been degraded into low molecular weight sugars that do not stain with iodine. It was found that the T1 kernels of pNOV6200 and pNOV6201 were self-hydrolyzing the corn starch. There was no detectable reduction in starch following incubation at 37° C.

Expression of the amylase was further analyzed by isolation of the hyperthermophilic protein fraction from the endosperm followed by PAGE/Coomassie staining. A segregating protein band of the appropriate molecular weight (50 kD) was observed. These samples are subjected to an α-amylase assay using commercially available dyed amylose (AMYLAZYME, from Megazyme, Ireland). High levels of hyperthermophilic amylase activity correlated with the presence of the 50 kD protein.

It was further found that starch in kernels from a majority of transgenic maize, which express hyperthermophilic α-amylase, targeted to the amyloplast, is sufficiently active at ambient temperature to hydrolyze most of the starch if the enzyme is allowed to be in direct contact with a starch granule. Of the eighty lines having hyperthermophilic α-amylase targeted to the amyloplast, four lines were identified that accumulate starch in the kernels. Three of these lines were analyzed for thermostable α-amylase activity using a colorimetric amylazyme assay (Megazyme). The amylase enzyme assay indicated that these three lines had low levels of thermostable amylase activity. When purified starch from these three lines was treated with appropriate conditions of moisture and heat, the starch was hydrolyzed indicating the presence of adequate levels of α-amylase to facilitate the auto-hydrolysis of the starch prepared from these lines.

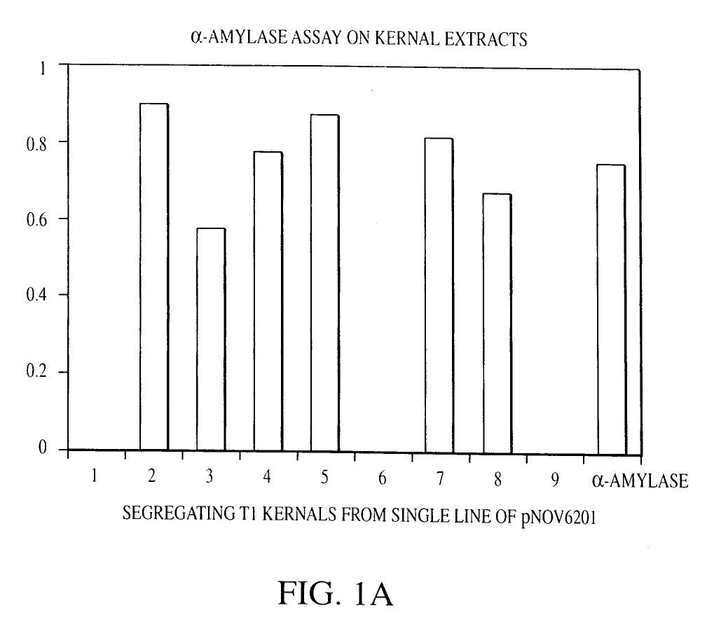

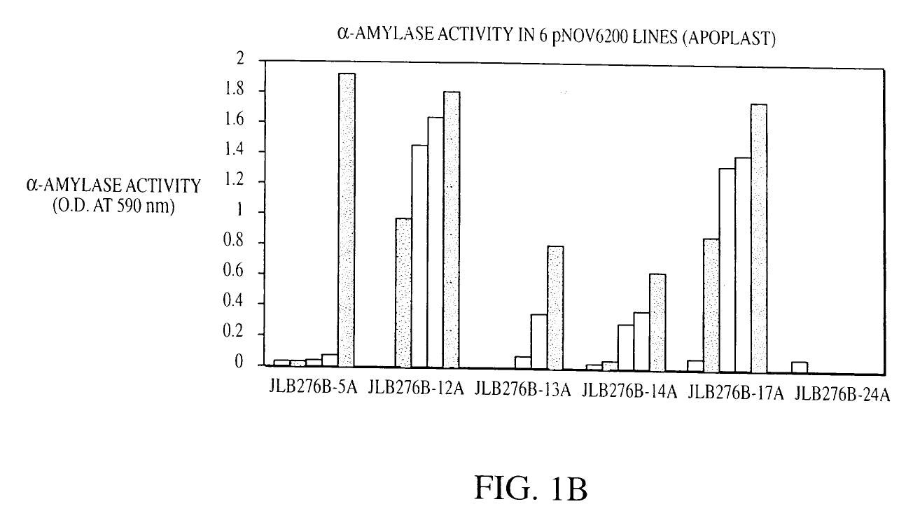

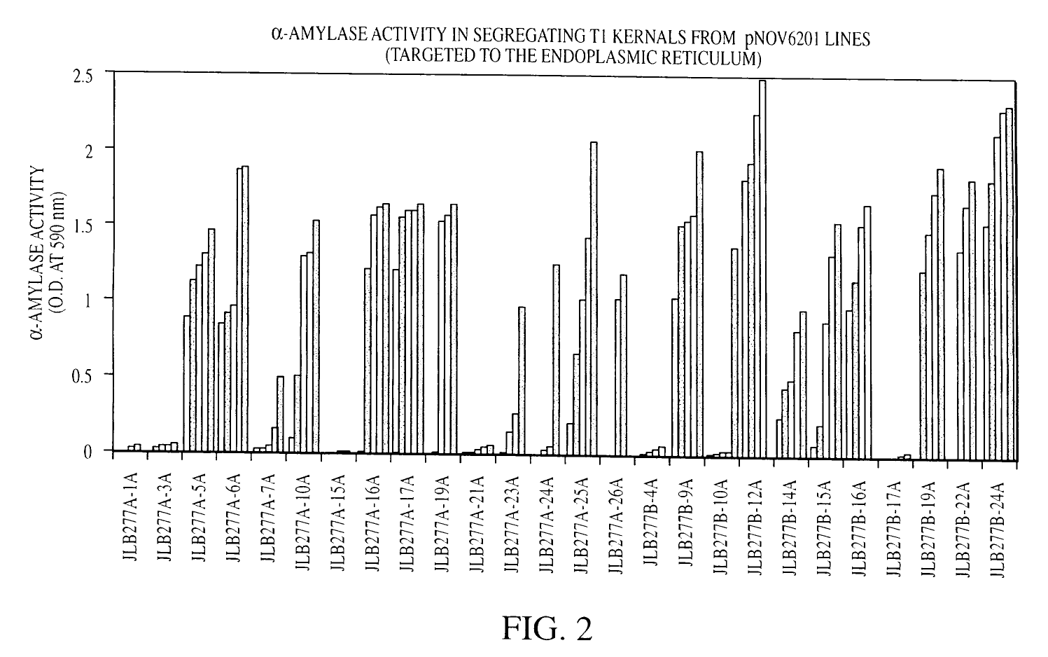

T1 seed from multiple independent lines of both pNOV6200 and pNOV6201 transformants was obtained. Individual kernels from each line were dissected and purified endosperms were homogenized individually in 300 μl of 50 mM NaPO₄ buffer. Aliquots of the endosperm suspensions were analyzed for α-amylase activity at 85° C. Approximately 80% of the lines segregate for hyperthermophilic activity (See FIGS. 1A, 1B, and 2).

Kernels from wild type plants or plants transformed with pNOV6201 were heated at 100° C. for 1, 2, 3, or 6 hours and then stained for starch with an iodine solution. Little or no starch was detected in mature kernels after 3 or 6 hours, respectively. Thus, starch in mature kernels from transgenic maize which express hyperthermophilic amylase that is targeted to the endoplasmic reticulum was hydrolyzed when incubated at high temperature.

In another experiment, partially purified starch from mature T1 kernels from pNOV6201 plants that were steeped at 50° C. for 16 hours was hydrolyzed after heating at 85° C. for 5 minutes. This illustrated that the α-amylase targeted to the endoplasmic reticulum binds to starch after grinding of the kernel, and is able to hydrolyze the starch upon heating. Iodine staining indicated that the starch remains intact in mature seeds after the 16 hour steep at 50° C.

In another experiment, segregating, mature kernels from plants transformed with pNOV6201 were heated at 95° C. for 16 hours and then dried. In seeds expressing the hyperthermophilic α-amylase, the hydrolysis of starch to sugar resulted in a wrinkled appearance following drying.

### Example 13

### Analysis of T1 Seed from Maize Plants Expressing the α-amylase Targeted to the Amyloplast

T1 seed from self-pollinated maize plants transformed with either pNOV4029 or pNOV4031 as described in Example 4 was obtained. Starch accumulation in kernels from these lines was clearly not normal. All lines segregated, with some variation in severity, for a very low or no starch phenotype. Endosperm purified from immature kernels stained only weakly with iodine prior to exposure to high temperatures. After 20 minutes at 85° C., there was no staining. When the ears were dried, the kernels shriveled up. This particular amylase clearly had sufficient activity at greenhouse temperatures to hydrolyze starch if allowed to be in direct contact with the granule

### Example 14

### Fermentation of Grain from Maize Plants Expressing α-amylase

100% Transgenic grain 85° C. vs. 95° C., varied liquefaction time.

Transgenic corn (pNOV6201) that contains a thermostable α-amylase performs well in fermentation without addition of exogenous α-amylase, requires much less time for liquefaction and results in more complete solubilization of starch. Laboratory scale fermentations were performed by a protocol with the following steps (detailed below): 1) grinding, 2) moisture analysis, 3) preparation of a slurry containing ground corn, water, backset and α-amylase, 4) liquefaction and 5) simultaneous saccharification and fermentation (SSF). In this example the temperature and time of the liquefaction step were varied as described below. In addition the transgenic corn was liquefied with and without exogenous α-amylase and the performance in ethanol production compared to control corn treated with commercially available α-amylase.

The transgenic corn used in this example was made in accordance with the procedures set out in Example 4 using a vector comprising the α-amylase gene and the PMI selectable marker, namely pNOV6201. The transgenic corn was produced by pollinating a commercial hybrid (N3030BT) with pollen from a transgenic line expressing a high level of thermostable α-amylase. The corn was dried to 11% moisture and stored at room temperature. The α-amylase content of the transgenic corn flour was 95 units/g where 1 unit of enzyme generates 1 micromole reducing ends per min from corn flour at 85° C. in pH 6.0 MES buffer. The control corn that was used was a yellow dent corn known to perform well in ethanol production.

1) Grinding: Transgenic corn (1180 g) was ground in a Perten 3100 hammer mill equipped with a 2.0 mm screen thus generating transgenic corn flour. Control corn was ground in the same mill after thoroughly cleaning to prevent contamination by the transgenic corn.

2) Moisture analysis: Samples (20 g) of transgenic and control corn were weighed into aluminum weigh boats and heated at 100 C for 4 h. The samples were weighed again and the moisture content calculated from the weight loss. The moisture content of transgenic flour was 9.26%; that of the control flour was 12.54%.

3) Preparation of slurries: The composition of slurries was designed to yield a mash with 36% solids at the beginning of SSF. Control samples were prepared in 100 ml plastic bottles and contained 21.50 g of control corn flour, 23 ml of de-ionized water, 6.0 ml of backset (8% solids by weight), and 0.30 ml of a commercially available α-amylase diluted 1/50 with water. The α-amylase dose was chosen as representative of industrial usage. When assayed under the conditions described above for assay of the transgenic α-amylase, the control α-amylase dose was 2 U/g corn flour. pH was adjusted to 6.0 by addition of ammonium hydroxide. Transgenic samples were prepared in the same fashion but contained 20 g of corn flour because of the lower moisture content of transgenic flour. Slurries of transgenic flour were prepared either with α-amylase at the same dose as the control samples or without exogenous α-amylase.

4) Liquefaction: The bottles containing slurries of transgenic corn flour were immersed in water baths at either 85° C. or 95° C. for times of 5, 15, 30, 45 or 60 min. Control slurries were incubated for 60 min at 85° C. During the high temperature incubation the slurries were mixed vigorously by hand every 5 min. After the high temperature step the slurries were cooled on ice.

5) Simultaneous saccharification and fermentation: The mash produced by liquefaction was mixed with glucoamylase (0.65 ml of a 1/50 dilution of a commercially available L-400 glucoamylase), protease (0.60 ml of a 1,000-fold dilution of a commercially available protease), 0.2 mg Lactocide & urea (0.85 ml of a 10-fold dilution of 50% Urea Liquor). A hole was cut into the cap of the 100 ml bottle containing the mash to allow CO₂ to vent. The mash was then inoculated with yeast (1.44 ml) and incubated in a water bath set at 90 F. After 24 hours of fermentation the temperature was lowered to 86 F; at 48 hours it was set to 82 F.

Yeast for inoculation was propagated by preparing a mixture that contained yeast (0.12 g) with 70 grams maltodextrin, 230 ml water, 100 ml backset, glucoamylase (0.88 ml of a 10-fold dilution of a commercially available glucoamylase), protease (1.76 ml of a 100-fold dilution of a commercially available enzyme), urea (1.07 grams), penicillin (0.67 mg) and zinc sulfate (0.13 g). The propagation culture was initiated the day before it was needed and was incubated with mixing at 90° F.

At 24, 48 & 72 hour samples were taken from each fermentation vessel, filtered through 0.2 μm filters and analyzed by HPLC for ethanol & sugars. At 72 h samples were analyzed for total dissolved solids and for residual starch.

HPLC analysis was performed on a binary gradient system equipped with refractive index detector, column heater & Bio-Rad Aminex HPX-87H column. The system was equilibrated with 0.005 M H₂SO₄ in water at 1 ml/min. Column temperature was 50° C. Sample injection volume was 5 μl; elution was in the same solvent. The RI response was calibrated by injection of known standards. Ethanol and glucose were both measured in each injection.

Residual starch was measured as follows. Samples and standards were dried at 50° C. in an oven, then ground to a powder in a sample mill. The powder (0.2 g) was weighed into a 15 ml graduated centrifuge tube. The powder was washed 3 times with 10 ml aqueous ethanol (80% v/v) by vortexing followed by centrifugation and discarding of the supernatant. DMSO (2.0 ml) was added to the pellet followed by 3.0 ml of a thermostable alpha-amylase (300 units) in MOPS buffer. After vigorous mixing, the tubes were incubated in a water bath at 85° C. for 60 min. During the incubation, the tubes were mixed four times. The samples were cooled and 4.0 ml sodium acetate buffer (200 mM, pH 4.5) was added followed by 0.1 ml of glucoamylase (20 U). Samples were incubated at 50° C. for 2 hours, mixed, then centrifuged for 5 min at 3,500 rpm. The supernatant was filtered through a 0.2 um filter and analyzed for glucose by the HPLC method described above. An injection size of 50 μl was used for samples with low residual starch (<20% of solids).

Results Transgenic corn performed well in fermentation without added α-amylase. The yield of ethanol at 72 hours was essentially the same with or without exogenous α-amylase as shown in Table I. These data also show that a higher yield of ethanol is achieved when the liquefaction temperature is higher; the present enzyme expressed in the transgenic corn has activity at higher temperatures than other enzymes used commercially such as the Bacillus liquefaciens α-amylase.

TABLE I

| Liquefaction | Liquefaction | | | Mean | Std. |
|---|---|---|---|---|---|
| temp | time | Exogenous | # | Ethanol % | Dev. |
| ° C. | min. | α-amylase | replicates | v/v | % v/v |
| 85 | 60 | Yes | 4 | 17.53 | 0.18 |
| 85 | 60 | No | 4 | 17.78 | 0.27 |
| 95 | 60 | Yes | 2 | 18.22 | ND |
| 95 | 60 | No | 2 | 18.25 | ND |

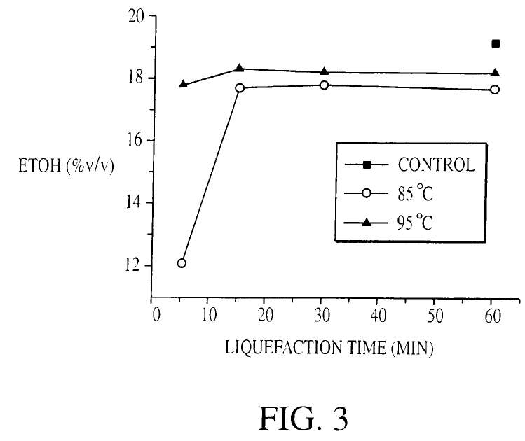

When the liquefaction time was varied, it was found that the liquefaction time required for efficient ethanol production was much less than the hour required by the conventional process. FIG. 3 shows that the ethanol yield at 72 hours fermentation was almost unchanged from 15 min to 60 min liquefaction. In addition liquefaction at 95° C. gave more ethanol at each time point than at the 85° C. liquefaction. This observation demonstrates the process improvement achieved by use of a hyperthermophilic enzyme.

The control corn gave a higher final ethanol yield than the transgenic corn, but the control was chosen because it performs very well in fermentation. In contrast the transgenic corn has a genetic background chosen to facilitate transformation. Introducing the α-amylase-trait into elite corn germplasm by well-known breeding techniques should eliminate this difference.

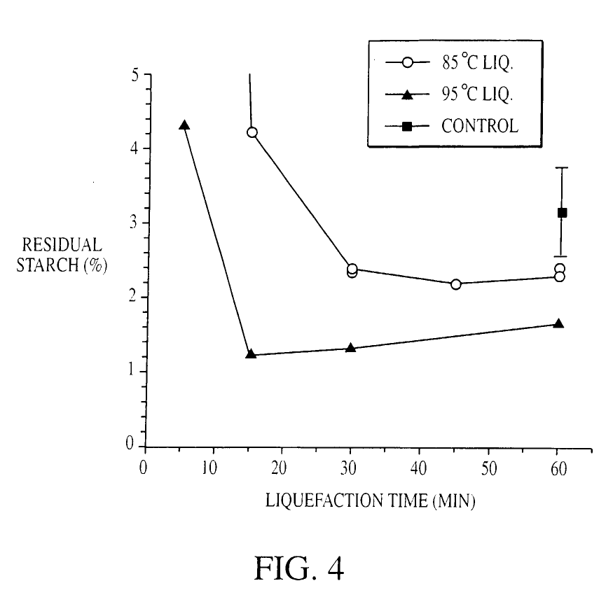

Examination of the residual starch levels of the beer produced at 72 hours (FIG. 4) shows that the transgenic α-amylase results in significant improvement in making starch available for fermentation; much less starch was left over after fermentation.

Using both ethanol levels and residual starch levels the optimal liquefaction times were 15 min at 95° C. and 30 min at 85° C. In the present experiments these times were the total time that the fermentation vessels were in the water bath and thus include a time period during which the temperature of the samples was increasing from room temperature to 85° C. or 95° C. Shorter liquefaction times may be optimal in large scale industrial processes that rapidly heat the mash by use of equipment such as jet cookers. Conventional industrial liquefaction processes require holding tanks to allow the mash to be incubated at high temperature for one or more hours. The present invention eliminates the need for such holding tanks and will increase the productivity of liquefaction equipment.

One important function of α-amylase in fermentation processes is to reduce the viscosity of the mash. At all time points the samples containing transgenic corn flour were markedly less viscous than the control sample. In addition the transgenic samples did not appear to go through the gelatinous phase observed with all control samples; gelatinization normally occurs when corn slurries are cooked. Thus having the α-amylase distributed throughout the fragments of the endosperm gives advantageous physical properties to the mash during cooking by preventing formation of large gels that slow diffusion and increase the energy costs of mixing and pumping the mash.

The high dose of α-amylase in the transgenic corn may also contribute to the favorable properties of the transgenic mash. At 85° C., the α-amylase activity of the transgenic corn was many times greater activity than the of the dose of exogenous α-amylase used in controls. The latter was chosen as representative of commercial use rates.

### Example 15

### Effective Function of Transgenic Corn when Mixed with Control Corn

Transgenic corn flour was mixed with control corn flour in various levels from 5% to 100% transgenic corn flour. These were treated as described in Example 14. The mashes containing transgenically expressed α-amylase were liquefied at 85° C. for 30 min or at 95° C. for 15 min; control mashes were prepared as described in Example 14 and were liquefied at 85° C. for 30 or 60 min (one each) or at 95° C. for 15 or 60 min (one each).

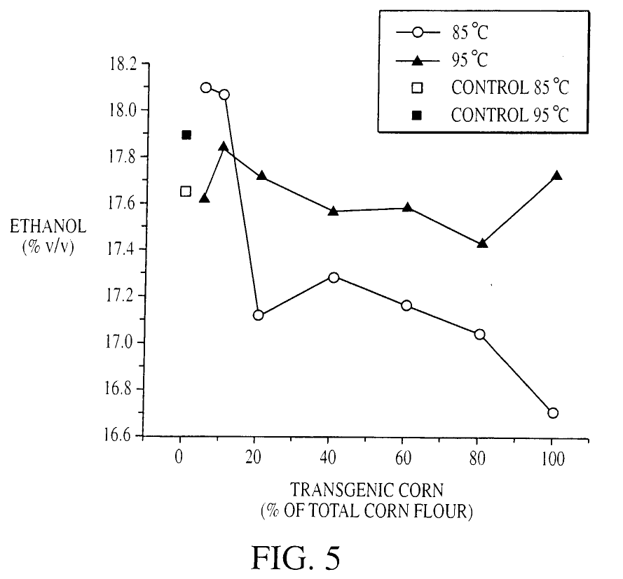

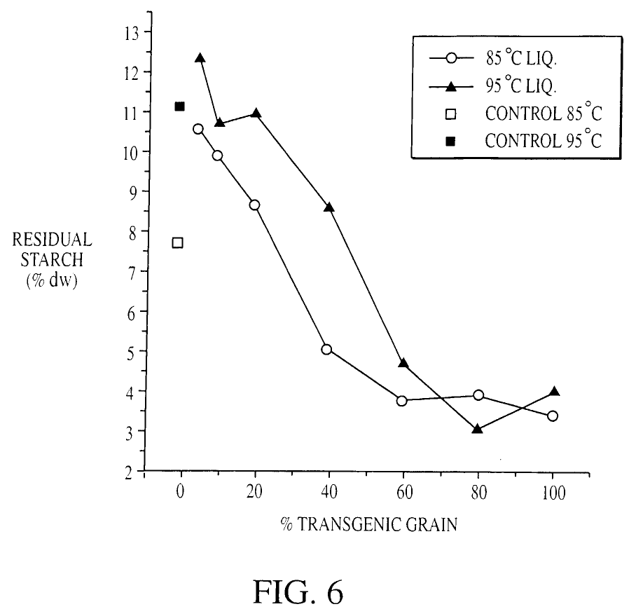

The data for ethanol at 48 and 72 hours and for residual starch are given in Table 2. The ethanol levels at 48 hours are graphed in FIG. 5; the residual starch determinations are shown in FIG. 6. These data show that transgenically expressed thermostable α-amylase gives very good performance in ethanol production even when the transgenic grain is only a small portion (as low as 5%) of the total grain in the mash. The data also show that residual starch is markedly lower than in control mash when the transgenic grain comprises at least 40% of the total grain.

TABLE 2

| Trans- | 85° C. Liquefaction | | | 95° C. Liquefaction | | |
|---|---|---|---|---|---|---|
| genic | | | Ethanol | | | Ethanol |
| grain | Residual | Ethanol | % v/v | Residual | Ethanol | % v/v |
| wt % | Starch | 48 h | 72 h | Starch | 48 h | 72 h |
| 100 | 3.58 | 16.71 | 18.32 | 4.19 | 17.72 | 21.14 |
| 80 | 4.06 | 17.04 | 19.2 | 3.15 | 17.42 | 19.45 |
| 60 | 3.86 | 17.16 | 19.67 | 4.81 | 17.58 | 19.57 |
| 40 | 5.14 | 17.28 | 19.83 | 8.69 | 17.56 | 19.51 |
| 20 | 8.77 | 17.11 | 19.5 | 11.05 | 17.71 | 19.36 |
| 10 | 10.03 | 18.05 | 19.76 | 10.8 | 17.83 | 19.28 |
| 5 | 10.67 | 18.08 | 19.41 | 12.44 | 17.61 | 19.38 |
| 0* | 7.79 | 17.64 | 20.11 | 11.23 | 17.88 | 19.87 |

*Control samples. Values the average of 2 determinations

### Example 16

### Ethanol Production as a Function of Liquefaction pH Using Transgenic Corn at a Rate of 1.5 to 12% of Total Corn

Because the transgenic corn performed well at a level of 5-10% of total corn in a fermentation, an additional series of fermentations in which the transgenic corn comprised 1.5 to 12% of the total corn was performed. The pH was varied from 6.4 to 5.2 and the α-amylase enzyme expressed in the transgenic corn was optimized for activity at lower pH than is conventionally used industrially.

The experiments were performed as described in Example 15 with the following exceptions:

1). Transgenic flour was mixed with control flour as a percent of total dry weight at the levels ranging from 1.5% to 12.0%.
2). Control corn was N3030BT which is more similar to the transgenic corn than the control used in examples 14 and 15.
3). No exogenous α-amylase was added to samples containing transgenic flour.
4). Samples were adjusted to pH 5.2, 5.6, 6.0 or 6.4 prior to liquefaction. At least 5 samples spanning the range from 0% transgenic corn flour to 12% transgenic corn flour were prepared for each pH.
5). Liquefaction for all samples was performed at 85° C. for 60 min.

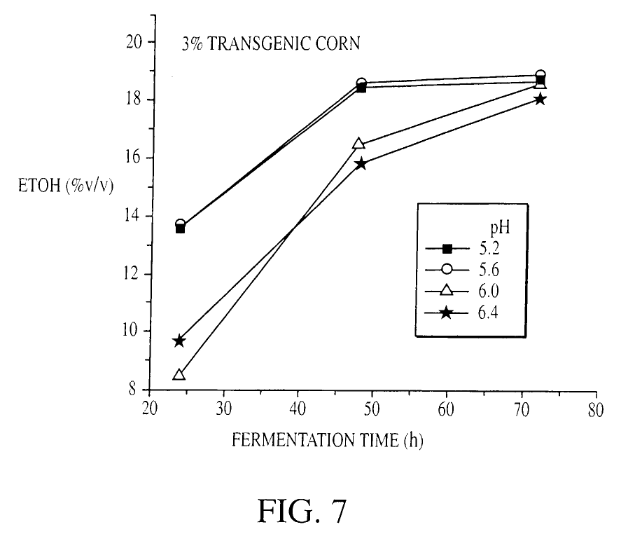

The change in ethanol content as a function of fermentation time are shown in FIG. 7. This figure shows the data obtained from samples that contained 3% transgenic corn. At the lower pH, the fermentation proceeds more quickly than at pH 6.0 and above; similar behavior was observed in samples with other doses of transgenic grain. The pH profile of activity of the transgenic enzyme combined with the high levels of expression will allow lower pH liquefactions resulting in more rapid fermentations and thus higher throughput than is possible at the conventional pH 6.0 process.

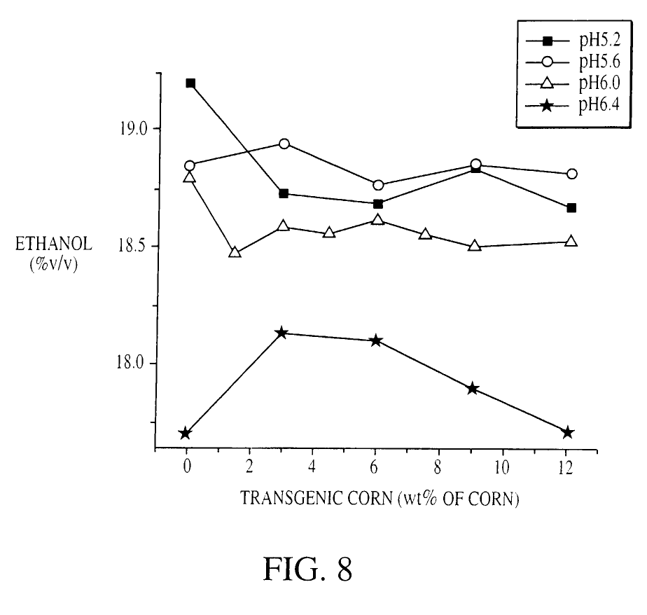

The ethanol yields at 72 hours are shown in FIG. 8. As can be seen, on the basis of ethanol yield, the results showed little dependence on the amount of transgenic grain included in the sample. Thus the grain contains abundant amylase to facilitate fermentative production of ethanol. It is also demonstrates that lower pH of liquefaction results in higher ethanol yield.

The viscosity of the samples after liquefaction was monitored and it was observed that at pH 6.0, 6% transgenic grain is sufficient for adequate reduction in viscosity. At pH 5.2 and 5.6, viscosity is equivalent to that of the control at 12% transgenic grain, but not at lower percentages of transgenic grain.

### Example 17

### Production of Fructose from Corn Flour Using Thermophilic Enzymes

Corn that expresses the hyperthermophilic α-amylase, 797GL3, was shown to facilitate production of fructose when mixed with an α-glucosidase (MalA) and a xylose isomerase (XylA).

Seed from pNOV6201 transgenic plants expressing 797GL3 were ground to a flour in a Kleco cell thus creating amylase flour. Non-transgenic corn kernels were ground in the same manner to generate control flour.

The α-glucosidase, MalA (from S. solfataricus), was expressed in E. coli. Harvested bacteria were suspended in 50 mM potassium phosphate buffer pH 7.0 containing 1 mM 4-(2-aminoethyl)benzenesulfonyl fluoride then lysed in a French pressure cell. The lysate was centrifuged at 23,000×g for 15 min at 4° C. The supernatant solution was removed, heated to 70° C. for 10 min, cooled on ice for 10 min, then centrifuged at 34,000×g for 30 min at 4° C. The supernatant solution was removed and the MalA concentrated two-fold in centricon 10 devices. The filtrate of the centricon 10 step was retained for use as a negative control for MalA.

Xylose (glucose) isomerase was prepared by expressing the xylA gene of T. neapolitana in E. coli. Bacteria were suspended in 100 mM sodium phosphate pH 7.0 and lysed by passage through a French pressure cell. After precipitation of cell debris, the extract was heated at 80° C. for 10 min then centrifuged. The supernatant solution contained the XylA enzymatic activity. An empty-vector control extract was prepared in parallel with the XylA extract.

Corn flour (60 mg per sample) was mixed with buffer and extracts from E. coli. As indicated in Table 3, samples contained amylase corn flour (amylase) or control corn flour (control), 50 μl of either MalA extract (+) or filtrate (−), and 20 μl of either XylA extract (+) or empty vector control (−). All samples also contained 230 μl of 50 mM MOPS, 10 mM MgSO4, and 1 mM CoCl2; pH of the buffer was 7.0 at room temperature.

Samples were incubated at 85° C. for 18 hours. At the end of the incubation time, samples were diluted with 0.9 ml of 85° C. water and centrifuged to remove insoluble material. The supernatant fraction was then filtered through a Centricon3 ultrafiltration device and analyzed by HPLC with ELSD detection.

The gradient HPLC system was equipped with Astec Polymer Amino Column, 5 micron particle size, 250×4.6 mm and an Alltech ELSD 2000 detector. The system was pre-equilibrated with a 15:85 mixture of water:acetonitrile. The flow rate was 1 ml/min. The initial conditions were maintained for 5 min after injection followed by a 20 min gradient to 50:50 water:acetonitrile followed by 10 minutes of the same solvent. The system was washed with 20 min of 80:20 water:acetonitrile and then re-equilibrated with the starting solvent. Fructose was eluted at 5.8 min and glucose at 8.7 min.

TABLE 3

| | | | | fructose | glucose |
|---|---|---|---|---|---|
| Sample | Corn flour | MalA | XylA | peak area × 10⁻⁶ | peak area × 10⁻⁶ |
| 1 | amylase | + | + | 25.9 | 110.3 |
| 2 | amylase | − | + | 7.0 | 12.4 |
| 3 | amylase | + | − | 0.1 | 147.5 |
| 4 | amylase | − | − | 0 | 25.9 |
| 5 | control | + | + | 0.8 | 0.5 |
| 6 | control | − | + | 0.3 | 0.2 |
| 7 | control | + | − | 1.3 | 1.7 |
| 8 | control | − | − | 0.2 | 0.3 |

The HPLC results also indicated the presence of larger maltooligosaccharides in all samples containing the α-amylase. These results demonstrate that the three thermophilic enzymes can function together to produce fructose from corn flour at a high temperature.

### Example 18

### Amylase Flour with Isomerase

In another example, amylase flour was mixed with purified MalA and each of twobacterial xylose isomerases: XylA of T. maritima, and an enzyme designated BD8037 obtained from Diversa. Amylase flour was prepared as described in Example 18.

S. solfataricus MalA with a 6His purification tag was expressed in E. coli. Cell lysate was prepared as described in Example 18, then purified to apparent homogeneity using a nickel affinity resin (Probond, Invitrogen) and following the manufacturer's instructions for native protein purification.

T. maritima XylA with the addition of an S tag and an ER retention signal was expressed in E. coli and prepared in the same manner as the T. neapolitana XylA described in Example 18.

Xylose isomerase BD8037 was obtained as a lyophilized powder and resuspended in 0.4× the original volume of water.

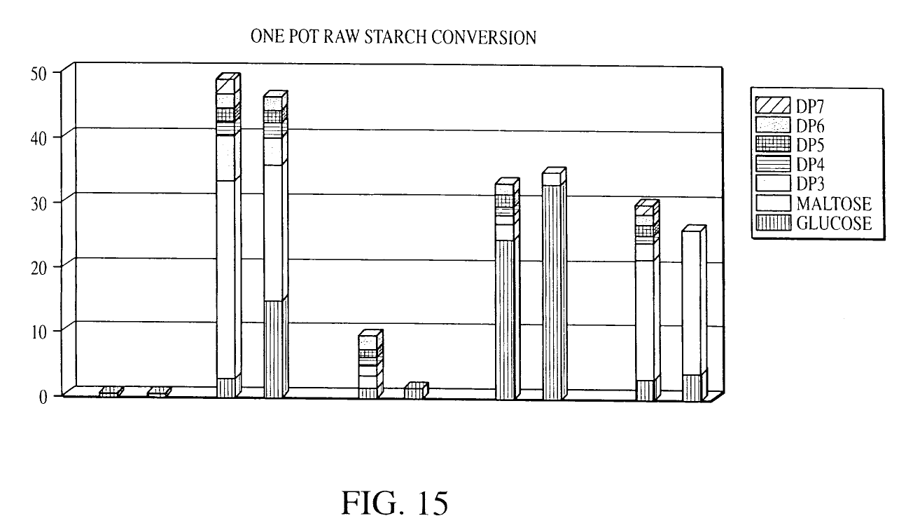

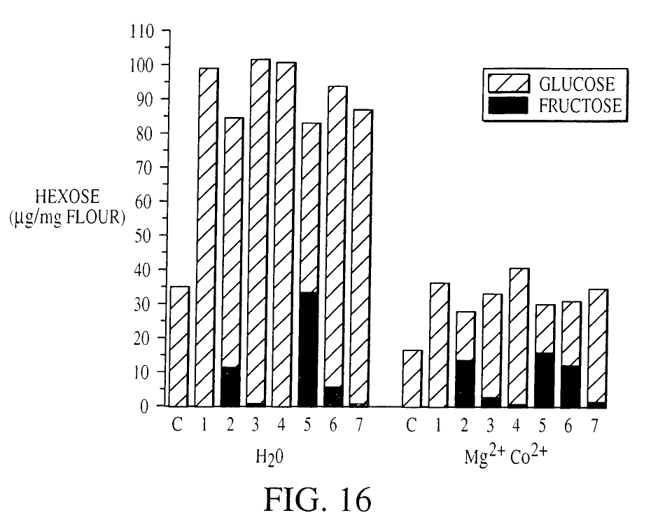

Amylase corn flour was mixed with enzyme solutions plus water or buffer. All reactions contained 60 mg amylase flour and a total of 600 μl of liquid. One set of reactions was buffered with 50 mM MOPS, pH 7.0 at room temperature, plus 10 mM MgSO4 and 1 mM CoCl2; in a second set of reactions the metal-containing buffer solution was replaced by water. Isomerase enzyme amounts were varied as indicated in Table 4. All reactions were incubated for 2 hours at 90° C. Reaction supernatant fractions were prepared by centrifugation. The pellets were washed with an additional 600 μl H₂O and recentrifuged. The supernatant fractions from each reaction were combined, filtered through a Centricon 10, and analyzed by HPLC with ELSD detection as described in Example 17. The amounts of glucose and fructose observed are graphed in FIG. 15.

TABLE 4

| Sample | Amylase flour | Mal A | Isomerase |
|---|---|---|---|
| 1 | 60 mg | + | none |
| 2 | 60 mg | + | T. maritima, 100 μl |
| 3 | 60 mg | + | T. maritima, 10 μl |
| 4 | 60 mg | + | T. maritima, 2 μl |
| 5 | 60 mg | + | BD8037, 100 μl |
| 7 | 60 mg | + | BD8037, 2 μl |
| C | 60 mg | none | none |

With each of the isomerases, fructose was produced from corn flour in a dose-dependent manner when α-amylase and α-glucosidase were present in the reaction. These results demonstrate that the grain-expressed amylase 797GL3 can function with MalA and a variety of different thermophilic isomerases, with or without added metal ions, to produce fructose from corn flour at a high temperature. In the presence of added divalent metal ions, the isomerases can achieve the predicted fructose: glucose equilibrium at 90° C. of approximately 55% fructose. This would be an improvement over the current process using mesophilic isomerases, which requires a chromatographic separation to increase the fructose concentration.

### Example 19

### Expression of a Pullulanase in Corn

Transgenic plants that were homozygous for either pNOV7013 or pNOV7005 were crossed to generate transgenic corn seed expressing both the 797GL3 α-amylase and 6GP3 pullulanase.

T1 or T2 seed from self-pollinated maize plants transformed with either pNOV 7005 or pNOV 4093 were obtained. pNOV4093 is a fusion of the maize optimized synthetic gene for 6GP3 (SEQ ID: 3,4) with the amyloplast targeting sequence (SEQ ID NO: 7,8) for localization of the fusion protein to the amyloplast. This fusion protein is under the control of the ADPgpp promoter (SEQ ID NO:11) for expression specifically in the endosperm. The pNOV7005 construct targets the expression of the pullulanase in the endoplasmic reticulum of the endosperm. Localization of this enzyme in the ER allows normal accumulation of the starch in the kernels. Normal staining for starch with an iodine solution was also observed, prior to any exposure to high temperature.

As described in the case of α-amylase the expression of pullulanase targeted to the amyloplast (pNOV4093) resulted in abnormal starch accumulation in the kernels. When the corn-ears are dried, the kernels shriveled up. Apparently, this thermophilic pullulanase is sufficiently active at low temperatures and hydrolyzes starch if allowed to be in direct contact with the starch granules in the seed endosperm.

Enzyme preparation or extraction of the enzyme from corn-flour: The pullulanase enzyme was extracted from the transgenic seeds by grinding them in Kleco grinder, followed by incubation of the flour in 50 mM NaOAc pH 5.5 buffer for 1 hr at RT, with continuous shaking. The incubated mixture was then spun for 15 min. at 14000 rpm. The supernatant was used as enzyme source.

Pullulanase assay: The assay reaction was carried out in 96-well plate. The enzyme extracted from the corn flour (100 μl) was diluted 10 fold with 900 μL of 50 mM NaOAc pH5.5 buffer, containing 40 mM CaCl₂. The mixture was vortexed, 1 tablet of Limit-Dextrizyme (azurine-crosslinked-pullulan, from Megazyme) was added to each reaction mixture and incubated at 75° C. for 30 min (or as mentioned). At the end of the incubation the reaction mixtures were spun at 3500 rpm for 15 min. The supernatants were diluted 5 fold and transferred into 96-well flat bottom plate for absorbance measurement at 590 nm. Hydrolysis of azurine-crosslinked-pullulan substrate by the pullulanase produces water-soluble dye fragments and the rate of release of these (measured as the increase in absorbance at 590 nm) is related directly to enzyme activity.

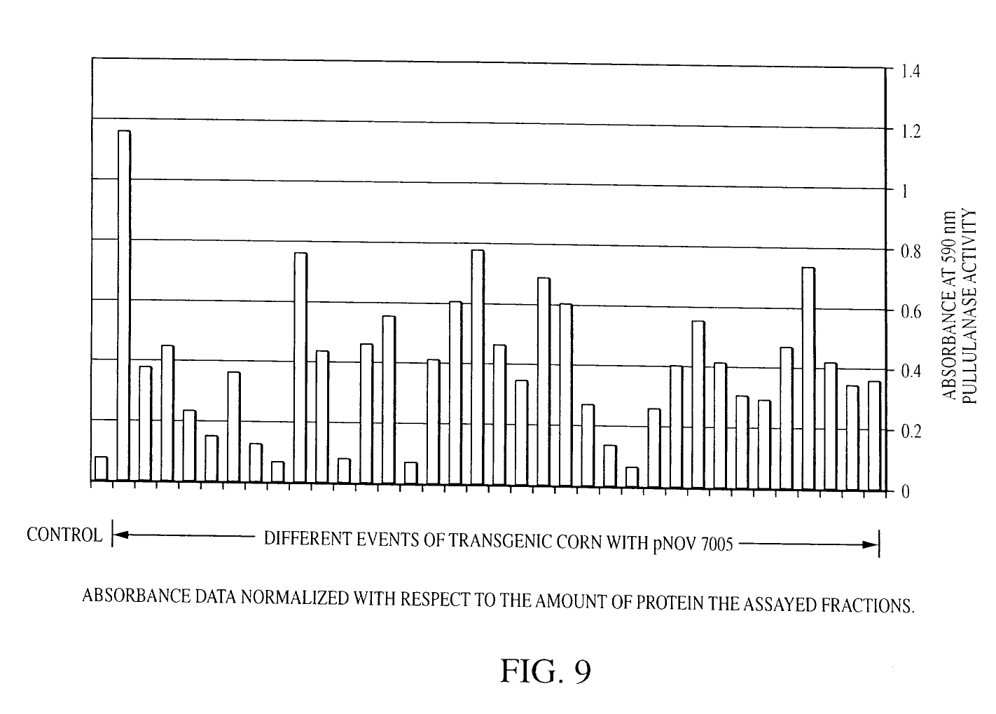

FIG. 9 shows the analysis of T2 seeds from different events transformed with pNOV 7005. High expression of pullulanase activity, compared to the non-transgenic control, can be detected in a number of events.

To a measured amount (˜100 μg) of dry corn flour from transgenic (expressing pullulanase, or amylase or both the enzymes) and/or control (non-transgenic) 1000 μl of 50 mM NaOAc pH 5.5 buffer containing 40 mM CaCl₂ was added. The reaction mixtures were vortexed and incubated on a shaker for 1 hr. The enzymatic reaction was started by transferring the incubation mixtures to high temperature (75° C., the optimum reaction temperature for pullulanase or as mentioned in the figures) for a period of time as indicated in the figures. The reactions were stopped by cooling them down on ice. The reaction mixtures were then centrifuged for 10 min. at 14000 rpm. An aliquot (100 μl) of the supernatant was diluted three fold, filtered through 0.2-micron filter for HPLC analysis.

The samples were analyzed by HPLC using the following conditions:

Column: Alltech Prevail Carbohydrate ES 5 micron 250×4.6 mm

Detector: Alltech ELSD 2000

Pump: Gilson 322

Injector: Gilson 215 injector/diluter

Solvents: HPLC grade Acetonitrile (Fisher Scientific) and Water (purified by Waters Millipore System)

Gradient used for oligosaccharides of low degree of polymerization (DP 1-15).

| Time | % Water | % Acetonitrile |
|---|---|---|
| 0 | 15 | 85 |
| 5 | 15 | 85 |
| 25 | 50 | 50 |
| 35 | 50 | 50 |
| 36 | 80 | 20 |
| 55 | 80 | 20 |
| 56 | 15 | 85 |
| 76 | 15 | 85 |

Gradient used for saccharides of high degree of polymerization (DP 20-100 and above).

| Time | % Water | % Acetonitrile |
|---|---|---|
| 0 | 35 | 65 |
| 60 | 85 | 15 |
| 70 | 85 | 15 |
| 85 | 35 | 65 |
| 100 | 35 | 65 |

System used for data analysis: Gilson Unipoint Software System Version 3.2

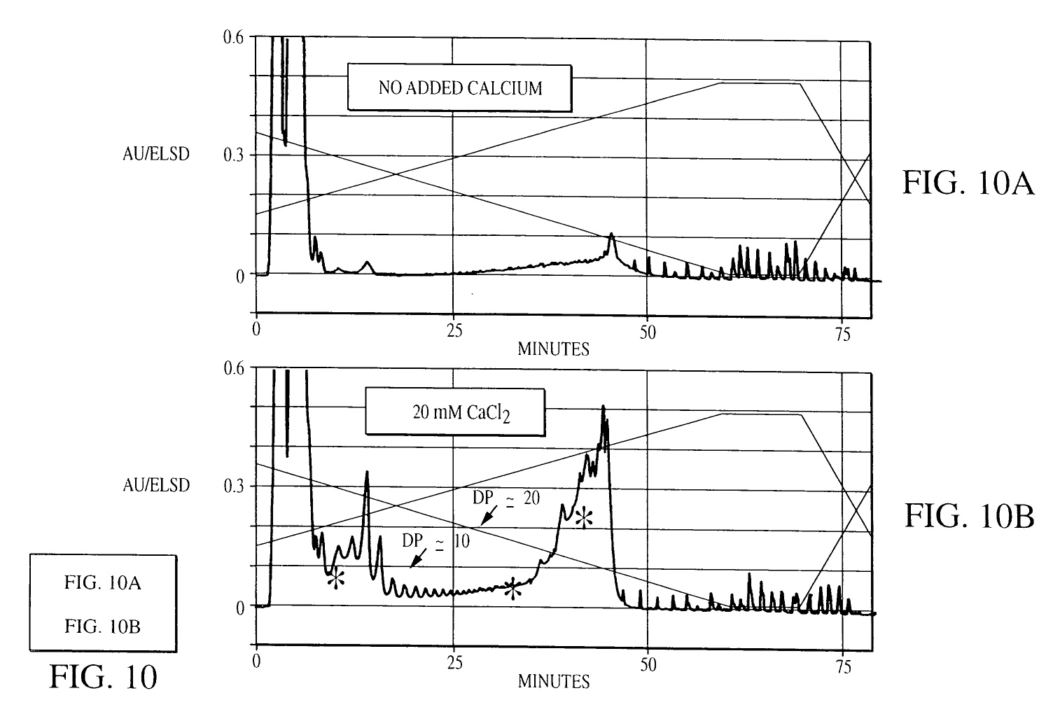

FIGS. 10A and 10B show the HPLC analysis of the hydrolytic products generated by expressed pullulanase from starch in the transgenic corn flour. Incubation of the flour of pullulanase expressing corn in reaction buffer at 75° C. for 30 minutes results in production of medium chain oligosaccharides (DP˜10-30) and short amylose chains (DP˜100-200) from cornstarch. This figure also shows the dependence of pullulanase activity on presence of calcium ions.

Transgenic corn expressing pullulanase can be used to produce modified-starch/dextrin that is debranched (α1-6 linkages cleaved) and hence will have high level of amylose/straight chain dextrin. Also depending on the kind of starch (e.g. waxy, high amylose etc.) used the chain length distribution of the amylose/dextrin generated by the pullulanase will vary, and so will the property of the modified-starch/dextrin.

Hydrolysis of α1-6 linkage was also demonstrated using pullulan as the substrate. The pullulanase isolated from corn flour efficiently hydrolyzed pullulan. HPLC analysis (as described) of the product generated at the end of incubation showed production of maltotriose, as expected, due to the hydrolysis of the α1-6 linkages in the pullulan molecules by the enzyme from the corn.

### Example 20

### Expression of Pullulanase in Corn

Expression of the 6gp3 pullulanase was further analyzed by extraction from corn flour followed by PAGE and Coomassie staining. Corn-flour was made by grinding seeds, for 30 sec., in the Kleco grinder. The enzyme was extracted from about 150 mg of flour with 1 ml of 50 mM NaOAc pH 5.5 buffer. The mixture was vortexed and incubated on a shaker at RT for 1 hr, followed by another 15 min incubation at 70° C. The mixture was then spun down (14000 rpm for 15 min at RT) and the supernatant was used as SDS-PAGE analysis. A protein band of the appropriate molecular weight (95 kDal) was observed. These samples are subjected to a pullulanase assay using commercially available dye-conjugated limit-dextrins (LIMIT-DEXTRIZYME, from Megazyme, Ireland). High levels of thermophilic pullulanase activity correlated with the presence of the 95 kD protein.

The Western blot and ELISA analysis of the transgenic corn seed also demonstrated the expression of ˜95 kD protein that reacted with antibody produced against the pullulanase (expressed in E. coli).

### Example 21

### Increase in the Rate of Starch Hydrolysis and Improved Yield of Small Chain (Fermentable) Oligosaccharides by the Addition of Pullulanase Expressing Corn

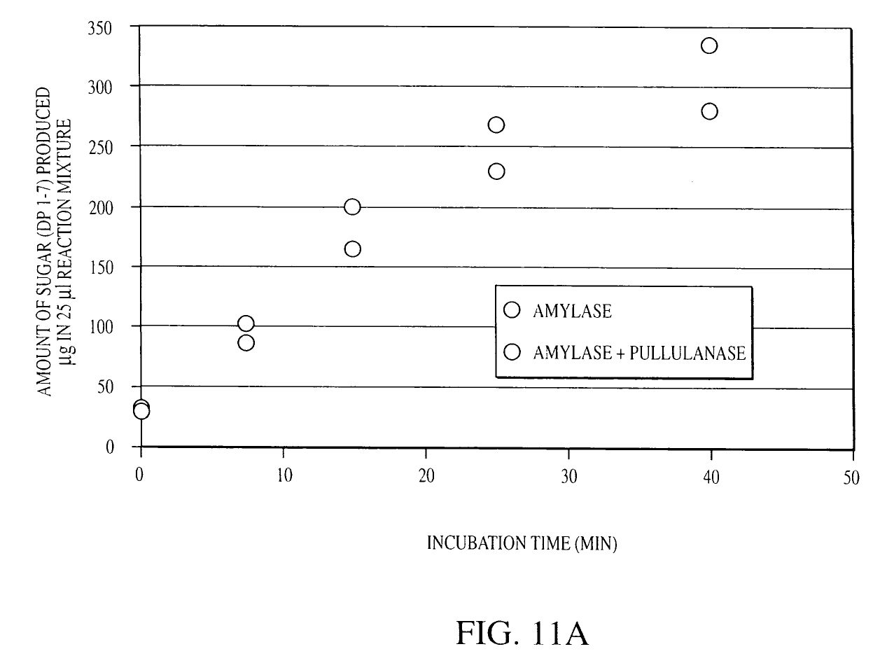

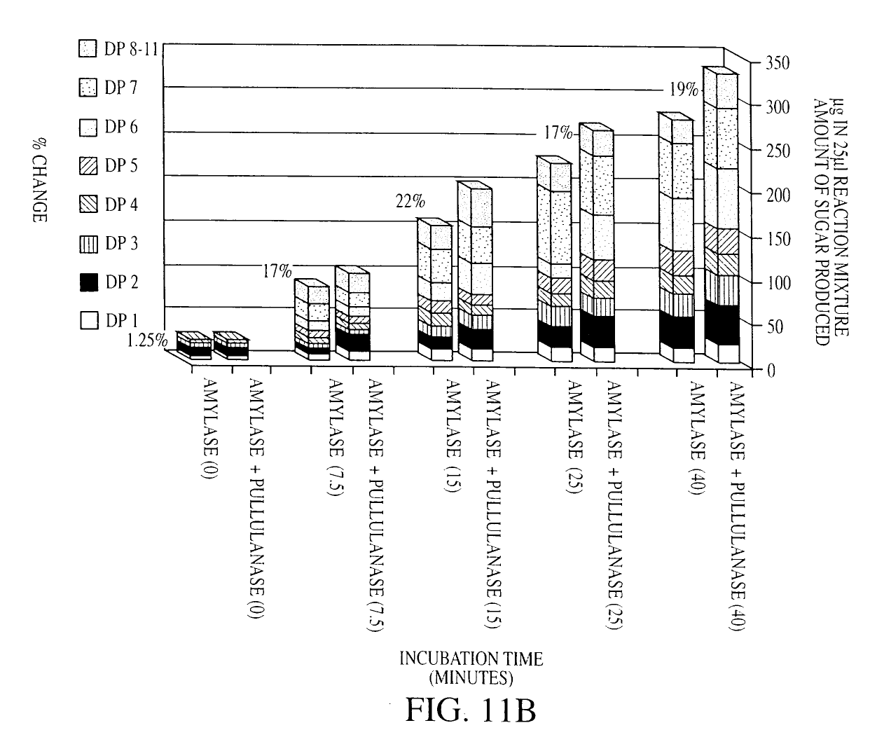

The data shown in FIGS. 11A and 11B was generated from HPLC analysis, as described above, of the starch hydrolysis products from two reaction mixtures. The first reaction indicated as ‘Amylase’ contains a mixture [1:1 (w/w)] of corn flour samples of α-amylase expressing transgenic corn made according to the method described in Example 4, for example, and non-transgenic corn A188; and the second reaction mixture ‘Amylase+Pullulanase’ contains a mixture [1:1 (w/w)] of corn flour samples of α-amylase expressing transgenic corn and pullulanase expressing transgenic corn made according to the method described in Example 19. The results obtained support the benefit of use of pullulanase in combination with α-amylase during the starch hydrolysis processes. The benefits are from the increased rate of starch hydrolysis (FIG. 11A) and increase yield of fermentable oligosaccharides with low DP (FIG. 11B).

It was found that α-amylase alone or α-amylase and pullulanase (or any other combination of starch hydrolytic enzymes) expressed in corn can be used to produce maltodextrin (straight or branched oligosaccharides) (FIGS. 11A, 11B, 12, and 13A). Depending on the reaction conditions, the type of hydrolytic enzymes and their combinations, and the type of starch used the composition of the maltodextrins produced, and hence their properties, will vary.

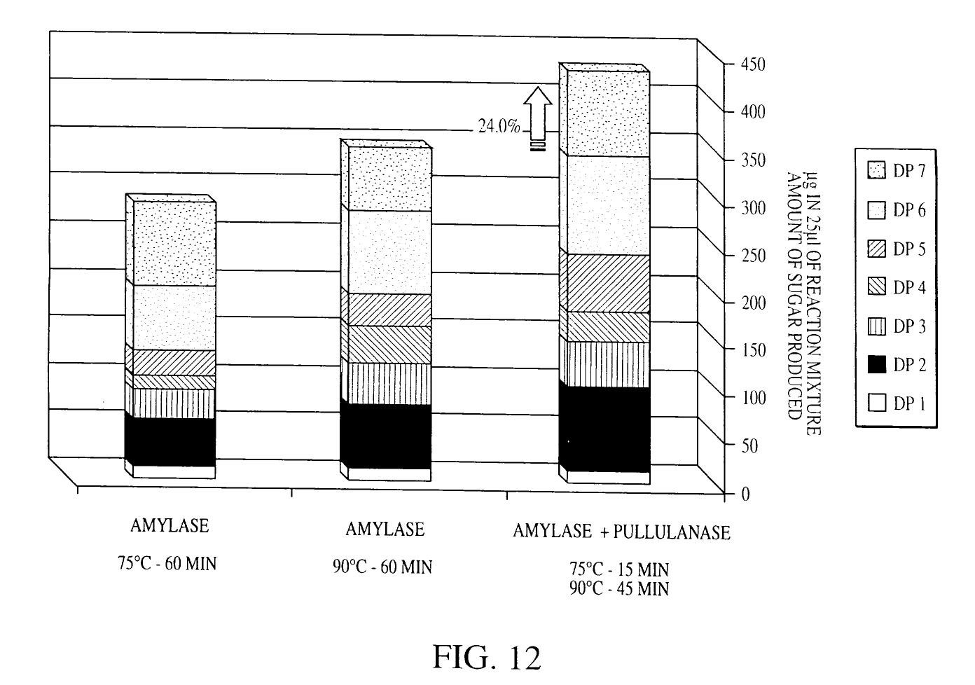

FIG. 12 depicts the results of an experiment carried out in a similar manner as described for FIG. 11. The different temperature and time schemes followed during incubation of the reactions are indicated in the figure. The optimum reaction temperature for pullulanase is 75° C. and for α-amylase it is >95° C. Hence, the indicated schemes were followed to provide scope to carry out catalysis by the pullulanase and/or the α-amylase at their respective optimum reaction temperature. It can be clearly deduced from the result shown that combination of α-amylase and pullulanase performed better in hydrolyzing cornstarch at the end of 60 min incubation period.

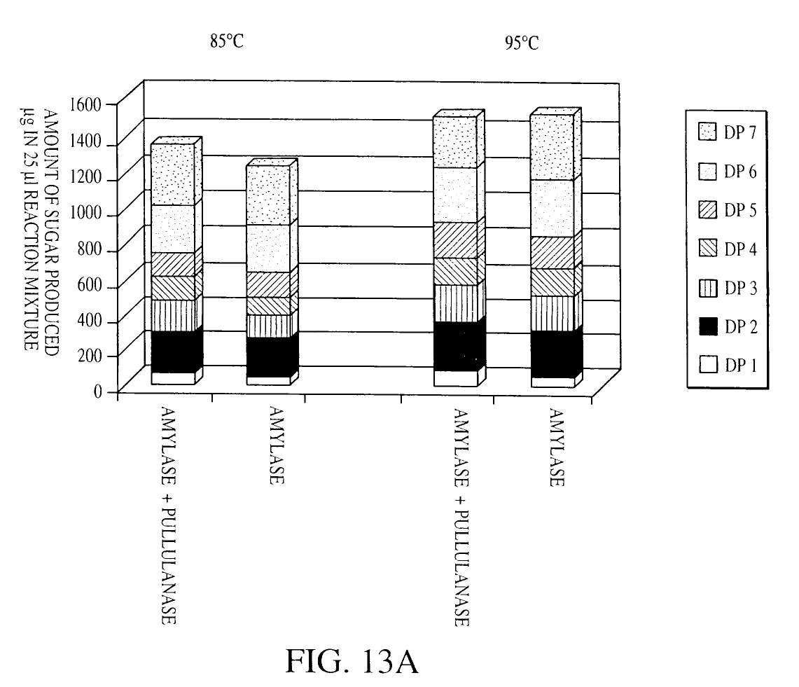

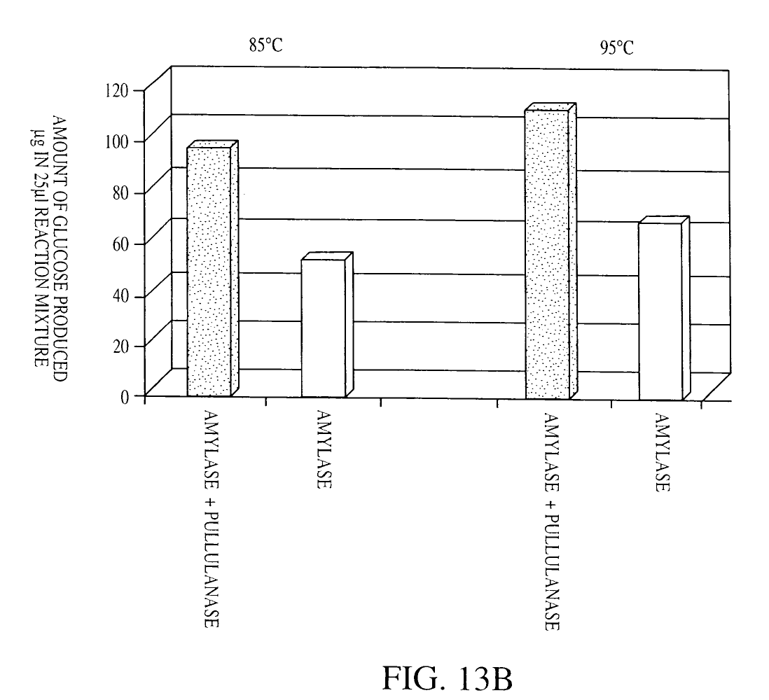

HPLC analysis, as described above (except ˜150 mg of corn flour was used in these reactions), of the starch hydrolysis product from two sets of reaction mixtures at the end of 30 min incubation is shown in FIGS. 13A and 13B. The first set of reactions was incubated at 85° C. and the second one was incubated at 95° C. For each set there are two reaction mixtures; the first reaction indicated as ‘Amylase X Pullulanase’ contains flour from transgenic corn (generated by cross pollination) expressing both the α-amylase and the pullulanase, and the second reaction indicated as ‘Amylase’ mixture of corn flour samples of α-amylase expressing transgenic corn and non-transgenic corn A188 in a ratio so as to obtain same amount of α-amylase activity as is observed in the cross (Amylase X Pullulanase). The total yield of low DP oligosaccharides was more in case of α-amylase and pullulanase cross compared to corn expressing α-amylase alone, when the corn flour samples were incubated at 85° C. The incubation temperature of 95° C. inactivates (at least partially) the pullulanase enzyme, hence little difference can be observed between ‘Amylase X Pullulanase’ and ‘Amylase’. However, the data for both the incubation temperatures shows significant improvement in the amount of glucose produced (FIG. 13B), at the end of the incubation period, when corn flour of α-amylase and pullulanase cross was used compared to corn expressing α-amylase alone. Hence use of corn expressing both α-amylase and pullulanase can be especially beneficial for the processes where complete hydrolysis of starch to glucose is important.

The above examples provide ample support that pullulanase expressed in corn seeds, when used in combination with α-amylase, improves the starch hydrolysis process. Pullulanase enzyme activity, being α1-6 linkage specific, debranches starch far more efficiently than α-amylase (an α-1-4 linkage specific enzyme) thereby reducing the amount of branched oligosaccharides (e.g. limit-dextrin, panose; these are usually non-fermentable) and increasing the amount of straight chain short oligosaccharides (easily fermentable to ethanol etc.). Secondly, fragmentation of starch molecules by pullulanase catalyzed debranching increases substrate accessibility for the α-amylase, hence an increase in the efficiency of the α-amylase catalyzed reaction results.

### Example 22

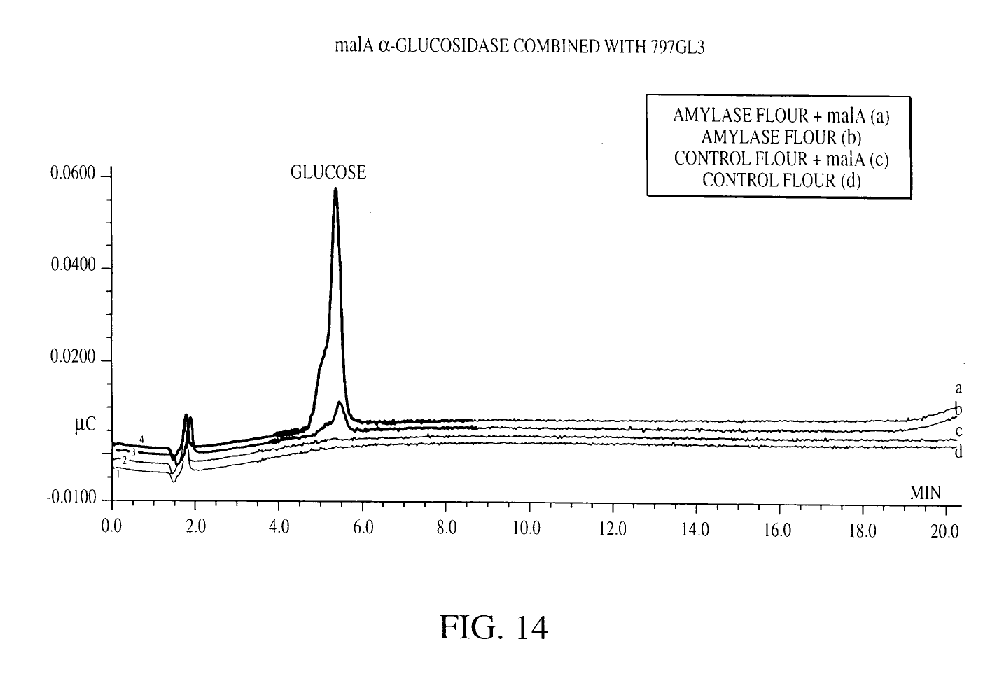

To determine whether the 797GL3 alpha amylase and malA alpha-glucosidase could function under similar pH and temperature conditions to generate an increased amount of glucose over that produced by either enzyme alone, approximately 0.35 ug of malA alpha glucosidase enzyme (produced in bacteria) was added to a solution containing 1% starch and starch purified from either non-transgenic corn seed (control) or 797GL3 transgenic corn seed (in 797GL3 corn seed the alpha amylase co-purifies with the starch). In addition, the purified starch from non-transgenic and 797GL3 transgenic corn seed was added to 1% corn starch in the absence of any malA enzyme. The mixtures were incubated at 90° C., pH 6.0 for 1 hour, spun down to remove any insoluble material, and the soluble fraction was analyzed by HPLC for glucose levels. As shown in FIG. 14, the 797GL3 alpha-amylase and malA alpha-glucosidase function at a similar pH and temperature to break down starch into glucose. The amount of glucose generated is significantly higher than that produced by either enzyme alone.

### Example 23

The utility of the Thermoanaerobacterium glucoamylase for raw starch hydrolysis was determined. As set forth in FIG. 15, the hydrolysis conversion of raw starch was tested with water, barley α-amylase (commercial preparation from Sigma), Thermoanaerobacterium glucoamylase, and combinations thereof were ascertained at room temperature and at 30° C. As shown, the combination of the barley α-amylase with the Thermoanaerobacterium glucoamylase was able to hydrolyze raw starch into glucose. Moreover, the amount of glucose produced by the barley amylase and thermoanaerobacter GA is significantly higher than that produced by either enzyme alone.

### Example 24

### Maize-Optimized Genes and Sequences for Raw-Starch Hydrolysis and Vectors for Plant Transformation

The enzymes were selected based on their ability to hydrolyze raw-starch at temperatures ranging from approximately 20°-50° C. The corresponding genes or gene fragments were then designed by using maize preferred codons for the construction of synthetic genes as set forth in Example 1.

Aspergillus shirousami α-amylase/glucoamylase fusion polypeptide (without signal sequence) was selected and has the amino acid sequence as set forth in SEQ ID NO: 45 as identified in Biosci. Biotech. Biochem., 56:884-889 (1992); Agric. Biol. Chem. 545:1905-14 (1990); Biosci. Biotechnol. Biochem. 56:174-79 (1992). The maize-optimized nucleic acid was designed and is represented in SEQ ID NO:46.

Similarly, Thermoanaerobacterium thermosaccharolyticum glucoamylase was selected, having the amino acid of SEQ ID NO:47 as published in Biosci. Biotech. Biochem., 62:302-308 (1998), was selected. The maize-optimized nucleic acid was designed (SEQ ID NO: 48).

Rhizopus oryzae glucoamylase was selected having the amino acid sequence (without signal sequence) (SEQ ID NO: 50), as described in the literature (Agric. Biol. Chem. (1986) 50, pg 957-964). The maize-optimized nucleic acid was designed and is represented in SEQ ID NO:51.

Moreover, the maize α-amylase was selected and the amino acid sequence (SEQ ID NO: 51) and nucleic acid sequence (SEQ ID NO:52) were obtained from the literature. See, e.g., Plant Physiol. 105:759-760 (1994).

Expression cassettes are constructed to express the Aspergillus shirousami α-amylase/glucoamylase fusion polypeptide from the maize-optimized nucleic acid was designed as represented in SEQ ID NO:46, the Thermoanaerobacterium thermosaccharolyticum glucoamylase from the maize-optimized nucleic acid was designed as represented in SEQ ID NO: 48, the Rhizopus oryzae glucoamylase was selected having the amino acid sequence (without signal sequence) (SEQ ID NO: 49) from the maize-optimized nucleic acid was designed and is represented in SEQ ID NO:50, and the maize α-amylase.

A plasmid comprising the maize γ-zein N-terminal signal sequence (MRVLLVALALLALAASATS) (SEQ ID NO:17) is fused to the synthetic gene encoding the enzyme. Optionally, the sequence SEKDEL is fused to the C-terminal of the synthetic gene for targeting to and retention in the ER. The fusion is cloned behined the maize γ-zein promoter for expression specifically in the endosperm in a plant transformation plasmid. The fusion is delivered to the corn tissue via Agrobacterium transfection.

### Example 25

Expression cassettes comprising the selected enzymes are constructed to express the enzymes. A plasmid comprising the sequence for a raw starch binding site is fused to the synthetic gene encoding the enzyme. The raw starch binding site allows the enzyme fusion to bind to non-gelatinized starch. The raw-starch binding site amino acid sequence (SEQ ID NO:53) was determined based on literature, and the nucleic acid sequence was maize-optimized to give SEQ ID NO:54. The maize-optimized nucleic acid sequence is fused to the synthetic gene encoding the enzyme in a plasmid for expression in a plant.

### Example 26

### Construction of Maize-Optimized Genes and Vectors for Plant Transformation

The genes or gene fragments were designed by using maize preferred codons for the construction of synthetic genes as set forth in Example 1.

Pyrococcus furiosus EGLA, hyperthermophilic endoglucanase amino acid sequence (without signal sequence) was selected and has the amino acid sequence as set forth in SEQ ID NO: 55, as identified in Journal of Bacteriology (1999) 181, pg 284-290.) The maize-optimized nucleic acid was designed and is represented in SEQ ID NO:56.

Thermus flavus xylose isomerase was selected and has the amino acid sequence as set forth in SEQ ID NO:57, as described in Applied Biochemistry and Biotechnology 62:15-27 (1997).

Expression cassettes are constructed to express the Pyrococcus furiosus EGLA (endoglucanase) from the maize-optimized nucleic acid (SEQ ID NO:56) and the Thermus flavus xylose isomerase from a maize-optimized nucleic acid encoding amino acid sequence SEQ ID NO:57 A plasmid comprising the maize γ-zein N-terminal signal sequence (MRVLLVALALLALAASATS) (SEQ ID NO:17) is fused to the synthetic maize-optimized gene encoding the enzyme. Optionally, the sequence SEKDEL is fused to the C-terminal of the synthetic gene for targeting to and retention in the ER. The fusion is cloned behined the maize γ-zein promoter for expression specifically in the endosperm in a plant transformation plasmid. The fusion is delivered to the corn tissue via Agrobacterium transfection.

### Example 27

### Production of Glucose from Corn Flour Using Thermophilic Enzymes Expressed in Corn

Expression of the hyperthermophilic α-amylase, 797GL3 and α-glucosidase (MalA) were shown to result in production of glucose when mixed with an aqueous solution and incubated at 90° C.

A transgenic corn line (line 168A10B, pNOV4831) expressing MalA enzyme was identified by measuring α-glucosidase activity as indicated by hydrolysis of p-nitrophenyl-α-glucoside.

Corn kernels from transgenic plants expressing 797GL3 were ground to a flour in a Kleco cell thus creating amylase flour. Corn kernels from transgenic plants expressing MalA were ground to a flour in a Kleco cell thus creating MalA flour Non-transgenic corn kernels were ground in the same manner to generate control flour.

Buffer was 50 mM MES buffer pH 6.0.

Corn flour hydrolysis reactions: Samples were prepared as indicated in Table 5 below. Corn flour (about 60 mg per sample) was mixed with 40 ml of 50 mM MES buffer, pH 6.0. Samples were incubated in a water bath set at 90° C. for 2.5 and 14 hours. At the indicated incubation times, samples were removed and analyzed for glucose content.

The samples were assayed for glucose by a glucose oxidase/horse radish peroxidase based assay. GOPOD reagent contained: 0.2 mg/ml o-dianisidine, 100 mM Tris pH 7.5, 100 U/ml glucose oxidase & 10 U/ml horse radish peroxidase. 20 μl of sample or diluted sample were arrayed in a 96 well plate along with glucose standards (which varied from 0 to 0.22 mg/ml). 100 μl of GOPOD reagent was added to each well with mixing and the plate incubated at 37° C. for 30 min. 100 μl of sulfuric acid (9M) was added and absorbance at 540 nm was read. The glucose concentration of the samples was determined by reference to the standard curve. The quantity of glucose observed in each sample is indicated in Table 5.

TABLE 5

| | | amylase | MalA | | Glucose | Glucose |
|---|---|---|---|---|---|---|
| | WT flour | flour | flour | Buffer | 2.5 h | 14 h |
| Sample | mg | mg | Mg | ml | mg | mg |
| 1 | 66 | 0 | 0 | 40 | 0 | 0 |
| 2 | 31 | 30 | 0 | 40 | 0.26 | 0.50 |
| 3 | 30 | 0 | 31.5 | 40 | 0 | 0.09 |
| 4 | 0 | 32.2 | 30.0 | 40 | 2.29 | 12.30 |
| 5 | 0 | 6.1 | 56.2 | 40 | 1.16 | 8.52 |

These data demonstrate that when expression of hyperthermophilic α-amylase and α-glucosidase in corn result in a corn product that will generate glucose when hydrated and heated under appropriate conditions.

### Example 28

### Production of Maltodextrins

Grain expressing thermophilic α-amylase was used to prepare maltodextrins. The exemplified process does not require prior isolation of the starch nor does it require addition of exogenous enzymes.

Corn kernels from transgenic plants expressing 797GL3 were ground to a flour in a Kleco cell to create “amylase flour”. A mixture of 10% transgenic/90% non-transgenic kernels was ground in the same manner to create “10% amylase flour.”

Amylase flour and 10% amylase flour (approximately 60 mg/sample) were mixed with water at a rate of 5 μl of water per mg of flour. The resulting slurries were incubated at 90° C. for up to 20 hours as indicated in Table 6. Reactions were stopped by addition of 0.9 ml of 50 mM EDTA at 85° C. and mixed by pipetting. Samples of 0.2 ml of slurry were removed, centrifuged to remove insoluble material and diluted 3× in water.

The samples were analyzed by HPLC with ELSD detection for sugars and maltodextrins. The gradient HPLC system was equipped with Astec Polymer Amino Column, 5 micron particle size, 250×4.6 mm and an Alltech ELSD 2000 detector. The system was pre-equilibrated with a 15:85 mixture of water:acetonitrile. The flow rate was 1 ml/min. The initial conditions were maintained for 5 min after injection followed by a 20 min gradient to 50:50 water:acetonitrile followed by 10 minutes of the same solvent. The system was washed with 20 min of 80:20 water:acetonitrile and then re-equilibrated with the starting solvent.

The resulting peak areas were normalized for volume and weight of flour. The response factor of ELSD per μg of carbohydrate decreases with increasing DP, thus the higher DP maltodextrins represent a higher percentage of the total than indicated by peak area.

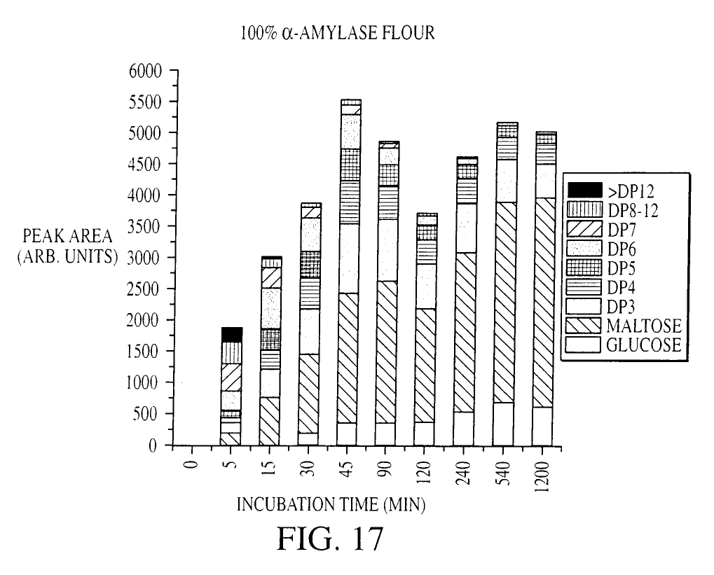

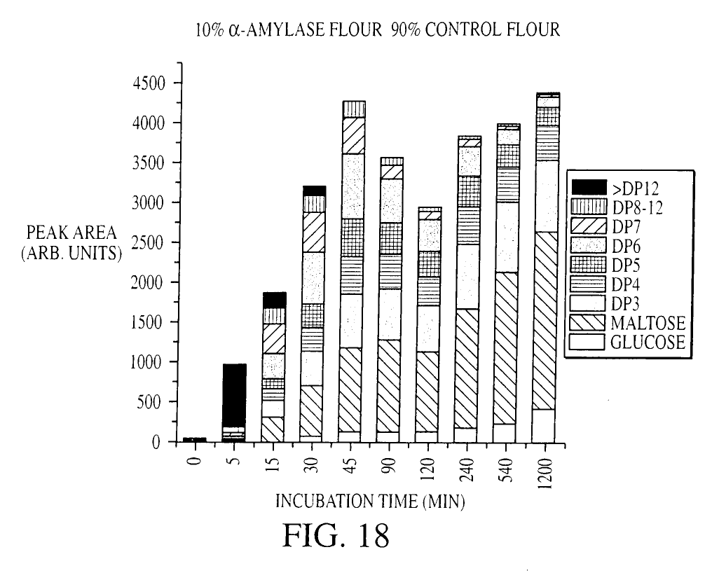

The relative peak areas of the products of reactions with 100% amylase flour are shown in FIG. 17. The relative peak areas of the products of reactions with 10% amylase flour are shown in FIG. 18.

These data demonstrate that a variety of maltodextrin mixtures can be produced by varying the time of heating. The level of α-amylase activity can be varied by mixing transgenic α-amylase-expressing corn with wild-type corn to alter the maltodextrin profile.

The products of the hydrolysis reactions described in this example can be concentrated and purified for food and other applications by use of a variety of well defined methods including: centrifugation, filtration, ion-exchange, gel permeation, ultrafiltration, nanofiltration, reverse osmosis, decolorizing with carbon particles, spray drying and other standard techniques known to the art.

### Example 29

### Effect of Time and Temperature on Maltodextrin Production

The composition of the maltodextrin products of autohydrolysis of grain containing thermophilic α-amylase may be altered by varying the time and temperature of the reaction.

In another experiment, amylase flour was produced as described in Example 28 above and mixed with water at a ratio of 300 μl water per 60 mg flour. Samples were incubated at 70°, 80°, 90°, or 100° C. for up to 90 minutes. Reactions were stopped by addition of 900 ml of 50 mM EDTA at 90° C., centrifuged to remove insoluble material and filtered through 0.45 μm nylon filters. Filtrates were analyzed by HPLC as described in Example 28.

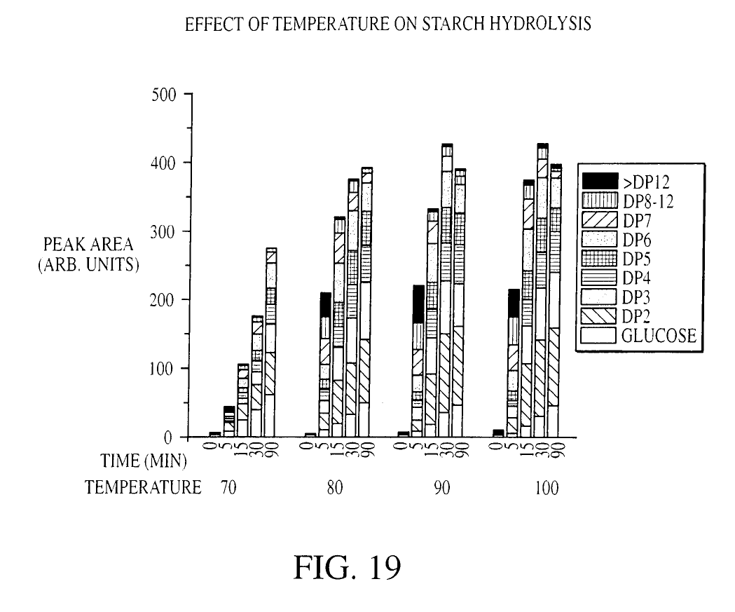

The result of this analysis is presented in FIG. 19. The DP number nomenclature refers to the degree of polymerization. DP2 is maltose; DP3 is maltotriose, etc. Larger DP maltodextrins eluted in a single peak near the end of the elution and are labeled “>DP12”. This aggregate includes dextrins that passed through 0.45 μm filters and through the guard column and does not include any very large starch fragments trapped by the filter or guard column.

This experiment demonstrates that the maltodextrin composition of the product can be altered by varying both temperature and incubation time to obtain the desired maltooligosaccharide or maltodextrin product.

### Example 30

### Maltodextrin Production

The composition of maltodextrin products from transgenic maize containing thermophilic α-amylase can also be altered by the addition of other enzymes such as α-glucosidase and xylose isomerase as well as by including salts in the aqueous flour mixture prior to treating with heat.

In another, amylase flour, prepared as described above, was mixed with purified MalA and/or a bacterial xylose isomerase, designated BD8037. S. sulfotaricus MalA with a 6His purification tag was expressed in E. coli. Cell lysate was prepared as described in Example 28, then purified to apparent homogeneity using a nickel affinity resin (Probond, Invitrogen) and following the manufacturer's instructions for native protein purification. Xylose isomerase BD8037 was obtained as a lyophilized powder from Diversa and resuspended in 0.4× the original volume of water.

Amylase corn flour was mixed with enzyme solutions plus water or buffer. All reactions contained 60 mg amylase flour and a total of 600 μl of liquid. One set of reactions was buffered with 50 mM MOPS, pH 7.0 at room temperature, plus 10 mM MgSO4 and 1 mM CoCl₂; in a second set of reactions the metal-containing buffer solution was replaced by water. All reactions were incubated for 2 hours at 90° C. Reaction supernatant fractions were prepared by centrifugation. The pellets were washed with an additional 600 μl H₂O and re-centrifuged. The supernatant fractions from each reaction were combined, filtered through a Centricon 10, and analyzed by HPLC with ELSD detection as described above.

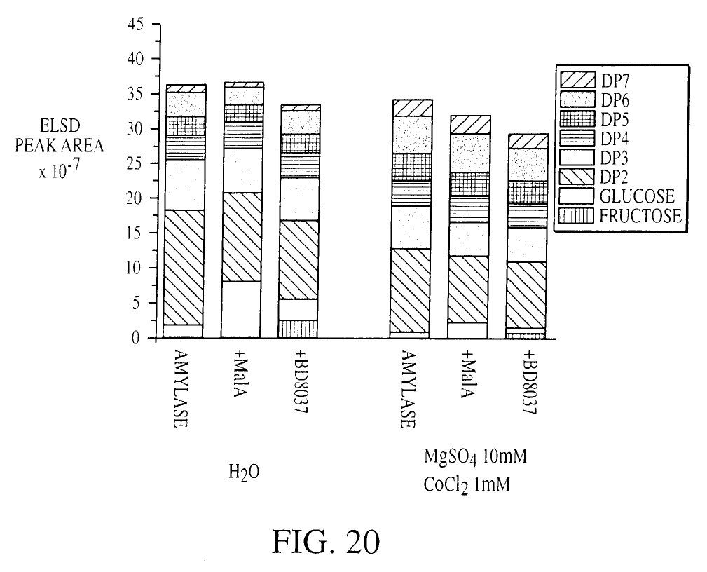

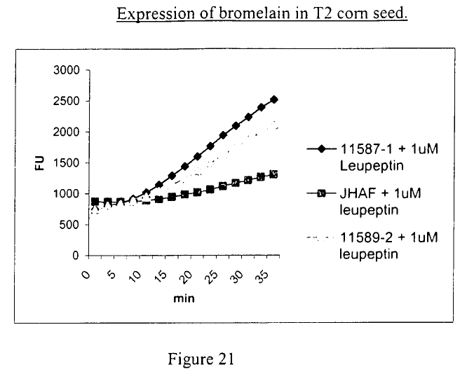

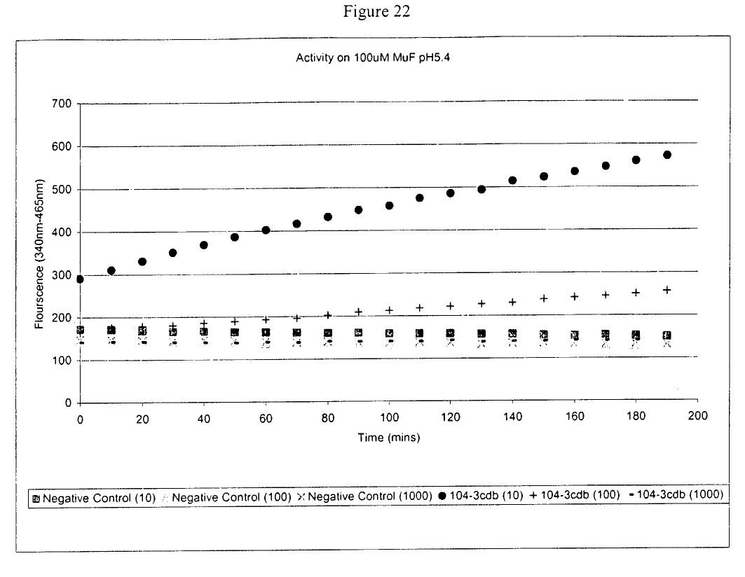

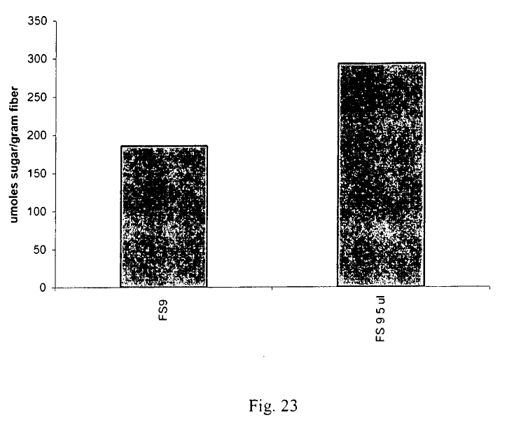

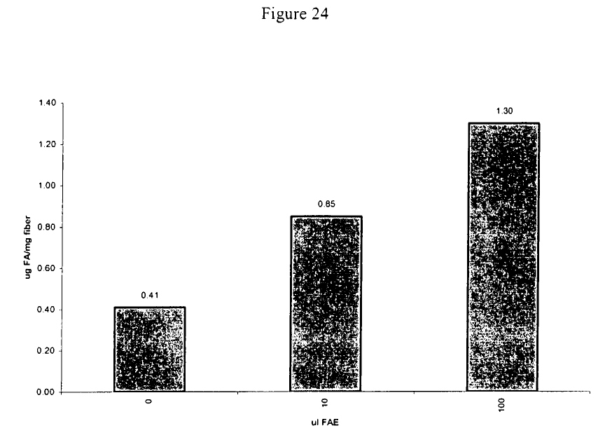

The results are graphed in FIG. 20. They demonstrate that the grain-expressed amylase 797GL3 can function with other thermophilic enzymes, with or without added metal ions, to produce a variety of maltodextrin mixtures from corn flour at a high temperature. In particular, the inclusion of a glucoamylase or α-glucosidase may result in a product with more glucose and other low DP products. Inclusion of an enzyme with glucose isomerase activity results in a product that has fructose and thus would be sweeter than that produced by amylase alone or amylase with α-glucosidase. In addition the data indicate that the proportion of DP5, DP6 and DP7 maltooligosaccharides can be increased by including divalent cationic salts, such as CoCl₂ and MgSO₄.

Other means of altering the maltodextrin composition produced by a reaction such as that described here include: varying the reaction pH, varying the starch type in the transgenic or non-transgenic grain, varying the solids ratio, or by addition of organic solvents.

### Example 31

### Preparing Dextrins or Sugars from Grain without Mechanical Disruption of the Grain Prior to Recovery of Starch-Derived Products

Sugars and maltodextrins were prepared by contacting the transgenic grain expressing the α-amylase, 797GL3, with water and heating to 90° C. overnight (>14 hours). Then the liquid was separated from the grain by filtration. The liquid product was analyzed by HPLC by the method described in Example 15. Table 6 presents the profile of products detected.

TABLE 6

| | Concentration of Products |
|---|---|
| Molecular species | μg/25 μl injection |
| Fructose | 0.4 |
| Glucose | 18.0 |
| Maltose | 56.0 |
| DP3* | 26.0 |
| DP4* | 15.9 |
| DP5* | 11.3 |
| DP6* | 5.3 |
| DP7* | 1.5 |

*Quantification of DP3 includes maltotriose and may include isomers of maltotriose that have an α(1→6) bond in place of an α(1→4) bond. Similarly DP4 to DP7 quantification includes the linear maltooligosaccarides of a given chain length as well as isomers that have one or more α(1→6) bonds in place of one or more α(1→4) bonds

These data demonstrate that sugars and maltodextrins can be prepared by contacting intact α-amylase-expressing grain with water and heating. The products can then be separated from the intact grain by filtration or centrifugation or by gravitational settling.

### Example 32

### Fermentation of Raw Starch in Corn Expressing Rhizopus oryzae Glucoamylase

Transgenic corn kernels are harvested from transgenic plants made as described in Example 29. The kernels are ground to a flour. The corn kernels express a protein that contains an active fragment of the glucoamylase of Rhizopus oryzae (Sequence ID NO: 49) targeted to the endoplasmic reticulum.

The corn kernels are ground to a flour as described in Example 15. Then a mash is prepared containing s 20 g of corn flour, 23 ml of de-ionized water, 6.0 ml of backset (8% solids by weight). pH is adjusted to 6.0 by addition of ammonium hydroxide. The following components are added to the mash: protease (0.60 ml of a 1,000-fold dilution of a commercially available protease), 0.2 mg Lactocide & urea (0.85 ml of a 10-fold dilution of 50% Urea Liquor). A hole is cut into the cap of the 100 ml bottle containing the mash to allow CO₂ to vent. The mash is then inoculated with yeast (1.44 ml) and incubated in a water bath set at 90° C. After 24 hours of fermentation the temperature is lowered to 86° C.; at 48 hours it is set to 82° C.

Yeast for inoculation is propagated as described in Example 14.

Samples are removed as described in example 14 and then analyzed by the methods described in Example 14.

### Example 33

Transgenic corn kernels are harvested from transgenic plants made as described in Example 28. The kernels are ground to a flour. The corn kernels express a protein that contains an active fragment of the glucoamylase of Rhizopus oryzae (Sequence ID NO: 49) targeted to the endoplasmic reticulum.

The corn kernels are ground to a flour as described in Example 15. Then a mash is prepared containing 20 g of corn flour, 23 ml of de-ionized water, 6.0 ml of backset (8% solids by weight). pH is adjusted to 6.0 by addition of ammonium hydroxide. The following components are added to the mash: protease (0.60 ml of a 1,000-fold dilution of a commercially available protease), 0.2 mg Lactocide & urea (0.85 ml of a 10-fold dilution of 50% Urea Liquor). A hole is cut into the cap of the 100 ml bottle containing the mash to allow CO₂ to vent. The mash is then inoculated with yeast (1.44 ml) and incubated in a water bath set at 90° C. After 24 hours of fermentation the temperature is lowered to 86° C.; at 48 hours it is set to 82° C.

Yeast for inoculation is propagated as described in Example 14.

Samples are removed as described in example 14 and then analyzed by the methods described in Example 14.

### Example 34

### Example of Fermentation of Raw Starch in Whole Kernels of Corn Expressing Rhizopus oryzae Glucoamylase with Addition of Exogenous α-amylase

Transgenic corn kernels are harvested from transgenic plants made as described in Example 28. The corn kernels express a protein that contains an active fragment of the glucoamylase of Rhizopus oryzae (Sequence ID NO: 49) targeted to the endoplasmic reticulum.

The corn kernels are contacted with 20 g of corn flour, 23 ml of de-ionized water, 6.0 ml of backset (8% solids by weight). pH is adjusted to 6.0 by addition of ammonium hydroxide. The following components are added: barley α-amylase purchased from Sigma (2 mg), protease (0.60 ml of a 1,000-fold dilution of a commercially available protease), 0.2 mg Lactocide & urea (0.85 ml of a 10-fold dilution of 50% Urea Liquor). A hole is cut into the cap of the 100 ml bottle containing the mixture in order to allow CO₂ to vent. The mixture is then inoculated with yeast (1.44 ml) and incubated in a water bath set at 90° C. After 24 hours of fermentation the temperature is lowered to 86° C.; at 48 hours it is set to 82° C.

Yeast for inoculation is propagated as described in Example 14.

Samples are removed as described in example 14 and then analyzed by the methods described in Example 14.

### Example 35

### Fermentation of Raw Starch in Corn expressing Rhizopus oryzae Glucoamylase and Zea mays Amylase

Transgenic corn kernels are harvested from transgenic plants made as described in Example 28. The corn kernels express a protein that contains an active fragment of the glucoamylase of Rhizopus oryzae (Sequence ID NO:49) targeted to the endoplasmic reticulum. The kernels also express the maize amylase with raw starch binding domain as described in Example 28.

The corn kernels are ground to a flour as described in Example 14. Then a mash is prepared containing 20 g of corn flour, 23 ml of de-ionized water, 6.0 ml of backset (8% solids by weight). pH is adjusted to 6.0 by addition of ammonium hydroxide. The following components are added to the mash: protease (0.60 ml of a 1,000-fold dilution of a commercially available protease), 0.2 mg Lactocide & urea (0.85 ml of a 10-fold dilution of 50% Urea Liquor). A hole is cut into the cap of the 100 ml bottle containing the mash to allow CO₂ to vent. The mash is then inoculated with yeast (1.44 ml) and incubated in a water bath set at 90 F. After 24 hours of fermentation the temperature is lowered to 86 F; at 48 hours it is set to 82 F.

Yeast for inoculation is propagated as described in Example 14.

Samples are removed as described in example 14 and then analyzed by the methods described in Example 14.

### Example 36

### Example of Fermentation of Raw Starch in Corn Expressing Thermoanaerobacter thermosaccharolyticum Glucoamylase

Transgenic corn kernels are harvested from transgenic plants made as described in Example 28. The corn kernels express a protein that contains an active fragment of the glucoamylase of Thermoanaerobacter thermosaccharolyticum (Sequence ID NO: 47) targeted to the endoplasmic reticulum.

The corn kernels are ground to a flour as described in Example 15. Then a mash is prepared containing 20 g of corn flour, 23 ml of de-ionized water, 6.0 ml of backset (8% solids by weight). pH is adjusted to 6.0 by addition of ammonium hydroxide. The following components are added to the mash: protease (0.60 ml of a 1,000-fold dilution of a commercially available protease), 0.2 mg Lactocide & urea (0.85 ml of a 10-fold dilution of 50% Urea Liquor). A hole is cut into the cap of the 100 ml bottle containing the mash to allow CO₂ to vent. The mash is then inoculated with yeast (1.44 ml) and incubated in a water bath set at 90° C. After 24 hours of fermentation the temperature is lowered to 86° C.; at 48 hours it is set to 82° C.

Yeast for inoculation is propagated as described in Example 14.

Samples are removed as described in example 14 and then analyzed by the methods described in Example 14.

### Example 37

### Example of Fermentation of Raw Starch in Corn Expressing Aspergillus niger Glucoamylase

Transgenic corn kernels are harvested from transgenic plants made as described in Example 28. The corn kernels express a protein that contains an active fragment of the glucoamylase of Aspergillus niger (Fiil, N. P. “Glucoamylases G1 and G2 from Aspergillus niger are synthesized from two different but closely related mRNAs” EMBO J. 3 (5), 1097-1102 (1984), Accession number P04064). The maize-optimized nucleic acid encoding the glucoamylase has SEQ ID NO:59 and is targeted to the endoplasmic reticulum.

The corn kernels are ground to a flour as described in Example 14. Then a mash is prepared containing 20 g of corn flour, 23 ml of de-ionized water, 6.0 ml of backset (8% solids by weight). pH is adjusted to 6.0 by addition of ammonium hydroxide. The following components are added to the mash: protease (0.60 ml of a 1,000-fold dilution of a commercially available protease), 0.2 mg Lactocide & urea (0.85 ml of a 10-fold dilution of 50% Urea Liquor). A hole is cut into the cap of the 100 ml bottle containing the mash to allow CO₂ to vent. The mash is then inoculated with yeast (1.44 ml) and incubated in a water bath set at 90° C. After 24 hours of fermentation the temperature is lowered to 86° C.; at 48 hours it is set to 82° C.

Yeast for inoculation is propagated as described in Example 14.

Samples are removed as described in example 14 and then analyzed by the methods described in Example 14.

### Example 38

### Example of Fermentation of Raw Starch in Corn Expressing Aspergillus niger Glucoamylase and Zea mays Amylase

Transgenic corn kernels are harvested from transgenic plants made as described in Example 28. The corn kernels express a protein that contains an active fragment of the glucoamylase of Aspergillus niger (Fiil, N. P. “Glucoamylases G1 and G2 from Aspergillus niger are synthesized from two different but closely related mRNAs” EMBO J. 3 (5), 1097-1102 (1984): Accession number P04064) (SEQ ID NO:59, maize-optimized nucleic acid) and is targeted to the endoplasmic reticulum. The kernels also express the maize amylase with raw starch binding domain as described in example 28.

The corn kernels are ground to a flour as described in Example 14. Then a mash is prepared containing 20 g of corn flour, 23 ml of de-ionized water, 6.0 ml of backset (8% solids by weight). pH is adjusted to 6.0 by addition of ammonium hydroxide. The following components are added to the mash: protease (0.60 ml of a 1,000-fold dilution of a commercially available protease), 0.2 mg Lactocide & urea (0.85 ml of a 10-fold dilution of 50% Urea Liquor). A hole is cut into the cap of the 100 ml bottle containing the mash to allow CO₂ to vent. The mash is then inoculated with yeast (1.44 ml) and incubated in a water bath set at 90° C. After 24 hours of fermentation the temperature is lowered to 86° C.; at 48 hours it is set to 82° C.

Yeast for inoculation is propagated as described in Example 14.

Samples are removed as described in example 14 and then analyzed by the methods described in Example 14.

### Example 39

### Example of Fermentation of Raw Starch in Corn Expressing Thermoanaerobacter thermosaccharolyticum Glucoamylase and Barley Amylase

Transgenic corn kernels are harvested from transgenic plants made as described in Example 28. The corn kernels express a protein that contains an active fragment of the glucoamylase of Thermoanaerobacter thermosaccharolyticum (Sequence ID NO: 47) targeted to the endoplasmic reticulum. The kernels also express the low pI barley amylase amyl gene (Rogers, J. C. and Milliman, C. “Isolation and sequence analysis of a barley alpha-amylase cDNA clone” J. Biol. Chem. 258 (13), 8169-8174 (1983) modified to target expression of the protein to the endoplasmic reticulum.

The corn kernels are ground to a flour as described in Example 14. Then a mash is prepared containing 20 g of corn flour, 23 ml of de-ionized water, 6.0 ml of backset (8% solids by weight). pH is adjusted to 6.0 by addition of ammonium hydroxide. The following components are added to the mash: protease (0.60 ml of a 1,000-fold dilution of a commercially available protease), 0.2 mg Lactocide & urea (0.85 ml of a 10-fold dilution of 50% Urea Liquor). A hole is cut into the cap of the 100 ml bottle containing the mash to allow CO₂ to vent. The mash is then inoculated with yeast (1.44 ml) and incubated in a water bath set at 90° C. After 24 hours of fermentation the temperature is lowered to 86° C.; at 48 hours it is set to 82° C.

Yeast for inoculation is propagated as described in Example 14.

Samples are removed as described in example 14 and then analyzed by the methods described in Example 14.

### Example 40

### Example of Fermentation of Raw Starch in Whole Kernals of Corn Expressing, Thermoanaerobacter thermosaccharolyticum Glucoamylase and Barley Amylase

Transgenic corn kernels are harvested from transgenic plants made as described in Example 28. The corn kernels express a protein that contains an active fragment of the glucoamylase of Thermoanaerobacter thermosaccharolyticum (Sequence ID NO: 47) targeted to the endoplasmic reticulum. The kernels also express the low pI barley amylase amyl gene (Rogers, J. C. and Milliman, C. “Isolation and sequence analysis of a barley alpha-amylase cDNA clone” J. Biol. Chem. 258 (13), 8169-8174 (1983) modified to target expression of the protein to the endoplasmic reticulum.

The corn kernels are contacted with 20 g of corn flour, 23 ml of de-ionized water, 6.0 ml of backset (8% solids by weight). pH is adjusted to 6.0 by addition of ammonium hydroxide. The following components are added to the mixture: protease (0.60 ml of a 1,000-fold dilution of a commercially available protease), 0.2 mg Lactocide & urea (0.85 ml of a 10-fold dilution of 50% Urea Liquor). A hole is cut into the cap of the 100 ml bottle containing the mash to allow CO₂ to vent. The mixture is then inoculated with yeast (1.44 ml) and incubated in a water bath set at 90° C. After 24 hours of fermentation the temperature is lowered to 86° C.; at 48 hours it is set to 82° C.

Yeast for inoculation is propagated as described in Example 14.

Samples are removed as described in example 14 and then analyzed by the methods described in Example 14.

### Example 41

### Example of Fermentation of Raw Starch in Corn Expressing an Alpha-Amylase and Glucoamylase Fusion

Transgenic corn kernels are harvested from transgenic plants made as described in Example 28. The corn kernels express a maize-optimized polynucleotide such as provided in SEQ ID NO: 46, encoding an alpha-amylase and glucoamylase fusion, such as provided in SEQ ID NO: 45, which are targeted to the endoplasmic reticulum.

The corn kernels are ground to a flour as described in Example 14. Then a mash is prepared containing 20 g of corn flour, 23 ml of de-ionized water, 6.0 ml of backset (8% solids by weight). pH is adjusted to 6.0 by addition of ammonium hydroxide. The following components are added to the mash: protease (0.60 ml of a 1,000-fold dilution of a commercially available protease), 0.2 mg Lactocide & urea (0.85 ml of a 10-fold dilution of 50% Urea Liquor). A hole is cut into the cap of the 100 ml bottle containing the mash to allow CO₂ to vent. The mash is then inoculated with yeast (1.44 ml) and incubated in a water bath set at 90° C. After 24 hours of fermentation the temperature is lowered to 86° C.; at 48 hours it is set to 82° C.

Yeast for inoculation is propagated as described in Example 14.

Samples are removed as described in example 14 and then analyzed by the methods described in Example 14.

### Example 42

### Construction of Transformation Vectors

Expression cassettes were constructed to express the hyperthermophilic beta-glucanase EglA in maize as follows:

pNOV4800 comprises the barley Amy32b signal peptide

(MGKNGNLCCFSLLLLLLAGLASGHQ) fused to the synthetic gene for the EglA beta-glucanase for targeting to the endoplasmic reticulum and secretion into the apoplast. The fusion was cloned behind the maize γ-zein promoter for expression specifically in the endosperm.

pNOV4803 comprises the barley Amy32b signal peptide fused to the synthetic gene for the EglA beta-glucanase for targeting to the endoplasmic reticulum and secretion into the apoplast. The fusion was cloned behind the maize ubiquitin promoter for expression throughout the plant.
Expression cassettes were constructed to express the thermophilic beta-glucanase/mannanase 6GP1 (SEQ ID NO: 85) in maize as follows:
pNOV4819 comprises the tobacco PR1a signal peptide
(MGFVLFSQLPSFLLVSTLLLFLVISHSCRA) fused to the synthetic gene for the 6GP1 beta-glucanase/mannanase for targeting to the endoplasmic reticulum and secretion into the apoplast. The fusion was cloned behind the maize γ-zein promoter for expression specifically in the endosperm.
pNOV4820 comprises the synthetic gene for 6GP1 cloned behind the maize γ-zein promoter for cytoplasmic localization and expression specifically in the endosperm.
pNOV4823 comprises the tobacco PR1a signal peptide fused to the synthetic gene for the 6GP1 beta-glucanase/mannanase with a C-terminal addition of the sequence KDEL for targeting to and retention in the endoplasmic reticulum. The fusion was cloned behind the maize γ-zein promoter for expression specifically in the endosperm.
pNOV4825 comprises the tobacco PR1a signal peptide fused to the synthetic gene for the 6GP1 beta-glucanase/mannanase with a C-terminal addition of the sequence KDEL for targeting to and retention in the endoplasmic reticulum. The fusion was cloned behind the maize ubiquitin promoter for expression throughout the plant.
Expression cassettes were constructed to express the barley Amyl alpha-amylase (SEQ ID NO: 87) in maize as follows:
pNOV4867 comprises the maize γ-zein N-terminal signal sequence fused to the barley AmyI alpha-amylase with a C-terminal addition of the sequence SEKDEL for targeting to and retention in the endoplasmic reticulum. The fusion was cloned behind the maize γ-zein promoter for expression specifically in the endosperm.
pNOV4879 comprises the maize γ-zein N-terminal signal sequence fused to the barley Amyl alpha-amylase with a C-terminal addition of the sequence SEKDEL for targeting to and retention in the endoplasmic reticulum. The fusion was cloned behind the maize globulin promoter for expression specifically in the embryo.
pNOV4897 comprises the maize γ-zein N-terminal signal sequence fused to the barley Amyl alpha-amylase for targeting to the endoplasmic reticulum and secretion into the apoplast. The fusion was cloned behind the maize globulin promoter for expression specifically in the embryo.
pNOV4895 comprises the maize γ-zein N-terminal signal sequence fused to the barley Amyl alpha-amylase for targeting to the endoplasmic reticulum and secretion into the apoplast. The fusion was cloned behind the maize γ-zein promoter for expression specifically in the endosperm
pNOV4901 comprises the gene for the barley Amyl alpha-amylase cloned behind the maize globulin promoter for cytoplasmic localization and expression specifically in the embryo. Expression cassettes were constructed to express the Rhizopus glucoamylase (SEQ ID NO: 50) in maize as follows:
pNOV4872 comprises the maize γ-zein N-terminal signal sequence fused to the synthetic gene for Rhizopus glucoamylase with a C-terminal addition of the sequence SEKDEL for targeting to and retention in the endoplasmic reticulum. The fusion was cloned behind the maize γ-zein promoter for expression specifically in the endosperm.
pNOV4880 comprises the maize γ-zein N-terminal signal sequence fused to the synthetic gene for Rhizopus glucoamylase with a C-terminal addition of the sequence SEKDEL for targeting to and retention in the endoplasmic reticulum. The fusion was cloned behind the maize globulin promoter for expression specifically in the embryo.
pNOV4889 comprises the maize γ-zein N-terminal signal sequence fused to the synthetic gene for Rhizopus glucoamylase for targeting to the endoplasmic reticulum and secretion into the apoplast. The fusion was cloned behind the maize globulin promoter for expression specifically in the embryo.
pNOV4890 comprises the maize γ-zein N-terminal signal sequence fused to the synthetic gene for Rhizopus glucoamylase for targeting to the endoplasmic reticulum and secretion into the apoplast. The fusion was cloned behind the maize γ-zein promoter for expression specifically in the endosperm.
pNOV4891 comprises the synthetic gene for Rhizopus glucoamylase cloned behind the maize γ-zein promoter for cytoplasmic localization and expression specifically in the endosperm.

### Example 43

### Expression of the Mesophilic Rhizopus Glucoamylase in Corn

A variety of constructs were generated for the expression of the Rhizopus glucoamylase in corn. The maize γ-zein and globulin promoters were used to express the glucoamylase specifically in the endosperm or embryo, respectively. In addition, the maize γ-zein signal sequence and a synthetic ER retention signal were used to regulate the subcellular localization of the glucoamylase protein. All 5 constructs (pNOV4872, pNOV4880, pNOV4889, pNOV4890, and pNOV4891) yielded transgenic plants with glucoamylase activity detected in the seed. Tables 7 and 8 show the results for individual transgenic seed (construct pNOV4872) and pooled seed (construct pNOV4889), respectively. No detrimental phenotype was observed for any transgenic plants expressing this Rhizopus glucoamylase.

Glucoamylase assay: Seed were ground to a flour and the flour was suspended in water. The samples were incubated at 30 degrees for 50 minutes to allow the glucoamylase to react with the starch. The insoluble material was pelleted and the glucose concentration was determined for the supernatants. The amount of glucose liberated in each sample was taken as an indication of the level of glucoamylase present. Glucose concentration was determined by incubating the samples with GOHOD reagent (300 mM Tris/Cl pH7.5, glucose oxidase (20 U/ml), horseradish peroxidase (20 U/ml), o-dianisidine 0.1 mg/ml) for 30 minutes at 37 degrees C., adding 0.5 volumes of 12N H2S04, and measuring the OD540.

Table 7 shows activity of the Rhizopus glucoamylase in individual transgenic corn seed (construct pNOV4872).

TABLE 7

| | U/g |
|---|---|
| Seed | flour |
| Wild Type #1 | 0.07 |
| Wild Type #2 | 0.55 |
| Wild Type #3 | 0.25 |
| Wild Type #4 | 0.33 |
| Wild Type #5 | 0.30 |
| Wild Type #6 | 0.42 |
| Wild Type #7 | −0.01 |
| Wild Type #8 | 0.31 |
| MD9L022156 #1 | 5.17 |
| MD9L022156 #2 | 1.66 |
| MD9L022156 #3 | 7.66 |
| MD9L022156 #4 | 1.77 |
| MD9L022156 #5 | 7.08 |
| MD9L022156 #6 | 4.46 |
| MD9L022156 #7 | 2.20 |
| MD9L022156 #8 | 3.50 |
| MD9L023377 #1 | 9.23 |
| MD9L023377 #2 | 4.30 |
| MD9L023377 #3 | 6.72 |
| MD9L023377 #4 | 3.35 |
| MD9L023377 #5 | 0.56 |
| MD9L023377 #6 | 4.79 |
| MD9L023377 #7 | 4.60 |
| MD9L023377 #8 | 6.01 |
| MD9L023043 #1 | 4.93 |
| MD9L023043 #2 | 8.74 |
| MD9L023043 #3 | 2.70 |
| MD9L023043 #4 | 0.72 |
| MD9L023043 #5 | 3.33 |
| MD9L023043 #6 | 3.53 |
| MD9L023043 #7 | 3.94 |
| MD9L023043 #8 | 11.51 |
| MD9L023334 #1 | 4.28 |
| MD9L023334 #2 | 2.86 |
| MD9L023334 #3 | 0.56 |
| MD9L023334 #4 | 6.96 |
| MD9L023334 #5 | 3.29 |
| MD9L023334 #6 | 3.18 |
| MD9L023334 #7 | 4.57 |
| MD9L023334 #8 | 7.44 |
| MD9L022039 #1 | 6.25 |
| MD9L022039 #2 | 2.85 |
| MD9L022039 #3 | 4.32 |
| MD9L022039 #4 | 2.51 |
| MD9L022039 #5 | 5.06 |
| MD9L022039 #6 | 5.03 |
| MD9L022039 #7 | 2.79 |
| MD9L022039 #8 | 2.98 |

Table 8 shows activity of the Rhizopus glucoamylase in pooled transgenic corn seed (construct pNOV4889).

TABLE 8

| | U/g |
|---|---|
| Seed | flour |
| Wild Type | 0.38 |
| MD9L023347 | 2.14 |
| MD9L023352 | 2.34 |
| MD9L023369 | 1.66 |
| MD9L023469 | 1.42 |
| MD9L023477 | 1.33 |
| MD9L023482 | 1.95 |
| MD9L023484 | 1.32 |
| MD9L024170 | 1.35 |
| MD9L024177 | 1.48 |
| MD9L024184 | 1.60 |
| MD9L024186 | 1.34 |
| MD9L024196 | 1.38 |
| MD9L024228 | 1.69 |
| MD9L024263 | 1.70 |
| MD9L024315 | 1.32 |
| MD9L024325 | 1.73 |
| MD9L024333 | 1.41 |
| MD9L024339 | 1.84 |

All expression cassettes were inserted into the binary vector pNOV2117 for transformation into maize via Agrobacterium infection. The binary vector contained the phosphomannose isomerase (PMI) gene which allows for selection of transgenic cells with mannose. Transformed maize plants were either self-pollinated or outcrossed and seed was collected for analysis.

### Example 44

### Expression of the Hyperthermophilic Beta-Glucanase EplA in Corn

For expression of the hyperthermophilic beta-glucanase EglA in corn we utilized the ubiquitin promoter for expression throughout the plant and the γ-zein promoter for expression specifically in the endosperm of corn seed. The barley Amy32b signal peptide was fused to EglA for localization in the apoplast.

Expression of the hyperthermophilic beta-glucanase EglA in transgenic corn seed and leaves was analysed using an enzymatic assay and western blotting.

Transgenic seed segregating for construct pNOV4800 or pNOV4803 were analysed using both western blotting and an enzymatic assay for beta-glucanase. Endosperm was isolated from individual seed after soaking in water for 48 hours. Protein was extracted by grinding the endosperm in 50 mM NaPO4 buffer (pH 6.0). Heat-stable proteins were isolated by heating the extracts at 100 degrees C. for 15 minutes, followed by pelleting of the insoluble material. The supernatant containing heat-stable proteins was analysed for beta glucanase activity using the azo-barley glucan method (megazyme). Samples were pre-incubated at 100 degrees C. for 10 minutes and assayed for 10 minutes at 100 degrees C. using the azo-barley glucan substrate. Following incubation, 3 volumes of precipitation solution were added to each sample, the samples were centrifuged for 1 minute, and the OD590 of each supernatant was determined. In addition, 5 ug of protein were separated by SDS-PAGE and blotted to nitrocellulose for western blot analysis using antibodies against the EglA protein. Western blot analysis detected a specific, heat-stable protein(s) in the EglA positive endosperm extracts, and not in negative extracts. The western blot signal correlates with the level of EglA activity detected enzymatically.

EglA activity was analysed in leaves and seed of plants containing the transgenic constructs pNOV4803 and pNOV4800, respectively. The assays (conducted as described above) showed that the heat-stable beta-glucanase EglA was expressed at various levels in the leaves (Table 9) and seed (Table 10) of transgenic plants while no activity was detected in non-transgenic control plants. Expression of EglA in corn utilizing constructs pNOV4800 and pNOV4803 did not result in any detectable negative phenotype.

Table 9 shows the activity of the hyperthermophilic beta-glucanase EglA in leaves of transgenic corn plants. Enzymatic assays were conducted on extracts from leaves of pNOV4803 transgenic plants to detect hyperthermophilic beta-glucanase activity. Assays were conducted at 100 degrees C. using the azo-barley glucan method (megazyme). The results indicate that the transgenic leaves have varying levels of hyperthermophilic beta-glucanase activity.

TABLE 9

| Plant | Abs590 |
|---|---|
| Wild Type | 0 |
| 266A-17D | 0.008 |
| 266A-18E | 0.184 |
| 266A-13C | 0.067 |
| 266A-15E | 0.003 |
| 266A-11E | 0 |
| 265C-1B | 0.024 |
| 265C-1C | 0.065 |
| 265C-2D | 0.145 |
| 265C-5C | 0.755 |
| 265C-5D | 0.133 |
| 265C-3A | 0.076 |
| 266A-4B | 0.045 |
| 266A-12B | 0.066 |
| 266A-11C | 0.096 |
| 266A-14B | 0.074 |
| 266A-4C | 0.107 |
| 266A-4A | 0.084 |
| 266A-12A | 0.054 |
| 266A-15B | 0.052 |
| 266A-11A | 0.109 |
| 266A-20C | 0.044 |
| 266A-19D | 0.02 |
| 266A-12C | 0.098 |
| 266A-4E | 0.248 |
| 266A-18B | 0.367 |
| 265C-3D | 0.066 |
| 266A-20E | 0.163 |
| 266A-13D | 0.084 |
| 265C-3B | 0.065 |
| 266A-15A | 0.131 |
| 266A-13A | 0.169 |
| 265C-3E | 0.116 |
| 266A-20A | 0.365 |
| 266A-20B | 0.521 |
| 266A-19C | 0.641 |
| 266A-20D | 0.561 |
| 266A-4D | 0.363 |
| 266A-18A | 0.676 |
| 265C-5E | 0.339 |
| 266A-17E | 0.221 |
| 266A-11B | 0.251 |
| 265C-4E | 0.138 |
| 265C-4D | 0.242 |

Table 10 shows the activity of the hyperthermophilic beta-glucanase EglA in seed of transgenic corn plants. Enzymatic assays were conducted on extracts from individual, segregating seed of pNOV4800 transgenic plants to detect hyperthermophilic beta-glucanase activity. Assays were conducted at 100 degrees C. using the azo-barley glucan method (megazyme). The results indicate that the transgenic seed have varying levels of hyperthermophilic beta-glucanase activity.

TABLE 10

| Seed | Abs 590 |
|---|---|
| Wild Type | 0 |
| 1A | 1.1 |
| 1B | 0 |
| 1C | 1.124 |
| 1D | 1.323 |
| 2A | 0 |
| 2B | 1.354 |
| 2C | 1.307 |
| 2D | 0 |
| 3A | 0.276 |
| 3B | 0.089 |
| 3C | 0.463 |
| 3D | 0 |
| 4A | 0.026 |
| 4B | 0.605 |
| 4C | 0.599 |
| 4D | 0.642 |
| 5A | 1.152 |
| 5B | 1.359 |
| 5C | 1.035 |
| 5D | 0 |
| 6A | 0.006 |
| 6B | 1.201 |
| 6C | 0.034 |
| 6D | 1.227 |
| 7A | 0.465 |
| 7B | 0 |
| 7C | 0.366 |
| 7D | 0.77 |
| 8A | 1.494 |
| 8B | 1.427 |
| 8C | 0.003 |
| 8D | 1.413 |

### Effect of Transgenic Expression of Endoglucanase EglA on Cell Wall Composition & In Vitro Digestibility Analysis

Five individual seed from each of two lines, #263 & #266, not expressing or expressing Egla (pNOV4803) respectively were grown in the greenhouse. Protein extracts made from small leaf samples from immature plants were used to verify that transgenic endoglucanase activity was present in #266 plants but not #263 plants. At full plant maturity, 30 days after pollination, the whole above ground plant was harvested, roughly chopped, and oven dried for 72 hours. Each sample was divided into 2 duplicate samples (labelled A & B respectively), and subjected to in vitro digestibility analysis using strained rumen fluid using common procedures (Forage fiber analysis apparatus, reagents, procedures, and some applications, by H. K. Goering and P. J. Van Soest, Goering, H. Keith 1941 (Washington, D.C.): Agricultural Research Service, U.S. Dept. of Agriculture, 1970. iv, 20 p.: ill.—Agriculture handbook; no. 379), except that material was treated by a pre-incubation at either 40° C. or 90° C. prior to in vitro digestibility analysis. In vitro digestibility analysis was performed as follows:

Samples were chopped to about 1 mm with a wiley mill, and then sub-divided into 16 weighed aliquots for analysis. Material was suspended in buffer and incubated at either 40° C. or 90° C. for 2 hours, then cooled overnight. Micronutrients, trypticase & casein & sodium sulfite were added, followed by strained rumen fluid, and incubated for 30 hours at 37° C. Analyses of neutral detergent fiber (NDF), acid detergent fiber (ADF) and acid detergent lignin (AD-L) were performed using standard gravimeteric methods (Van Soest & Wine, Use of Detergents in the Analysis of fibrous Feeds. IV. Determination of plant cell-wall constituents. P. J. Van Soest & R. H. Wine. (1967). Journal of The AOAC, 50: 50-55; see also Methods for dietry fiber, neutral detergent fiber and nonstarch polysaccharides in relation to animal nutrition (1991). P. J. Van Soest, J. B. Robertson & B. A. Lewis. J. Dairy Science, 74: 3583-3597.).

Data show that transgenic plants expressing EglA (#266) contain more NDF than control plants (#233), whilst ADF & lignin are relatively unchanged. The NDF fraction of transgenic plants is more readily digested than that of non-transgenic plants, and this is due to an increase in the digestibility of cellulose (NDF-ADF-AD-L), consistent with “self-digestion” of the cell-wall cellulose by the transgenically expressed endoglucanase enzyme.

### Example 45

### Expression of the Thermophilic Beta-Glucanase/Mannanase (6GP1) in Corn

Transgenic seed for pNOV4820 and pNOV4823 were analysed for 6GP1 beta glucanase activity using the azo-barley glucan method (megazyme). Enzymatic assays conducted at 50 degrees C. indicate that the transgenic seed have thermophilic 6GP1 beta-glucanase activity while no activity was detected in non-transgenic seed (positive signal represents background noise associated with this assay).

Table 11 shows activity of the thermophilic beta-glucanase/mannanase 6GP1 in transgenic corn seed. Transgenic seed for pNOV4820 (events 1-6) and pNOV4823 (events 7-9) were analysed for 6GP1 beta-glucanase activity using the azo-barley glucan method (megazyme). Enzymatic assays were conducted at 50 degrees C. and the results indicate that the transgenic seed have thermophilic 6GP1 beta-glucanase activity while no activity is detected in non-transgenic seed.

TABLE 11

| Seed | Abs 590 |
|---|---|
| Wild Type | 0 |
| 1 | 0.21 |
| 2 | 0.31 |
| 3 | 0.36 |
| 4 | 0.23 |
| 5 | 0.16 |
| 6 | 0.14 |
| 7 | 0.52 |
| 8 | 0.54 |
| 9 | 0.49 |

### Example 46

### Expression of the Mesophilic Barley Amyl Amylase in Corn

A variety of constructs were generated for the expression of the barley Amyl alpha-amylase in corn. The maize γ-zein and globulin promoters were used to express the amylase specifically in the endosperm or embryo, respectively. In addition, the maize γ-zein signal sequence and a synthetic ER retention signal were used to regulate the subcellular localization of the amylase protein. All 5 constructs (pNOV4867, pNOV4879, pNOV4897, pNOV4895, pNOV4901) yielded transgenic plants with alpha-amylase activity detected in the seed. Table 12 shows the activity in individual seed for 5 independent, segregating events (constructs pNOV4879 and pNOV4897). All of the constructs produced some transgenic events with a shrivelled seed phenotype indicating that synthesis of the barley AmyI amylase could effect starch formation, accumulation, or breakdown.

Table 12 shows activity of the barley Amyl alpha-amylase in individual corn seed (constructs pNOV4879 and pNOV4897). Individual, segregating seed for constructs pNOV4879 (seed samples 1 and 2) and pNOV4897 (seed samples 3-5) were analysed for alpha-amylase activity as described previously.

TABLE 12

| Seed | U/g corn flour |
|---|---|
| 1A | 19.29 |
| 1B | 1.49 |
| 1C | 18.36 |
| 1D | 1.15 |
| 1E | 1.62 |
| 1F | 14.99 |
| 1G | 1.88 |
| 1H | 1.83 |
| 2A | 2.05 |
| 2B | 36.79 |
| 2C | 30.11 |
| 2D | 2.25 |
| 2E | 32.37 |
| 2F | 1.92 |
| 2G | 20.24 |
| 2H | 35.76 |
| 3A | 22.99 |
| 3B | 1.72 |
| 3C | 25.38 |
| 3D | 18.41 |
| 3E | 28.51 |
| 3F | 2.11 |
| 3G | 16.67 |
| 3H | 1.89 |
| 4A | 1.57 |
| 4B | 36.14 |
| 4C | 23.35 |
| 4D | 1.70 |
| 4E | 1.94 |
| 4F | 14.38 |
| 4G | 2.09 |
| 4H | 1.83 |
| 5A | 11.64 |
| 5B | 18.20 |
| 5C | 1.87 |
| 5D | 2.07 |
| 5E | 1.71 |
| 5F | 1.92 |
| 5G | 12.94 |
| 5H | 15.25 |

### Example 47

### Preparation of Xylanase Constructs

Table 13 lists 9 binary vectors that each contain a unique xylanase expression cassette. The xylanase expression cassettes include a promoter, a synthetic xylanase gene (coding sequence), an intron (PEPC, inverted), and a terminator (35S).

Two synthetic maize-optimized endo-xylanase genes were cloned into binary vector pNOV2117. These two xylanase genes were designated BD7436 (SEQ ID NO: 61) and BD6002A (SEQ ID NO:63). Additional binary vectors containing a third maize-optimized sequence, BD6002B (SEQ ID NO:65) can be made.

Two promoters were used: the maize glutelin-2 promoter (27-kD gamma-zein promoter (SEQ ID NO: 12) and the rice glutelin-1 (Osgt1) promoter (SEQ ID NO: 67). The first 6 vectors listed in Table 1 have been used to generate transgenic plants. The last 3 vectors can also be made and used to generate transgenic plants.

Vector 11560 and 11562 encode the polypeptide shown in SEQ ID NO: 62 (BD7436). Constructs 11559 and 11561 encode a polypeptide consisting of SEQ ID NO: 17 fused to the N-terminus of SEQ ID NO: 62. SEQ ID NO: 17 is the 19 amino acid signal sequence from the 27-kD gamma-zein protein.

Vector 12175 encodes the polypeptide shown in SEQ ID NO: 64 (BD6002A). Vector 12174 encodes a fusion protein consisting of the gamma-zein signal sequence (SEQ ID NO: 17) fused to the N-terminus of SEQ ID NO: 64.

Vectors pWIN062 and pWIN064 encode the polypeptide shown in SEQ ID NO: 66 (BD6002B). Vector pWIN058 encodes a fusion protein consisting of the chloroplast transit peptide of maize waxy protein (SEQ ID NO:68) fused to the N-terminus of SEQ ID NO: 66.

TABLE 13
Xylanase binary vectors

| | | Signal | |
|---|---|---|---|
| Vector | Promoter | Sequence Source | Xylanase Gene |
| 11559 | 27 kD Gamma-zein | 27 kD Gamma-zein | BD7436 |
| 11560 | 27 kD Gamma-zein | None | BD7436 |
| 11561 | OsGt1 | 27 kD Gamma-zein | BD7436 |
| 11562 | OsGt1 | None | BD7436 |
| 12174 | 27 kD Gamma-zein | 27 kD Gamma-zein | BD6002A |
| 12175 | 27 kD Gamma-zein | None | BD6002A |
| PWIN058 | 27 kD Gamma-zein | Maize waxy protein | BD6002B |
| PWIN062 | OsGt1 | None | BD6002B |
| PWIN064 | 27 kD Gamma-zein | None | BD6002B |

All constructs include an expression cassette for PMI, to allow positive selection of regenerated transgenic tissue on mannose-containing media.

### Example 48

### Xylanase Activity Assay Results

The data shown in Tables 14 and 15 demonstrate that xylanase activity accumulates in T1 generation seed harvested from regenerated (T0) maize plants stably transformed with binary vectors containing xylanase genes BD7436 (SEQ ID NO: 61 in Example 47) and BD6002A (SEQ ID NO:63 in Example 47). Using an Azo-WAXY assay (Megazyme), activity was detected in extracts from both pooled (segregating) transgenic seed and single transgenic seed.

T1 seed were pulverized and soluble proteins were extracted from flour samples using citrate-phosphate buffer (pH 5.4). Flour suspensions were stirred at room temperature for 60 minutes, and insoluble material was removed by centrifugation. The xylanase activity of the supernatant fraction was measured using the Azo-WAXY assay (McCleary, B. V. “Problems in the measurement of beta-xylanase, beta-glucanase and alpha-amylase in feed enzymes and animal feeds”. In proceedings of Second European Symposium on Feed Enzymes” (W. van Hartingsveldt, M. Hessing, J. P. van der Jugt, and W. A. C Somers Eds.), Noordwiijkerhout, Netherlands, 25-27 Oct. 1995). Extracts and substrate were pre-incubated at 37° C. To 1 volume of 1× extract supernatant, 1 volume of substrate (1% Azo-Wheat Arabinoxylan S-AWAXP) was added and then incubated at 37° C. for 5 minutes. Xylanase activity in the corn flour extract depolymerizes the Azo-Wheat Arabinoxylan by an endo-mechanism and produces low molecular weight dyed fragments in the form of xylo-oligomers. After the 5 minute incubation, the reaction was terminated by the addition of 5 volumes of 95% EtOH. Addition of alcohol causes the non-depolymerized dyed substrate to precipitate so that only the lower molecular weight xylo-oligomers remain in solution. Insoluble material was removed by centrifugation. The absorbance of the supernatant fraction was measured at 590 nm, and the units of xylanase per gram of flour were determined by comparison to the absorbance values from identical assays using a xylanase standard of known activity. The activity of this standard was determined by a BCA assay. The enzyme activity of the standard was determined using wheat arabinoxylan as substrate and measuring the release of reducing ends by reaction of the reducing ends with 2,2′-bicinchoninic acid (BCA). The substrate was prepared as a 1.4% w/w solution of wheat arabinoxylan (Megazyme P-WAXYM) in 100 mM sodium acetate buffer pH5.30 containing 0.02% sodium azide. The BCA reagent was prepared by combining 50 parts reagent A with 1 part reagent B (reagents A and B were from Pierce, product numbers 23223 and 23224, respectively). These reagents were combined no more than four hours before use. The assay was performed by combining 200 microliters of substrate to 80 microliters of enzyme sample. After incubation at the desired temperature for the desired length of time, 2.80 milliliters of BCA reagent was added. The contents were mixed and placed at 80° C. for 30-45 minutes. The contents were allowed to cool and then transferred to cuvettes and the absorbance at 560 nm was measured relative to known concentrations of xylose. The choice of enzyme dilution, incubation time, and incubation temperature could be varied by one skilled in the art.

The experimental results shown in Table 14 demonstrate the presence of recombinant xylanase activity in flour prepared from T1 generation corn seed. Seed from 12 T0 plants (derived from independent T-DNA integration events) were analyzed. The 12 transgenic events were derived from 6 different vectors as indicated (refer to Table 13 in Example 47 for description of vectors). Extracts of non-transgenic (negative control) corn flour do not contain measurable xylanase activity (see Table 15). The xylanase activity in these 12 samples ranged from 10-87 units/gram of flour.

TABLE 14
Analysis of pooled T1 seed.

| Vector | Sample | Xylanase Units/Gram of Flour |
|---|---|---|
| 11559 | MD9L013800 | 63 |
| 11559 | MD9L012428 | 58 |
| 11560 | MD9L011296 | 33 |
| 11560 | MD9L011322 | 21 |
| 11561 | MD9L012413 | 87 |
| 11561 | MD9L012443 | 83 |
| 11562 | MD9L012890 | 13 |
| 11562 | MD9L013788 | 12 |
| 12174 | MD9L022080 | 16 |
| 12174 | MD9L022195 | 10 |
| 12175 | MD9L022061 | 74 |
| 12175 | MD9L022134 | 69 |

The results in Table 15 demonstrate the presence of xylanase activity in corn flour derived from single kernels. T1 seed from two T0 plants containing vectors 11561 and 11559 were analyzed. These vectors are described in Example 47. Eight seed from each of the two plants were pulverized and flour samples from each seed were extracted. The table shows results of single assays of each extract. No xylanase activity was found in assays of extracts of seeds 1, 5, and 8 for both transgenic events. These seed represent null segregants. Seed 2, 3, 4, 6, and 7 for both transgenic events accumulated measurable xylanase activity attributable to expression of the recombinant BD7436 gene. All 10 seed that tested positive for xylanase activity (>10 unit/gram flour) had an obvious shriveled or shrunken appearance. By contrast the 6 seed that tested negative for xylanase activity (≦1 unit/gram flour) had a normal appearance. This result suggests that the recombinant xylanase depolymerized endogenous (arabino)xylan substrate during seed development and/or maturation.

TABLE 15
Analysis of single T1 seed.

| | |
|---|---|
| Vector 11561 | Vector 11559 |

| Seed | Xylanase Units/ | Seed | Xylanase Units/ |
|---|---|---|---|
| Number | Gram of Flour | Number | Gram of Flour |
| 1 | 0 | 1 | 1 |
| 2 | 45 | 2 | 52 |
| 3 | 38 | 3 | 21 |
| 4 | 40 | 4 | 13 |
| 5 | 0 | 5 | 0 |
| 6 | 40 | 6 | 28 |
| 7 | 32 | 7 | 23 |
| 8 | 0 | 8 | 0 |

### Example 49

### Enhanced Starch Recovery from Corn Seed Using Enzymes

Corn wet-milling includes the steps of steeping the corn kernel, grinding the corn kernel, and separating the components of the kernel. A bench top assay (the Cracked Corn Assay) was developed to mimic the corn wet-milling process

The “Cracked Corn Assay” was used for identifying enzymes that enhance starch yield from maize seed resulting in an improved efficiency of the corn wet milling process. Enzyme delivery was either by exogenous addition, transgenic corn seed, or a combination of both. In addition to the use of enzymes to facilitate separation of the corn components, elimination of SO₂ from the process is also shown.

### Cracked Corn Assay.

One gram of seed was steeped overnight in 4000, 2000, 1000, 500, 400, 40, or 0 ppm SO₂ at 50 degrees C. or 37 degrees C. Seeds were cut in half and the germ removed. Each half seed was cut in half again. Steep water from each steeped seed sample was retained and diluted to a final concentrations ranging from 400 ppm to 0 ppm SO₂. Two milliliters of the steep water with or without enzymes was added to the de-germed seeds and the samples placed at 50 degrees C. or 37 degrees C. for 2-3 hours. Each enzyme was added at 10 units per sample. All samples were vortexed approximately every 15 minutes. After 2-3 hours the samples were filtered through mira cloth into a 50 ml centrifuge tube. The seeds were washed with 2 ml of water and the sample pooled with the first supernatant. The samples were centrifuged for 15 minutes at 3000 rpm. Following centrifugation, the supernatant was poured off and the pellet placed at 37 degrees C. to dry. All pellet weights were recorded. Starch and protein determinations ware also carried out on samples for determining the starch:protein ratios released during the treatments (data not shown).

Analysis of T1 and T2 Seed from Maize Plants Expressing 6GP1 Endoglucanase in Cracked Corn Assay

Transgenic corn (pNOV4819 and pNOV4823) containing a thermostable endoglucanse performed well when analyzed in the Cracked Corn Assay. Recovery of starch from the pNOV4819 line was found to be 2 fold higher in seeds expressing the endoglucanase when steeped in 2000 ppm SO₂. Addition of a protease and cellobiohydrolase to the endoglucanse seed increased the starch recovery approximately 7 fold over control seeds. See Table 16.

TABLE 16
Crack Corn Assay results for cytosolic expressed
Endoglucanase (pNOV4820). Control line, A188/HiII
PNOV4819 lines, 42C6A-1-21 and 27.

| | | Starch Pellet |
|---|---|---|
| Maize Line | Treatment | Wt. (mg) |
| A188/HiII Control | No Enzyme | 28.4 |
| A188/HiII Control | Bromelain/C8546 10U | 109.3 |
| 42C6A-1-21 | No Enzyme | 52.6 |
| 42C6A-1-21 | Bromelain/C8546 10U | 170.4 |
| 42C6A-1-27 | No Enzyme | 60.5 |
| 42C6A-1-27 | Bromelain/C8546 10U | 207.5 |

Similar results were seen in transgenic seed containing endoglucanase targeted to the ER of the endosperm (pNOV4823), again resulting in a 2-7 fold increase in starch recovery when compared to control seed. See Table 17.

TABLE 17
Crack Corn Assay results for ER expressed
endoglucanase (pNOV4823). Control line, A188/HiII;
PNOV4823 line, 101D11A-1-28.

| | | Starch | Starch | |
|---|---|---|---|---|
| | | Pellet Wt | Pellet Wt | Mean |
| Line | Treatment | (mg) | (mg) | Wt. |
| A188/HiII | No Enzyme | 22.5 | 19.1 | 20.8 |
| 101D11A-1-28 | No Enzyme | 41.2 | 32 | 36.6 |
| A188/HiII | 10U Bromelian/C8546 | 78.6 | 73.8 | 76.2 |
| 101D11A-1-28 | 10U Bromelian/C8546 | 169.8 | 132.6 | 151.2 |

These results confirm that expression of an endoglucanase enhances the separation of starch and protein components of the corn seed. Further more it could be shown that reduction or removal of SO2 during the steeping process resulted in starch recovery that was comparable to or better than normally steeped control seeds. See Table 18. Removal of high levels of SO2 from the wet-milling process can provide value-added benefits.

TABLE 18
Comparison of various concentrations of SO2 on starch recovery from
transgenic 6GP1 seed.

| | | Starch Pellet Wt |
|---|---|---|
| Line | Treatment | (mg) |
| A188 Control | 2000 ppm SO2 | 18.5 |
| JHAF Control | 2000 ppm SO2 | 29.1 |
| 42C (pNOV4820) | 2000 ppm SO2 | 29.5 |
| 101C (pNOV4823) | 2000 ppm SO2 | 73.1 |
| 101D (pNOV4823) | 2000 ppm SO2 | 42.5 |
| 136A (pNOV4825) | 2000 ppm SO2 | 36.6 |
| 137A (pNOV4825) | 2000 ppm SO2 | 38.6 |
| 42C (pNOV4820) | 400 ppm SO2 | 18.5 |
| 101C (pNOV4823) | 400 ppm SO2 | 20.4 |
| 101D (pNOV4823) | 400 ppm SO2 | 39.7 |
| 136A (pNOV4825) | 400 ppm SO2 | 26 |
| 137A (pNOV4825) | 400 ppm SO2 | 26.9 |
| 42C (pNOV4820) | 0 ppm SO2 | 21.9 |
| 101C (pNOV4823) | 0 ppm SO2 | 32.5 |
| 101D (pNOV4823) | 0 ppm SO2 | 39 |
| 136A (pNOV4825) | 0 ppm SO2 | 17.8 |
| 137A (pNOV4825) | 0 ppm SO2 | 29.2 |

### Example 50

### Construction of Transformation Vectors for Maize Optimized Bromelain

Expression cassettes were constructed to express the maize optimized bromelain in maize endosperm with various targeting signals as follows:

pSYN11000 (SEQ ID NO. 73) comprises the bromelain signal sequence (MAWKVQVVFLFLFLCVMWASPSAASA) (SEQ ID NO: 72) and synthetic bromelain sequence fused with a C-terminal addition of the sequence VFAEAIAANSTLVAE for targeting to and retention in the PVS (Vitale and Raikhel Trends in Plant Science Vol 4 no. 4 pg 149-155). The fusion was cloned behind the maize gamma zein promoter for expression specifically in the endosperm.

pSYN11587 (SEQ ID NO:75) comprises the bromelain N-terminal signal sequence (MAWKVQVVFLFLFLCVMWASPSAASA) and synthetic bromelain sequence with a C-terminal addition of the sequence SEKDEL for targeting to and retention in the endoplasmic reticulum (ER) (Munro and Pelham, 1987). The fusion was cloned behind the maize gamma zein promoter_for expression specifically in the endosperm.

pSYN11589 (SEQ ID NO. 74) comprises the bromelain signal sequence (MAWKVQVVFLFLFLCVMWASPSAASA) (SEQ ID NO: 72) fused to the lytic vacuolar targeting sequence SSSSFADSNPIRVTDRAAST (Neuhaus and Rogers Plant Molecular Biology 38:127-144, 1998) and synthetic bromelain for targeting to the lytic vacuole. The fusion was cloned behind the maize gamma zein promoter for expression specifically in the endosperm.

pSYN12169 (SEQ ID NO: 76) comprises the maize γ-zein N-terminal signal sequence (MRVLLVALALLALAASATS) (SEQ ID NO:17) fused to the synthetic bromelain for targeting to the endoplasmic reticulum and secretion into the apoplast (Torrent et al. 1997). The fusion was cloned behind the maize gamma zein promoter for expression specifically in the endosperm.

pSYN12575 (SEQ ID NO:77) comprises the waxy amyloplast targeting peptide (Klosgen et al., 1986) fused to the synthetic bromelain for targeting to the amyloplast. The fusion was cloned behind the gamma zein promoter for expression specifically in the endosperm.

pSM270 (SEQ ID NO.78) comprises the bromelain N-terminal signal sequence fused to the lytic vacuolar targeting sequence SSSSFADSNPIRVTDRAAST (Neuhaus and Rogers Plant Molecular Biology 38:127-144, 1998) and synthetic bromelain for targeting to the lytic vacuole. The fusion was cloned behind the aleurone specific promoter P19 (U.S. Pat. No. 6,392,123) for expression specifically in the aleurone.

### Example 51

### Expression of Bromelain in Corn

Seeds from T1 transgenic lines transformed with vectors containing the synthetic bromelain gene with targeting sequences for expression in various subcellular location of the seed were analyzed for protease activity. Corn-flour was made by grinding seeds, for 30 sec., in the Kleco grinder. The enzyme was extracted from 100 mg of flour with 1 ml of 50 mM NaOAc pH4.8 or 50 mM Tris pH 7.0 buffer containing 1 mM EDTA and 5 mM DTT. Samples were vortexed, then placed at 4 C with continuous shaking for 30 min. Extracts from each transgenic line was assayed using resorufin labeled casein (Roche, Cat. No. 1 080 733) as outlined in the product brochure. Flour from T2 seeds were assayed using a bromelain specific assay as outlined in Methods in Enzymology Vol. 244: Pg 557-558 with the following modifications. 100 mg of corn seed flour was extracted with 1 ml of 50 mM Na₂HPO₄/50 mM NaH₂PO₄, pH 7.0, 1 mM EDTA+/−1 μM leupeptin for 15 min at 4° C. Extracts were centrifuged for 5 min at 14,000 rpm at 4° C. Extracts were done in duplicates. Flour from T2 Transgenic lines was assayed for bromelain activity using Z-Arg-Arg-NHMec (Sigma) as a substrate. Four aliquots of 100 μl/corn seed extracts were added to 96 well flat bottom plates (Corning) containing 50 μl 100 mMNa₂HPO₄/100 mM NaH₂PO₄, pH 7.0, 2 mM EDTA, 8 mM DTT/well. The reaction was started by the addition of 50 μl of 20 μM Z-Arg-Arg-NHMec. The reaction rate was monitor using a SpectraFluorPlus (Tecan) fitted with a 360 nm excitation and 465 nm emission filters at 40° C. at 2.5 min intervals.

Table 19 shows the analysis of seed from different T1 bromelain events. Bromelain expression was found to be 2-7 fold higher than the A188 and JHAF control lines. T1 transgenic lines were replanted and T2 seeds obtained. Analysis of T2 seeds showed expression of bromelain. FIG. 21 shows bromelain activity assay using Z-Arg-Arg-NHMec_in T2 seed for ER targeted (11587) and lytic vacuolar targeted (11589) bromelain.

Analysis of T2 Seed from Maize Plants Expressing Bromelain

Seed from T2 transgenic bromelain line, 11587-2 was analyzed in the Cracked Corn assay for enhanced starch recovery. Previous experiments using exogenously added bromelain showed an increased starch recovery when tested alone and in combination with other enzymes, particularly cellulases. The T2 seed from line 11587-2 showed a 1.3 fold increase in starch recovered over control seed when steeped at 37 C/2000 ppm SO2 overnight. More importantly, there was the 2 fold increase in starch from the T2 bromelain line, 11587-2 when a cellulase (C8546) was added when seeds were steeped at 37 C/2000 ppm SO2.

The transgenic line showed a similar trend in increased starch over control seed when seeds were steeped at 37 C/400 ppm SO2. A 1.6 fold increase starch recovered over control was seen in the transgenic seed and a 2.1 fold increase of starch with addition of a cellulase (C8546). See Table 20.

These results are significant in showing that it is possible to reduced temperature and SO2 levels while also enhancing the starch recovery during the wet-milling process when transgenic seed expressing a bromelain is used.

TABLE 19
Summary of Grain Specific Expression of Bromelain in T1 corn.

| | | | “Specific |
|---|---|---|---|
| Line | | | Activity” ng |
| Number | Targeting | Construct | Bromelain/protein |
| 11000-1 | Vacuolar | GZP/probromelain/barleyPVS | 252 |
| 11000-2 | Vacuolar | GZP/probromelain/barleyPVS | 277 |
| 11000-3 | Vacuolar | GZP/probromelain/barleyPVS | 284 |
| 11587-1 | ER | GZP/probromelain/KDEL | 174 |
| 11587-1 | ER | GZP/probromelain/KDEL | 153 |
| 11589-1 | Lytic | GZP/aleurainSS/probromelain | 547 |
| | Vacuolar | | |
| 11589-2 | Lytic | GZP/aleurainSS/probromelain | 223 |
| | Vacuolar | | |
| | | A188 Control | 56 |
| | | JHAF Control | 75 |

TABLE 20
Cracked Corn Assay results for T2 Bromelain seed

| Steep Conditions | Line | Starch Pellet Wt. (mg) |
|---|---|---|
| 2000 ppm SO2 | A188 | 41.3 |
| 2000 ppm SO2 | A188/C8546 (10 units) | 44 |
| 2000 ppm SO2 | 11587-2 | 57.4 |
| 2000 ppm SO2 | 11587-2/C8546 (10 units) | 94.6 |
| 400 ppm | A188 | 30.7 |
| 400 ppm | A188/C8546 (10 units) | 35.8 |
| 400 ppm | 11587-2 | 50.5 |
| 400 ppm | 11587-2/C8546 (10 units) | 86.6 |

### Example 52

### Construction of Transformation Vectors for Maize Optimized Ferulic Acid Esterase

Expression cassettes were constructed to express the maize optimize ferulic acid esterase in maize endosperm with or without various targeting signals as follows:

Plasmid 13036 (SEQ ID NO: 101) comprises the maize optimize ferulic acid esterase (FAE) sequence (SEQ ID NO: 99). The sequence was cloned behind the maize gamma zein promoter without any targeting sequences for expression specifically in the cytosol of the endosperm.

Plasmid 13038 (SEQ ID NO: 103) comprises the maize γ-zein N-terminal signal sequence (MRVLLVALALLALAASATS) (SEQ ID NO: 17) fused to the synthetic FAE for targeting to the endoplasmic reticulum and secretion into the apoplast (Torrent et al. 1997). The fusion was cloned behind the maize gamma zein promoter for expression specifically in the endosperm.

Plasmid 13039 (SEQ ID NO: 105) comprises the waxy amyloplast targeting peptide (MLAALATSQLVATRAGLGVPDASTFRRGAAQGLRGARASAAAD TLSMRTSARAAPRHQHQQARRGARFPSLVVCASAGA) (Klosgen et al., 1986) fused to the synthetic FAE for targeting to the amyloplast. The fusion was cloned behind the gamma zein promoter for expression specifically in the endosperm.

Plasmid 13347 (SEQ ID NO: 107) comprises the maize γ-zein N-terminal signal sequence (MRVLLVALALLALAASATS) (SEQ ID NO:17) fused to the synthetic FAE sequence with a C-terminal addition of the sequence SEKDEL for targeting to and retention in the endoplasmic reticulum (ER) (Munro and Pelham, 1987). The fusion was cloned behind the maize gamma zein promoter_for expression specifically in the endosperm.

All expression cassettes were moved into a binary vector pNOV2117 for transformation into maize via Agrobacterium infection. The binary vector contained the phosphomannose isomerase (PMI) gene which allows for selection of transgenic cells with mannose. Transformed maize plants were either self-pollinated or outcrossed and seed was collected for analysis.

Combinations of the enzymes can be produced either by crossing plants expressing the individual enzymes or by cloning several expression cassettes into the same binary vector to enable cotransformation.

Synthetic Ferulic Acid Esterase Sequence
(SEQ ID NO: 99)
atggccgcctccctcccgaccatgccgccgtccggctacgaccaggtgcg
caacggcgtgccgcgcggccagtggtgaacatctcctacttctccaccgc
caccaactccacccgcccggcccgcgtgtacctcccgccgggctactcca
aggacaagaagtactccgtgctctacctcctccacggcatcggcggctcc
gagaacgactggttcgagggcggcggccgcgccaacgtgatcgccgacaa
cctcatcgccgagggcaagatcaagccgctcatcatcgtgaccccgaaca
ccaacgccgccggcccgggcatcgccgacggctacgagaacttcaccaag
gacctcctcaactcccgtacatcgagtccaactactccgtgtacaccgac
cgcgagcaccgcgccatcgccggcctctctatgggcggcggccagtcctt
caacatcggcctcaccaacctcgacaagttcgcctacatcggcccgatct
ccgccgccccgaacacctacccgaacgagcgcctcttcccggacggcggc
aaggccgcccgcgagaagctcaagctcctcttcatcgcctgcggcaccaa
cgactccctcatcggcttcggccagcgcgtgcacgagtactgcgtggcca
acaacatcaaccacgtgtactggctcatccagggcggcggccacgacttc
aacgtgtggaagccgggcctctggaacttcctccagatggccgacgaggc
cggcctcacccgcgacggcaacaccccggtgccgaccccgtccccgaagc
cggccaacacccgcatcgaggccgaggactacgacggcatcaactcctcc
tccatcgagatcatcggcgtgccgccggagggcggccgcggcatcggcta
catcacctccggcgactacctcgtgtacaagtccatcgacttcggcaacg
gcgccacctccttcaaggccaaggtggccaacgccaacacctccaacatc
gagcttcgcctcaacggcccgaacggcaccctcatcggcaccctctccgt
gaagtccaccggcgactggaacacctacgaggagcagacctgctccatct
ccaaggtgaccggcatcaacgacctctacctcgtgttcaagggcccggtg
aacatcgactggttcaccttcggcgtgtag
Synthetic Ferulic Acid Esterase Amino Acid
Sequence (SEQ ID NO: 100)
maaslptmppsgydqvrngvprgqvvnisyfstatnstrparvylppgys
kdkkysvlyllhgiggsendwfegggranviadnliaegkikpliivtpn
tnaagpgiadgyenftkdllnslipyiesnysvytdrehraiaglsmggg
qsfnigltnldkfayigpisaapntypnerlfpdggkaareklkllfiac
gtndsligfgqrvheycvanninhvywliqggghdfnvwkpglwnflqma
deagltrdgntpvptpspkpantrieaedydginsssieiigvppeggrg
igyitsgdylvyksidfgngatsfkakvanantsnielrlngpngtligt
lsvkstgdwntyeeqtcsiskvtgindlylvfkgpvnidwftfgv*
13036 Sequence (SED ID NO: 101)
atggccgcctccctcccgaccatgccgccgtccggctacgaccaggtgcg
caacggcgtgccgcgcggccagtggtgaacatctcctacttctccaccgc
caccaactccacccgcccggcccgcgtgtacctcccgccgggctactcca
aggacaagaagtactccgtgctctacctcctccacggcatcggcggctcc
gagaacgactggttcgagggcggcggccgcgccaacgtgatcgccgacaa
cctcatcgccgagggcaagatcaagccgctcatcatcgtgaccccgaaca
ccaacgccgccggcccgggcatcgccgacggctacgagaacttcaccaag
gacctcctcaactcccgtacatcgagtccaactactccgtgtacaccgac
cgcgagcaccgcgccatcgccggcctctctatgggcggcggccagtcctt
caacatcggcctcaccaacctcgacaagttcgcctacatcggcccgatct
ccgccgccccgaacacctacccgaacgagcgcctcttcccggacggcggc
aaggccgcccgcgagaagctcaagctcctcttcatcgcctgcggcaccaa
cgactccctcatcggcttcggccagcgcgtgcacgagtactgcgtggcca
acaacatcaaccacgtgtactggctcatccagggcggcggccacgacttc
aacgtgtggaagccgggcctctggaacttcctccagatggccgacgaggc
cggcctcacccgcgacggcaacaccccggtgccgaccccgtccccgaagc
cggccaacacccgcatcgaggccgaggactacgacggcatcaactcctcc
tccatcgagatcatcggcgtgccgccggagggcggccgcggcatcggcta
catcacctccggcgactacctcgtgtacaagtccatcgacttcggcaacg
gcgccacctccttcaaggccaaggtggccaacgccaacacctccaacatc
gagcttcgcctcaacggcccgaacggcaccctcatcggcaccctctccgt
gaagtccaccggcgactggaacacctacgaggagcagacctgctccatct
ccaaggtgaccggcatcaacgacctctacctcgtgttcaagggcccggtg
aacatcgactggttcaccttcggcgtgtag
13036 AA Sequence (SED ID NO: 102)
maaslptmppsgydqvrngvprgqvvnisyfstatnstrparvylppgys
kdkkysvlyllhgiggsendwfegggranviadnliaegkikpliivtpn
tnaagpgiadgyenftkdllnslipyiesnysvytdrehraiaglsmggg
qsfnigltnldkfayigpisaapntypnerlfpdggkaareklkllfiac
gtndsligfgqrvheycvanninhvywliqggghdfnvwkpglwnflqma
deagltrdgntpvptpspkpantrieaedydginsssieiigvppeggrg
igyitsgdylvyksidfgngatsfkakvanantsnielrlngpngtligt
lsvkstgdwntyeeqtcsiskvtgindlylvfkgpvnidwftfgv*
13038 Sequence (SEQ ID NO: 103)
atgagggtgttgctcgttgccctcgctctcctggctctcgctgcgagcgc
cacctccatggccgcctccctcccgaccatgccgccgtccggctacgacc
aggtgcgcaacggcgtgccgcgcggccaggtggtgaacatctcctacttc
tccaccgccaccaactccacccgcccggcccgcgtgtacctcccgccggg
ctactccaaggacaagaagtactccgtgctctacctcctccacggcatcg
gcggctccgagaacgactggttcgagggcggcggccgcgccaacgtgatc
gccgacaacctcatcgccgagggcaagatcaagccgctcatcatcgtgac
cccgaacaccaacgccgccggcccgggcatcgccgacggctacgagaact
tcaccaaggacctcctcaactccctcatcccgtacatcgagtccaactac
tccgtgtacaccgaccgcgagcaccgcgccatcgccggcctctctatggg
cggcggccagtccttcaacatcggcctcaccaacctcgacaagttcgcct
acatcggcccgatctccgccgccccgaacacctacccgaacgagcgcctc
ttcccggacggcggcaaggccgcccgcgagaagctcaagctcctcttcat
cgcctgcggcaccaacgactccctcatcggcttcggccagcgcgtgcacg
agtactgcgtggccaacaacatcaaccacgtgtactggctcatccagggc
ggcggccacgacttcaacgtgtggaagccgggcctctggaacttcctcca
gatggccgacgaggccggcctcacccgcgacggcaacaccccggtgccga
ccccgtccccgaagccggccaacacccgcatcgaggccgaggactacgac
ggcatcaactcctcctccatcgagatcatcggcgtgccgccggagggcgg
ccgcggcatcggctacatcacctccggcgactacctcgtgtacaagtcca
tcgacttcggcaacggcgccacctccttcaaggccaaggtggccaacgcc
aacacctccaacatcgagcttcgcctcaacggcccgaacggcaccctcat
cggcaccctctccgtgaagtccaccggcgagactggaacacctacgagga
gcagacctgctccatctccaaggtgaccggcatcaacgacctctacctcg
tgttcaagggcccggtgaacatcgactggttcaccttcggcgtgtag
13038 AA Sequence (SEQ ID NO: 104)
mrvllvalallalaasatsmaaslptmppsgydqvrngvprgqvvnisyf
statnstrparvylppgyskdkkysvlyllhgiggsendwfegggranvi
adnliaegkikpliivtpntnaagpgiadgyenftkdllnslipyiesny
svytdrehraiaglsmgggqsfnigltnldkfayigpisaapntypnerl
fpdggkaareklkllfiacgtndsligfgqrvheycvanninhvywliqg
gghdfnvwkpglwnflqmadeagltrdgntpvptpspkpantrieaedyd
ginsssieiigvppeggrgigyitsgdylvyksidfgngatsfkakvana
ntsnielrlngpngtligtlsvkstgdwntyeeqtcsiskvtgindlylv
fkgpvnidwftfgv*
13039 Sequence (SEQ ID NO: 105)
atgctggcggctctggccacgtcgcagctcgtcgcaacgcgcgccggcct
gggcgtcccggacgcgtccacgttccgccgcggcgccgcgcagggcctga
ggggggcccgggcgtcggcggcggcggacacgctcagcatgcggaccagc
gcgcgcgcggcgcccaggcaccagcaccagcaggcgcgccgcggggccag
gttcccgtcgctcgtcgtgtgcgccagcgccggcgccatggccgcctccc
tcccgaccatgccgccgtccggctacgaccaggtgcgcaacggcgtgccg
cgcggccaggtggtgaacatctcctacttctccaccgccaccaactccac
ccgcccggcccgcgtgtacctcccgccgggctactccaaggacaagaagt
actccgtgctctacctcctccacggcatcggcggctccgagaacgactgg
ttcgagggcggcggccgcgccaacgtgatcgccgacaacctcatcgccga
gggcaagatcaagccgctcatcatcgtgaccccgaacaccaacgccgccg
gcccgggcatcgccgacggctacgagaacttcaccaaggacctcctcaac
tccctcatcccgtacatcgagtccaactactccgtgtacaccgaccgcga
gcaccgcgccatcgccggcctctctatgggcggcggccagtccttcaaca
tcggcctcaccaacctcgacaagttcgcctacatcggcccgatctccgcc
gccccgaacacctacccgaacgagcgcctcttcccggacggcggcaaggc
cgcccgcgagaagctcaagctcctcttcatcgcctgcggcaccaacgact
ccctcatcggcttcggccagcgcgtgcacgagtactgcgtggccaacaac
atcaaccacgtgtactgctcatccagggcggcggccacgacttcaacgtg
tggaagccgggcctctggaacttcctccagatggccgacgaggccggcct
cacccgcgacggcaacaccccggtgccgaccccgtccccgaagccggcca
acacccgcatcgaggccgaggactacgacggcatcaactcctcctccatc
gagatcatcggcgtgccgccggagggcggccgcggcatcggctacatcac
ctccggcgactacctcgtgtacaagtccatcgacttcggcaacggcgcca
cctccttcaaggccaaggtggccaacgccaacacctccaacatcgagctt
cgcctcaacggcccgaacggcaccctcatcggcaccctctccgtgaagtc
caccggcgactggaacacctacgaggagcagacctgctccatctccaagg
tgacggcatcaacgacctctacctcgtgttcaagggcccggtgaacatcg
actggttcaccttcggcgtgtag
13039 AA Sequence (SEQ ID NO: 106)
mlaalatsqlvatraglgvpdastfrrgaaqglrgarasaaadtlsmrts
araaprhqhqqarrgarfpslvvcasagamaaslptmppsgydqvrngvp
rgqvvnisyfstatnstrparvylppgyskdkkysvlyllhgiggsendw
fegggranviadnliaegkikpliivtpntnaagpgiadgyenftkdlln
slipyiesnysvytdrehraiaglsmgggqsfnigltnldkfayigpisa
apntypnerlfpdggkaareklkllfiacgtndsligfgqrvheycvann
inhvywliqggghdfnvwkpglwnflqmadeagltrdgntpvptpspkpa
ntrieaedydginsssieiigvppeggrgigyitsgdylvyksidfgnga
tsfkakvanantsnielrlngpngtliglsvkstgdwntyeeqtcsiskv
tgindlylvfkgpvnidwftfgv*
13347 Sequence (SEQ ID NO: 107)
atgagggtgttgctcgttgccctcgctctcctggctctcgctgcgagcgc
cacctccatggccgcctccctcccgaccatgccgccgtccggctacgacc
aggtgcgcaacggcgtgccgcgcggccaggtggtgaacatctcctacttc
tccaccgccaccaactccacccgcccggcccgcgtgtacctcccgccggg
ctactccaaggacaagaagtactccgtgctctacctcctccacggcatcg
cggctccgagaacgactggttcgagggcggcggccgcgccaacgtgatcg
ccgacaacctcatcgccgagggcaagatcaagccgctcatcatcgtgacc
ccgaacaccaacgccgccggcccgggcatcgccgacggctacgagaactt
caccaaggacctcctcaactccctcatcccgtacatcgagtccaactact
ccgtgtacaccgaccgcgagcaccgcgccatcgccggcctctctatgggc
ggcggccagtccttcaacatcggcctcaccaacctcgacaagttcgccta
catcggcccgatctccgccgccccgaacacctacccgaacgagcgcctct
tcccggacggcggcaaggccgcccgcgagaagctcaagctcctcttcatc
gcctgcggcaccaacgactccctcatcggcttcggccagcgcgtgcacga
gtactgcgtggccaacaacatcaaccacgtgtactggctcatccagggcg
gcggccacgacttcaacgtgtggaagccgggcctctggaacttcctccag
atggccgacgaggccggcctcacccgcgacggcaacaccccggtgccgac
cccgtccccgaagccggccaacacccgcatcgaggccgaggactacgacg
gcatcaactcctcctccatcgagatcatcggcgtgccgccggagggcggc
cgcggcatcggctacatcacctccggcgactacctcgtgtacaagtccat
cgacttcggcaacggcgccacctccttcaaggccaaggtggccaacgcca
acacctccaacatcgagcttcgcctcaacggcccgaacggcaccctcatc
ggcaccctctccgtgaagtccaccggcgactggaacacctacgaggagca
gacctgctccatctccaaggtgaccggcatcaacgacctctacctcgtgt
tcaagggcccggtgaacatcgactggttcaccttcggcgtgtccgagaag
gacgaactctag
13347 Sequence (SEQ ID NO: 108)
mrvllvalallalaasatsmaaslptmppsgydqvrngvprgqvynisyf
statnstrparvylppgyskdkkysvlyllhgiggsendwfegggranvi
adnliaegkikpliivtpntnaagpgiadgyenftkdllnslipyiesny
svytdrehraiaglsmgggqsfnigltnldkfayigpisaapntypnerl
fpdggkaareklkllfiacgtndsligfgqrvheycvanninhvywliqg
gghdfnvwkpglwnflqmadeagltrdgntpvptpspkpantrieaedyd
ginsssieiigvppeggrgigyitsgdylvyksidfgngatsfkakvana
ntsnielrlngpngtligtlsvkstgdwntyeeqtcsiskvtgindlylv
fkgpvnidwftfgvsekdel*

### Example 53

### Hydrolytic Degradation of Corn Fiber by Ferulic Acid Esterase

Corn fiber is a major by-product of corn wet and dry milling. The fiber component is composed primarily of course fiber arising from the seed pericarp (hull) and aleurone, with a smaller fraction of fine fiber coming from the endosperm cell walls. Ferulic acid, a hydroxycinnamic acid, is found in high concentrations in the cell walls of cereal grains resulting in a cross linking of lignin, hemicellulose and cellulose components of the cell wall. Enzymatic degradation of ferulate cross-linking is an important step in the hydrolysis of corn fiber and may result in the accessibility of further enzymatic degradation by other hydrolytic enzymes.

### Ferulic Acid Esterase Activity Assay

Ferulic acid esterase, FAE-1, (maize optimised synthetic gene from C. thermocellum) was expressed in E. coli. Cells were harvested and stored at −80° C. overnight. Harvested bacteria was suspended in 50 mM Tris buffer pH7.5. Lysozyme was added to a final concentration of 200 ug/mL and the sample incubated 10 minutes at room temperature with gently shaking. The sample was centrifuged at 4° C. for 15 minutes at 4000 rpm. Following centrifugation, the supernatant was transferred to a 50 mL conical tube, and placed in 70 degree Celsius water bath for 30 minutes. The sample was then centrifuged for 15 minutes at 4000 rpm and the cleared supernatant transferred to a conical tube (Blum et al. J Bacteriology, March 2000, pg 1346-1351.)

The recombinant FAE-1 was tested for activity using 4-methylumbelliferyl ferulate as described in Mastihubova et al (2002) Analytical Biochemistry 309 96-101. Recombinant protein FAE-1 (104-3) was diluted 10, 100, and 1000 fold and assayed. Activity assay results are shown in FIG. 22.

### Preparation of Corn Seed Fiber

Corn pericarp coarse fiber was isolated by steeping yellow dent #2 kernels for 48 hrs at 50° C. in 2000 ppm sodium metabisulfite ((Aldrich). Kernels were mixed with water in equal parts and blended in a Waring laboratory heavy duty blender with the blade in reverse orientation. Blender was controlled with a variable autotransformer (Staco Energy) at 50% voltage output for 2 min. Blended material was washed with tap water over a standard test sieve #7 (Fisher scientific) to separate coarse fiber from starch fractions. Coarse fiber and embryos were separated by floating the fiber way from the embryos with hot tap water in a 4 L beaker (Fisher scientific). The fiber was then soaked in ethanol prior to drying overnight in a vacuum oven (Precision) at 60° C. Corn coarse fiber derived form corn kernel pericarp was milled with a laboratory mill 3100 fitted with a mill feeder 3170 (Perten instruments) to 0.5 mm particle size.

Corn Fiber Hydrolysis Assay

Course fiber (CF) was suspended in 50 mM citrate-phosphate buffer, pH 5.2 at 30 mg/5 ml buffer. The CF stock was vortexed and transferred to a 40 ml modular reservoir (Beckman, Cat. No. 372790). The solution was mixed well then 100 ul transferred to a 96 well plate (Corning Inc., Cat. No. 9017, polystyrene, flat bottom). Enzyme was added at 1-10 ul/well and the final volume adjusted to 110 ul with buffer. CF background controls contained 10 ul of buffer only. Plates were sealed with aluminum foil and incubated at 37° C. with constant shaking for 18 hours. The plates were centrifuged for 15 min at 4000 rpm. 1-10 ul of CF supernatant was transferred to a 96 well plate preloaded with 100 ul of BCA reagents (BCA-reagents: Reagent A (Pierce, Prod. #23223), Reagent B (Pierce, Prod.#23224). The final volume was adjusted to 110 ul. The plate was sealed with aluminum foil and placed at 85° C. for 30 min. Following incubation at 85° C., the plate was centrifuged for 5 min at 2500 rpm. Absorbance values were read at 562 nm (Molecular Devices, Spectramax Plus). Samples were quantified with D-glucose and D-xylose (Sigma) calibration curves. Assay results are reported as total sugar released.

### Measurement of Total Sugar Released by Ferulic Acid Esterase in Corn Seed Fiber Hydrolysis Assay

Results from the recombinant FAE-1 fiber hydrolysis assay showed no increase in total reducing sugars (data not shown). These results were not unexpected since it has been reported in the literature that an increase in total reducing sugars is detectable only when other hydrolytic enzymes are used in combination with the FAE (Yu et al J. Agric. Food Chem. 2003, 51, 218-223). FIG. 23 shows that addition of FAE-2 to a fungal supernatant which had been grown on corn fiber, shows and increase in total reducing sugars. This suggests that FAE does play an important role in corn fiber hydrolysis.

FIG. 23 shows Corn Fiber Hydrolysis assay results showing increase in release of total reducing sugars from corn fiber with addition of FAE-2 to fungal supernatant (FS9).

Analysis of Ferulic Acid Released from Corn Seed Fiber by FAE-1

FAE activity on corn fiber was tested by following the release of ferulic acid as described in Walfron and Parr (1996) (Waldron, K W, Parr A J 1996 Vol 7 pages 305-312 Phytochem Anal) with slight modification. Corn coarse fiber derived from corn kernel pericarp was milled with a laboratory mill 3100 fitted with a mill feeder 3170 (Perten instruments) to 0.5 mm particle size and used as substrate at a concentration of 10 mg/ml. 1 ml assays were conducted in 24 well Becton Dickenson Multiwell™. Substrate was incubated in 50 mM citrate phosphate pH 5.4 at 50° C. at 110 rpm for 18 hrs in the presence and absence of recombinant FAE. After the incubation period, samples were centrifuged for 10 minutes at 13,000 rpm prior to ethyl acetate extraction. All solvents and acids used were from Fisher Scientific. 0.8 ml of supernatant was acidified with 0.5 ml acetic glacial acid and extracted three times with equivalent volume of ethyl acetate. Organic fractions were combined and speed vac to dryness (Savant) at 40° C. Samples were then suspended with 100 μl of methanol and used for HPLC analysis.

HPLC chromatography was carried out as follows. Ferulic acid (ICN Biomedicals) was used as standard in HPLC analysis (data not shown). HPLC analysis was conducted with a Hewlett Packard series 1100 HPLC system. The procedure employed a C₁₈ fully capped reverse phase column (XterraRp₁₈, 150 mm×3.9 mm i.d. 5 μm particle size) operated in 1.0 ml min m⁻¹ at 40° C. Ferulic Acid was eluted with a gradient of 25 to 70% B in 32 min (solvent A: H₂O, 0.01% b TFA; solvent B: MeCN, 0.0075%).

As shown in FIG. 24, FA released from corn fiber was 2-3 fold higher than control when treated with 10 or 100 ul of FAE-1. These results clearly show that FAE-1 is capable of hydrolyzing corn fiber.

### Example 54

### Functionality in Fermentation of Maize Expressed Glucoamylase and Amylase

This example demonstrates that maize-expressed enzymes will support fermentation of starch in a corn slurry in the absence of added enzyme and without cooking the corn slurry. Maize kernels that contain Rhizopus ozyzae glucoamylase (ROGA) (SEQ ID NO: 49) were produced as described in Example 32. Maize kernels that contain the barley low-pI α-amylase (AMYI) (SEQ ID NO: 88) are produced as described in Example 46. The following materials are used in this example:
Aspergillus niger glucoamylase (ANGA) was purchased from Sigma.
Rhizopus species glucoamylase (RxGA) was purchased from Wako as a dry crystalline powder and made up in 10 mM NaAcetate pH 5.2, 5 mM CaCl₂. at 10 mg/ml.
MAMYI Microbially produced AMYI was prepared at approximately 0.25 mg/ml in 10 mM NaAcetate pH 5.2, 5 mM CaCl₂.
Yeast was Saccharomyces cereviceae
YE was a sterile 5% solution of yeast extract in water
Yeast starter contained 50 g maltodextrin, 1.5 g yeast extract, 0.2 mg ZnSO₄ in a total volume of 300 ml of water. the medium was sterilized by autoclaving after preparation. After cooling to room temperature, 1 ml of tetracycline (10 mg/ml in ethanol), 100 μl AMG300 glucoamylase and 155 mg active dry yeast. were added. The mixture was then shaken at 30° C. for 22 h. The overnight yeast culture was diluted 1/10 with water and A600 measured to determine the yeast number, as described in Current Protocols in Molecular Biology.
ROGA flour Kernels were pooled from several T0 lines shown to have active glucoamylase The seeds were ground in the Kleco, and all flour was pooled.
AMYI flour Kernels from T0 corn expressing AMYI were pooled and ground as above.
Control flour Kernels from with similar genetic background were ground in the same fashion as the ROGA expressing corn
   An inoculation mixture was prepared in a sterile tube; it contained per 1.65 ml: yeast cells (1×10⁷), yeast extract (8.6 mg), tetracycline (55 μg). 1.65 ml was added/g flour to each fermentation tube.
   Fermentation preparation: Flour was weighed out at 1.8 g/tube into tared 17×100 mm sterile polypropylene. 50 μl of 0.9 M H₂SO₄ was added to bring the final pH prior to fermentation to 5. The inoculation mixture (2.1 ml) was added/tube. along with RXGA, AMYI-P and amylase desalting buffer as indicated below. The quantity of buffer was adjusted based on moisture content of each flour so that the total solids content was constant in each tube. The tubes were mixed thoroughly, weighed and placed into a plastic bag and incubated at 30° C.

TABLE 21

| | Flours | | | Innoculation | Microbial enzymes | | Amylase desalting |
|---|---|---|---|---|---|---|---|
| | Control | ROGA | AMYI | Mix | RXGA | AMYI-P | Buffer |
| Tube | g | g | g | ml | ml | ml | ml |
| A | 1.8 | | | 2.1 | 0 | 0 | |
| B | 1.8 | | | 2.1 | 0.036 | 0 | 1 |
| C | 1.8 | | | 2.1 | 0.036 | 1 | 0 |
| D | 1.8 | | | 2.1 | 0 | 1 | 0.036 |
| E | 1.6 | | 0.2 | 2.1 | 0.036 | 0 | 1 |
| F | 0.2 | 1.6 | | 2.1 | | | 1 |
| G | 0.2 | 1.6 | | 2.1 | 0 | 1 | 0 |
| H | 0 | 1.6 | 0.2 | 2.1 | | 0 | 1 |

The fermentation tubes were weighed at intervals over the 67 h time course. Loss of weight corresponds to evolution of CO₂ during fermentation. The ethanol content of the samples was determined after 67 h of fermentation by the DCL ethanol assay method. The kit (catalogue #229-29) was purchased from Diagnostic Chemicals Limited, Charlottetown, PE, Canada, DIE IB0. Samples (10 μl) were drawn in triplicate from each fermentation tube and diluted into 990 μl of water. 10 μl of the diluted samples were mixed with 1.25 ml of a 12.5/1 mixture of assay buffer/ADH-NAD reagent. Standards (0, 5, 10, 15 & 20% v/v ETOH) were diluted and assayed in parallel. Reactions were incubated at 37° C. for 10 min, then A340 read. Standards were prepared in duplicate, samples from each fermentation were prepared in triplicate (including the initial dilution). The weight of the samples changed with time as detailed in table below. The weight loss is expressed as a percentage of the initial sample weight at time 0.

TABLE 22

| | | Time (h) | | | | | |
|---|---|---|---|---|---|---|---|
| | | 0 | 18 | 24 | 42 | 48 | 67 |
| Sample | Flour Composition | % wgt loss | | | | | |
| A | Control | 0.00 | 8.09 | 9.38 | 12.96 | 13.83 | 16.85 |
| B | Control + RXGA | 0.00 | 11.48 | 14.20 | 21.79 | 23.83 | 24.63 |
| C | Control + RXGA + | 0.00 | 17.90 | 23.27 | 36.48 | 39.07 | 47.59 |
| | MAMYI | | | | | | |
| D | Control + MAMYI | 0.00 | 13.70 | 17.72 | 28.27 | 30.80 | 38.27 |
| E | Control + RXGA + | 0.00 | 16.85 | 21.60 | 33.95 | 36.98 | 45.74 |
| | AMYI flour | | | | | | |
| F | ROGA flour | 0.00 | 9.81 | 11.74 | 16.96 | 18.39 | 23.17 |
| G | ROGA flour + | 0.00 | 15.53 | 19.69 | 29.75 | 32.11 | 39.94 |
| | MAMYI | | | | | | |
| H | ROGA flour + | 0.00 | 13.35 | 16.27 | 23.60 | 25.53 | 31.68 |
| | AMYI flour | | | | | | |

These data show that the ROGA enzyme expressed in maize increases fermentation rate as compared to the no-enzyme control. It also confirms previous data indicating that the AMYI enzyme expressed in maize kernels is a potent activator of fermentation of the starch in corn. The ethanol contents are detailed below.

TABLE 23

| | Flour | ETOH | Standard |
|---|---|---|---|
| Sample | Composition | % v/v | deviation |
| A | Control | 2.09 | 0.08 |
| B | Control + RXGA | 7.97 | 0.18 |
| C | Control + RXGA + MAMYI | 13.47 | 0.27 |
| D | Control + MAMYI | 11.26 | 0.12 |
| E | Control + RXGA + AMYI flour | 12.28 | 0.08 |
| F | ROGA flour | 3.55 | 0.05 |
| G | ROGA flour + MAMYI | 11.29 | 0.18 |
| H | ROGA flour + AMYI flour | 8.58 | 0.13 |

These data also demonstrate that expressing Rhizopus oryzae glucoamylase in maize facilitates increased fermentation of the starch in corn. Similarly, expression of the barley amylase in maize makes corn starch more fermentable with out adding exogenous enzymes.

### Example 55

### Cellobiohydrolase I

The Trichoderma reesei cellobiohydrolase I (CBH I) gene was amplified and cloned by RT-PCR based on a published database sequence (accession # E00389). The cDNA sequence was analyzed for the presence of a signal sequence using the SignalP program, which predicted a 17 amino acid signal sequence. The DNA sequence encoding the signal sequence was replaced with an ATG by PCR, as shown in the sequence (SEQ ID NO: 79). This cDNA sequence was used to make subsequent constructs. Additional constructs are made by substituting a maize optimised version of the gene (SEQ ID NO: 93).

### Example 56

### Cellobiohydrolase II

The Trichoderma reesei cellobiohydrolase II (CBH II) gene was amplified and cloned by RT-PCR based on a published database sequence (accession # M55080). The cDNA sequence was analyzed for the presence of a signal sequence using the SignalP program, which predicted an 18 amino acid signal sequence. The DNA sequence encoding the signal sequence was replaced with an ATG by PCR, as shown in the sequence (SEQ ID NO: 81). This cDNA sequence was used to make subsequent constructs. Additional constructs are made by substituting a maize optimised version (SEQ ID NO: 94) of the gene.

### Example 57

### Construction of Transformation Vectors for the Trichoderma reesii Cellobiohydrolase I and Cellobiohydrolase II

Cloning of the Trichoderma reesii cellobiohydrolase I (cbhi) cDNA without the native N-terminal signal sequence is described in Example 55. Expression cassettes were constructed to express the Trichoderma reesii cellobiohydrolase I cDNA in maize endosperm with various targeting signals as follows:

Plasmid 12392 comprises the Trichoderma reesii cbhi cDNA cloned behind the γ zein promoter for expression specifically in the endosperm for expression in the cytoplasm.

Plasmid 12391 comprises the maize γ-zein N-terminal signal sequence (MRVLLVALALLALAASATS) (SEQ ID NO:17) fused to Trichoderma reesii cbhi cDNA as described above in Example 1 for targeting to the endoplasmic reticulum and secretion into the apoplast (Torrent et al. 1997). The fusion was cloned behind the γ zein promoter for expression specifically in the endosperm.

Plasmid 12392 comprises the γ-zein N-terminal signal sequence fused to the Trichoderma reesii cbhi cDNA with a C-terminal addition of the sequence KDEL for targeting to and retention in the endoplasmic reticulum (ER) (Munro and Pelham, 1987). The fusion was cloned behind the maize γ zein promoter for expression specifically in the endosperm.

Plasmid 12656 comprises the waxy amyloplast targeting peptide (Klosgen et al., 1986) fused to the Trichoderma reesii cbhi cDNA for targeting to the amyloplast. The fusion was cloned behind the maize γ zein promoter for expression specifically in the endosperm.

All expression cassettes were moved into a binary vector (pNOV2117) for transformation into maize via Agrobacterium infection. The binary vector contained the phosphomannose isomerase (PMI) gene which allows for selection of transgenic cells with mannose. Transformed maize plants were either self-pollinated or outcrossed and seed was collected for analysis.

Additional constructs (plasmids 12652, 12653, 12654 and 12655) were made with the targeting signals described above fused to Trichoderma reesii cellobiohydrolaseII (cbhii) cDNA in precisely the same manner as described for the Trichoderma reesii cbhi cDNA. These fusions were cloned behind the maize Q protein promoter (50 Kd γ zein) (SEQ ID NO: 98) for expression specifically in the endosperm and transformed into maize as described above. Transformed maize plants were either self-pollinated or outcrossed and seed was collected for analysis.

Combinations of the enzymes can be produced either by crossing plants expressing the individual enzymes or by cloning several expression cassettes into the same binary vector to enable co-transformation.

### Example 58

### Expression of a Cbhi in Corn

T1 seed from self-pollinated maize plants transformed with either plasmid 12390, 12391 or 12392 was obtained. The 12390 construct targets the expression of the CbhI in the endoplasmic reticulum of the endosperm, the 12391 construct targets the expression of the CbhI in the apoplast of the endosperm and the 12392 construct targets the expression of the CbhI in the cytoplasm of the endosperm.

Extraction and detection of the CbhI from corn-flour: Polyclonal antibodies to CbhI and CbhII were produced in goat according to established protocols. Flour from the CbhI transgenic seeds was obtained by grinding them in an Autogizer grinder. Approximately 50 mg of flour was resuspended in 0.5 ml of 20 mM NaPO₄ buffer (pH 7.4), 150 mM NaCl followed by incubation for 15 minutes at RT with continuous shaking. The incubated mixture was then spun for 10 min. at 10,000×g. The supernatant was used as enzyme source. 30 μl of this extract was loaded on a 4-12% NuPAGE gel (invitrogen) and separated in the NuPAGE MES running buffer (invitrogen). Protein was blotted onto nitrocellulose membranes and Western blot analysis was done following established protocols using the specific antibodies described above followed by alkaline phosphatase conjugated rabbit antigoat IgG (H+L). Alkaline phosphatase activity was detected by incubation of the membranes with ready to use BCIP/MBT (plus) substrate from Moss Inc.

Western Blot analysis was done of T1 seeds from different events transformed with plasmid 12390. Expression of CbhI protein was compared to the non-transgenic control, and was detected in a number of events.

The Cracked Corn Assay was performed essentially as described in Example 49, using transgenic seed expressing Cbhi. Starch recovery from the transgenic seed was measured and the results are set forth in Table 24.

TABLE 24

| | Line 3-non | Line |
|---|---|---|
| | expressing control | 4-CBHI expressing |
| Conditions | Starch (mg) | |
| 400 ppm SO2-No Bromelain | 40.2 | 78.1 |
| 400 ppm SO2-Plus Bromelain | 48.1 | 118.7 |
| 2000 ppm SO2-No Bromelain | 47.5 | 73.1 |
| 2000 ppm SO2-Plus Bromelain | 49.2 | 109 |

### Example 59

### Preparation of Endoglucanase I Constructs

A Trichoderma reesei endoglucanase I (EGLI) gene was amplified and cloned by PCR based on a published database sequence (Accession # M15665; Penttila et al., 1986). Because only genomic sequences could be obtained, the cDNA was generated from the genomic sequence by removing 2 introns using Overlap PCR. The resulting cDNA sequence was analyzed for the presence of a signal sequence using the SignalP program, which predicted a 22 amino acid signal sequence. The DNA sequence encoding the signal sequence was replaced with an ATG by PCR, as shown in the sequence (SEQ ID NO: 83). This cDNA sequence was used to make subsequent constructs as set forth below.

Overlap PCR

Overlap PCR is a technique (Ho et al., 1989) used to fuse complementary ends of two or more PCR products, and can be used to make base pair (bp) changes, add bp, or delete bp. At the site of the intended bp change, forward and reverse mutagenic primers (Mut-F and Mut-R) are made that contain the intended change and 15 bp of sequence on either side of the change. For example, to remove an intron, the primers would consist of the final 15 bp of exon 1 fused to the first 15 bp of exon 2. Primers are also prepared that anneal to the ends of the sequence to be amplified, e.g ATG and STOP codon primers. PCR amplification of the products proceeds with the ATG/Mut-R primer pair and the Mut-F/STOP primer pair in independent reactions. The products are gel purified and fused together in a PCR without added primers. The fusion reaction is separated on a gel, and the band of the correct size is gel purified and cloned. Multiple changes can be accomplished simultaneously through the addition of additional mutagenic primer pairs.

### EGLI Plant Expression Constructs

Expression cassettes were made to express the Trichoderma reesei EGLI cDNA in maize endosperm as follows:

13025 comprises the T. reesei EGLI gene cloned behind the maize γ-zein promoter for cytoplasmic localization and expression specifically in the endosperm.
13026 comprises the maize γ-zein N-terminal signal peptide (MRVLLVALALLALAASATS) fused to the T. reesei EGLI gene for targeting to the endoplasmic reticulum and secretion into the apoplast. The fusion was cloned behind the maize γ-zein promoter for expression specifically in the endosperm.
13027 comprises the maize γ-zein N-terminal signal peptide fused to the T. reesei EGLI gene with a C-terminal addition of the sequence KDEL for targeting to and retention in the endoplasmic reticulum. The fusion was cloned behind the maize γ-zein promoter for expression specifically in the endosperm.
13028 comprises the maize Granule Bound Starch Synthase I (GBSSI) N-terminal signal peptide (N-terminal 77 amino acids) fused to the T. reesei EGLI gene for targeting to the lumen of the amyloplast. The fusion was cloned behind the maize γ-zein promoter for expression specifically in the endosperm.
13029 comprises the maize GBSSI N-terminal signal peptide fused to the T. reesei EGLI gene with a C-terminal addition of the starch binding domain (C-terminal 301 amino acids) of the maize GBSSI gene for targeting to the starch granule. The fusion was cloned behind the maize γ-zein promoter for expression specifically in the endosperm.

Additional Expression cassettes are generated using a maize optimised version of EGLI (SEQ ID NO: 95)

EGLI Enzyme Assays

EGLI enzyme activity is measured in maize transgenics using the Malt Beta-Glucanase Assay Kit (Cat # K-MBGL) (Megazyme International Ireland Ltd.) The enzymatic activity of EGL I expressors is tested in the Corn Fiber Hydrolysis Assay as described in Example 53.

### Example 60

### β-Glucosidase 2

A Trichoderma reesei β-Glucosidase 2 (BGL2) gene was amplified and cloned by RT-PCR based on sequence Accession # AB003110 (Takashima et al., 1999).

### BGL2 Plant Expression Constructs

Expression cassettes were made to express the Trichoderma reesei BGL2 cDNA (SEQ ID NO: 89) in maize endosperm as follows:

13030 comprises the T. reesei BGL2 gene cloned behind the maize γ-zein promoter for cytoplasmic localization and expression specifically in the endosperm.
13031 comprises the maize γ-zein N-terminal signal peptide (MRVLLVALALLALAASATS) fused to the T. reesei BGL2 gene for targeting to the endoplasmic reticulum and secretion into the apoplast. The fusion was cloned behind the maize γ-zein promoter for expression specifically in the endosperm.
13032 comprises the maize γ-zein N-terminal signal peptide fused to the T. reesei BGL2 gene with a C-terminal addition of the sequence KDEL for targeting to and retention in the endoplasmic reticulum. The fusion was cloned behind the maize γ-zein promoter for expression specifically in the endosperm.
13033 comprises the maize Granule Bound Starch Synthase I (GBSSI) N-terminal signal peptide (N-terminal 77 amino acids) fused to the T. reesei BGL2 gene for targeting to the lumen of the amyloplast. The fusion was cloned behind the maize γ-zein promoter for expression specifically in the endosperm.
13034 comprises the maize GBSSI N-terminal signal peptide fused to the T. reesei BGL2 gene with a C-terminal addition of the starch binding domain (C-terminal 301 amino acids) of the maize GBSSI gene for targeting to the starch granule. The fusion was cloned behind the maize γ-zein promoter for expression specifically in the endosperm.

Additional Expression cassettes are generated by substituting a maize optimized version of BGL2 (SEQ ID NO: 96).

All expression cassettes are inserted into the binary vector pNOV2117 for transformation into maize via Agrobacterium infection. The binary vector contained the phosphomannose isomerase (PMI) gene which allows for selection of transgenic cells with mannose. Transformed maize plants were either self-pollinated or outcrossed and seed was collected for analysis.

BGL2 Enzyme Assays

BGL2 enzyme activity is measured in transgenic maize using a protocol modified from Bauer and Kelly (Bauer, M. W. and Kelly, R. M. 1998. The family 1β-glucosidases from Pyrococcus furiosus and Agrobacterium faecalis share a common catalytic mechanism. Biochemistry 37: 17170-17178). The protocol can be modified to incubate samples at 37° C. instead of 100° C. The enzymatic activity of BGL2-expressors is tested in the Fiber Hydrolysis Assay.

### Example 61

### β-Glucosidase D

The Trichoderma reesei β-Glucosidase D (CEL3D) gene was amplified and cloned by PCR based on a published database sequence (accession #AY281378; Foreman et al., 2003). Because only genomic sequences could be obtained, the cDNA was generated from the genomic sequence by removing an intron using Overlap PCR, as described in Example 58. The resulting cDNA (SEQ ID NO: 91) may be used for subsequent constructs. A maize optimised version (SEQ ID NO: 97) of the resulting cDNA may also be used for constructs.

Plant constructs can be generated and β-glucosidase assays can be performed as described for BGL2 in Example 60, replacing BGL2 with CEL3D.

### Example 62

### Lipases

cDNAs encoding lipases are generated using sequences from Accession # D85895, AF04488, and AF04489 (Tsuchiya et al., 1996; Yu et al., 2003) and methodology set forth in Examples 59-60.

Lipase enzyme activity can be measured in transgenic maize using the Fluorescent Lipase Assay Kit (Cat # M0612) (Marker Gene Technologies, Inc.). Lipase activity can also be measured in vivo using the fluorescent substrate 1,2-dioleoyl-3-(pyren-1-yl)decanoyl-rac glycerol (M0258), also from Marker Gene Technologies, Inc.

### Example 63

### Expression of Phytase in Rice

Vectors 11267 and 11268 comprise binary vectors that encode Nov9x phytase. Expression of the Nov9x phytase gene in both vectors is under the control of the rice glutelin-1 promoter (SEQ ID NO:67). Vectors 11267 and 11268 are derived from pNOV2117.

The Nov9x phytase expression cassette in vector 11267 comprises the rice glutelin-1 promoter, the Nov9x phytase gene with apoplast targeting signal, a PEPC intron, and the 35S terminator. The product of the Nov9x phytase coding sequence in vector 11267 is shown in SEQ ID NO: 110.

The Nov9x phytase expression cassette in vector 11268 comprises the rice glutelin-1 promoter, the Nov9x phytase gene with ER retention (SEQ ID NO:111), a PEPC intron, and the 35S terminator. The product of the Nov9x phytase coding sequence in vector 11268 is shown in SEQ ID NO: 112.

11267 Nov9x phytase with apoplast targeting DNA
sequence (SEQ ID NO: 109).
Translation start and stop codons are underlined.
The sequence encoding the signal sequence of the
27-kD gamma-zein protein is in bold.
atgagggtgttgctcgttgccctcgctctcctggctctcgctgcgagcgc
caccagcgctgcgcagtccgagccggagctgaagctggagtccgtggtga
tcgtgtcccgccacggcgtgcgcgccccgaccaaggccacccagctcatg
caggacgtgaccccggacgcctggccgacctggccggtgaagctcggcga
gctgaccccgcgcggcggcgagctgatcgcctacctcggccactactggc
gccagcgcctcgtggccgacggcctcctcccgaagtgcggctgcccgcag
tccggccaggtggccatcatcgccgacgtggacgagcgcacccgcaagac
cggcgaggccttcgccgccggcctcgccccggactgcgccatcaccgtgc
acacccaggccgacacctcctccccggacccgctcttcaacccgctcaag
accggcgtgtgccagctcgacaacgccaacgtgaccgacgccatcctgga
gcgcgccggcggctccatcgccgacttcaccggccactaccagaccgcct
tccgcgagctggagcgcgtgctcaacttcccgcagtccaacctctgcctc
aagcgcgagaagcaggacgagtcctgctccctcacccaggccctcccgtc
cgagctgaaggtgtccgccgactgcgtgtccctcaccggcgccgtgtccc
tcgcctccatgctcaccgaaatcttcctcctccagcaggcccagggcatg
ccggagccgggctggggccgcatcaccgactcccaccagtggaacaccct
cctctccctccacaacgcccagttcgacctcctccagcgcaccccggagg
tggcccgctcccgcgccaccccgctcctcgacctcatcaagaccgccctc
accccgcacccgccgcagaagcaggcctacggcgtgaccctcccgacctc
cgtgctcttcatcgccggccacgacaccaacctcgccaacctcggcggcg
ccctggagctgaactggaccctcccgggccagccggacaacaccccgccg
ggcggcgagctggtgttcgagcgctggcgccgcctctccgacaactccca
gtggattcaggtgtccctcgtgttccagaccctccagcagatgcgcgaca
agaccccgctctccctcaacaccccgccgggcgaggtgaagctcaccctc
gccggctgcgaggagcgcaacgcccagggcatgtgctccctcgccggctt
cacccagatcgtgaacgaggcccgcatcccggcctgctccctctaa
11267 Nov9x phytase with apoplast targeting gene
product (SEQ ID NO: 110). The signal sequence of
the 27-kD gamma-zein protein is in bold.
mrvllvalallalaasatsaaqsepelklesvvivsrhgvraptkatqlm
qdvtpdawptwpvklgeltprggeliaylghywrqrlvadgllpkcgcpq
sgqvaiiadvdertrktgeafaaglapdcaitvhtqadtsspdplfnplk
tgvcqldnanvtdaileraggsiadftghyqtafrelervlnfpqsnlcl
krekqdescsltqalpselkvsadcvsltgavslasmlteifllqqaqgm
pepgwgritdshqwntllslhnaqfdllqrtpevarsratplldliktal
tphppqkqaygvtlptsvlfiaghdtnlanlggalelnwtlpgqpdntpp
ggelvferwrrlsdnsqwiqvslvfqtlqqmrdktplslntppgevkltl
agceernaqgmcslagftqivnearipacsl
11268 Nov9x phytase with ER retention DNA sequence
(SEQ ID NO: 111). The sequence encoding the signal
sequence of the 27-kD gamma-zein protein is in
bold. The sequence encoding the SEKDEL hexapeptide
ER retention signal is underlined.
atgagggtgttgctcgttgccctcgctctcctggctctcgctgcgagcgc
caccagcgctgcgcagtccgagccggagctgaagctggagtccgtggtga
tcgtgtcccgccacggcgtgcgcgccccgaccaaggccacccagctcatg
caggacgtgaccccggacgcctggccgacctggccggtgaagctcggcga
gctgaccccgcgcggcggcgagctgatcgcctacctcggccactactggc
gccagcgcctcgtggccgacggcctcctcccgaagtgcggctgcccgcag
tccggccaggtggccatcatcgccgacgtggacgagcgcacccgcaagac
cggcgaggccttcgccgccggcctcgccccggactgcgccatcaccgtgc
acacccaggccgacacctcctccccggacccgctcttcaacccgctcaag
accggcgtgtgccagctcgacaacgccaacgtgaccgacgccatcctgga
gcgcgccggcggctccatcgccgacttcaccggccactaccagaccgcct
tccgcgagctggagcgcgtgctcaacttcccgcagtccaacctctgcctc
aagcgcgagaagcaggacgagtcctgctccctcacccaggccctcccgtc
cgagctgaaggtgtccgccgactgcgtgtccctcaccggcgccgtgtccc
tcgcctccatgctcaccgaaatcttcctcctccagcaggcccagggcatg
ccggagccgggctggggccgcatcaccgactcccaccagtggaacaccct
cctctccctccacaacgcccagttcgacctcctccagcgcaccccggagg
tggcccgctcccgcgccaccccgctcctcgacctcatcaagaccgccctc
accccgcacccgccgcagaagcaggcctacggcgtgaccctcccgacctc
cgtgctcttcatcgccggccacgacaccaacctcgccaacctcggcggcg
ccctggagctgaactggaccctcccgggccagccggacaacaccccgccg
ggcggcgagctggtgttcgagcgctggcgccgcctctccgacaactccca
gtggattcaggtgtccctcgtgttccagaccctccagcagatgcgcgaca
agaccccgctctccctcaacaccccgccgggcgaggtgaagctcaccctc
gccggctgcgaggagcgcaacgcccagggcatgtgctccctcgccggctt
cacccagatcgtgaacgaggcccgcatcccggcctgctccctctccgaga
aggacgagctgtaa
11268 Nov9x phytase with ER retention, gene
product (SEQ ID NO: 112). The signal sequence of
the 27-kD gamma-zein protein is in bold. The ER
retention signal is underlined.
mrvllvalallalaasatsaaqsepelklesvvivsrhgvraptkatqlm
qdvtpdawptwpvklgeltprggeliaylghywrqrlvadgllpkcgcpq
sgqvaiiadvdertrktgeafaaglapdcaitvhtqadtsspdplfhplk
tgvcqldnanvtdaileraggsiadftghyqtafrelervlnfpqsnlcl
krekqdescsltqalpselkvsadcvsltgavslasmlteifllqqaqgm
pepgwgfltdshqwntllslhnaqfdllqrtpevarsratplldliktal
tphppqkqaygvtlptsvlfiaghdtnlanlggalelnwtlpgqpdntpp
ggelvferwrrlsdnsqwiqvslvfqtlqqmrdktplslntppgevkltl
agceernaqgmcslagftqivnearipacslsekdel

### Generation of Transgenic Rice Plants

Rice (Oryza sativa) is used for generating transgenic plants. Various rice cultivars can be used (Hiei et al., 1994, Plant Journal 6:271-282; Dong et al., 1996, Molecular Breeding 2:267-276; Hiei et al., 1997, Plant Molecular Biology, 35:205-218). Also, the various media constituents described below may be either varied in concentration or substituted. Embryogenic responses are initiated and/or cultures are established from mature embryos by culturing on MS-CIM medium (MS basal salts, 4.3 g/liter; B5 vitamins (200×), 5 ml/liter; Sucrose, 30 g/liter; proline, 500 mg/liter; glutamine, 500 mg/liter; casein hydrolysate, 300 mg/liter; 2,4-D (1 mg/ml), 2 ml/liter; adjust pH to 5.8 with 1 N KOH; Phytagel, 3 g/liter). Either mature embryos at the initial stages of culture response or established culture lines are inoculated and co-cultivated with the Agrobacterium strain LBA4404 containing the desired vector construction. Agrobacterium is cultured from glycerol stocks on solid YPC medium (100 mg/L spectinomycin and any other appropriate antibiotic) for ˜2 days at 28° C. Agrobacterium is re-suspended in liquid MS-CIM medium. The Agrobacterium culture is diluted to an OD600 of 0.2-0.3 and acetosyringone is added to a final concentration of 200 uM. Agrobacterium is induced with acetosyringone before mixing the solution with the rice cultures. For inoculation, the cultures are immersed in the bacterial suspension. The liquid bacterial suspension is removed and the inoculated cultures are placed on co-cultivation medium and incubated at 22° C. for two days. The cultures are then transferred to MS-CIM medium with Ticarcillin (400 mg/liter) to inhibit the growth of Agrobacterium. For constructs utilizing the PMI selectable marker gene (Reed et al., In Vitro Cell. Dev. Biol.-Plant 37:127-132), cultures are transferred to selection medium containing Mannose as a carbohydrate source (MS with 2% Mannose, 300 mg/liter Ticarcillin) after 7 days, and cultured for 3-4 weeks in the dark. Resistant colonies are then transferred to regeneration induction medium (MS with no 2,4-D, 0.5 mg/liter IAA, 1 mg/liter zeatin, 200 mg/liter Ticarcillin 2% Mannose and 3% Sorbitol) and grown in the dark for 14 days. Proliferating colonies are then transferred to another round of regeneration induction media and moved to the light growth room. Regenerated shoots are transferred to GA7-1 medium (MS with no hormones and 2% Sorbitol) for 2 weeks and then moved to the greenhouse when they are large enough and have adequate roots. Plants are transplanted to soil in the greenhouse and grown to maturity.

### Example 64

### Analysis of Transgenic Rice Seed Expressing Nov9X Phytase

ELISA for the Quantitation of Nov9X Phytase from Rice Seed

Quantitation of phytase expressed in transgenic rice seed was assayed by ELISA. One (1 g) rice seed was ground to flour in a Kleco seed grinder. 50 mg of flour was resuspended in the sodium acetate buffer described in example—for Nov9X phytase activity assay and diluted as required for the immunoassay. The Nov9X immunoassay is a quantitative sandwich assay for the detection of phytase that employs two polyclonal antibodies. The rabbit antibody was purified using protein A, and the goat antibody was immunoaffinity purified against recombinant phytase (Nov9X) protein produced in E. coli inclusion bodies. Using these highly specific antibodies, the assay can measure picogram levels of phytase in transgenic plants. There are three basic parts to the assay. The phytase protein in the sample is captured onto the solid phase microtiter well using the rabbit antibody. Then a “sandwich” is formed between the solid phase antibody, the phytase protein, and the secondary antibody that has been added to the well. After a wash step, where unbound secondary antibody has been removed, the bound antibody is detected using an alkaline phosphatase-labeled antibody. Substrate for the enzyme is added and color development is measured by reading the absorbance of each well. The standard curve uses a four-parameter curve fit to plot the concentrations versus the absorbance.

### Phytase Activity Assay

Determination of phytase activity, based upon the estimation of inorganic phosphate released on hydrolysis of phytic acid, can be performed at 37° C. following the method of Engelen, A. J. et al., J. AOAC. Inter. 84, 629 (2001). One unit of enzyme activity is defined as the amount of enzyme that liberates 1 μmol of inorganic phosphate per minute under assay conditions. For example, phytase activity may be measured by incubating 2.0 ml of the enzyme preparation with 4.0 ml of 9.1 mM sodium phytate in 250 mM sodium acetate buffer pH 5.5, supplemented with 1 mM CaCl2 for 60 minutes at 37° C. After incubation, the reaction is stopped by adding 4.0 ml of a color-stop reagent consisting of equal parts of a 10% (w/v) ammonium molybdate and a 0.235% (w/v) ammonium vanadate stock solution. Precipitate is removed by centrifugation, and phosphate released is measured against a set of phosphate standards spectrophotometrically at 415 nm. Phytase activity is calculated by interpolating the A415 nm absorbance values obtained for phytase containing samples using the generated phosphate standard curve.

This procedure may be scaled down to accommodate smaller volumes and adapted to preferred containers. Preferred containers include glass test tubes and plastic microplates. Partial submersion of the reaction vessel(s) in a water bath is essential to maintain constant temperature during the enzyme reaction.

TABLE 24

| | | | Endogenous inorganic | Endogenous inorganic |
|---|---|---|---|---|
| | μg | | phosphate released by | phosphate released by cooking |
| Trans-genic | phytase/g | Phytase activity | cooking of dehusked rice | of dehusked, polished rice |
| line | flour* | units per g flour** | seed (μmol/gseed) | seed (μmol/gseed) |
| Wild type | 0 | 0 | 1.442 | 0.469 |
| 1 | 510 | 916 | 1.934 | 0.840 |
| 2 | 1518 | 2800 | 2.894 | 1.073 |

*μg phytase was assayed by a sandwich ELISA
**Phytase activity was assayed by Phytase activity assay as described above.

### Assay of Inorganic Phosphate Release During Cooking of Transgenic Rice Expressing Phytase

Two samples of 1 g seed from selected rice transgenic lines and a control wildtype line was dehusked using a benchtop Kett TR200 automatic rice husker. One sample was then polished for 30 seconds in a Kett Rice polisher. Two volumes of H2O was added to each sample and the rice was cooked by immersing the tubes into a beaker of water. The water was brought to a boil and held in a full rolling boil for 10 minutes. The “cooked” rice seed was then ground to a paste with water bringing the total volume of the slurry to 6 ml. The slurry was centrifuged at 15,000×g for 10 minutes and the clear supernatant assayed for released endogenous inorganic phosphate. The assay of released phosphate is based on color formation as a result of molybdate and vanadate ions complexing with inorganic phosphate and is measured spectrophotometrically at 415 nm as described in example—for phytase enzymatic activity. The results are in Table 24.

All publications, patents and patent applications are incorporated herein by reference. While in the foregoing specification this invention has been described in relation to certain preferred embodiments thereof, and many details have been set forth for purposes of illustration, it will be apparent to those skilled in the art that the invention is susceptible to additional embodiments and that certain of the details described herein may be varied considerably without departing from the basic principles of the invention.

1-234. (canceled)

235. An isolated polynucleotide a) comprising SEQ ID NO: 61, 63, 65, 69, 73, 74, 75, 76, 77, 78, 79, 81, 83, 85, 87, 89, 91, 93, 94, 95, 96, 97, 99, 101, 103, 105, 107, 109, or 111, or the complement thereof, or a polynucleotide which hybridizes to the complement of any one of SEQ ID NO: 61, 63, 65, 73, 74, 75, 76, 77, 78, 79, 81, 83, 85, 87, 89, 91, 93, 94, 95, 96, 97, 99, 101, 103, 105, 107, 109, or 111, under low stringency hybridization conditions and encodes a polypeptide having cellulase, hemicellulase, exo-1,4-β-cellobiohydrolase, exo-1,3-β-D-glucanase, β-glucosidase, endoglucanase, L-arabinase, α-arabinosidase, galactanase, galactosidase, mannanase, mannosidase, xylanase, xylosidase, protease, glucanase, or esterase activity or b) encoding a polypeptide comprising SEQ ID NO: 62, 64, 66, 70, 80, 82, 84, 86, 88, 90, 92, 100, 102, 104, 106, 108, 110, or 112, or an enzymatically active fragment thereof.

236. The isolated polynucleotide of claim 235, wherein said polynucleotide encodes a fusion polypeptide comprising a first polypeptide and a second peptide, wherein said first polypeptide has cellulase, hemicellulase, exo-1,4-β-cellobiohydrolase, exo-1,3-β-D-glucanase, β-glucosidase, endoglucanase, L-arabinase, α-arabinosidase, galactanase, galactosidase, mannanase, mannosidase, xylanase, xylosidase, protease, glucanase, or esterase activity.

237. The isolated polynucleotide of claim 236, wherein said second peptide comprises a targeting sequence peptide.

238. The isolated polynucleotide of claim 237, wherein said targeting sequence peptide targets the first polypeptide to a vacuole, endoplasmic reticulum, chloroplast, starch granule, or cell wall of a plant.

239. The isolated polynucleotide of claim 237, wherein said targeting sequence is an N-terminal signal sequence from Waxy, an N-terminal signal sequence from γ-zein, a starch binding domain, or a C-terminal starch binding domain.

240. An expression cassette comprising the isolated polynucleotide of claim 235.

241. The expression cassette of claim 240, which is operably linked to a promoter.

242. The expression cassette of claim 241, wherein the promoter is an inducible, tissue-specific, constitutive, or endosperm-specific promoter.

243. The expression cassette of claim 242, wherein the endosperm-specific promoter is a maize γ-zein, rice glutenin-1, or a maize ADP-gpp promoter.

244. The expression cassette of claim 243, wherein the maize γ-zein promoter is a 27-kD or 55 kD gamma-zein promoter.

245. The expression cassette of claim 243, wherein the promoter comprises SEQ ID NO: 11 or SEQ ID NO: 12.

246. The expression cassette of claim 240, wherein the polynucleotide is oriented in sense orientation relative to the promoter.

247. The expression cassette of claim 240, wherein the polynucleotide further encodes a targeting sequence which is operably linked to the polypeptide encoded by the polynucleotide.

248. The expression cassette of claim 247, wherein the targeting sequence targets the operably linked polypeptide to a vacuole, endoplasmic reticulum, chloroplast, starch granule, or cell wall of a plant.

249. The expression cassette of claim 248, wherein the targeting sequence is an N-terminal signal sequence from Waxy, an N-terminal signal sequence from γ-zein, or a starch binding domain.

250. A cell comprising the expression cassette of claim 240.

251. A plant, or part thereof, comprising the cell of claim 250.

252. The cell of claim 250, wherein the cell is selected from the group consisting of an Agrobacterium, a monocot cell, a dicot cell, a Liliopsida cell, a Panicoideae cell, a maize cell, and a cereal cell.

253. Seed, fruit, stem, leaf, stalk, or grain from the plant of claim 251.

254. The plant of claim 235, which is sugar beet, sugarcane, oats, barley, wheat, rye, corn, or rice.

255. A method for preparing fermentable sugar, monosaccharide, or oligosaccharide comprising the steps of treating a transgenic monocot plant part comprising an enzyme cellulase, hemicellulase, exo-1,4-b-cellobiohydrolase, exo-1,3-b-D-glucanase, b-glucosidase, endoglucanase, L-arabinase, α-arabinosidase, galactanase, galactosidase, mannanase, mannosidase, xylanase, xylosidase, protease, glucanase, or esterase under conditions to activate the enzyme thereby digesting polysaccharide to form oligosaccharide, monosaccharide, or fermentable sugar, wherein the plant part is obtained from a transformed monocot plant, the genome of which is augmented with an expression cassette comprising a promoter operably linked to a polynucleotide encoding the enzyme and a targeting sequence.

256. The method for preparing fermentable sugar, monosaccharide, or oligosaccharide according to claim 255, wherein the promoter is an inducible, tissue specific, endosperm-specific, or a constitutive promoter.

257. The method for preparing fermentable sugar, monosaccharide, or oligosaccharide according to claim 256, wherein the endosperm-specific promoter is a maize γ-zein, rice glutenin-1, or a maize ADP-gpp promoter.

258. The expression cassette of claim 257, wherein the maize γ-zein promoter is a 27-kD or 55 kD gamma-zein promoter.

259. The method for preparing fermentable sugar, monosaccharide, or oligosaccharide according to claim 257, wherein the promoter comprises SEQ ID NO: 11 or SEQ ID NO: 12.

260. The method for preparing fermentable sugar, monosaccharide, or oligosaccharide according to claim 255, wherein the plant part is a seed, fruit, stem, leaf, stalk, or grain.

261. The method for preparing fermentable sugar, monosaccharide, or oligosaccharide according to claim 255, wherein the plant part is obtained from sugar beet, sugarcane oats, barley, wheat, rye, corn, or rice.

262. The method for preparing fermentable sugar, monosaccharide, or oligosaccharide according to claim 255, wherein treating comprises heating the plant part for an amount of time and under conditions to activate the enzyme thereby digesting polysaccharide to form monosaccharide, oligosaccharide, or fermentable sugar.

263. The method for preparing fermentable sugar, monosaccharide, or oligosaccharide according to claim 255, wherein said enzyme is encoded by the polynucleotide sequence comprising SEQ ID NO. 61, 63, 65, 69, 73, 74, 75, 76, 77, 78, 79, 81, 83, 85, 87, 89, 91, 93, 94, 95, 96, 97, 99, 101, 103, 105, 107, 109, or 111.

264. The method for preparing fermentable sugar, monosaccharide, or oligosaccharide according to claim 255, wherein said polynucleotide sequence encodes a polypeptide comprising SEQ ID NO: 62, 64, 66, 70, 80, 82, 84, 86, 88, 90, 92, 100, 102, 104, 106, 108, 110, or 112.

265. The method of claim 255, further comprising the step of incubating the monosaccharide, oligosaccharide, or fermentable sugar under conditions that promote the conversion of the oligosaccharide or fermentable sugar into ethanol.
